SEQUENCE LISTING (66 sequences)

SEQ ID NO: 1 - protein, 18 aa, synthetic construct
TEFKSGAGTELSVRAKGG

SEQ ID NO: 2 - protein, 470 aa, synthetic construct
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSEEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAK

SEQ ID NO: 3 - protein, 494 aa, synthetic construct
MDMRVPAQLLGLLLLWLRGARCEEELQIIQPDKSVLVAAGETATLRCTITSLFPVGPIQWFRGAGPGRVLIYNQRQGPFPRVTTVSDTTKRNNMDFSIRIGNITPADAGTYYCIKFRKGSPDDVEFKSGAGTELSVRAKGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSEEELQIIQPDKSVLVAAGETATLRCTITSLFPVGPIQWFRGAGPGRVLIYNQRQGPFPRVTTVSDTTKRNNMDFSIRIGNITPADAGTYYCIKFRKGSPDDVEFKSGAGTELSVRAK

SEQ ID NO: 4 - protein, 345 aa, synthetic construct
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 345 aa, synthetic construct
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 346 aa, synthetic construct
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 346 aa, synthetic construct
EEELQIIQPDKSVLVAAGETATLRCTITSLFPVGPIQWFRGAGPGRVLIYNQRQGPFPRVTTVSDTTKRNNMDFSIRIGNITPADAGTYYCIKFRKGSPDDVEFKSGAGTELSVRAKGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 346 aa, synthetic construct
EEELQIIQPDKSVLVAAGETATLRCTITSLFPVGPIQWFRGAGPGRVLIYNQRQGPFPRVTTVSDTTKRNNMDFSIRIGNITPADAGTYYCIKFRKGSPDDVEFKSGAGTELSVRAKGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 9 - protein, 478 aa, synthetic construct
DVLMTQSPLSLPVTPGEPASISCRSSRNIVHINGDTYLEWYLQKPGQSPQLLIYKVSNRFSGVPDRFSGSGSGTDFTLKISRVEAEDVGVYYCFQGSLLPWTFGQGTKVEIKRGGGGSGGGGSGGGGSEVQLVESGGDLVQPGRSLRLSCAASGFIFSNYGMSWVRQAPGKGLEWVATISSASTYSYYPDSVKGRFTISRDNAKNSLYLQMNSLRVEDTALYYCGRHSDGNFAFGYWGQGTLVTVSSGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 603 aa, synthetic construct
EVQLVESGGDLVQPGRSLRLSCAASGFIFSNYGMSWVRQAPGKGLEWVATISSASTYSYYPDSVKGRFTISRDNAKNSLYLQMNSLRVEDTALYYCGRHSDGNFAFGYWGQGTLVTVSSGGGGSGGGGSGGGGSDVLMTQSPLSLPVTPGEPASISCRSSRNIVHINGDTYLEWYLQKPGQSPQLLIYKVSNRFSGVPDRFSGSGSGTDFTLKISRVEAEDVGVYYCFQGSLLPWTFGQGTKVEIKRGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSEEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAK

SEQ ID NO: 11 - protein, 470 aa, synthetic construct
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSEEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAK

SEQ ID NO: 12 - protein, 596 aa, synthetic construct
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKGGGGSGGGGSEEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSEEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAK

SEQ ID NO: 13 - protein, 722 aa, synthetic construct
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKGGGGSGGGGSEEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSEEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKGGGGSGGGGSEEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAK

SEQ ID NO: 14 - protein, 448 aa, synthetic construct
EVQLVESGGDLVQPGRSLRLSCAASGFIFSNYGMSWVRQAPGKGLEWVATISSASTYSYYPDSVKGRFTISRDNAKNSLYLQMNSLRVEDTALYYCGRHSDGNFAFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 15 - protein, 575 aa, synthetic construct
EVQLVESGGDLVQPGRSLRLSCAASGFIFSNYGMSWVRQAPGKGLEWVATISSASTYSYYPDSVKGRFTISRDNAKNSLYLQMNSLRVEDTALYYCGRHSDGNFAFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSEEELQIIQPDKSVLVAAGETATLRCTITSLFPVGPIQWFRGAGPGRVLIYNQRQGPFPRVTTVSDTTKRNNMDFSIRIGNITPADAGTYYCIKFRKGSPDDVEFKSGAGTELSVRAK

SEQ ID NO: 16 - protein, 574 aa, synthetic construct
EVQLVESGGDLVQPGRSLRLSCAASGFIFSNYGMSWVRQAPGKGLEWVATISSASTYSYYPDSVKGRFTISRDNAKNSLYLQMNSLRVEDTALYYCGRHSDGNFAFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSEEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAK

SEQ ID NO: 17 - protein, 219 aa, synthetic construct
DVLMTQSPLSLPVTPGEPASISCRSSRNIVHINGDTYLEWYLQKPGQSPQLLIYKVSNRFSGVPDRFSGSGSGTDFTLKISRVEAEDVGVYYCFQGSLLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 345 aa, synthetic construct
DVLMTQSPLSLPVTPGEPASISCRSSRNIVHINGDTYLEWYLQKPGQSPQLLIYKVSNRFSGVPDRFSGSGSGTDFTLKISRVEAEDVGVYYCFQGSLLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSEEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAK

SEQ ID NO: 19 - protein, 470 aa, synthetic construct
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSEEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAK

SEQ ID NO: 20 - protein, 574 aa, synthetic construct
EVQLVESGGDLVQPGRSLRLSCAASGFIFSNYGMSWVRQAPGKGLEWVATISSASTYSYYPDSVKGRFTISRDNAKNSLYLQMNSLRVEDTALYYCGRHSDGNFAFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSEEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAK

SEQ ID NO: 21 - protein, 574 aa, synthetic construct
EVQLVESGGDLVQPGRSLRLSCAASGFIFSNYGMSWVRQAPGKGLEWVATISSASTYSYYPDSVKGRFTISRDNAKNSLYLQMNSLRVEDTALYYCGRHSDGNFAFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSEEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAK

SEQ ID NO: 22 - protein, 117 aa, synthetic construct
EEELQIIQPDKSVLVAAGETATLRCTITSLFPVGPIQWFRGAGPGRVLIYNQRQGPFPRVTTVSDTTKRNNMDFSIRIGNITPADAGTYYCIKFRKGSPDDVEFKSGAGTELSVRAK

SEQ ID NO: 23 - protein, 116 aa, synthetic construct
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAK

SEQ ID NO: 24 - protein, 345 aa, synthetic construct
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 25 - protein, 478 aa, synthetic construct
EVQLVESGGDLVQPGRSLRLSCAASGFIFSNYGMSWVRQAPGKGLEWVATISSASTYSYYPDSVKGRFTISRDNAKNSLYLQMNSLRVEDTALYYCGRHSDGNFAFGYWGQGTLVTVSSGGGGSGGGGSGGGGSDVLMTQSPLSLPVTPGEPASISCRSSRNIVHINGDTYLEWYLQKPGQSPQLLIYKVSNRFSGVPDRFSGSGSGTDFTLKISRVEAEDVGVYYCFQGSLLPWTFGQGTKVEIKRGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 352 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSEEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAK

SEQ ID NO: 27 - protein, 603 aa, synthetic construct
DVLMTQSPLSLPVTPGEPASISCRSSRNIVHINGDTYLEWYLQKPGQSPQLLIYKVSNRFSGVPDRFSGSGSGTDFTLKISRVEAEDVGVYYCFQGSLLPWTFGQGTKVEIKRGGGGSGGGGSGGGGSEVQLVESGGDLVQPGRSLRLSCAASGFIFSNYGMSWVRQAPGKGLEWVATISSASTYSYYPDSVKGRFTISRDNAKNSLYLQMNSLRVEDTALYYCGRHSDGNFAFGYWGQGTLVTVSSGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSEEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAK

SEQ ID NO: 28 - protein, 603 aa, synthetic construct
EVQLVESGGDLVQPGRSLRLSCAASGFIFSNYGMSWVRQAPGKGLEWVATISSASTYSYYPDSVKGRFTISRDNAKNSLYLQMNSLRVEDTALYYCGRHSDGNFAFGYWGQGTLVTVSSGGGGSGGGGSGGGGSDVLMTQSPLSLPVTPGEPASISCRSSRNIVHINGDTYLEWYLQKPGQSPQLLIYKVSNRFSGVPDRFSGSGSGTDFTLKISRVEAEDVGVYYCFQGSLLPWTFGQGTKVEIKRGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSEEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAK

SEQ ID NO: 29 - protein, 372 aa, synthetic construct
MDMRVPAQLLGLLLLWLRGARCEEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKGGGGSGGGGSGGGGSDVLMTQSPLSLPVTPGEPASISCRSSRNIVHINGDTYLEWYLQKPGQSPQLLIYKVSNRFSGVPDRFSGSGSGTDFTLKISRVEAEDVGVYYCFQGSLLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 601 aa, synthetic construct
MDMRVPAQLLGLLLLWLRGARCEEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKGGGGSGGGGSGGGGSEVQLVESGGDLVQPGRSLRLSCAASGFIFSNYGMSWVRQAPGKGLEWVATISSASTYSYYPDSVKGRFTISRDNAKNSLYLQMNSLRVEDTALYYCGRHSDGNFAFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 31 - protein, 727 aa, synthetic construct
MDMRVPAQLLGLLLLWLRGARCEEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKGGGGSGGGGSGGGGSEVQLVESGGDLVQPGRSLRLSCAASGFIFSNYGMSWVRQAPGKGLEWVATISSASTYSYYPDSVKGRFTISRDNAKNSLYLQMNSLRVEDTALYYCGRHSDGNFAFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSEEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAK

SEQ ID NO: 32 - protein, 377 aa, synthetic construct
MDMRVPAQLLGLLLLWLRGARCDVLMTQSPLSLPVTPGEPASISCRSSRNIVHINGDTYLEWYLQKPGQSPQLLIYKVSNRFSGVPDRFSGSGSGTDFTLKISRVEAEDVGVYYCFQGSLLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSEEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAK

SEQ ID NO: 33 - protein, 502 aa, synthetic construct
MDMRVPAQLLGLLLLWLRGARCDVLMTQSPLSLPVTPGEPASISCRSSRNIVHINGDTYLEWYLQKPGQSPQLLIYKVSNRFSGVPDRFSGSGSGTDFTLKISRVEAEDVGVYYCFQGSLLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSEVQLVESGGDLVQPGRSLRLSCAASGFIFSNYGMSWVRQAPGKGLEWVATISSASTYSYYPDSVKGRFTISRDNAKNSLYLQMNSLRVEDTALYYCGRHSDGNFAFGYWGQGTLVTVSSGGGGSGGGGSGGGGSDVLMTQSPLSLPVTPGEPASISCRSSRNIVHINGDTYLEWYLQKPGQSPQLLIYKVSNRFSGVPDRFSGSGSGTDFTLKISRVEAEDVGVYYCFQGSLLPWTFGQGTKVEIK

SEQ ID NO: 34 - protein, 497 aa, synthetic construct
MDMRVPAQLLGLLLLWLRGARCEVQLVESGGDLVQPGRSLRLSCAASGFIFSNYGMSWVRQAPGKGLEWVATISSASTYSYYPDSVKGRFTISRDNAKNSLYLQMNSLRVEDTALYYCGRHSDGNFAFGYWGQGTLVTVSSGGGGSGGGGSGGGGSDVLMTQSPLSLPVTPGEPASISCRSSRNIVHINGDTYLEWYLQKPGQSPQLLIYKVSNRFSGVPDRFSGSGSGTDFTLKISRVEAEDVGVYYCFQGSLLPWTFGQGTKVEIKGGGGSGGGGSDVLMTQSPLSLPVTPGEPASISCRSSRNIVHINGDTYLEWYLQKPGQSPQLLIYKVSNRFSGVPDRFSGSGSGTDFTLKISRVEAEDVGVYYCFQGSLLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 627 aa, synthetic construct
MDMRVPAQLLGLLLLWLRGARCEEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKGGGGSSSASTDKTHTGGGSEEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSEEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAK

SEQ ID NO: 36 - protein, 492 aa, synthetic construct
MDMRVPAQLLGLLLLWLRGARCEEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSEEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAK

SEQ ID NO: 37 - RNA, 36 nt, synthetic construct
  source 1..36: mol_type = other RNA
  5'UTR 1..36
  modified_base order(3,9,24,28): mod_base = OTHER; note = Uracil
catagaagtcgagacacagccactaagtaagccacc

SEQ ID NO: 38 - RNA, 105 nt, synthetic construct
  source 1..105: mol_type = other RNA
  3'UTR 1..105
  modified_base order(9,19,30,39,46,49,55,56,69,70,76,77,80,88,96): mod_base = OTHER; note = Uracil
gcgccgcctccgggacagtgcacccaggctgcggcccctcccccgtcctggaggttccccagccccacttaccgcttaatgcgccaataaaccaatgaacgaagc

SEQ ID NO: 39 - RNA, 1482 nt, synthetic construct
  source 1..1482: mol_type = other RNA
  modified_base order(2,6,8,14,26,28,29,34,35,38,41,44,46,50,51,64,77,83,86,87,96,104,110,131,133,134,138,139,141,146,151,155,158,159,162,164,171,173,174,178,181,182,183,189,209,212,214,216,235,236,245,254,255,256,264,281,285,286,287,293,294,295,299,301,306,308,309,321,331,334,336,337,341,346,347,357,363,369,373,374,381,387,390,398,400,404,423,429,432,436,441,444,445,460,461,464,479,481,482,483,485,487,488,489,501,507,512,515,518,520,536,537,541,545,548,551,552,555,557,559,570,578,583,584,585,588,589,592,596,597,603,605,611,618,646,648,651,658,665,668,670,674,676,677,681,683,686,696,697,701,705,715,721,723,728,730,735,741,742,743,753,755,756,767,769,783,803,804,805,807,810,812,816,833,843,848,854,856,859,861,863,866,874,875,876,877,879,888,890,891,896,897,901,907,912,927,930,931,945,950,953,957,963,966,970,971,972,973,974,975,977,979,982,988,989,995,999,1003,1005,1009,1023,1025,1027,1028,1029,1033,1036,1038,1040,1041,1043,1047,1053,1055,1062,1066,1068,1082,1084,1088,1092,1095,1111,1119,1125,1126,1138,1139,1145,1148,1155,1158,1164,1166,1168,1172,1188,1193,1194,1196,1201,1208,1215,1217,1218,1220,1226,1230,1235,1236,1240,1243,1244,1260,1270,1271,1274,1275,1276,1281,1297,1298,1299,1307,1316,1324,1329,1343,1347,1348,1349,1350,1351,1353,1355,1357,1361,1362,1370,1386,1393,1395,1396,1398,1399,1401,1403,1408,1409,1410,1420,1428,1435,1436,1437,1446,1460,1464,1466,1477,1480): mod_base = OTHER; note = Uracil
atggatatgagagtacccgcccaactgttgggcttgctgctgctgtggcttagaggggccagatgcgaggaggagctgcaggtaattcagccagataagagcgtcagcgtggcggcaggggagagcgcgatcttgcattgtacagtcacatcactgattcctgtgggccctattcagtggtttcggggtgcaggccccgccagagagctgatctataaccaaaaagaggggcacttcccgcgggtaacaaccgtttcggagagtacaaagagagaaaacatggatttcagcatttcgatctcaaatattacccccgccgatgccggcacctactattgcgtgaagttccggaaaggtagccctgacactgagttcaagagtggggctggtacagagctgtcagtcagagccaagggaggagataaaactcatacctgccctccttgccccgccccagagttgctggggggccccagcgtatttctgtttccgcccaaacctaaagataccctgatgatctcgcggacgccagaggttacctgcgtcgtcgttgatgtatcccacgaggatcccgaagtgaaatttaattggtacgttgacggtgtcgaagtgcacaatgccaagacaaaaccccgggaggagcaatataatagcacctacagagtcgtctccgtattgactgtactgcaccaagattggctgaatggaaaggaatacaaatgtaaggtatccaataaggctttaccggcacctattgagaagaccatatccaaagccaaaggtcaacccagggaaccccaagtttatactctgcctccaagcagggaggagatgaccaagaatcaggtcagcctgtggtgtctcgtgaaaggcttttatcccagcgatattgccgttgagtgggaatcgaatgggcagcccgaaaataattacaaaaccacccctcccgtcctggatagcgatggtagcttttttctgtactccaaattgaccgtggataagtctagatggcagcagggcaatgtgtttagctgctctgttatgcatgaggctctgcacaatcactatacgcagaaaagcctgtccctgagtcctggcggagggggcggatccggaggtggcggttccgaagaagagttgcaggtaatccagcctgataaaagtgtgtcggtcgcggccggcgagagtgcaattctgcactgcaccgtgaccagtcttataccggtaggtcccattcagtggttcagaggcgccggccctgccagggaattgatttacaatcagaaggagggacactttcccagagtgacgaccgtcagcgaatccactaaaagggagaacatggatttttctatctccattagcaacatcaccccggccgacgctggcacgtattattgtgtgaaatttcgaaagggctcccccgatacagagtttaaaagcggtgcaggaaccgagctgagtgtccgggcgaagtgatga

SEQ ID NO: 40 - RNA, 1794 nt, synthetic construct
  source 1..1794: mol_type = other RNA
  modified_base order(2,6,8,14,18,26,29,35,38,41,44,46,50,64,71,77,80,98,101,108,119,120,125,130,145,146,147,149,151,152,154,156,160,167,172,176,189,200,205,209,218,223,225,235,238,241,243,244,249,257,268,269,275,297,302,304,308,314,323,329,344,346,349,352,364,366,369,375,376,377,382,383,388,391,407,410,416,418,421,446,448,449,455,462,466,472,497,502,504,506,509,517,520,521,533,539,541,544,560,572,576,580,581,582,588,590,591,593,608,610,612,613,615,617,626,629,635,639,643,650,664,668,670,677,684,690,704,716,723,727,729,730,748,750,753,757,768,773,776,786,791,793,794,795,797,799,800,804,807,813,824,827,830,843,848,853,857,860,863,869,873,882,885,890,895,896,897,901,904,908,917,923,930,958,970,977,980,984,986,989,995,998,1009,1013,1027,1033,1040,1044,1055,1059,1062,1065,1067,1068,1079,1101,1110,1115,1117,1124,1128,1132,1145,1160,1166,1171,1173,1178,1186,1187,1189,1195,1197,1200,1202,1208,1213,1219,1221,1224,1243,1257,1260,1262,1265,1282,1283,1285,1286,1289,1292,1294,1296,1301,1307,1315,1321,1337,1339,1340,1345,1352,1355,1367,1378,1390,1392,1394,1400,1402,1404,1423,1425,1428,1438,1440,1451,1457,1460,1467,1470,1474,1478,1484,1491,1498,1503,1505,1508,1513,1520,1529,1532,1538,1547,1552,1555,1556,1575,1583,1586,1588,1609,1610,1619,1628,1629,1655,1660,1661,1667,1668,1669,1671,1673,1680,1682,1689,1692,1695,1705,1708,1710,1711,1713,1715,1720,1721,1740,1747,1748,1761,1772,1774,1778,1789,1792): mod_base = OTHER; note = Uracil
atggatatgcgggtgcctgcccagctgctgggcctgctgctgctgtggctgagaggcgcccggtgcgaggtccagctggtagagagcggaggcgacctggtgcagcctggcagaagccttagactgagctgcgccgcaagcggctttatcttctctaactacggcatgagctgggtcagacaggcccctggaaagggcctggaatgggtggccaccatcagctctgccagcacctactcatattaccctgacagcgtgaagggcagattcaccatcagccgggacaacgccaaaaatagcctgtacctgcaaatgaacagcctccgggtcgaggacaccgccctgtactactgcggccggcactctgatggaaatttcgccttcggctactggggccagggaacactggtgacagtgtcgtcagccagcaccaagggacccagcgtgttccccctggcccctagctccaagtccaccagcggcggaacagccgccctgggctgtctggtgaaagactacttccccgagcccgtcaccgtgtcctggaacagcggcgccctcaccagcggcgtgcatacctttcccgctgttctgcagagcagcggactgtattctctgagcagcgtggtgaccgtgcctagctccagcctgggcacccaaacatacatctgcaacgtgaaccataagcctagcaacaccaaggtggacaagaaggtggaacctaagtcttgcgacaagacccacacctgtcctccgtgccccgcccctgagctcctgggaggccctagcgtgtttctgttccctcctaaacctaaggacaccctgatgatcagccgcacccctgaggtgacatgcgtggtcgtggacgtgagtcacgaggatcctgaagtgaagtttaactggtacgtggacggagtggaagtgcacaatgccaagacaaaacccagagaagagcagtacaacagcacatacagagtggtgagtgtgctgaccgtgctgcaccaggactggctgaacggcaaggaatacaagtgcaaggtgagtaacaaggccctgcctgctcctattgagaagacgatcagcaaggccaagggccagcctagggagcctcaggtgtacaccctgcctccatccagagaagagatgacaaagaaccaggtgagcctcagctgtgccgtgaagggcttctacccctctgatatcgccgtggaatgggagtctaatggacagcccgagaacaactacaagacaacccctcctgtgctggacagcgacggcagcttcttcctggtgtctaaactgacagtggacaagtccagatggcagcagggcaacgtgttcagctgcagcgtgatgcacgaggccctgcacaaccactacacccagaaatctctgagcctgtctccaggaggcggcggcggctctggtggcggcggctctgaggaagaactgcaagtcatccagcctgataaatcggtcagcgtcgccgctggcgagtccgctatcctgcactgcaccgtgaccagcctgatccccgtgggcccgatccagtggttccggggcgcaggacccgctagagagctcatctacaaccagaaggaaggccacttcccaagagtgacaacagttagcgagagcaccaagagagaaaacatggacttcagcatttctatcagcaatatcacccctgctgatgccgggacatactattgtgtgaagttcagaaagggcagcccagatacagagttcaagagcggcgctggcaccgagctgtccgtgcgggccaaatgataa

SEQ ID NO: 41 - RNA, 1107 nt, synthetic construct
  source 1..1107: mol_type = other RNA
  modified_base order(2,8,14,21,26,28,29,35,38,41,44,46,50,64,71,74,77,90,92,94,98,102,104,111,120,123,128,133,152,155,159,161,175,179,184,187,191,208,210,218,221,224,226,228,233,235,240,244,245,254,261,265,266,267,274,276,291,292,293,294,299,305,314,329,335,337,340,343,346,347,359,362,367,373,374,392,393,398,410,420,425,427,428,431,433,434,438,442,444,447,455,460,476,479,481,485,488,496,497,499,513,518,523,530,542,555,556,558,569,594,598,605,617,623,625,627,637,653,655,661,668,683,685,693,695,706,707,721,741,753,764,770,773,780,783,791,793,795,797,804,818,821,826,828,833,842,843,845,851,860,865,868,869,885,896,899,901,922,923,927,932,941,943,945,949,951,968,973,974,975,980,981,982,984,986,995,996,1002,1008,1018,1021,1023,1024,1026,1028,1029,1033,1034,1045,1050,1060,1061,1085,1091,1102,1105): mod_base = OTHER; note = Uracil
atggacatgagagtgccagctcagctgttgggcctgctgctgctgtggctgagaggcgcccggtgcgacgtgctgatgacccagagccctctgtccctgcctgtgacacctggcgagcctgctagcatcagctgcagaagcagccgcaacatcgtgcatatcaacggcgacacctacctggaatggtacctgcagaagcccggccagtctccccagctgctcatctataaggtctccaatagattcagcggcgtgcccgatagatttagcggctctggcagcggcacagattttaccctgaaaatcagcagagtggaagccgaggacgtgggagtgtactactgcttccagggcagcctgctgccctggaccttcggccagggaacaaaggttgagatcaagagaaccgtcgccgcccctagcgtgttcatcttccctccatctgatgagcaactgaagtccggaaccgccagcgtggtgtgcctgctgaacaacttctaccccagagaagctaaagtgcagtggaaggtggacaacgccctgcagagcggaaattctcaggagagcgtgaccgagcaggacagcaaggacagtacctacagcctgagcagcaccctgacactgtctaaagccgactacgagaagcacaaggtgtacgcctgcgaggtgacccaccagggcctgtccagccctgtgaccaaaagcttcaaccggggcgaatgcggcggaggcggcagcggtggcggcggcagtgaagaggaactgcaagtgatccaacctgataagagcgtctctgtggccgctggagaaagcgccatcctgcactgtacagtgacgagccttatccccgtgggccccatccagtggttccggggagccggacctgccagagagctgatctacaaccagaaagagggccacttccctagagtgaccaccgtgtctgagtctacaaagcgggaaaacatggactttagcatttctatcagcaacattacccctgcagatgccggcacatactattgtgttaagttcaggaagggctcccctgacaccgaattcaagagcggcgccggcacagagctgagcgtgcgggccaagtgatga

SEQ ID NO: 42 - RNA, 1416 nt, synthetic construct
  source 1..1416: mol_type = other RNA
  modified_base order(2,8,14,21,26,29,35,38,41,44,46,50,64,71,77,80,81,98,101,102,119,125,127,129,130,145,146,149,151,152,160,167,172,176,189,200,205,209,218,229,231,235,241,244,257,268,269,275,285,302,304,308,314,319,321,323,329,336,344,346,348,349,352,363,364,366,369,376,377,382,383,388,391,407,410,416,418,420,421,423,441,446,448,449,450,453,455,462,492,495,497,501,502,504,506,509,517,520,521,533,539,541,544,558,560,572,579,580,581,582,585,588,590,593,608,610,617,619,622,624,626,629,635,639,642,646,648,650,664,668,670,677,690,691,696,704,716,723,730,748,750,753,757,762,765,768,773,776,783,787,789,791,793,794,797,799,800,819,824,827,830,843,848,853,855,857,860,863,869,890,895,896,901,904,908,917,923,930,958,964,970,972,977,980,982,986,989,995,998,1009,1013,1027,1033,1040,1042,1055,1065,1067,1079,1080,1081,1083,1110,1115,1117,1119,1124,1128,1131,1145,1160,1162,1166,1167,1171,1175,1178,1186,1187,1189,1194,1195,1197,1200,1202,1208,1213,1224,1233,1243,1257,1262,1265,1282,1283,1285,1286,1289,1291,1294,1296,1301,1307,1311,1315,1321,1335,1337,1339,1340,1341,1342,1345,1347,1352,1355,1367,1378,1394,1400,1407,1411,1414): mod_base = OTHER; note = Uracil
atggacatgcgggtgcccgctcagctgctgggactgctgctcctgtggctgcgcggagccagatgcgaggtgcagctggttgaaagcggcggcgacctggttcaacccggcagaagcctgagactgtcttgcgccgccagcggattcatcttcagcaactacggcatgagctgggtgcggcaggcccctggcaagggcctggaatgggtcgccacaatcagcagcgcctctacctacagctactaccccgacagcgtgaaaggcagattcaccatcagcagagataacgccaagaacagcctgtacctgcagatgaactctctccgggtggaagataccgccctgtattactgcggcaggcattctgatggcaacttcgccttcggctactggggccaaggcaccctggtgaccgtgtcttctgccagcaccaagggccctagcgtctttcctctggcccctagcagcaagagcacaagcggaggcacagctgctctgggttgtctggtcaaggactacttccccgagcccgtgaccgtgtcctggaacagcggcgctctgacaagcggagtgcacacttttcctgctgtgctgcaaagcagcggcctgtacagcctgtcatctgtggtcaccgtgcctagtagctctctcggaacccagacctacatctgcaacgtgaaccacaagccttccaataccaaggtggacaagaaggtggagcctaaaagctgcgacaagacacacacatgtcctccgtgccctgctcctgagctgctgggcggtccctctgtgttcctgttcccaccaaagcccaaggataccctgatgatcagccggacccctgaggtgacgtgtgtggtggtggacgtgagccacgaggaccccgaggtgaagttcaactggtacgtggacggcgtggaagtgcacaatgccaaaacaaagccaagagaagagcagtacaactccacctatagagtggtctccgtgctgaccgtgctgcaccaggactggctgaacggcaaggaatacaaatgcaaggtgtccaacaaggccctgcccgcccctatcgagaagacaatttctaaagccaagggccagcccagagagcctcaggtgtataccctgcctcctagcagagaggaaatgaccaaaaaccaggtgtcccttacatgcctggtgaaaggcttctacccttctgatatcgccgtggaatgggagagcaatggccagcctgagaacaactacaagaccacccctccagtgctggacagcgacggcagcttcttcctgtactctaagctgaccgtggataagtcccggtggcagcagggcaatgtgttttcctgtagcgtgatgcacgaggccctgcacaaccactacacacagaagagcctgagcctgagccctggctgataa

SEQ ID NO: 43 - RNA, 1794 nt, synthetic construct
  source 1..1794: mol_type = other RNA
  modified_base order(2,8,14,26,29,35,38,41,44,46,50,64,66,71,77,80,98,101,108,119,125,129,130,132,145,146,149,151,152,160,167,172,176,200,205,209,218,229,231,235,237,241,244,246,249,252,257,258,268,269,275,302,304,308,314,323,329,336,344,346,349,352,364,366,369,375,376,377,382,383,388,391,407,410,416,421,423,426,441,442,444,446,447,448,449,453,455,463,465,478,480,483,497,502,506,509,517,520,521,533,537,539,544,559,560,572,580,581,588,590,593,608,610,613,615,617,619,622,624,626,627,629,635,639,640,642,643,646,648,650,664,668,670,677,691,693,696,704,716,723,730,748,750,753,757,768,773,776,791,793,794,795,797,799,800,801,813,824,827,830,843,848,853,857,860,863,869,871,890,895,896,897,901,904,908,912,917,923,945,958,970,972,977,980,986,989,995,998,1009,1013,1027,1033,1040,1055,1059,1062,1067,1079,1101,1115,1116,1117,1124,1128,1131,1134,1145,1160,1162,1166,1171,1175,1178,1186,1187,1188,1189,1195,1200,1202,1208,1213,1219,1233,1243,1257,1262,1265,1282,1283,1285,1286,1289,1291,1294,1301,1307,1321,1335,1337,1339,1340,1341,1345,1352,1355,1367,1371,1374,1378,1390,1394,1400,1402,1404,1407,1423,1425,1451,1457,1460,1467,1478,1480,1482,1484,1491,1505,1508,1513,1520,1525,1529,1532,1538,1545,1547,1548,1552,1555,1556,1572,1575,1583,1586,1588,1609,1610,1614,1619,1628,1630,1655,1659,1660,1661,1667,1673,1680,1682,1689,1692,1695,1705,1708,1711,1713,1715,1720,1721,1737,1747,1748,1753,1755,1764,1772,1774,1778,1789,1792): mod_base = OTHER; note = Uracil
atggacatgagagtgccagcccagctgctgggactgctgctcctgtggctgcgaggagccagatgtgaagtgcagctggtggaaagcggcggcgacctggtccagcctggcagaagcctgcggctgagttgtgccgccagcggcttcatcttcagcaactacggcatgagctgggtgcggcaggcccccggcaagggcctggaatgggtcgccaccatcagcagcgcctctacctatagctactatcctgatagcgttaagggaagattcaccatcagccgcgacaacgccaagaacagcctgtacctgcagatgaacagcctgagagtcgaagataccgccctgtactactgcggccggcactctgatggaaatttcgccttcggctactggggccagggcacgctggtgacagtcagctctgctagcacaaaaggaccttctgttttccctctggccccatctagcaagagcacatctggtggcaccgccgccctgggctgcctggtgaaggactacttccccgagcccgtgactgtgagctggaacagcggcgccttgacaagcggcgtgcacaccttcccagctgtgctgcaaagcagcggcctgtactctctgtcctctgttgtgacagtgccttcttcctctctgggcacacagacctacatctgcaacgtgaaccacaagccctctaataccaaggtggacaaaaaggtggaacctaagagctgcgacaagacacacacatgtcctccatgccccgcacctgagctgctgggcggccccagcgtgtttctgtttcccccgaagcctaaggacaccctgatgatcagccggacccctgaagtgacctgcgtggtagtggacgtgtcccacgaggaccccgaggtgaaatttaactggtacgtggatggcgtggaggtgcacaacgccaaaacaaaacctagagaggaacagtacaacagcacctatagagtggtcagcgtgctgaccgtgctgcaccaggactggctgaacggcaaagagtacaagtgcaaggtgagcaacaaggccctgcctgctcccatcgagaagaccatcagcaaggccaagggacaacctagagagccccaggtttacacactgcctcctagtagggaggaaatgaccaagaaccaggtgtccctgacatgcctggtcaaaggcttttacccctccgatatcgccgtggaatgggagtccaacggccagcctgaaaacaactacaagaccacccctccagtgctggacagcgacggcagcttcttcctgtactccaagctgaccgtggacaagagcagatggcagcagggcaatgtgtttagctgcagcgtgatgcacgaggccctgcataatcactacacccagaagtccctgagcctctctcctggaggcggcggaggatctggcggaggcggcagcgaggaagagctgcaggtgatccagcctgacaaaagcgtgtctgtggccgctggcgagagcgccatcctgcactgcaccgtgacctccctgatccccgtgggccctattcagtggttccggggcgccggacctgctagagagctgatctacaaccaaaaggaaggccacttccctagagtgaccaccgtgtccgagagcaccaaaagagagaacatggatttcagcatcagcatcagcaatatcacccctgctgatgccggcacatactactgtgtgaagttcagaaagggcagccctgacaccgagttcaagtctggagccggtacagagctgtccgtgcgggccaagtgataa

SEQ ID NO: 44 - RNA, 1482 nt, synthetic construct
  source 1..1482: mol_type = other RNA
  modified_base order(2,6,8,14,26,29,35,38,41,44,46,50,64,77,83,86,93,100,102,104,105,106,108,110,117,124,126,131,134,135,139,146,155,158,164,171,173,174,178,181,182,209,212,214,235,236,237,245,254,262,279,281,286,287,293,299,308,309,315,318,321,331,334,337,341,346,347,363,366,373,374,398,400,402,404,436,445,450,453,456,461,464,475,477,479,481,482,485,487,488,495,501,512,515,518,520,531,536,541,543,545,548,551,557,570,573,578,579,583,584,589,592,596,605,611,633,646,658,660,665,668,670,674,677,683,686,690,697,701,715,721,728,730,732,743,747,753,755,767,789,803,805,812,816,833,848,850,854,859,863,866,874,875,877,890,896,901,907,909,912,931,945,948,950,951,952,953,963,967,969,970,971,972,973,974,977,979,989,995,999,1009,1025,1027,1028,1029,1033,1040,1043,1055,1066,1082,1088,1111,1113,1125,1128,1139,1145,1148,1155,1166,1172,1193,1196,1201,1203,1208,1217,1220,1224,1226,1235,1240,1243,1244,1245,1260,1263,1271,1274,1276,1278,1281,1297,1298,1302,1307,1311,1316,1318,1343,1348,1349,1355,1361,1368,1370,1393,1396,1399,1401,1403,1408,1409,1435,1436,1441,1449,1460,1466,1477,1480): mod_base = OTHER; note = Uracil
atggatatgcgggtccccgcccagctgctgggcctgctgctgctgtggctgagaggagcccggtgcgaggaggaactgcaggtcatccagcctgacaagtctgtttctgtggccgctggcgaatctgccatccttcactgcaccgtgacaagcctgatcccggtgggacctattcaatggttccggggcgccggccccgccagagagctgatctacaaccagaaagagggccactttccacgcgtgacaaccgtgagcgagtccaccaagcgggagaatatggacttcagcatcagcatcagcaacattacacctgctgatgccggcacatactactgcgtgaaattcagaaagggcagccctgataccgagttcaagagcggagccggcaccgagctgtctgtgcgggccaagggcggcgacaagacccacacatgccccccctgccctgctcctgaactgctgggcgggccatctgtgttcctgttcccccctaagcctaaagacaccctgatgatctccaggacccctgaggtgacctgtgtggtggtcgacgtgagccacgaagatcctgaggttaagttcaactggtacgtggacggcgtggaagtgcacaacgccaagaccaaacctagagaagagcagtacaacagcacctatagagtggtgtccgtgctgacagtgctgcatcaggactggctcaacggcaaggaatacaagtgcaaggtgtctaacaaggcactgcctgcccctatcgagaagaccatcagcaaggccaaaggccagcctagagaaccccaggtgtacacactgcctccaagcagagaggaaatgaccaagaaccaggtgtccctgacctgcctggtgaagggcttctaccccagcgacatcgccgtcgaatgggagtctaatggccagccagaaaacaactacaaaaccacccctcctgttttggacagcgatggatcttttttcctgtacagcaagctgaccgtggataagagcagatggcagcagggcaacgtgtttagctgcagcgtgatgcacgaggccctgcacaaccactacacccagaagagcctgagcctgagccccggcggcggcggcggatctggcggaggaggtagtgaggaagaactccaggtgatccaacctgacaagagcgtgagcgtggccgccggcgagagcgccatcctgcactgtacagtgaccagcctgatccctgtgggccccatccagtggtttcggggcgcgggccctgctagagagctgatctataatcaaaaagaaggccacttccctagagtgactaccgtgtccgagagcacaaagagagagaacatggacttcagcatcagcatcagcaatatcacacccgccgacgccggcacctactactgtgtgaagttcagaaagggcagcccggacacagagttcaaatccggcgctggaaccgagctgagcgtcagagccaagtgataa

SEQ ID NO: 45 - RNA, 2232 nt, synthetic construct
  source 1..2232: mol_type = other RNA
  modified_base order(2,6,8,14,26,28,29,33,35,36,38,39,41,44,46,50,51,64,77,78,83,86,93,96,100,102,104,106,110,111,124,131,134,138,139,141,146,147,150,155,156,158,164,168,171,173,174,178,181,182,189,198,209,212,214,216,234,235,236,237,245,252,254,258,273,281,285,286,287,289,293,295,299,300,301,306,308,331,333,334,336,337,339,341,342,346,347,357,358,360,373,374,375,379,387,390,398,404,411,417,426,427,439,441,452,458,461,475,479,481,483,485,486,499,506,507,509,513,514,516,521,526,529,530,533,539,540,543,548,549,553,556,557,558,570,573,584,587,589,594,609,610,611,612,618,620,627,629,631,633,637,654,656,660,661,662,663,664,668,670,674,681,683,684,687,706,709,711,712,716,721,722,723,732,733,735,738,741,748,749,750,754,772,773,775,777,779,783,786,795,798,811,813,820,822,831,835,836,838,839,843,846,854,856,857,858,860,862,863,864,882,887,888,890,893,895,897,911,916,920,923,926,932,934,939,953,958,959,963,964,967,969,971,980,981,986,990,1021,1032,1033,1035,1040,1043,1044,1045,1047,1049,1051,1052,1058,1060,1061,1065,1071,1072,1076,1090,1096,1098,1103,1105,1110,1116,1118,1125,1130,1142,1144,1152,1178,1180,1187,1195,1197,1208,1212,1223,1225,1227,1229,1234,1237,1238,1241,1248,1249,1250,1252,1254,1257,1258,1263,1265,1271,1276,1282,1306,1308,1314,1325,1326,1327,1328,1342,1345,1346,1347,1348,1349,1352,1353,1354,1356,1357,1364,1370,1378,1384,1400,1402,1403,1408,1410,1413,1415,1418,1422,1429,1430,1434,1437,1441,1455,1457,1458,1459,1463,1485,1486,1488,1494,1514,1520,1523,1530,1539,1541,1542,1545,1547,1548,1554,1561,1568,1569,1571,1575,1576,1583,1592,1595,1601,1602,1605,1610,1615,1618,1619,1620,1646,1647,1649,1650,1651,1653,1672,1673,1674,1682,1683,1686,1691,1693,1699,1716,1718,1723,1724,1730,1732,1734,1736,1737,1745,1758,1768,1770,1771,1773,1774,1778,1783,1784,1785,1788,1803,1806,1810,1811,1812,1816,1818,1821,1824,1827,1835,1837,1841,1854,1864,1866,1872,1876,1878,1889,1895,1896,1898,1899,1912,1914,1916,1917,1918,1922,1936,1943,1946,1951,1953,1956,1958,1959,1963,1967,1970,1976,1985,1990,1993,1994,1995,2001,2021,2022,2024,2026,2028,2031,2043,2046,2047,2048,2055,2057,2066,2076,2093,2098,2099,2100,2103,2105,2109,2111,2115,2118,2120,2121,2133,2139,2143,2146,2149,2153,2158,2159,2169,2185,2186,2187,2193,2196,2202,2209,2210,2214,2216,2227,2230): mod_base = OTHER; note = Uracil
atggatatgagagtacccgcccaactgttgggtcttcttctactgtggcttagaggcgcaaggtgcgaagaggaacttcaagtaatccagcctgataagtctgtgtcagttgcggccggcgagtccgcaatcctacattgtacggttactagccttatccccgtaggtcctattcagtggttcaggggtgcgggacctgccagagagctgatatataaccaaaaagaaggacattttccgagagtcacgactgtgagtgagagcaccaagcgtgagaacatggatttctcgatctccatttccaatatcaccccagccgacgccggcacatattattgtgttaagttcagaaagggttctcccgacacagagtttaagtcgggcgctggtacagagctaagcgtccgggctaaaggtggaggcggttccggcggaggctctgaggaagagctgcaagtaatccaaccggacaaatcggtatctgttgccgccggggagtcagccattctgcattgtaccgtgacgtcgttaataccagttggtcccattcaatggtttcgcggggccggtcctgccagagaactgatctacaatcagaaggaagggcattttccacgtgtgaccactgtatctgaatcaaccaagagggagaatatggatttttcaatctcgatcagcaatattactcccgccgacgcagggacctactattgcgtaaagtttcggaaaggttctcctgataccgagtttaagtcaggagccgggaccgagttgtctgtacgtgctaagggaggtgataagacccacacgtgtccaccatgtcccgcccctgagttgttaggtggtcccagcgtgtttctctttccgcccaagcccaaggatacccttatgatctctaggacgcccgaagtaacctgcgtcgtcgtggacgtctcgcatgaggaccccgaggtcaagttcaattggtatgtcgacggagttgaggtgcataacgcaaaaacgaaaccgcgagaagagcagtacaacagcacttatcgcgtggtttctgtgttaaccgtcttgcatcaggattggctgaacgggaaagaatacaagtgtaaagtatcgaataaagctctcccagctccaatcgagaaaacgatatcgaaggctaaggggcagcccagggaaccgcaggtgtacacactcccgccgtctcgggaggagatgactaaaaaccaagtatctctaacatgcttggtgaaaggtttctatccttcagatatcgccgtagaatgggagtcaaacggccagccagaaaacaactataaaactacccccccggttttggacagcgacgggtcgtttttcctttattccaaactgaccgtagacaagtcccgatggcagcaaggaaacgtgttcagctgtagtgtgatgcatgaggccttacataatcactacacccagaagagtctttccctgagcccaggaggagggggaggttctggaggtggcgggagcgaggaagaactgcaagtgatacagcctgacaagagtgttagtgttgccgctggagaatccgcgattctacattgcacggtcaccagcctcatccccgttggtccgatccaatggtttagaggcgccggcccggcaagggagcttatttataaccaaaaggagggccactttccccgagttactaccgtgtccgaatccacaaaacgcgagaatatggacttcagcatatctattagcaacatcacgccggcagatgccgggacatattattgcgtcaagtttcgtaaggggagcccagatactgagtttaagtctggtgctggtaccgagctgtccgtgcgcgcaaaaggtggagggggctctgggggtggctctgaagaagagctgcaggttattcaacccgacaagtctgtttcggtcgccgcgggcgaatccgccatactgcactgtactgttacctcactgatccccgtgggaccaatacagtggtttaggggtgcaggccccgcgagggaacttatctataatcaaaaggagggtcatttcccccgtgtcacaaccgtcagcgaaagtaccaaaagggagaacatggactttagtatcagtatcagtaatattaccccagcagatgcgggtacgtactactgcgtgaaattccgaaaaggtagcccggacacggaatttaaaagtggtgcgggtacggagttgagtgtgcgcgccaagtgatga

SEQ ID NO: 46 - RNA, 1857 nt, synthetic construct
  source 1..1857: mol_type = other RNA
  modified_base order(2,8,14,26,29,35,38,41,44,46,50,64,77,83,86,87,93,104,106,108,110,114,117,129,131,134,138,139,146,151,153,155,158,164,171,173,178,181,182,183,198,201,209,212,213,214,235,236,245,254,256,281,286,287,289,293,299,301,303,306,308,315,318,321,331,334,337,341,346,347,373,374,375,398,404,405,426,455,461,464,482,484,488,509,512,517,524,533,536,540,542,543,551,556,559,560,576,587,590,592,612,613,614,623,632,640,642,657,659,664,665,671,677,686,693,709,712,714,715,717,719,724,725,741,744,751,752,757,776,778,780,782,801,813,814,816,819,823,828,834,839,842,852,857,859,860,861,863,865,866,870,890,893,896,898,900,909,914,919,923,926,929,933,935,948,951,956,961,962,967,970,974,981,983,984,989,1011,1024,1036,1038,1043,1046,1052,1055,1061,1064,1075,1079,1093,1099,1106,1108,1110,1121,1133,1145,1147,1167,1181,1182,1183,1190,1194,1197,1198,1200,1211,1226,1232,1237,1239,1241,1244,1252,1253,1255,1260,1261,1263,1266,1268,1269,1274,1279,1299,1309,1323,1328,1331,1345,1348,1349,1351,1352,1355,1357,1367,1373,1387,1403,1405,1406,1411,1418,1421,1433,1444,1460,1466,1468,1470,1517,1523,1526,1533,1536,1544,1545,1546,1548,1550,1571,1574,1579,1581,1586,1595,1598,1604,1611,1613,1618,1621,1622,1632,1638,1641,1649,1652,1654,1656,1659,1675,1676,1677,1680,1685,1694,1719,1721,1725,1726,1727,1728,1729,1731,1733,1735,1739,1741,1748,1771,1774,1777,1781,1786,1787,1813,1814,1827,1838,1844,1855): mod_base = OTHER; note = Uracil
atggacatgcgggtccccgcccagctgctgggcctgctgctgctgtggctgagaggagcccggtgcgaggaagagctgcaggtgattcaacctgacaagagcgtgtctgtggctgctggggagagcgctatcctccattgcacagtgacatctctgatccccgtgggccctatccagtggtttagaggcgccggccctgctagggaactgatttacaaccagaaagagggccacttccccagagtgacaaccgtgtccgagagcaccaagagagaaaacatggacttctccatcagcatctctaatatcacccctgctgatgccggaacctactactgcgtgaagttcagaaagggcagccccgacaccgagtttaagagcggcgccggaaccgagctgagcgttagagccaaaggcggcggcggtagcggcggcggcggcagcgaggaagagctgcaggtgatccagccagacaagagcgtgtccgtggccgccggagagagcgccatcctgcactgcaccgtgacaagcctgatccctgttggccccatccagtggttcaggggcgcaggccctgccagagaactgatctacaaccagaaggaaggccatttcccacgggtgacaacagtcagcgagtctaccaagcgggaaaatatggacttcagcatcagcatcagcaacatcacccctgccgacgccggcacctactattgtgtgaaattccggaaaggaagccctgataccgaattcaagtccggagccggcaccgagctgtctgtgagagccaagggcggagataagacccacacttgtcctccgtgccctgcccctgagctgctcggcggacctagcgtctttctgttccctccaaagcccaaggacaccctgatgatctctagaacccctgaggtgacctgcgtggtcgtcgatgtgagccacgaagatcctgaagtgaagttcaactggtacgtggacggtgttgaggtgcacaacgccaagaccaagcctcgcgaggaacagtacaacagcacatatagagtggtgagcgtgctgaccgtgctgcaccaggactggctgaacggcaaggaatacaagtgcaaggtgtctaacaaggccctgcccgcccccatcgagaagaccatctccaaggccaagggccagcctcgggagccccaggtttacaccctgcctccttctagagaagagatgaccaagaaccaggtgagcctgacatgtctggtgaagggcttctacccttctgatattgccgtggaatgggagagcaacggccagcctgagaacaactacaagacaacccctccagtgctggacagcgacggctcattcttcctgtacagcaagctgaccgtggacaaaagcagatggcagcagggcaacgtgttcagctgcagcgtgatgcacgaggccctccacaaccactacacccagaaaagcctgagcctgtctcccggcggcggcggaggcagcggcggaggcggaagcgaggaagagctgcaagtgatccagcctgataagagcgtttctgtcgccgccggagaaagcgccatcctgcactgtacagtgaccagcctgatccccgtgggccctatccaatggttccggggcgctggccctgctagagaactgatctataatcagaaggaaggccactttcctcgggtgaccaccgtgagcgaaagcaccaaaagagagaatatggatttttctatctccatctccaacatcacaccagccgacgccggcacatactactgcgtgaaattcagaaaaggcagccccgacacagagttcaagagcggcgctggaacagagctgagcgtgcgggccaagtga

SEQ ID NO: 47 - RNA, 1482 nt, synthetic construct
  source 1..1482: mol_type = other RNA
  modified_base order(2,6,8,14,26,28,29,33,35,38,41,43,44,46,50,57,60,64,66,77,83,86,87,93,96,100,102,104,110,114,117,131,133,134,139,146,150,151,155,158,162,164,171,173,178,181,182,195,198,209,212,214,231,234,235,236,245,254,262,281,286,287,288,293,299,301,306,308,309,312,315,330,331,334,337,339,341,346,347,358,363,373,374,375,379,381,384,387,393,398,400,402,404,420,429,436,441,444,445,453,461,464,468,479,481,482,483,485,487,488,489,492,495,507,510,512,515,518,528,531,536,541,545,548,551,557,573,578,583,584,589,592,594,596,603,605,611,646,654,658,660,665,668,670,674,677,683,686,696,697,701,715,721,728,735,743,750,753,755,767,769,771,803,805,810,812,833,848,850,852,854,856,859,863,866,873,874,875,877,882,883,890,894,896,901,907,909,915,927,930,931,933,945,948,950,953,960,963,967,969,970,971,973,974,975,976,977,979,981,989,993,995,1003,1005,1009,1020,1025,1027,1028,1030,1032,1033,1036,1038,1040,1043,1055,1062,1066,1068,1071,1080,1082,1086,1088,1090,1095,1104,1119,1125,1126,1138,1139,1145,1146,1148,1155,1166,1172,1182,1186,1188,1193,1196,1200,1201,1208,1216,1217,1220,1221,1224,1226,1235,1240,1243,1244,1257,1263,1271,1274,1275,1276,1297,1298,1302,1307,1311,1316,1318,1320,1326,1343,1347,1348,1349,1351,1353,1355,1356,1357,1359,1361,1363,1368,1370,1380,1389,1393,1396,1398,1399,1403,1408,1409,1410,1419,1428,1435,1436,1437,1441,1443,1446,1452,1460,1462,1464,1466,1473,1477,1480): mod_base = OTHER; note = Uracil
atggatatgagagtacccgcccaactgttgggtctgctgctgttgtggctgcggggtgctagatgtgaggaggagctgcaggtgattcagcctgataaatctgtgagcgtggctgctggggagagcgccatcttgcactgcaccgtgacttcactgatccctgtggggcctatccaatggttccggggcgccggtcctgccagagagctcatctacaaccagaaggagggtcatttccccagagtaaccaccgtaagcgagtccaccaagcgggagaacatggactttagcatcagcatctccaatattactcctgccgacgccgggacttactactgtgtgaagttcaggaagggctcccctgacaccgagtttaagtctggtgctggcactgagctgtctgtgagagccaaggggggtgacaagactcacacctgccctccttgccccgctccagagctgctgggtgggcccagcgtgtttctgtttcctcctaagcccaaggatactctgatgatcagccggactcctgaggtcacctgcgtggtagtggacgtcagccacgaggaccctgaggtgaagttcaactggtatgtagacggtgtggaggtacacaacgccaagaccaagcccagagaggagcagtacaacagtacctatagggtggtgtcggtgctgaccgtgctgcaccaggattggctgaacggcaaggagtacaagtgcaaggtgagcaataaggccctgcccgctcctatcgagaagaccatctctaaggccaaggggcagccccgggaaccccaggtatacactctgccccccagcagggaggagatgaccaagaaccaagtatctctgtggtgcctggtcaagggtttctacccttccgacatcgctgtggagtgggagtctaacggtcagcccgagaataattataagaccacccctcctgtcctggacagtgatgggtctttctttttgtatagcaagctgactgtggacaagtctaggtggcagcagggtaacgtgttctcttgctctgtgatgcacgaggccctgcacaatcactatactcagaagagtctgagtctgtcccctggcgggggtgggggaagcggcggtgggggttccgaggaggagttgcaagttatacagcctgacaagagcgtgagcgtggccgccggtgagtctgccatcctgcattgcaccgtcaccagcttgattcctgtgggccccatccagtggttcagaggggccggtcccgctcgggagctgatttacaaccagaaagagggccacttccctagggtgactaccgtgtctgagagtaccaagagagagaacatggatttctctatttctatctccaatatcacccccgctgacgccggtacctactattgcgtcaagtttcgcaagggtagccccgataccgagtttaaatctggtgccggtacggagctgtctgtccgggctaagtgatga

SEQ ID NO: 48 - RNA, 2232 nt, synthetic construct
  source 1..2232: mol_type = other RNA
  modified_base order(2,6,8,14,26,28,29,33,35,38,41,44,46,50,60,64,66,76,77,83,86,87,100,102,104,105,110,111,114,120,131,134,139,144,146,151,153,155,158,159,162,164,171,173,178,181,182,186,189,192,209,212,213,214,216,219,231,235,236,245,246,254,256,262,279,281,285,286,287,293,295,299,301,308,309,318,321,331,333,334,336,337,341,342,346,347,348,358,360,366,373,374,375,379,381,398,400,404,405,423,426,429,432,439,452,458,461,462,471,479,481,485,489,495,499,501,506,507,509,513,514,516,521,530,533,537,539,548,553,556,557,558,570,576,584,587,589,594,606,610,611,612,620,629,631,633,656,660,661,662,664,666,668,674,675,683,696,699,706,708,709,712,716,721,722,723,735,738,744,748,749,754,762,765,773,779,795,798,811,819,820,825,831,836,838,839,846,850,852,854,855,856,857,860,862,863,864,887,890,893,895,897,911,915,916,920,923,926,930,932,934,936,953,958,959,960,963,964,967,971,975,980,986,993,1002,1021,1027,1032,1033,1040,1043,1049,1050,1052,1056,1058,1061,1065,1071,1072,1075,1076,1080,1083,1090,1096,1103,1116,1118,1125,1130,1142,1143,1152,1158,1164,1178,1180,1187,1195,1208,1223,1225,1229,1234,1238,1241,1248,1249,1250,1252,1258,1265,1269,1271,1276,1306,1320,1325,1327,1328,1332,1333,1338,1342,1344,1345,1346,1347,1348,1349,1351,1352,1354,1364,1370,1378,1380,1384,1398,1400,1402,1403,1405,1408,1411,1415,1418,1428,1430,1440,1441,1457,1461,1463,1473,1479,1485,1494,1497,1513,1514,1520,1521,1523,1530,1541,1543,1547,1548,1561,1568,1571,1575,1576,1583,1588,1590,1592,1595,1596,1601,1605,1610,1611,1615,1618,1619,1626,1632,1635,1646,1649,1651,1671,1672,1673,1674,1682,1691,1699,1701,1716,1718,1722,1723,1724,1725,1726,1728,1730,1732,1736,1737,1738,1740,1743,1745,1755,1758,1768,1770,1771,1774,1778,1783,1784,1785,1794,1795,1803,1810,1811,1830,1835,1837,1841,1857,1864,1866,1872,1878,1889,1895,1898,1899,1912,1914,1916,1920,1922,1929,1943,1944,1946,1951,1958,1965,1967,1970,1976,1977,1983,1985,1986,1990,1993,1994,2001,2010,2020,2021,2024,2026,2028,2031,2046,2047,2048,2049,2057,2061,2066,2067,2068,2070,2074,2076,2091,2093,2098,2099,2100,2101,2105,2109,2111,2113,2120,2127,2130,2133,2143,2146,2149,2153,2158,2159,2185,2186,2187,2191,2193,2202,2210,2214,2216,2223,2227,2230): mod_base = OTHER; note = Uracil
atggatatgagagtacccgcccaactgttgggtctgctgctgctgtggctgagaggcgctcggtgtgaggaagagttgcaagtcattcaaccggacaagtctgttagcgttgctgcaggtgagagcgccatcctccactgcactgtcacgtctctcattcctgtaggccctatccagtggttccgtggtgctggccccgccagggaactgatttataatcagaaagaaggtcacttccccagagttaccaccgtctccgagtccaccaagcgggagaatatggatttcagcatctccatctcgaacattacccccgctgatgccggcacgtattattgcgttaagtttagaaagggctctccagataccgaatttaagtctggcgccggcaccgagctctccgttagagccaaaggcggcggtggtagtggtgggggctccgaggaggagctccaggtgattcaaccggataagagcgtgtccgtggctgccggtgagtctgccattctgcattgtacagtgaccagcctgatccctgtggggcccatccagtggtttcggggagccggtccggctcgggaactgatctacaatcagaaggagggtcactttcccagagtgacgaccgtgtctgagagcaccaagagggagaacatggatttctctatcagcattagcaacatcacgcccgccgatgctgggacctattactgcgtgaagtttaggaagggcagtcctgacactgagttcaagtccggggctggtaccgagctgagcgtcagggccaaaggcggtgataagacccacacctgcccgccttgccctgcccctgagctgttgggcggtccgtctgttttcctgtttccgccaaagcccaaagacaccctgatgatctctagaaccccggaggtcacttgcgtcgtggtcgatgtgtctcacgaggaccccgaggtaaagtttaattggtacgtcgatggcgtggaggtgcacaatgccaaaactaagcccagggaggagcaatacaactccacttacagggtggtgagcgttctgactgtgctgcatcaggattggttgaatggtaaggagtacaaatgcaaggtcagcaacaaagctctgccggctcccatcgagaaaaccattagcaaggctaagggtcagcctagggaaccccaagtgtacaccctgcccccctccagagaggagatgacgaagaaccaagtatccctgacgtgcctggtaaagggtttctacccatcggacatcgctgtggagtgggagagcaacggccagcccgagaacaactacaagacgacccctccggtcttggattccgatggctcttttttcttgtacagcaagctgaccgtggacaaatctaggtggcaacaagggaatgtgttctcctgctccgtgatgcacgaggctctacacaaccattacacccagaaaagcctgagtctgagccccggtgggggtgggggtagcggcggtggtggcagcgaggaggagttgcaggttatacagcctgacaagagcgtgtcagttgccgccggcgagtccgcaatcctccattgcaccgtgacatctctgattcccgtgggtcccattcagtggttcagaggtgcaggtcctgcgagagagctgatctacaaccagaaggaggggcattttccgagggtaacaacagtcagcgaatctaccaagcgggaaaatatggatttttctatctccatttctaatatcacgcccgctgatgcgggcacatattactgcgtcaagtttaggaaaggttcaccggatacagagttcaagagcggggccggcactgagctgtcggtgcgggccaagggcggtggcggctctggcggtgggagtgaggaggagctgcaagtgattcagcccgacaagtctgtaagtgtagccgctggcgagagcgccattctgcactgcaccgtgaccagtctgatccccgttggccctattcaatggttccggggtgccggacctgccagggagttgatctataatcaaaaggagggccattttccgcgggtcactacagtttctgagtctaccaagagagaaaatatggacttttccatcagtatctccaacatcacccctgctgatgccggaacgtactactgcgtgaaattcagaaaaggcagccccgacaccgaatttaaatctggcgccggtaccgaactgagtgtgagagctaagtgatga

SEQ ID NO: 49 - RNA, 1416 nt, synthetic construct
  source 1..1416: mol_type = other RNA
  modified_base order(11,17,20,21,30,38,44,65,67,68,72,73,75,80,85,89,92,93,96,98,105,107,108,112,115,116,117,123,143,146,148,150,169,170,179,188,189,190,198,215,219,220,221,227,228,229,233,235,240,242,243,255,265,268,270,271,275,280,281,291,297,303,307,308,315,321,324,332,334,338,357,363,366,370,375,378,379,394,395,398,413,415,416,417,419,421,422,423,435,441,446,449,452,454,470,471,475,479,482,485,486,489,491,493,504,512,517,518,519,522,523,526,530,531,537,539,545,552,580,582,585,592,599,602,604,608,610,611,615,617,620,630,631,635,639,649,655,657,662,664,669,675,676,677,687,689,690,701,703,717,737,738,739,741,744,746,750,767,777,782,788,790,793,795,797,800,808,809,810,811,813,822,824,825,830,831,835,841,846,861,864,865,879,884,887,891,897,900,904,905,906,907,908,909,911,913,916,922,923,929,933,937,939,943,957,959,961,962,963,967,970,972,974,975,977,981,987,989,996,1000,1002,1016,1018,1022,1026,1029,1045,1053,1059,1060,1072,1073,1079,1082,1089,1092,1098,1100,1102,1106,1122,1127,1128,1130,1135,1142,1149,1151,1152,1154,1160,1164,1169,1170,1174,1177,1178,1194,1204,1205,1208,1209,1210,1215,1231,1232,1233,1241,1250,1258,1263,1277,1281,1282,1283,1284,1285,1287,1289,1291,1295,1296,1304,1320,1327,1329,1330,1332,1333,1335,1337,1342,1343,1344,1354,1362,1369,1370,1371,1380,1394,1398,1400,1411,1414): mod_base = OTHER; note = Uracil
gaggaggagctgcaggtaattcagccagataagagcgtcagcgtggcggcaggggagagcgcgatcttgcattgtacagtcacatcactgattcctgtgggccctattcagtggtttcggggtgcaggccccgccagagagctgatctataaccaaaaagaggggcacttcccgcgggtaacaaccgtttcggagagtacaaagagagaaaacatggatttcagcatttcgatctcaaatattacccccgccgatgccggcacctactattgcgtgaagttccggaaaggtagccctgacactgagttcaagagtggggctggtacagagctgtcagtcagagccaagggaggagataaaactcatacctgccctccttgccccgccccagagttgctggggggccccagcgtatttctgtttccgcccaaacctaaagataccctgatgatctcgcggacgccagaggttacctgcgtcgtcgttgatgtatcccacgaggatcccgaagtgaaatttaattggtacgttgacggtgtcgaagtgcacaatgccaagacaaaaccccgggaggagcaatataatagcacctacagagtcgtctccgtattgactgtactgcaccaagattggctgaatggaaaggaatacaaatgtaaggtatccaataaggctttaccggcacctattgagaagaccatatccaaagccaaaggtcaacccagggaaccccaagtttatactctgcctccaagcagggaggagatgaccaagaatcaggtcagcctgtggtgtctcgtgaaaggcttttatcccagcgatattgccgttgagtgggaatcgaatgggcagcccgaaaataattacaaaaccacccctcccgtcctggatagcgatggtagcttttttctgtactccaaattgaccgtggataagtctagatggcagcagggcaatgtgtttagctgctctgttatgcatgaggctctgcacaatcactatacgcagaaaagcctgtccctgagtcctggcggagggggcggatccggaggtggcggttccgaagaagagttgcaggtaatccagcctgataaaagtgtgtcggtcgcggccggcgagagtgcaattctgcactgcaccgtgaccagtcttataccggtaggtcccattcagtggttcagaggcgccggccctgccagggaattgatttacaatcagaaggagggacactttcccagagtgacgaccgtcagcgaatccactaaaagggagaacatggatttttctatctccattagcaacatcaccccggccgacgctggcacgtattattgtgtgaaatttcgaaagggctcccccgatacagagtttaaaagcggtgcaggaaccgagctgagtgtccgggcgaagtgatga

SEQ ID NO: 50 - RNA, 1728 nt, synthetic construct
  source 1..1728: mol_type = other RNA
  modified_base order(5,11,14,32,35,42,53,54,59,64,79,80,81,83,85,86,88,90,94,101,106,110,123,134,139,143,152,157,159,169,172,175,177,178,183,191,202,203,209,231,236,238,242,248,257,263,278,280,283,286,298,300,303,309,310,311,316,317,322,325,341,344,350,352,355,380,382,383,389,396,400,406,431,436,438,440,443,451,454,455,467,473,475,478,494,506,510,514,515,516,522,524,525,527,542,544,546,547,549,551,560,563,569,573,577,584,598,602,604,611,618,624,638,650,657,661,663,664,682,684,687,691,702,707,710,720,725,727,728,729,731,733,734,738,741,747,758,761,764,777,782,787,791,794,797,803,807,816,819,824,829,830,831,835,838,842,851,857,864,892,904,911,914,918,920,923,929,932,943,947,961,967,974,978,989,993,996,999,1001,1002,1013,1035,1044,1049,1051,1058,1062,1066,1079,1094,1100,1105,1107,1112,1120,1121,1123,1129,1131,1134,1136,1142,1147,1153,1155,1158,1177,1191,1194,1196,1199,1216,1217,1219,1220,1223,1226,1228,1230,1235,1241,1249,1255,1271,1273,1274,1279,1286,1289,1301,1312,1324,1326,1328,1334,1336,1338,1357,1359,1362,1372,1374,1385,1391,1394,1401,1404,1408,1412,1418,1425,1432,1437,1439,1442,1447,1454,1463,1466,1472,1481,1486,1489,1490,1509,1517,1520,1522,1543,1544,1553,1562,1563,1589,1594,1595,1601,1602,1603,1605,1607,1614,1616,1623,1626,1629,1639,1642,1644,1645,1647,1649,1654,1655,1674,1681,1682,1695,1706,1708,1712,1723,1726): mod_base = OTHER; note = Uracil
gaggtccagctggtagagagcggaggcgacctggtgcagcctggcagaagccttagactgagctgcgccgcaagcggctttatcttctctaactacggcatgagctgggtcagacaggcccctggaaagggcctggaatgggtggccaccatcagctctgccagcacctactcatattaccctgacagcgtgaagggcagattcaccatcagccgggacaacgccaaaaatagcctgtacctgcaaatgaacagcctccgggtcgaggacaccgccctgtactactgcggccggcactctgatggaaatttcgccttcggctactggggccagggaacactggtgacagtgtcgtcagccagcaccaagggacccagcgtgttccccctggcccctagctccaagtccaccagcggcggaacagccgccctgggctgtctggtgaaagactacttccccgagcccgtcaccgtgtcctggaacagcggcgccctcaccagcggcgtgcatacctttcccgctgttctgcagagcagcggactgtattctctgagcagcgtggtgaccgtgcctagctccagcctgggcacccaaacatacatctgcaacgtgaaccataagcctagcaacaccaaggtggacaagaaggtggaacctaagtcttgcgacaagacccacacctgtcctccgtgccccgcccctgagctcctgggaggccctagcgtgtttctgttccctcctaaacctaaggacaccctgatgatcagccgcacccctgaggtgacatgcgtggtcgtggacgtgagtcacgaggatcctgaagtgaagtttaactggtacgtggacggagtggaagtgcacaatgccaagacaaaacccagagaagagcagtacaacagcacatacagagtggtgagtgtgctgaccgtgctgcaccaggactggctgaacggcaaggaatacaagtgcaaggtgagtaacaaggccctgcctgctcctattgagaagacgatcagcaaggccaagggccagcctagggagcctcaggtgtacaccctgcctccatccagagaagagatgacaaagaaccaggtgagcctcagctgtgccgtgaagggcttctacccctctgatatcgccgtggaatgggagtctaatggacagcccgagaacaactacaagacaacccctcctgtgctggacagcgacggcagcttcttcctggtgtctaaactgacagtggacaagtccagatggcagcagggcaacgtgttcagctgcagcgtgatgcacgaggccctgcacaaccactacacccagaaatctctgagcctgtctccaggaggcggcggcggctctggtggcggcggctctgaggaagaactgcaagtcatccagcctgataaatcggtcagcgtcgccgctggcgagtccgctatcctgcactgcaccgtgaccagcctgatccccgtgggcccgatccagtggttccggggcgcaggacccgctagagagctcatctacaaccagaaggaaggccacttcccaagagtgacaacagttagcgagagcaccaagagagaaaacatggacttcagcatttctatcagcaatatcacccctgctgatgccgggacatactattgtgtgaagttcagaaagggcagcccagatacagagttcaagagcggcgctggcaccgagctgtccgtgcgggccaaatgataa

SEQ ID NO: 51 - RNA, 1041 nt, synthetic construct
  source 1..1041: mol_type = other RNA
  modified_base order(5,8,11,24,26,28,32,36,38,45,54,57,62,67,86,89,93,95,109,113,118,121,125,142,144,152,155,158,160,162,167,169,174,178,179,188,195,199,200,201,208,210,225,226,227,228,233,239,248,263,269,271,274,277,280,281,293,296,301,307,308,326,327,332,344,354,359,361,362,365,367,368,372,376,378,381,389,394,410,413,415,419,422,430,431,433,447,452,457,464,476,489,490,492,503,528,532,539,551,557,559,561,571,587,589,595,602,617,619,627,629,640,641,655,675,687,698,704,707,714,717,725,727,729,731,738,752,755,760,762,767,776,777,779,785,794,799,802,803,819,830,833,835,856,857,861,866,875,877,879,883,885,902,907,908,909,914,915,916,918,920,929,930,936,942,952,955,957,958,960,962,963,967,968,979,984,994,995,1019,1025,1036,1039): mod_base = OTHER; note = Uracil
gacgtgctgatgacccagagccctctgtccctgcctgtgacacctggcgagcctgctagcatcagctgcagaagcagccgcaacatcgtgcatatcaacggcgacacctacctggaatggtacctgcagaagcccggccagtctccccagctgctcatctataaggtctccaatagattcagcggcgtgcccgatagatttagcggctctggcagcggcacagattttaccctgaaaatcagcagagtggaagccgaggacgtgggagtgtactactgcttccagggcagcctgctgccctggaccttcggccagggaacaaaggttgagatcaagagaaccgtcgccgcccctagcgtgttcatcttccctccatctgatgagcaactgaagtccggaaccgccagcgtggtgtgcctgctgaacaacttctaccccagagaagctaaagtgcagtggaaggtggacaacgccctgcagagcggaaattctcaggagagcgtgaccgagcaggacagcaaggacagtacctacagcctgagcagcaccctgacactgtctaaagccgactacgagaagcacaaggtgtacgcctgcgaggtgacccaccagggcctgtccagccctgtgaccaaaagcttcaaccggggcgaatgcggcggaggcggcagcggtggcggcggcagtgaagaggaactgcaagtgatccaacctgataagagcgtctctgtggccgctggagaaagcgccatcctgcactgtacagtgacgagccttatccccgtgggccccatccagtggttccggggagccggacctgccagagagctgatctacaaccagaaagagggccacttccctagagtgaccaccgtgtctgagtctacaaagcgggaaaacatggactttagcatttctatcagcaacattacccctgcagatgccggcacatactattgtgttaagttcaggaagggctcccctgacaccgaattcaagagcggcgccggcacagagctgagcgtgcgggccaagtgatga

SEQ ID NO: 52 - RNA, 1350 nt, synthetic construct
  source 1..1350: mol_type = other RNA
  modified_base order(5,11,14,15,32,35,36,53,59,61,63,64,79,80,83,85,86,94,101,106,110,123,134,139,143,152,163,165,169,175,178,191,202,203,209,219,236,238,242,248,253,255,257,263,270,278,280,282,283,286,297,298,300,303,310,311,316,317,322,325,341,344,350,352,354,355,357,375,380,382,383,384,387,389,396,426,429,431,435,436,438,440,443,451,454,455,467,473,475,478,492,494,506,513,514,515,516,519,522,524,527,542,544,551,553,556,558,560,563,569,573,576,580,582,584,598,602,604,611,624,625,630,638,650,657,664,682,684,687,691,696,699,702,707,710,717,721,723,725,727,728,731,733,734,753,758,761,764,777,782,787,789,791,794,797,803,824,829,830,835,838,842,851,857,864,892,898,904,906,911,914,916,920,923,929,932,943,947,961,967,974,976,989,999,1001,1013,1014,1015,1017,1044,1049,1051,1053,1058,1062,1065,1079,1094,1096,1100,1101,1105,1109,1112,1120,1121,1123,1128,1129,1131,1134,1136,1142,1147,1158,1167,1177,1191,1196,1199,1216,1217,1219,1220,1223,1225,1228,1230,1235,1241,1245,1249,1255,1269,1271,1273,1274,1275,1276,1279,1281,1286,1289,1301,1312,1328,1334,1341,1345,1348): mod_base = OTHER; note = Uracil
gaggtgcagctggttgaaagcggcggcgacctggttcaacccggcagaagcctgagactgtcttgcgccgccagcggattcatcttcagcaactacggcatgagctgggtgcggcaggcccctggcaagggcctggaatgggtcgccacaatcagcagcgcctctacctacagctactaccccgacagcgtgaaaggcagattcaccatcagcagagataacgccaagaacagcctgtacctgcagatgaactctctccgggtggaagataccgccctgtattactgcggcaggcattctgatggcaacttcgccttcggctactggggccaaggcaccctggtgaccgtgtcttctgccagcaccaagggccctagcgtctttcctctggcccctagcagcaagagcacaagcggaggcacagctgctctgggttgtctggtcaaggactacttccccgagcccgtgaccgtgtcctggaacagcggcgctctgacaagcggagtgcacacttttcctgctgtgctgcaaagcagcggcctgtacagcctgtcatctgtggtcaccgtgcctagtagctctctcggaacccagacctacatctgcaacgtgaaccacaagccttccaataccaaggtggacaagaaggtggagcctaaaagctgcgacaagacacacacatgtcctccgtgccctgctcctgagctgctgggcggtccctctgtgttcctgttcccaccaaagcccaaggataccctgatgatcagccggacccctgaggtgacgtgtgtggtggtggacgtgagccacgaggaccccgaggtgaagttcaactggtacgtggacggcgtggaagtgcacaatgccaaaacaaagccaagagaagagcagtacaactccacctatagagtggtctccgtgctgaccgtgctgcaccaggactggctgaacggcaaggaatacaaatgcaaggtgtccaacaaggccctgcccgcccctatcgagaagacaatttctaaagccaagggccagcccagagagcctcaggtgtataccctgcctcctagcagagaggaaatgaccaaaaaccaggtgtcccttacatgcctggtgaaaggcttctacccttctgatatcgccgtggaatgggagagcaatggccagcctgagaacaactacaagaccacccctccagtgctggacagcgacggcagcttcttcctgtactctaagctgaccgtggataagtcccggtggcagcagggcaatgtgttttcctgtagcgtgatgcacgaggccctgcacaaccactacacacagaagagcctgagcctgagccctggctgataa

SEQ ID NO: 53 - RNA, 1728 nt, synthetic construct
  source 1..1728: mol_type = other RNA
  modified_base order(5,11,14,32,35,42,53,59,63,64,66,79,80,83,85,86,94,101,106,110,134,139,143,152,163,165,169,171,175,178,180,183,186,191,192,202,203,209,236,238,242,248,257,263,270,278,280,283,286,298,300,303,309,310,311,316,317,322,325,341,344,350,355,357,360,375,376,378,380,381,382,383,387,389,397,399,412,414,417,431,436,440,443,451,454,455,467,471,473,478,493,494,506,514,515,522,524,527,542,544,547,549,551,553,556,558,560,561,563,569,573,574,576,577,580,582,584,598,602,604,611,625,627,630,638,650,657,664,682,684,687,691,702,707,710,725,727,728,729,731,733,734,735,747,758,761,764,777,782,787,791,794,797,803,805,824,829,830,831,835,838,842,846,851,857,879,892,904,906,911,914,920,923,929,932,943,947,961,967,974,989,993,996,1001,1013,1035,1049,1050,1051,1058,1062,1065,1068,1079,1094,1096,1100,1105,1109,1112,1120,1121,1122,1123,1129,1134,1136,1142,1147,1153,1167,1177,1191,1196,1199,1216,1217,1219,1220,1223,1225,1228,1235,1241,1255,1269,1271,1273,1274,1275,1279,1286,1289,1301,1305,1308,1312,1324,1328,1334,1336,1338,1341,1357,1359,1385,1391,1394,1401,1412,1414,1416,1418,1425,1439,1442,1447,1454,1459,1463,1466,1472,1479,1481,1482,1486,1489,1490,1506,1509,1517,1520,1522,1543,1544,1548,1553,1562,1564,1589,1593,1594,1595,1601,1607,1614,1616,1623,1626,1629,1639,1642,1645,1647,1649,1654,1655,1671,1681,1682,1687,1689,1698,1706,1708,1712,1723,1726): mod_base = OTHER; note = Uracil
gaagtgcagctggtggaaagcggcggcgacctggtccagcctggcagaagcctgcggctgagttgtgccgccagcggcttcatcttcagcaactacggcatgagctgggtgcggcaggcccccggcaagggcctggaatgggtcgccaccatcagcagcgcctctacctatagctactatcctgatagcgttaagggaagattcaccatcagccgcgacaacgccaagaacagcctgtacctgcagatgaacagcctgagagtcgaagataccgccctgtactactgcggccggcactctgatggaaatttcgccttcggctactggggccagggcacgctggtgacagtcagctctgctagcacaaaaggaccttctgttttccctctggccccatctagcaagagcacatctggtggcaccgccgccctgggctgcctggtgaaggactacttccccgagcccgtgactgtgagctggaacagcggcgccttgacaagcggcgtgcacaccttcccagctgtgctgcaaagcagcggcctgtactctctgtcctctgttgtgacagtgccttcttcctctctgggcacacagacctacatctgcaacgtgaaccacaagccctctaataccaaggtggacaaaaaggtggaacctaagagctgcgacaagacacacacatgtcctccatgccccgcacctgagctgctgggcggccccagcgtgtttctgtttcccccgaagcctaaggacaccctgatgatcagccggacccctgaagtgacctgcgtggtagtggacgtgtcccacgaggaccccgaggtgaaatttaactggtacgtggatggcgtggaggtgcacaacgccaaaacaaaacctagagaggaacagtacaacagcacctatagagtggtcagcgtgctgaccgtgctgcaccaggactggctgaacggcaaagagtacaagtgcaaggtgagcaacaaggccctgcctgctcccatcgagaagaccatcagcaaggccaagggacaacctagagagccccaggtttacacactgcctcctagtagggaggaaatgaccaagaaccaggtgtccctgacatgcctggtcaaaggcttttacccctccgatatcgccgtggaatgggagtccaacggccagcctgaaaacaactacaagaccacccctccagtgctggacagcgacggcagcttcttcctgtactccaagctgaccgtggacaagagcagatggcagcagggcaatgtgtttagctgcagcgtgatgcacgaggccctgcataatcactacacccagaagtccctgagcctctctcctggaggcggcggaggatctggcggaggcggcagcgaggaagagctgcaggtgatccagcctgacaaaagcgtgtctgtggccgctggcgagagcgccatcctgcactgcaccgtgacctccctgatccccgtgggccctattcagtggttccggggcgccggacctgctagagagctgatctacaaccaaaaggaaggccacttccctagagtgaccaccgtgtccgagagcaccaaaagagagaacatggatttcagcatcagcatcagcaatatcacccctgctgatgccggcacatactactgtgtgaagttcagaaagggcagccctgacaccgagttcaagtctggagccggtacagagctgtccgtgcgggccaagtgataa

SEQ ID NO: 54 - RNA, 1416 nt, synthetic construct
  source 1..1416: mol_type = other RNA
  modified_base order(11,17,20,27,34,36,38,39,40,42,44,51,58,60,65,68,69,73,80,89,92,98,105,107,108,112,115,116,143,146,148,169,170,171,179,188,196,213,215,220,221,227,233,242,243,249,252,255,265,268,271,275,280,281,297,300,307,308,332,334,336,338,370,379,384,387,390,395,398,409,411,413,415,416,419,421,422,429,435,446,449,452,454,465,470,475,477,479,482,485,491,504,507,512,513,517,518,523,526,530,539,545,567,580,592,594,599,602,604,608,611,617,620,624,631,635,649,655,662,664,666,677,681,687,689,701,723,737,739,746,750,767,782,784,788,793,797,800,808,809,811,824,830,835,841,843,846,865,879,882,884,885,886,887,897,901,903,904,905,906,907,908,911,913,923,929,933,943,959,961,962,963,967,974,977,989,1000,1016,1022,1045,1047,1059,1062,1073,1079,1082,1089,1100,1106,1127,1130,1135,1137,1142,1151,1154,1158,1160,1169,1174,1177,1178,1179,1194,1197,1205,1208,1210,1212,1215,1231,1232,1236,1241,1245,1250,1252,1277,1282,1283,1289,1295,1302,1304,1327,1330,1333,1335,1337,1342,1343,1369,1370,1375,1383,1394,1400,1411,1414): mod_base = OTHER; note = Uracil
gaggaggaactgcaggtcatccagcctgacaagtctgtttctgtggccgctggcgaatctgccatccttcactgcaccgtgacaagcctgatcccggtgggacctattcaatggttccggggcgccggccccgccagagagctgatctacaaccagaaagagggccactttccacgcgtgacaaccgtgagcgagtccaccaagcgggagaatatggacttcagcatcagcatcagcaacattacacctgctgatgccggcacatactactgcgtgaaattcagaaagggcagccctgataccgagttcaagagcggagccggcaccgagctgtctgtgcgggccaagggcggcgacaagacccacacatgccccccctgccctgctcctgaactgctgggcgggccatctgtgttcctgttcccccctaagcctaaagacaccctgatgatctccaggacccctgaggtgacctgtgtggtggtcgacgtgagccacgaagatcctgaggttaagttcaactggtacgtggacggcgtggaagtgcacaacgccaagaccaaacctagagaagagcagtacaacagcacctatagagtggtgtccgtgctgacagtgctgcatcaggactggctcaacggcaaggaatacaagtgcaaggtgtctaacaaggcactgcctgcccctatcgagaagaccatcagcaaggccaaaggccagcctagagaaccccaggtgtacacactgcctccaagcagagaggaaatgaccaagaaccaggtgtccctgacctgcctggtgaagggcttctaccccagcgacatcgccgtcgaatgggagtctaatggccagccagaaaacaactacaaaaccacccctcctgttttggacagcgatggatcttttttcctgtacagcaagctgaccgtggataagagcagatggcagcagggcaacgtgtttagctgcagcgtgatgcacgaggccctgcacaaccactacacccagaagagcctgagcctgagccccggcggcggcggcggatctggcggaggaggtagtgaggaagaactccaggtgatccaacctgacaagagcgtgagcgtggccgccggcgagagcgccatcctgcactgtacagtgaccagcctgatccctgtgggccccatccagtggtttcggggcgcgggccctgctagagagctgatctataatcaaaaagaaggccacttccctagagtgactaccgtgtccgagagcacaaagagagagaacatggacttcagcatcagcatcagcaatatcacacccgccgacgccggcacctactactgtgtgaagttcagaaagggcagcccggacacagagttcaaatccggcgctggaaccgagctgagcgtcagagccaagtgataa

SEQ ID NO: 55 - RNA, 2166 nt, synthetic construct
  source 1..2166: mol_type = other RNA
  modified_base order(11,12,17,20,27,30,34,36,38,40,44,45,58,65,68,72,73,75,80,81,84,89,90,92,98,102,105,107,108,112,115,116,123,132,143,146,148,150,168,169,170,171,179,186,188,192,207,215,219,220,221,223,227,229,233,234,235,240,242,265,267,268,270,271,273,275,276,280,281,291,292,294,307,308,309,313,321,324,332,338,345,351,360,361,373,375,386,392,395,409,413,415,417,419,420,433,440,441,443,447,448,450,455,460,463,464,467,473,474,477,482,483,487,490,491,492,504,507,518,521,523,528,543,544,545,546,552,554,561,563,565,567,571,588,590,594,595,596,597,598,602,604,608,615,617,618,621,640,643,645,646,650,655,656,657,666,667,669,672,675,682,683,684,688,706,707,709,711,713,717,720,729,732,745,747,754,756,765,769,770,772,773,777,780,788,790,791,792,794,796,797,798,816,821,822,824,827,829,831,845,850,854,857,860,866,868,873,887,892,893,897,898,901,903,905,914,915,920,924,955,966,967,969,974,977,978,979,981,983,985,986,992,994,995,999,1005,1006,1010,1024,1030,1032,1037,1039,1044,1050,1052,1059,1064,1076,1078,1086,1112,1114,1121,1129,1131,1142,1146,1157,1159,1161,1163,1168,1171,1172,1175,1182,1183,1184,1186,1188,1191,1192,1197,1199,1205,1210,1216,1240,1242,1248,1259,1260,1261,1262,1276,1279,1280,1281,1282,1283,1286,1287,1288,1290,1291,1298,1304,1312,1318,1334,1336,1337,1342,1344,1347,1349,1352,1356,1363,1364,1368,1371,1375,1389,1391,1392,1393,1397,1419,1420,1422,1428,1448,1454,1457,1464,1473,1475,1476,1479,1481,1482,1488,1495,1502,1503,1505,1509,1510,1517,1526,1529,1535,1536,1539,1544,1549,1552,1553,1554,1580,1581,1583,1584,1585,1587,1606,1607,1608,1616,1617,1620,1625,1627,1633,1650,1652,1657,1658,1664,1666,1668,1670,1671,1679,1692,1702,1704,1705,1707,1708,1712,1717,1718,1719,1722,1737,1740,1744,1745,1746,1750,1752,1755,1758,1761,1769,1771,1775,1788,1798,1800,1806,1810,1812,1823,1829,1830,1832,1833,1846,1848,1850,1851,1852,1856,1870,1877,1880,1885,1887,1890,1892,1893,1897,1901,1904,1910,1919,1924,1927,1928,1929,1935,1955,1956,1958,1960,1962,1965,1977,1980,1981,1982,1989,1991,2000,2010,2027,2032,2033,2034,2037,2039,2043,2045,2049,2052,2054,2055,2067,2073,2077,2080,2083,2087,2092,2093,2103,2119,2120,2121,2127,2130,2136,2143,2144,2148,2150,2161,2164): mod_base = OTHER; note = Uracil
gaagaggaacttcaagtaatccagcctgataagtctgtgtcagttgcggccggcgagtccgcaatcctacattgtacggttactagccttatccccgtaggtcctattcagtggttcaggggtgcgggacctgccagagagctgatatataaccaaaaagaaggacattttccgagagtcacgactgtgagtgagagcaccaagcgtgagaacatggatttctcgatctccatttccaatatcaccccagccgacgccggcacatattattgtgttaagttcagaaagggttctcccgacacagagtttaagtcgggcgctggtacagagctaagcgtccgggctaaaggtggaggcggttccggcggaggctctgaggaagagctgcaagtaatccaaccggacaaatcggtatctgttgccgccggggagtcagccattctgcattgtaccgtgacgtcgttaataccagttggtcccattcaatggtttcgcggggccggtcctgccagagaactgatctacaatcagaaggaagggcattttccacgtgtgaccactgtatctgaatcaaccaagagggagaatatggatttttcaatctcgatcagcaatattactcccgccgacgcagggacctactattgcgtaaagtttcggaaaggttctcctgataccgagtttaagtcaggagccgggaccgagttgtctgtacgtgctaagggaggtgataagacccacacgtgtccaccatgtcccgcccctgagttgttaggtggtcccagcgtgtttctctttccgcccaagcccaaggatacccttatgatctctaggacgcccgaagtaacctgcgtcgtcgtggacgtctcgcatgaggaccccgaggtcaagttcaattggtatgtcgacggagttgaggtgcataacgcaaaaacgaaaccgcgagaagagcagtacaacagcacttatcgcgtggtttctgtgttaaccgtcttgcatcaggattggctgaacgggaaagaatacaagtgtaaagtatcgaataaagctctcccagctccaatcgagaaaacgatatcgaaggctaaggggcagcccagggaaccgcaggtgtacacactcccgccgtctcgggaggagatgactaaaaaccaagtatctctaacatgcttggtgaaaggtttctatccttcagatatcgccgtagaatgggagtcaaacggccagccagaaaacaactataaaactacccccccggttttggacagcgacgggtcgtttttcctttattccaaactgaccgtagacaagtcccgatggcagcaaggaaacgtgttcagctgtagtgtgatgcatgaggccttacataatcactacacccagaagagtctttccctgagcccaggaggagggggaggttctggaggtggcgggagcgaggaagaactgcaagtgatacagcctgacaagagtgttagtgttgccgctggagaatccgcgattctacattgcacggtcaccagcctcatccccgttggtccgatccaatggtttagaggcgccggcccggcaagggagcttatttataaccaaaaggagggccactttccccgagttactaccgtgtccgaatccacaaaacgcgagaatatggacttcagcatatctattagcaacatcacgccggcagatgccgggacatattattgcgtcaagtttcgtaaggggagcccagatactgagtttaagtctggtgctggtaccgagctgtccgtgcgcgcaaaaggtggagggggctctgggggtggctctgaagaagagctgcaggttattcaacccgacaagtctgtttcggtcgccgcgggcgaatccgccatactgcactgtactgttacctcactgatccccgtgggaccaatacagtggtttaggggtgcaggccccgcgagggaacttatctataatcaaaaggagggtcatttcccccgtgtcacaaccgtcagcgaaagtaccaaaagggagaacatggactttagtatcagtatcagtaatattaccccagcagatgcgggtacgtactactgcgtgaaattccgaaaaggtagcccggacacggaatttaaaagtggtgcgggtacggagttgagtgtgcgcgccaagtgatga

SEQ ID NO: 56 - RNA, 1791 nt, synthetic construct
  source 1..1791: mol_type = other RNA
  modified_base order(11,17,20,21,27,38,40,42,44,48,51,63,65,68,72,73,80,85,87,89,92,98,105,107,112,115,116,117,132,135,143,146,147,148,169,170,179,188,190,215,220,221,223,227,233,235,237,240,242,249,252,255,265,268,271,275,280,281,307,308,309,332,338,339,360,386,392,395,413,415,419,440,443,448,455,464,467,471,473,474,482,487,490,491,507,518,521,523,543,544,545,554,563,571,573,588,590,595,596,602,608,617,624,640,643,645,646,648,650,655,656,672,675,682,683,688,707,709,711,713,732,744,745,747,750,754,759,765,770,773,783,788,790,791,792,794,796,797,801,821,824,827,829,831,840,845,850,854,857,860,864,866,879,882,887,892,893,898,901,905,912,914,915,920,942,955,967,969,974,977,983,986,992,995,1006,1010,1024,1030,1037,1039,1041,1052,1064,1076,1078,1098,1112,1113,1114,1121,1125,1128,1129,1131,1142,1157,1163,1168,1170,1172,1175,1183,1184,1186,1191,1192,1194,1197,1199,1200,1205,1210,1230,1240,1254,1259,1262,1276,1279,1280,1282,1283,1286,1288,1298,1304,1318,1334,1336,1337,1342,1349,1352,1364,1375,1391,1397,1399,1401,1448,1454,1457,1464,1467,1475,1476,1477,1479,1481,1502,1505,1510,1512,1517,1526,1529,1535,1542,1544,1549,1552,1553,1563,1569,1572,1580,1583,1585,1587,1590,1606,1607,1608,1611,1616,1625,1650,1652,1656,1657,1658,1659,1660,1662,1664,1666,1670,1672,1679,1702,1705,1708,1712,1717,1718,1744,1745,1758,1769,1775,1786,1789): mod_base = OTHER; note = Uracil
gaggaagagctgcaggtgattcaacctgacaagagcgtgtctgtggctgctggggagagcgctatcctccattgcacagtgacatctctgatccccgtgggccctatccagtggtttagaggcgccggccctgctagggaactgatttacaaccagaaagagggccacttccccagagtgacaaccgtgtccgagagcaccaagagagaaaacatggacttctccatcagcatctctaatatcacccctgctgatgccggaacctactactgcgtgaagttcagaaagggcagccccgacaccgagtttaagagcggcgccggaaccgagctgagcgttagagccaaaggcggcggcggtagcggcggcggcagcgaggaagagctgcaggtgatccagccagacaagagcgtgtccgtggccgccggagagagcgccatcctgcactgcaccgtgacaagcctgatccctgttggccccatccagtggttcaggggcgcaggccctgccagagaactgatctacaaccagaaggaaggccatttcccacgggtgacaacagtcagcgagtctaccaagcgggaaaatatggacttcagcatcagcatcagcaacatcacccctgccgacgccggcacctactattgtgtgaaattccggaaaggaagccctgataccgaattcaagtccggagccggcaccgagctgtctgtgagagccaagggcggagataagacccacacttgtcctccgtgccctgcccctgagctgctcggcggacctagcgtctttctgttccctccaaagcccaaggacaccctgatgatctctagaacccctgaggtgacctgcgtggtcgtcgatgtgagccacgaagatcctgaagtgaagttcaactggtacgtggacggtgttgaggtgcacaacgccaagaccaagcctcgcgaggaacagtacaacagcacatatagagtggtgagcgtgctgaccgtgctgcaccaggactggctgaacggcaaggaatacaagtgcaaggtgtctaacaaggccctgcccgcccccatcgagaagaccatctccaaggccaagggccagcctcgggagccccaggtttacaccctgcctccttctagagaagagatgaccaagaaccaggtgagcctgacatgtctggtgaagggcttctacccttctgatattgccgtggaatgggagagcaacggccagcctgagaacaactacaagacaacccctccagtgctggacagcgacggctcattcttcctgtacagcaagctgaccgtggacaaaagcagatggcagcagggcaacgtgttcagctgcagcgtgatgcacgaggccctccacaaccactacacccagaaaagcctgagcctgtctcccggcggcggcggaggcagcggcggaggcggaagcgaggaagagctgcaagtgatccagcctgataagagcgtttctgtcgccgccggagaaagcgccatcctgcactgtacagtgaccagcctgatccccgtgggccctatccaatggttccggggcgctggccctgctagagaactgatctataatcagaaggaaggccactttcctcgggtgaccaccgtgagcgaaagcaccaaaagagagaatatggatttttctatctccatctccaacatcacaccagccgacgccggcacatactactgcgtgaaattcagaaaaggcagccccgacacagagttcaagagcggcgctggaacagagctgagcgtgcgggccaagtgataa

SEQ ID NO: 57 - RNA, 1416 nt, synthetic construct
  source 1..1416: mol_type = other RNA
  modified_base order(11,17,20,21,27,30,34,36,38,44,48,51,65,67,68,73,80,84,85,89,92,96,98,105,107,112,115,116,129,132,143,146,148,165,168,169,170,179,188,196,215,220,221,222,227,233,235,240,242,243,246,249,264,265,268,271,273,275,280,281,292,297,307,308,309,313,315,318,321,327,332,334,336,338,354,363,370,375,378,379,387,395,398,402,413,415,416,417,419,421,422,423,426,429,441,444,446,449,452,462,465,470,475,479,482,485,491,507,512,517,518,523,526,528,530,537,539,545,580,588,592,594,599,602,604,608,611,617,620,630,631,635,649,655,662,669,677,684,687,689,701,703,705,737,739,744,746,767,782,784,786,788,790,793,797,800,807,808,809,811,816,817,824,828,830,835,841,843,849,861,864,865,867,879,882,884,887,894,897,901,903,904,905,907,908,909,910,911,913,915,923,927,929,937,939,943,954,959,961,962,964,966,967,970,972,974,977,989,996,1000,1002,1005,1014,1016,1020,1022,1024,1029,1038,1053,1059,1060,1072,1073,1079,1080,1082,1089,1100,1106,1116,1120,1122,1127,1130,1134,1135,1142,1150,1151,1154,1155,1158,1160,1169,1174,1177,1178,1191,1197,1205,1208,1209,1210,1231,1232,1236,1241,1245,1250,1252,1254,1260,1277,1281,1282,1283,1285,1287,1289,1290,1291,1293,1295,1297,1302,1304,1314,1323,1327,1330,1332,1333,1337,1342,1343,1344,1353,1362,1369,1370,1371,1375,1377,1380,1386,1394,1396,1398,1400,1407,1411,1414): mod_base = OTHER; note = Uracil
gaggaggagctgcaggtgattcagcctgataaatctgtgagcgtggctgctggggagagcgccatcttgcactgcaccgtgacttcactgatccctgtggggcctatccaatggttccggggcgccggtcctgccagagagctcatctacaaccagaaggagggtcatttccccagagtaaccaccgtaagcgagtccaccaagcgggagaacatggactttagcatcagcatctccaatattactcctgccgacgccgggacttactactgtgtgaagttcaggaagggctcccctgacaccgagtttaagtctggtgctggcactgagctgtctgtgagagccaaggggggtgacaagactcacacctgccctccttgccccgctccagagctgctgggtgggcccagcgtgtttctgtttcctcctaagcccaaggatactctgatgatcagccggactcctgaggtcacctgcgtggtagtggacgtcagccacgaggaccctgaggtgaagttcaactggtatgtagacggtgtggaggtacacaacgccaagaccaagcccagagaggagcagtacaacagtacctatagggtggtgtcggtgctgaccgtgctgcaccaggattggctgaacggcaaggagtacaagtgcaaggtgagcaataaggccctgcccgctcctatcgagaagaccatctctaaggccaaggggcagccccgggaaccccaggtatacactctgccccccagcagggaggagatgaccaagaaccaagtatctctgtggtgcctggtcaagggtttctacccttccgacatcgctgtggagtgggagtctaacggtcagcccgagaataattataagaccacccctcctgtcctggacagtgatgggtctttctttttgtatagcaagctgactgtggacaagtctaggtggcagcagggtaacgtgttctcttgctctgtgatgcacgaggccctgcacaatcactatactcagaagagtctgagtctgtcccctggcgggggtgggggaagcggcggtgggggttccgaggaggagttgcaagttatacagcctgacaagagcgtgagcgtggccgccggtgagtctgccatcctgcattgcaccgtcaccagcttgattcctgtgggccccatccagtggttcagaggggccggtcccgctcgggagctgatttacaaccagaaagagggccacttccctagggtgactaccgtgtctgagagtaccaagagagagaacatggatttctctatttctatctccaatatcacccccgctgacgccggtacctactattgcgtcaagtttcgcaagggtagccccgataccgagtttaaatctggtgccggtacggagctgtctgtccgggctaagtgatga

SEQ ID NO: 58 - RNA, 2166 nt, synthetic construct
  source 1..2166: mol_type = other RNA
  modified_base order(10,11,17,20,21,34,36,38,39,44,45,48,54,65,68,73,78,80,85,87,89,92,93,96,98,105,107,112,115,116,120,123,126,143,146,147,148,150,153,165,169,170,179,180,188,190,196,213,215,219,220,221,227,229,233,235,242,243,252,255,265,267,268,270,271,275,276,280,281,282,292,294,300,307,308,309,313,315,332,334,338,339,357,360,363,366,373,386,392,395,396,405,413,415,419,423,429,433,435,440,441,443,447,448,450,455,464,467,471,473,482,487,490,491,492,504,510,518,521,523,528,540,544,545,546,554,563,565,567,590,594,595,596,598,600,602,608,609,617,630,633,640,642,643,646,650,655,656,657,669,672,678,682,683,688,696,699,707,713,729,732,745,753,754,759,765,770,772,773,780,784,786,788,789,790,791,794,796,797,798,821,824,827,829,831,845,849,850,854,857,860,864,866,868,870,887,892,893,894,897,898,901,905,909,914,920,927,936,955,961,966,967,974,977,983,984,986,990,992,995,999,1005,1006,1009,1010,1014,1017,1024,1030,1037,1050,1052,1059,1064,1076,1077,1086,1092,1098,1112,1114,1121,1129,1142,1157,1159,1163,1168,1172,1175,1182,1183,1184,1186,1192,1199,1203,1205,1210,1240,1254,1259,1261,1262,1266,1267,1272,1276,1278,1279,1280,1281,1282,1283,1285,1286,1288,1298,1304,1312,1314,1318,1332,1334,1336,1337,1339,1342,1345,1349,1352,1362,1364,1374,1375,1391,1395,1397,1407,1413,1419,1428,1431,1447,1448,1454,1455,1457,1464,1475,1477,1481,1482,1495,1502,1505,1509,1510,1517,1522,1524,1526,1529,1530,1535,1539,1544,1545,1549,1552,1553,1560,1566,1569,1580,1583,1585,1605,1606,1607,1608,1616,1625,1633,1635,1650,1652,1656,1657,1658,1659,1660,1662,1664,1666,1670,1671,1672,1674,1677,1679,1689,1692,1702,1704,1705,1708,1712,1717,1718,1719,1728,1729,1737,1744,1745,1764,1769,1771,1775,1791,1798,1800,1806,1812,1823,1829,1832,1833,1846,1848,1850,1854,1856,1863,1877,1878,1880,1885,1892,1899,1901,1904,1910,1911,1917,1919,1920,1924,1927,1928,1935,1944,1954,1955,1958,1960,1962,1965,1980,1981,1982,1983,1991,1995,2000,2001,2002,2004,2008,2010,2025,2027,2032,2033,2034,2035,2039,2043,2045,2047,2054,2061,2064,2067,2077,2080,2083,2087,2092,2093,2119,2120,2121,2125,2127,2136,2144,2148,2150,2157,2161,2164): mod_base = OTHER; note = Uracil
gaggaagagttgcaagtcattcaaccggacaagtctgttagcgttgctgcaggtgagagcgccatcctccactgcactgtcacgtctctcattcctgtaggccctatccagtggttccgtggtgctggccccgccagggaactgatttataatcagaaagaaggtcacttccccagagttaccaccgtctccgagtccaccaagcgggagaatatggatttcagcatctccatctcgaacattacccccgctgatgccggcacgtattattgcgttaagtttagaaagggctctccagataccgaatttaagtctggcgccggcaccgagctctccgttagagccaaaggcggcggtggtagtggtgggggctccgaggaggagctccaggtgattcaaccggataagagcgtgtccgtggctgccggtgagtctgccattctgcattgtacagtgaccagcctgatccctgtggggcccatccagtggtttcggggagccggtccggctcgggaactgatctacaatcagaaggagggtcactttcccagagtgacgaccgtgtctgagagcaccaagagggagaacatggatttctctatcagcattagcaacatcacgcccgccgatgctgggacctattactgcgtgaagtttaggaagggcagtcctgacactgagttcaagtccggggctggtaccgagctgagcgtcagggccaaaggcggtgataagacccacacctgcccgccttgccctgcccctgagctgttgggcggtccgtctgttttcctgtttccgccaaagcccaaagacaccctgatgatctctagaaccccggaggtcacttgcgtcgtggtcgatgtgtctcacgaggaccccgaggtaaagtttaattggtacgtcgatggcgtggaggtgcacaatgccaaaactaagcccagggaggagcaatacaactccacttacagggtggtgagcgttctgactgtgctgcatcaggattggttgaatggtaaggagtacaaatgcaaggtcagcaacaaagctctgccggctcccatcgagaaaaccattagcaaggctaagggtcagcctagggaaccccaagtgtacaccctgcccccctccagagaggagatgacgaagaaccaagtatccctgacgtgcctggtaaagggtttctacccatcggacatcgctgtggagtgggagagcaacggccagcccgagaacaactacaagacgacccctccggtcttggattccgatggctcttttttcttgtacagcaagctgaccgtggacaaatctaggtggcaacaagggaatgtgttctcctgctccgtgatgcacgaggctctacacaaccattacacccagaaaagcctgagtctgagccccggtgggggtgggggtagcggcggtggtggcagcgaggaggagttgcaggttatacagcctgacaagagcgtgtcagttgccgccggcgagtccgcaatcctccattgcaccgtgacatctctgattcccgtgggtcccattcagtggttcagaggtgcaggtcctgcgagagagctgatctacaaccagaaggaggggcattttccgagggtaacaacagtcagcgaatctaccaagcgggaaaatatggatttttctatctccatttctaatatcacgcccgctgatgcgggcacatattactgcgtcaagtttaggaaaggttcaccggatacagagttcaagagcggggccggcactgagctgtcggtgcgggccaagggcggtggcggctctggcggtgggagtgaggaggagctgcaagtgattcagcccgacaagtctgtaagtgtagccgctggcgagagcgccattctgcactgcaccgtgaccagtctgatccccgttggccctattcaatggttccggggtgccggacctgccagggagttgatctataatcaaaaggagggccattttccgcgggtcactacagtttctgagtctaccaagagagaaaatatggacttttccatcagtatctccaacatcacccctgctgatgccggaacgtactactgcgtgaaattcagaaaaggcagccccgacaccgaatttaaatctggcgccggtaccgaactgagtgtgagagctaagtgatga

SEQ ID NO: 59 - protein, 617 aa, synthetic construct
MDMRVPAQLLGLLLLWLRGARCEEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKGGGGSGGGSEEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSEEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAK

SEQ ID NO: 60 - protein, 742 aa, synthetic construct
MDMRVPAQLLGLLLLWLRGARCEEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKGGGGSGGGSEEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSEEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKGGGGSGGGSEEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAK

SEQ ID NO: 61 - RNA, 2235 nt, synthetic construct
  source 1..2235: mol_type = other RNA; note = Exemplary RNA sequence (minus UTRs) encoding an amino acid sequence containing the amino acid sequence represented by SEQ ID NO: 13 (Octavalent)
  modified_base order(2,6,8,14,26,28,29,33,35,38,41,44,46,50,60,64,66,76,77,83,86,87,100,102,104,105,110,111,114,120,131,134,139,144,146,151,153,155,158,159,162,164,171,173,178,181,182,186,189,192,209,212,213,214,216,219,231,235,236,245,246,254,256,262,279,281,285,286,287,293,295,299,301,308,309,318,321,331,333,334,336,337,341,342,346,347,348,358,360,366,373,374,375,379,381,398,400,404,405,423,426,429,432,442,455,461,464,465,474,482,484,488,492,498,502,504,509,510,512,516,517,519,524,533,536,540,542,551,556,559,560,561,573,579,587,590,592,597,609,613,614,615,623,632,634,636,659,663,664,665,667,669,671,677,678,686,699,702,709,711,712,715,719,724,725,726,738,741,747,751,752,757,765,768,776,782,798,801,814,822,823,828,834,839,841,842,849,853,855,857,858,859,860,863,865,866,867,890,893,896,898,900,914,918,919,923,926,929,933,935,937,939,956,961,962,963,966,967,970,974,978,983,989,996,1005,1024,1030,1035,1036,1043,1046,1052,1053,1055,1059,1061,1064,1068,1074,1075,1078,1079,1083,1086,1093,1099,1106,1119,1121,1128,1133,1145,1146,1155,1161,1167,1181,1183,1190,1198,1211,1226,1228,1232,1237,1241,1244,1251,1252,1253,1255,1261,1268,1272,1274,1279,1309,1323,1328,1330,1331,1335,1336,1341,1345,1347,1348,1349,1350,1351,1352,1354,1355,1357,1367,1373,1381,1383,1387,1401,1403,1405,1406,1408,1411,1414,1418,1421,1431,1433,1443,1444,1460,1464,1466,1476,1482,1488,1497,1500,1516,1517,1523,1524,1526,1533,1544,1546,1550,1551,1564,1571,1574,1578,1579,1586,1591,1593,1595,1598,1599,1604,1608,1613,1614,1618,1621,1622,1629,1635,1638,1649,1652,1654,1674,1675,1676,1677,1685,1694,1702,1704,1719,1721,1725,1726,1727,1728,1729,1731,1733,1735,1739,1740,1741,1743,1746,1748,1758,1761,1771,1773,1774,1777,1781,1786,1787,1788,1797,1798,1806,1813,1814,1833,1838,1840,1844,1860,1867,1869,1875,1884,1895,1901,1904,1905,1918,1920,1922,1926,1928,1935,1949,1950,1952,1957,1964,1971,1973,1976,1982,1983,1989,1991,1992,1996,1999,2000,2007,2016,2026,2027,2030,2032,2034,2037,2052,2053,2054,2055,2063,2067,2072,2073,2074,2076,2080,2082,2097,2099,2104,2105,2106,2107,2111,2115,2117,2119,2126,2133,2136,2139,2149,2152,2155,2159,2164,2165,2191,2192,2193,2197,2199,2208,2216,2220,2222,2229,2233): mod_base = OTHER; note = Uracil
atggatatgagagtacccgcccaactgttgggtctgctgctgctgtggctgagaggcgctcggtgtgaggaagagttgcaagtcattcaaccggacaagtctgttagcgttgctgcaggtgagagcgccatcctccactgcactgtcacgtctctcattcctgtaggccctatccagtggttccgtggtgctggccccgccagggaactgatttataatcagaaagaaggtcacttccccagagttaccaccgtctccgagtccaccaagcgggagaatatggatttcagcatctccatctcgaacattacccccgctgatgccggcacgtattattgcgttaagtttagaaagggctctccagataccgaatttaagtctggcgccggcaccgagctctccgttagagccaaaggcggcggtggtagtggtgggggcggctccgaggaggagctccaggtgattcaaccggataagagcgtgtccgtggctgccggtgagtctgccattctgcattgtacagtgaccagcctgatccctgtggggcccatccagtggtttcggggagccggtccggctcgggaactgatctacaatcagaaggagggtcactttcccagagtgacgaccgtgtctgagagcaccaagagggagaacatggatttctctatcagcattagcaacatcacgcccgccgatgctgggacctattactgcgtgaagtttaggaagggcagtcctgacactgagttcaagtccggggctggtaccgagctgagcgtcagggccaaaggcggtgataagacccacacctgcccgccttgccctgcccctgagctgttgggcggtccgtctgttttcctgtttccgccaaagcccaaagacaccctgatgatctctagaaccccggaggtcacttgcgtcgtggtcgatgtgtctcacgaggaccccgaggtaaagtttaattggtacgtcgatggcgtggaggtgcacaatgccaaaactaagcccagggaggagcaatacaactccacttacagggtggtgagcgttctgactgtgctgcatcaggattggttgaatggtaaggagtacaaatgcaaggtcagcaacaaagctctgccggctcccatcgagaaaaccattagcaaggctaagggtcagcctagggaaccccaagtgtacaccctgcccccctccagagaggagatgacgaagaaccaagtatccctgacgtgcctggtaaagggtttctacccatcggacatcgctgtggagtgggagagcaacggccagcccgagaacaactacaagacgacccctccggtcttggattccgatggctcttttttcttgtacagcaagctgaccgtggacaaatctaggtggcaacaagggaatgtgttctcctgctccgtgatgcacgaggctctacacaaccattacacccagaaaagcctgagtctgagccccggtgggggtgggggtagcggcggtggtggcagcgaggaggagttgcaggttatacagcctgacaagagcgtgtcagttgccgccggcgagtccgcaatcctccattgcaccgtgacatctctgattcccgtgggtcccattcagtggttcagaggtgcaggtcctgcgagagagctgatctacaaccagaaggaggggcattttccgagggtaacaacagtcagcgaatctaccaagcgggaaaatatggatttttctatctccatttctaatatcacgcccgctgatgcgggcacatattactgcgtcaagtttaggaaaggttcaccggatacagagttcaagagcggggccggcactgagctgtcggtgcgggccaagggcggtggcggctctggcggtgggggcagtgaggaggagctgcaagtgattcagcccgacaagtctgtaagtgtagccgctggcgagagcgccattctgcactgcaccgtgaccagtctgatccccgttggccctattcaatggttccggggtgccggacctgccagggagttgatctataatcaaaaggagggccattttccgcgggtcactacagtttctgagtctaccaagagagaaaatatggacttttccatcagtatctccaacatcacccctgctgatgccggaacgtactactgcgtgaaattcagaaaaggcagccccgacaccgaatttaaatctggcgccggtaccgaactgagtgtgagagctaagtga

SEQ ID NO: 62 - RNA, 1791 nt, synthetic construct
  source 1..1791: mol_type = other RNA; note = Exemplary RNA sequence (minus UTRs) encoding the amino acid sequence represented by SEQ ID NO: 12 (Hexavalent)
  modified_base order(11,17,20,21,27,38,40,42,44,48,51,63,65,68,72,73,80,85,87,89,92,98,105,107,112,115,116,117,132,135,143,146,147,148,169,170,179,188,190,215,220,221,223,227,233,235,237,240,242,249,252,255,265,268,271,275,280,281,307,308,309,332,338,339,360,389,395,398,416,418,422,443,446,451,458,467,470,474,476,477,485,490,493,494,510,521,524,526,546,547,548,557,566,574,576,591,593,598,599,605,611,620,627,643,646,648,649,651,653,658,659,675,678,685,686,691,710,712,714,716,735,747,748,750,753,757,762,768,773,776,786,791,793,794,795,797,799,800,804,824,827,830,832,834,843,848,853,857,860,863,867,869,882,885,890,895,896,901,904,908,915,917,918,923,945,958,970,972,977,980,986,989,995,998,1009,1013,1027,1033,1040,1042,1044,1055,1067,1079,1081,1101,1115,1116,1117,1124,1128,1131,1132,1134,1145,1160,1166,1171,1173,1175,1178,1186,1187,1189,1194,1195,1197,1200,1202,1203,1208,1213,1233,1243,1257,1262,1265,1279,1282,1283,1285,1286,1289,1291,1301,1307,1321,1337,1339,1340,1345,1352,1355,1367,1378,1394,1400,1402,1404,1451,1457,1460,1467,1470,1478,1479,1480,1482,1484,1505,1508,1513,1515,1520,1529,1532,1538,1545,1547,1552,1555,1556,1566,1572,1575,1583,1586,1588,1590,1593,1609,1610,1611,1614,1619,1628,1653,1655,1659,1660,1661,1662,1663,1665,1667,1669,1673,1675,1682,1705,1708,1711,1715,1720,1721,1747,1748,1761,1772,1778,1789): mod_base = OTHER; note = Uracil
gaggaagagctgcaggtgattcaacctgacaagagcgtgtctgtggctgctggggagagcgctatcctccattgcacagtgacatctctgatccccgtgggccctatccagtggtttagaggcgccggccctgctagggaactgatttacaaccagaaagagggccacttccccagagtgacaaccgtgtccgagagcaccaagagagaaaacatggacttctccatcagcatctctaatatcacccctgctgatgccggaacctactactgcgtgaagttcagaaagggcagccccgacaccgagtttaagagcggcgccggaaccgagctgagcgttagagccaaaggcggcggcggtagcggcggcggcggcagcgaggaagagctgcaggtgatccagccagacaagagcgtgtccgtggccgccggagagagcgccatcctgcactgcaccgtgacaagcctgatccctgttggccccatccagtggttcaggggcgcaggccctgccagagaactgatctacaaccagaaggaaggccatttcccacgggtgacaacagtcagcgagtctaccaagcgggaaaatatggacttcagcatcagcatcagcaacatcacccctgccgacgccggcacctactattgtgtgaaattccggaaaggaagccctgataccgaattcaagtccggagccggcaccgagctgtctgtgagagccaagggcggagataagacccacacttgtcctccgtgccctgcccctgagctgctcggcggacctagcgtctttctgttccctccaaagcccaaggacaccctgatgatctctagaacccctgaggtgacctgcgtggtcgtcgatgtgagccacgaagatcctgaagtgaagttcaactggtacgtggacggtgttgaggtgcacaacgccaagaccaagcctcgcgaggaacagtacaacagcacatatagagtggtgagcgtgctgaccgtgctgcaccaggactggctgaacggcaaggaatacaagtgcaaggtgtctaacaaggccctgcccgcccccatcgagaagaccatctccaaggccaagggccagcctcgggagccccaggtttacaccctgcctccttctagagaagagatgaccaagaaccaggtgagcctgacatgtctggtgaagggcttctacccttctgatattgccgtggaatgggagagcaacggccagcctgagaacaactacaagacaacccctccagtgctggacagcgacggctcattcttcctgtacagcaagctgaccgtggacaaaagcagatggcagcagggcaacgtgttcagctgcagcgtgatgcacgaggccctccacaaccactacacccagaaaagcctgagcctgtctcccggcggcggcggaggcagcggcggaggcggaagcgaggaagagctgcaagtgatccagcctgataagagcgtttctgtcgccgccggagaaagcgccatcctgcactgtacagtgaccagcctgatccccgtgggccctatccaatggttccggggcgctggccctgctagagaactgatctataatcagaaggaaggccactttcctcgggtgaccaccgtgagcgaaagcaccaaaagagagaatatggatttttctatctccatctccaacatcacaccagccgacgccggcacatactactgcgtgaaattcagaaaaggcagccccgacacagagttcaagagcggcgctggaacagagctgagcgtgcgggccaagtga

SEQ ID NO: 63 - RNA, 2169 nt, synthetic construct
  source 1..2169: mol_type = other RNA; note = Exemplary RNA sequence (minus UTRs) encoding the amino acid sequence represented by SEQ ID NO: 13 (Octavalent)
  modified_base order(10,11,17,20,21,34,36,38,39,44,45,48,54,65,68,73,78,80,85,87,89,92,93,96,98,105,107,112,115,116,120,123,126,143,146,147,148,150,153,165,169,170,179,180,188,190,196,213,215,219,220,221,227,229,233,235,242,243,252,255,265,267,268,270,271,275,276,280,281,282,292,294,300,307,308,309,313,315,332,334,338,339,357,360,363,366,376,389,395,398,399,408,416,418,422,426,432,436,438,443,444,446,450,451,453,458,467,470,474,476,485,490,493,494,495,507,513,521,524,526,531,543,547,548,549,557,566,568,570,593,597,598,599,601,603,605,611,612,620,633,636,643,645,646,649,653,658,659,660,672,675,681,685,686,691,699,702,710,716,732,735,748,756,757,762,768,773,775,776,783,787,789,791,792,793,794,797,799,800,801,824,827,830,832,834,848,852,853,857,860,863,867,869,871,873,890,895,896,897,900,901,904,908,912,917,923,930,939,958,964,969,970,977,980,986,987,989,993,995,998,1002,1008,1009,1012,1013,1017,1020,1027,1033,1040,1053,1055,1062,1067,1079,1080,1089,1095,1101,1115,1117,1124,1132,1145,1160,1162,1166,1171,1175,1178,1185,1186,1187,1189,1195,1202,1206,1208,1213,1243,1257,1262,1264,1265,1269,1270,1275,1279,1281,1282,1283,1284,1285,1286,1288,1289,1291,1301,1307,1315,1317,1321,1335,1337,1339,1340,1342,1345,1348,1352,1355,1365,1367,1377,1378,1394,1398,1400,1410,1416,1422,1431,1434,1450,1451,1457,1458,1460,1467,1478,1480,1484,1485,1498,1505,1508,1512,1513,1520,1525,1527,1529,1532,1533,1538,1542,1547,1548,1552,1555,1556,1563,1569,1572,1583,1586,1588,1608,1609,1610,1611,1619,1628,1636,1638,1653,1655,1659,1660,1661,1662,1663,1665,1667,1669,1673,1674,1675,1677,1680,1682,1692,1695,1705,1707,1708,1711,1715,1720,1721,1722,1731,1732,1740,1747,1748,1767,1772,1774,1778,1794,1801,1803,1809,1818,1829,1835,1838,1839,1852,1854,1856,1860,1862,1869,1883,1884,1886,1891,1898,1905,1907,1910,1916,1917,1923,1925,1926,1930,1933,1934,1941,1950,1960,1961,1964,1966,1968,1971,1986,1987,1988,1989,1997,2001,2006,2007,2008,2010,2014,2016,2031,2033,2038,2039,2040,2041,2045,2049,2051,2053,2060,2067,2070,2073,2083,2086,2089,2093,2098,2099,2125,2126,2127,2131,2133,2142,2150,2154,2156,2163,2167): mod_base = OTHER; note = Uracil
gaggaagagttgcaagtcattcaaccggacaagtctgttagcgttgctgcaggtgagagcgccatcctccactgcactgtcacgtctctcattcctgtaggccctatccagtggttccgtggtgctggccccgccagggaactgatttataatcagaaagaaggtcacttccccagagttaccaccgtctccgagtccaccaagcgggagaatatggatttcagcatctccatctcgaacattacccccgctgatgccggcacgtattattgcgttaagtttagaaagggctctccagataccgaatttaagtctggcgccggcaccgagctctccgttagagccaaaggcggcggtggtagtggtgggggcggctccgaggaggagctccaggtgattcaaccggataagagcgtgtccgtggctgccggtgagtctgccattctgcattgtacagtgaccagcctgatccctgtggggcccatccagtggtttcggggagccggtccggctcgggaactgatctacaatcagaaggagggtcactttcccagagtgacgaccgtgtctgagagcaccaagagggagaacatggatttctctatcagcattagcaacatcacgcccgccgatgctgggacctattactgcgtgaagtttaggaagggcagtcctgacactgagttcaagtccggggctggtaccgagctgagcgtcagggccaaaggcggtgataagacccacacctgcccgccttgccctgcccctgagctgttgggcggtccgtctgttttcctgtttccgccaaagcccaaagacaccctgatgatctctagaaccccggaggtcacttgcgtcgtggtcgatgtgtctcacgaggaccccgaggtaaagtttaattggtacgtcgatggcgtggaggtgcacaatgccaaaactaagcccagggaggagcaatacaactccacttacagggtggtgagcgttctgactgtgctgcatcaggattggttgaatggtaaggagtacaaatgcaaggtcagcaacaaagctctgccggctcccatcgagaaaaccattagcaaggctaagggtcagcctagggaaccccaagtgtacaccctgcccccctccagagaggagatgacgaagaaccaagtatccctgacgtgcctggtaaagggtttctacccatcggacatcgctgtggagtgggagagcaacggccagcccgagaacaactacaagacgacccctccggtcttggattccgatggctcttttttcttgtacagcaagctgaccgtggacaaatctaggtggcaacaagggaatgtgttctcctgctccgtgatgcacgaggctctacacaaccattacacccagaaaagcctgagtctgagccccggtgggggtgggggtagcggcggtggtggcagcgaggaggagttgcaggttatacagcctgacaagagcgtgtcagttgccgccggcgagtccgcaatcctccattgcaccgtgacatctctgattcccgtgggtcccattcagtggttcagaggtgcaggtcctgcgagagagctgatctacaaccagaaggaggggcattttccgagggtaacaacagtcagcgaatctaccaagcgggaaaatatggatttttctatctccatttctaatatcacgcccgctgatgcgggcacatattactgcgtcaagtttaggaaaggttcaccggatacagagttcaagagcggggccggcactgagctgtcggtgcgggccaagggcggtggcggctctggcggtgggggcagtgaggaggagctgcaagtgattcagcccgacaagtctgtaagtgtagccgctggcgagagcgccattctgcactgcaccgtgaccagtctgatccccgttggccctattcaatggttccggggtgccggacctgccagggagttgatctataatcaaaaggagggccattttccgcgggtcactacagtttctgagtctaccaagagagaaaatatggacttttccatcagtatctccaacatcacccctgctgatgccggaacgtactactgcgtgaaattcagaaaaggcagccccgacaccgaatttaaatctggcgccggtaccgaactgagtgtgagagctaagtga

SEQ ID NO: 64 - RNA, 1887 nt, synthetic construct
  source 1..1887: mol_type = other RNA; note = Exemplary RNA sequence (minus UTRs) encoding the amino acid sequence containing the amino acid sequence represented by SEQ ID NO: 35 (1540)
  modified_base order(2,8,14,18,26,29,35,38,41,44,46,50,64,66,77,83,86,104,106,108,110,111,124,126,129,131,132,134,139,141,146,151,153,155,158,162,164,173,178,181,182,198,201,209,210,212,214,235,236,240,245,254,255,262,264,281,285,286,287,288,289,291,293,295,299,301,306,308,315,318,331,334,337,341,346,347,366,373,374,375,398,404,426,439,441,482,488,491,498,509,511,515,519,536,539,544,551,560,563,569,576,578,583,586,587,588,614,617,619,640,641,650,659,661,667,686,690,691,692,694,696,698,700,704,713,729,736,739,742,744,746,751,752,768,771,778,779,803,809,841,850,855,861,866,869,879,880,882,884,886,887,888,890,892,893,906,917,920,923,941,945,946,950,953,956,962,975,978,983,988,989,994,997,1001,1010,1016,1038,1051,1062,1063,1065,1070,1073,1075,1077,1079,1082,1088,1091,1102,1106,1120,1126,1133,1148,1155,1158,1160,1172,1203,1208,1210,1217,1221,1224,1238,1253,1259,1264,1266,1268,1271,1279,1280,1282,1295,1296,1301,1306,1317,1326,1336,1355,1358,1368,1375,1376,1378,1379,1382,1384,1387,1389,1394,1400,1413,1414,1430,1432,1433,1438,1445,1448,1460,1464,1467,1471,1483,1485,1487,1493,1495,1497,1500,1515,1531,1544,1550,1553,1560,1563,1571,1577,1598,1601,1606,1613,1621,1622,1625,1629,1631,1640,1645,1648,1649,1665,1676,1679,1681,1683,1701,1702,1703,1712,1721,1723,1725,1748,1753,1754,1760,1766,1767,1768,1770,1773,1775,1782,1785,1788,1798,1801,1804,1808,1813,1814,1815,1830,1840,1841,1854,1865,1867,1871,1872,1882,1885): mod_base = OTHER; note = Uracil
atggacatgcgggtgcctgcccagctgctgggcctgctgctcctgtggctgagaggagccaggtgtgaagaggaactgcaggtgatccaacccgacaagagcgtgtctgttgccgccggcgagtctgctattctgcactgtaccgtgacatctctcatccctgtgggaccgatccagtggttccggggcgccggccctgctagagagcttatctacaaccagaaagaaggccacttccctcgggtgaccacagttagcgagtctaccaagagagaaaacatggatttttctatctcaatctccaatatcacccctgctgacgccggaacatactactgcgtgaagttcagaaagggcagcccagatacagagtttaagagcggagccggcacagagctgagcgtgcgggccaagggcggcggcggtagcagcagcgcatctaccgacaagacacacacaggcggcggcagcgaggaggagctgcaagtgatccagcctgacaaaagcgtgtccgtggctgccggcgagagcgccatcctgcactgcaccgtgaccagcctgatccccgtgggccctatccagtggtttagaggcgccggccccgccagagaactgatctacaaccagaaagagggacacttccccagggtgacaaccgtgtccgagtccaccaaaagagagaacatggatttctctatctccatcagcaacatcaccccagccgacgctggaacctactactgtgtgaagttcagaaagggaagccctgataccgagttcaagagcggcgccggcaccgagctgagcgtcagagccaagggcggcgacaagacccacacctgccccccatgccctgcccctgagctgctgggcggaccttctgtgtttctgttcccccccaagcctaaggacaccctgatgatcagcagaacccccgaggtgacttgcgtggtggtcgacgtgagccacgaagatcctgaggtgaagttcaactggtacgtggacggcgtggaagtgcacaacgccaaaacaaaacctagagaggaacagtacaacagcacttatagagtggtgtctgtcctgaccgtgctgcaccaggactggctgaacggcaaggagtacaagtgcaaggtgagcaacaaggccctgcccgctcctatcgagaagaccatcagcaaggccaagggccagccaagagagcctcaggtgtacaccctgcctcctagccgggaggagatgacaaagaaccaggtcagcctgacatgtctggtgaagggcttctaccccagcgacattgccgtggaatgggagagcaatggccagcctgagaacaactacaagaccaccccccccgtgctggacagcgatggcagcttcttcctgtactctaagctgaccgtggacaaaagccgttggcagcagggcaacgtgttcagctgcagcgtgatgcacgaggccctgcataatcactacacccagaagtctctgagcctgtctcctggcggcggaggcggtagcggcggaggcggctccgaagaagagctgcaggtgatccaacctgataagagcgtcagcgtggccgccggagaaagcgccatcctgcactgcaccgtgaccagcttgatccctgtgggcccaatccagtggttccggggcgccggccctgcccgggaactgatctataaccagaaggaaggccatttcccacgggtgacaaccgtctctgaaagcacaaagagagagaacatggacttcagcatcagcatttctaatatcacccctgctgatgccggcacctactactgcgtgaaatttagaaaaggaagccctgacaccgagttcaagagcggcgctggcacggaactctccgttagagccaagtagtag

SEQ ID NO: 65 - RNA, 1821 nt, synthetic construct
  source 1..1821: mol_type = other RNA; note = Exemplary RNA sequence (minus UTRs) encoding the amino acid sequence represented by SEQ ID NO: 35 (1540)
  modified_base order(11,17,20,38,40,42,44,45,58,60,63,65,66,68,73,75,80,85,87,89,92,96,98,107,112,115,116,132,135,143,144,146,148,169,170,174,179,188,189,196,198,215,219,220,221,222,223,225,227,229,233,235,240,242,249,252,265,268,271,275,280,281,300,307,308,309,332,338,360,373,375,416,422,425,432,443,445,449,453,470,473,478,485,494,497,503,510,512,517,520,521,522,548,551,553,574,575,584,593,595,601,620,624,625,626,628,630,632,634,638,647,663,670,673,676,678,680,685,686,702,705,712,713,737,743,775,784,789,795,800,803,813,814,816,818,820,821,822,824,826,827,840,851,854,857,875,879,880,884,887,890,896,909,912,917,922,923,928,931,935,944,950,972,985,996,997,999,1004,1007,1009,1011,1013,1016,1022,1025,1036,1040,1054,1060,1067,1082,1089,1092,1094,1106,1137,1142,1144,1151,1155,1158,1172,1187,1193,1198,1200,1202,1205,1213,1214,1216,1229,1230,1235,1240,1251,1260,1270,1289,1292,1302,1309,1310,1312,1313,1316,1318,1321,1323,1328,1334,1347,1348,1364,1366,1367,1372,1379,1382,1394,1398,1401,1405,1417,1419,1421,1427,1429,1431,1434,1449,1465,1478,1484,1487,1494,1497,1505,1511,1532,1535,1540,1547,1555,1556,1559,1563,1565,1574,1579,1582,1583,1599,1610,1613,1615,1617,1635,1636,1637,1646,1655,1657,1659,1682,1687,1688,1694,1700,1701,1702,1704,1707,1709,1716,1719,1722,1732,1735,1738,1742,1747,1748,1749,1764,1774,1775,1788,1799,1801,1805,1806,1816,1819): mod_base = OTHER; note = Uracil
gaagaggaactgcaggtgatccaacccgacaagagcgtgtctgttgccgccggcgagtctgctattctgcactgtaccgtgacatctctcatccctgtgggaccgatccagtggttccggggcgccggccctgctagagagcttatctacaaccagaaagaaggccacttccctcgggtgaccacagttagcgagtctaccaagagagaaaacatggatttttctatctcaatctccaatatcacccctgctgacgccggaacatactactgcgtgaagttcagaaagggcagcccagatacagagtttaagagcggagccggcacagagctgagcgtgcgggccaagggcggcggcggtagcagcagcgcatctaccgacaagacacacacaggcggcggcagcgaggaggagctgcaagtgatccagcctgacaaaagcgtgtccgtggctgccggcgagagcgccatcctgcactgcaccgtgaccagcctgatccccgtgggccctatccagtggtttagaggcgccggccccgccagagaactgatctacaaccagaaagagggacacttccccagggtgacaaccgtgtccgagtccaccaaaagagagaacatggatttctctatctccatcagcaacatcaccccagccgacgctggaacctactactgtgtgaagttcagaaagggaagccctgataccgagttcaagagcggcgccggcaccgagctgagcgtcagagccaagggcggcgacaagacccacacctgccccccatgccctgcccctgagctgctgggcggaccttctgtgtttctgttcccccccaagcctaaggacaccctgatgatcagcagaacccccgaggtgacttgcgtggtggtcgacgtgagccacgaagatcctgaggtgaagttcaactggtacgtggacggcgtggaagtgcacaacgccaaaacaaaacctagagaggaacagtacaacagcacttatagagtggtgtctgtcctgaccgtgctgcaccaggactggctgaacggcaaggagtacaagtgcaaggtgagcaacaaggccctgcccgctcctatcgagaagaccatcagcaaggccaagggccagccaagagagcctcaggtgtacaccctgcctcctagccgggaggagatgacaaagaaccaggtcagcctgacatgtctggtgaagggcttctaccccagcgacattgccgtggaatgggagagcaatggccagcctgagaacaactacaagaccaccccccccgtgctggacagcgatggcagcttcttcctgtactctaagctgaccgtggacaaaagccgttggcagcagggcaacgtgttcagctgcagcgtgatgcacgaggccctgcataatcactacacccagaagtctctgagcctgtctcctggcggcggaggcggtagcggcggaggcggctccgaagaagagctgcaggtgatccaacctgataagagcgtcagcgtggccgccggagaaagcgccatcctgcactgcaccgtgaccagcttgatccctgtgggcccaatccagtggttccggggcgccggccctgcccgggaactgatctataaccagaaggaaggccatttcccacgggtgacaaccgtctctgaaagcacaaagagagagaacatggacttcagcatcagcatttctaatatcacccctgctgatgccggcacctactactgcgtgaaatttagaaaaggaagccctgacaccgagttcaagagcggcgctggcacggaactctccgttagagccaagtagtag

SEQ ID NO: 66 - protein, 22 aa, synthetic construct
  source 1..22: note = Signal Sequence
MDMRVPAQLLGLLLLWLRGARC

(19) United States
(12) Patent Application Publication
(10) Pub. No.: US 2026/0103501 A1
(43) Pub. Date: Apr. 16, 2026

(54) MRNA Therapies Including SIRP-ALPHA

(71) Applicant: NUTCRACKER THERAPEUTICS, INC., Emeryville, CA (US)

(72) Inventors: Gunasekaran Kannan, Emeryville, CA (US); Meredith Leong, Emeryville, CA (US); Samuel Deutsch, Emeryville, CA (US); Ole Haabeth, Emeryville, CA (US); Colin James McKinlay, Emeryville, CA (US); Srinivasa Bandi, Emeryville, CA (US); Adrienne Sallets, Emeryville, CA (US); Jenna Triplett, Emeryville, CA (US)

(21) Appl. No.: 18/995,050

(22) PCT Filed: Jul. 17, 2023
(86) PCT No.: PCT/US2023/027923

§ 371 Date: Jan. 15, 2025

Related U.S. Application Data
(60) Provisional application No. 63/389,820, filed on Jul. 15, 2022.
(60) Provisional application No. 63/480,596, filed on Jan. 19, 2023.
(60) Provisional application No. 63/483,881, filed on Feb. 8, 2023.

(51) Int. Cl. C07K 14/705; A61K 38/00

(52) U.S. Cl. CPC C07K 14/70503; A61K 38/00; C07K 2319/30

(57) ABSTRACT

Human SIRPa fusion proteins having an enhanced affinity to CD47 via increased binding valency. The human SIR-Pa fusion proteins described herein may form tetramer, hexamer, octamers, etc. These fusion proteins may be configured to form heterodimers with other fusion proteins, including C-C chemokine receptor type 4 (CCR4) binding fusion proteins.

This application claims priority to U.S. Provisional Application No. 63/389,820 filed Jul. 15, 2022, U.S. Provisional Application No. 63/480,596 filed Jan. 19, 2023 and U.S. Provisional Application No. 63/483,881 filed Feb. 8, 2023, all of which are incorporated by reference herein in their entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Synthetic mRNA-based therapeutics (e.g., mRNA vaccines or mRNA encoding fusion proteins) provide a template for the synthesis of proteins, protein fragments or peptides with the potential for significant benefit in a broad range of therapeutic applications. Engineered mRNA polynucleotides encoding fusion proteins provide selectively advantageous variations to physiological activity relative to native or wild-type proteins.

Signal regulatory protein alpha (SIRPα) is a transmembrane protein belonging to the immunoglobulin superfamily, and a receptor for CD47. Cloning and expression of a human form of SIRPα has been described by Ullrich et al in U.S. Pat. No. 6,541,615. SIRPα and CD47 are implicated in the etiology of cancer and other diseases. For example, the interaction between SIRPα and CD47 may play an important role in regulating the phagocytosis of leukemia cells and leukemia stem cells (LSCs) by macrophages.

The extracellular region of SIRPα generally comprises 3 immunoglobulin (Ig)-like domains and the cytoplasmic region contains immunoreceptor tyrosine-based inhibition motifs (ITIMs). SIRPα is especially abundant in myeloid cells such as macrophages. The extracellular region of SIRPα interacts with the ligand Cluster of Differentiation 47 (CD47) to mediate phagocytosis as a component of an immune response.

CD47 is a transmembrane glycoprotein and known ligand of signal regulatory proteins (e.g., SIRPα) active in negatively regulating phagocytotic activity of macrophages. CD47 is expressed in most normal cell types and may be overexpressed in various disease states including cancer cells. The interaction of SIRPα with CD47 is an inhibitory immune checkpoint negatively regulating phagocytotic activity of macrophages. The CD47-SIRPα interaction transmits inhibitory “don't eat me signals” to the phagocyte resulting in inhibition of phagocytosis following phosphorylation of its cytoplasmic ITIMs. For example, the CD47-SIRPα interaction has also been demonstrated to function as an inhibitory signal for phagocytosis of cancer cells by macrophages.

Blocking fusion proteins against CD47 have been shown to promote phagocytosis of LSCs by macrophages. This SIRPα/CD47 axis may also be implicated in immune disorders. Thus, there is a need for immunomodulatory therapeutics which inhibit signaling via the SIRPα/CD47 axis for use in the treatment of cancer and other diseases.

### SUMMARY OF THE DISCLOSURE

Provided herein are improved immunomodulatory mRNA-based anti-CD47 therapeutics such as immunomodulatory mRNA-based anti-CD47 therapeutics which increase target affinity through engineered SIRPα sequences. For example, provided are immunomodulatory mRNA-based anti-CD47 therapeutics encode fusion proteins comprising SIRPα sequences that increase target affinity and higher avidity to CD47 through modification of the fusion protein valency.

Described herein are engineered molecules comprising multi-valent SIRPα regions or domains that bind to CD47 and the synthetic mRNA sequences encoding these molecules. These engineered molecules include fusion protein comprising multi-valent SIRPαdomains, (including but not limited to bivalent, tetravalent, hexavalent, octavalent SIRPαdomains). These molecules may be referred to as multi-valent CD47-binding agents. In some exemplary molecules, the mRNA sequences encode amino acid substitutions that increase binding affinity of the SIRPα binding domains to CD47.

In general, these fusion proteins may form dimers (e.g., homodimers or heterodimers); in particular, described herein are heterodimers that may include one or more regions binding C-C chemokine receptor type 4 (CCR4) referred to herein as an anti-CCR4 binding domain.

As described herein, engineered signal regulatory protein alpha (SIRPα) fusion proteins with modified structural binding capacity (e.g., multimeric fusion proteins with increased valency) may enhance SIRPα binding to CD47 and may enhance immunomodulatory functions mediated by SIRPα, including phagocytosis. Current developments in SIRPα-related antibodies are deficient in targeted binding affinity requiring higher dosage and higher associated cytotoxicity. The present disclosure describes high affinity SIRPα constructs (and synthetic mRNAs encoding such constructs) useful in the disruption of acquired defensive mechanisms of diseased cells overexpressing CD47 as an immune checkpoint for negative regulation of phagocytotic activity of myeloid cells. Also described herein are additional structural improvements useful in promoting higher binding affinity of a fusion protein encoded by a synthetic mRNA via improved valency. Therapeutic methods using the engineered molecules described herein are also provided.

The terms “SIRPα fusion protein” and “SIRPα constructs” refer to an engineered molecule comprising at least one SIRPα domain that binds to CD47 (also referred to herein as a “SIRPα CD47 binding domain”).

The disclosure provides for a multi-valent fusion protein comprising i) at least two SIRPα domains that bind to CD47 and ii) an IgG Fc receptor (also referred to as an Fc region or FC domain herein). For example, the fusion protein comprises a human IgG1 Fc receptor comprising the CH2 and CH3 domains, and in some multi-valent fusion proteins, the IgG Fc receptor comprises the knob and/or the hole domain to assist in heterodimerization. In some embodiments, the multi-valent fusion protein comprises the amino acid sequence of SEQ ID NO: 1. The disclosure provides for polynucleotides, such as mRNA molecules, that encode any of the multi-valent fusion proteins described herein.

The disclosure provides multi-valent fusion proteins comprising three, four, five, six, seven, or eight SIRPα domains and a human IgG1 Fc domain, and polynucleotides, such as mRNA molecules, the encode any of the multi-valent fusion proteins described herein.

The disclosure provides for multi-valent fusion proteins comprising an amino acid sequence that is at least 85% homologous, at least 90% homologous or at least 95% homologous to an amino acid sequence selected from the group consisting of SEQ ID NO: 4, SEQ ID NO: 5, and SEQ ID NO: 6. In addition, the disclosure provides for multi-valent fusion proteins comprising an amino acid sequence selected from the group consisting of SEQ ID NO: 4, SEQ ID NO: 5, and SEQ ID NO: 6.

The polypeptides (or domains) of the disclosed multi-valent fusion proteins may be enjoined with any linker sequence known in the art. An exemplary linker is a peptide comprising two consecutive glycines (G-G) or a peptide comprising the amino acids proline and serine (P-S).

For example, the disclosed multi-valent fusion proteins comprise at least one amino acid sequences that is at least 85% homologous, at least 90% homologous or at least 95% homologous to the amino acid sequence of SEQ ID NO: 2, SEQ ID NO: 12, SEQ ID NO: 13, SEQ ID NO: 16, SEQ ID NO: 18, SEQ ID NO: 19, or SEQ ID NO: 20 or a sequence comprising amino acids 23 to 627 of SEQ ID NO: 35, amino acids 23 to 617 of SEQ ID NO: 59 or amino acids 23 to 742 or SEQ ID NO: 60, wherein the amino acids sequences comprise a SIRPα domain that binds to CD47 and a IgG Fc receptor. In addition, the disclosed multi-valent fusion proteins comprise the amino acid sequence of SEQ ID NO: 2, SEQ ID NO: 12, SEQ ID NO: 13, SEQ ID NO: 16, SEQ ID NO: 18, SEQ ID NO: 19, or SEQ ID NO: 20 or a sequence comprising amino acids 23 to 627 of SEQ ID NO: 35, amino acids 23 to 617 of SEQ ID NO: 59 or amino acids 23 to 742 or SEQ ID NO: 60.

The disclosure also provides for polynucleotide sequence encoding these multi-valent fusion proteins, such as polynucleotides, such as mRNA, comprising the nucleotide sequence of SEQ ID NO: 54, SEQ ID NO: 62, SEQ ID NO: 63, SEQ ID NO: 56, SEQ ID NO: 55, SEQ ID NO: 58, SEQ ID NO: 53, SEQ ID NO: 51, SEQ ID NO: 29, SEQ ID NO: 49, SEQ ID NO: 57, or SEQ ID NO: 50. The disclosure also provides for polynucleotides, such as mRNA, comprising a nucleotide sequence encoding the fusion proteins having a signal sequence such as the nucleotide sequences of SEQ ID NO:44, SEQ ID NO:46, SEQ ID NO: 61, SEQ ID NO:45, SEQ ID NO:48, SEQ ID NO: 43, SEQ ID NO: 41, SEQ ID NO: 39 or SEQ ID NO: 47, and inclusion of the signal sequence facilitates secretion of the fusion protein.

In some embodiments, the SIRPα fusion protein is a bivalent fusion protein comprising two SIRPα domains and a human IgG1 Fc domain. In other embodiments, the SIRPα fusion protein is a tetravalent fusion protein comprising four SIRPα domains and a human IgG1 Fc domain. For example, the tetravalent fusion protein comprises an amino acid sequence that is at least 85% homologous, at least 90% homologous or at least 95% homologous to the amino acid sequence of SEQ ID NO: 2, SEQ ID NO: 16, SEQ ID NO: 17, SEQ ID NO: 18, SEQ ID NO: 19, SEQ ID NO: 20, or SEQ ID NO: 21 or a sequence comprising amino acids 23 to 372 or SEQ ID NO: 29, amino acids 23 to 601 of SEQ ID NO: 30, amino acids 23 to 23 to 727 of SEQ ID NO: 31, amino acids 23 to 497 of SEQ ID NO: 34, amino acids 23 to 627 of SEQ ID NO: 35 or amino acids 23 to 492 of SEQ ID NO: 36 or a combination thereof, wherein the amino acids sequences comprise a SIRPα domain that binds to CD47 and a IgG Fc receptor. In addition, the tetravalent fusion proteins comprise the amino acid sequence of SEQ ID NO: 2, SEQ ID NO: 16, SEQ ID NO: 17, SEQ ID NO: 18, SEQ ID NO: 19, SEQ ID NO: 20, or SEQ ID NO: 21 or a sequence comprising amino acids 23 to 372 or SEQ ID NO: 29, amino acids 23 to 601 of SEQ ID NO: 30, amino acids 23 to 23 to 727 of SEQ ID NO: 31, amino acids 23 to 497 of SEQ ID NO: 34, amino acids 23 to 627 of SEQ ID NO: 35 or amino acids 23 to 492 of SEQ ID NO: 36 or a combination thereof. In these tetravalent fusion proteins, these amino acid sequences may be homodimers or heterodimers.

The disclosure also provides for polynucleotide sequence encoding these tetravalent fusion proteins, such as polynucleotides, such as mRNA, comprising the nucleotide sequence of SEQ ID NO: 53, SEQ ID NO: 54, SEQ ID NO: 51, SEQ ID NO: 49, SEQ ID NO: 57, SEQ ID NO: 50 or SEQ ID NO: 65. The disclosure also provides for polynucleotides, such as mRNA, comprising a nucleotide sequence encoding the fusion proteins having a signal sequence such as the nucleotide sequences of SEQ ID NO: 43, SEQ ID NO: 44, SEQ ID NO: 41, SEQ ID NO: 39, SEQ ID NO: 47, SEQ ID NO: 40, SEQ ID NO: 64, SEQ ID NO: 47, or SEQ ID NO: 40, and inclusion of the signal sequence facilitates secretion of the fusion protein.

In further embodiments, the disclosure provides for a hexavalent fusion protein comprising six SIRPα domains and a human IgG1 Fc domain. For example, the fusion protein comprises a dimer of an amino acid sequence that is at least 85% homologous, at least 90% homologous or at least 95% homologous to the amino acid sequence of SEQ ID NO: 12 or a sequence comprising amino acids 23 to 627 of SEQ ID NO: 35 or amino acids 23 to 617 or SEQ ID NO: 59, wherein the amino acids sequences comprise a SIRPα domain that binds to CD47 and an IgG Fc receptor or a combination thereof. In these hexavalent fusion proteins, these amino acid sequences may be homodimers or heterodimers. The disclosure also provides for polynucleotide sequence encoding these hexavalent fusion proteins, such as polynucleotides, such as mRNA, comprising the nucleotide sequence of SEQ ID NO: 58 or 62.

In further embodiments, the disclosure provides for an octovalent fusion protein comprising eight SIRPα domains and a human IgG1 Fc domain. For example, the fusion protein comprises a dimer of an amino acid sequence that is at least 85% homologous, at least 90% homologous or at least 95% homologous to the amino acid sequence of SEQ ID NO: 13 or a sequence comprising amino acids 23 to 742 of SEQ ID NO: 60 or a combination thereof, wherein the amino acids sequences comprise a SIRPα domain that binds to CD47 and a IgG Fc receptor. In addition, the octovalent fusion proteins comprise the amino acid sequence of SEQ ID NO: 13 or a sequence comprising amino acids 23 to 742 of SEQ ID NO: 60, or a combination thereof. In these octavalent fusion proteins, these amino acid sequences may be homodimers or heterodimers.

The disclosure also provides for polynucleotide sequence encoding these octovalent fusion proteins, such as polynucleotides, such as mRNA, comprising the nucleotide sequence of SEQ ID NO: 55, SEQ ID NO: 58 or SEQ ID NO: 63. The disclosure also provides for polynucleotides, such as mRNA, comprising a nucleotide sequence encoding the fusion proteins having a signal sequence such as the nucleotide sequences of SEQ ID NO: 45, SEQ ID NO: 48 or SEQ ID NO: 61, and inclusion of the signal sequence facilitates secretion of the fusion protein.

In one embodiment, the disclosure provides for SIRPα fusion proteins comprising an amino acid sequence of SEQ ID NO: 2 or a sequence comprising amino acids 23 to 494 of SEQ ID NO: 3. Also described herein are SIRPα fusion proteins encoded by synthetic mRNAs comprising an amino acid sequence that is at least 95% homologous to the amino acid sequence of SEQ ID NO: 2 or a sequence comprising amino acids 23 to 494 of SEQ ID NO: 3, having at least four CD47 binding domains. Also provided are nucleotide sequences encoding any of the disclosed SIRPα fusion proteins having an enhanced affinity to CD47 via increased binding valency.

Also described herein are isolated fusion proteins. For example, described herein are SIRPα fusion proteins comprising an amino acid sequence of SEQ ID NO: 2 or a sequence comprising amino acids 23 to 494 of SEQ ID NO: 3. In some examples the SIRPα fusion protein comprises an amino acid sequence that is at least 95% homologous to the amino acid sequence of SEQ ID NO: 2 or a sequence comprising amino acids 23 to 494 of SEQ ID NO: 3, wherein the fusion protein comprises a tetravalent structure. In some aspects, the SIRPα fusion protein comprises ten or fewer amino acid substitutions relative to SEQ ID NO: 23, or the SIRPα fusion protein comprises five or fewer amino acid substitutions relative to SEQ ID NO: 23. In some examples, the SIRPα fusion protein comprises 1, 2, or 3 amino acid substitutions relative to SEQ ID NO: 23. The amino acid sequence may be greater than 98% homologous with the amino acid sequence set forth in SEQ ID NO: 2 or SEQ ID NO: 3.

For example, the disclosure provides for SIRPα fusion proteins encoded by a synthetic mRNA may have an enhanced affinity to CD47 via increased binding valency. SEQ ID NOs: 22 and 23 illustrate examples of CD47 binding domains. SEQ ID NO: 23 is an example of a wild-type SIRPα IgV (e.g., of a wild-type CD47 binding domain) and SEQ ID NO: 22 is an example of a SIRPα IgV (CD47 binding domain) that has higher affinity than wild type.

The SIRPα fusion protein encoded by a synthetic mRNA may form a tetravalent dimer having four CD47 binding domains. For example, the SIRPα fusion protein may form a hexavalent dimer having six CD47 binding domains or eight CD47 binding domains. The fusion protein may comprise ten or fewer (e.g., 5 or fewer, 3 or fewer, etc.) amino acid substitutions within residues 23 and 139 relative to a wild-type SIRPα IgV (e.g., SEQ ID NO: 23) sequence. In some embodiments, the amino acid sequence of the SIRPα fusion protein is at least 98% homologous to the amino acid sequence of SEQ ID NO: 2 or a sequence comprising amino acids 23 to 494 of SEQ ID NO: 3. In some examples, the amino acid sequence comprises the amino acid sequence of SEQ ID NO: 2; alternatively, the amino acid sequence comprises the a sequence comprising amino acids 23 to 494 of SEQ ID NO: 3.

In some embodiments, a SIRPα fusion protein, encoded by a synthetic mRNA, comprises an amino acid sequence that is at least 95% homologous to the amino acid sequence of SEQ ID NO: 2 or a sequence comprising amino acids 23 to 494 of SEQ ID NO: 3, wherein the fusion protein comprises a tetravalent structure having multiple binding domains to CD47. The isolated fusion protein may comprise ten or fewer (e.g., 5 or fewer, 3 or fewer, etc.) amino acid substitutions between residues 1 and 116 relative to a wild-type SIRPα IgV sequence (e.g., SEQ ID NO: 23). For example, the amino acid sequence is at least 98% homologous to the amino acid sequence set forth in SEQ ID NO: 2 or a sequence comprising amino acids 23 to 494 of SEQ ID NO: 3.

For example, a SIRPα fusion protein encoded by a synthetic mRNA comprises an antigen binding region having an amino acid sequence that is at least 95% homologous to the amino acid sequence of SEQ ID NO:1 wherein the fusion protein has at least two high affinity binding domains to CD47. The SIRPα fusion protein may be bivalent with two high affinity binding domains to CD47. The fusion protein comprises ten or fewer amino acid substitutions within residues 23 and 139 of SEQ ID NO: 1, relative to a wild-type SIRPα IgV sequence (e.g., SEQ ID NO: 23). The isolated SIRPα fusion protein comprises five or fewer amino acid substitutions within residues 23 and 139 of SEQ ID NO: 1, relative to a wild-type SIRPα IgV sequence of SIRPα (e.g., SEQ ID NO: 23). The SIRPα fusion protein comprises 1, 2, or 3 amino acid substitutions between position 23 and 139 of SEQ ID NO: 1 relative to a wild-type sequence of SIRPα IgV of SIRPα (e.g., SEQ ID NO: 23). In some examples, the SIRPα fusion protein comprises an amino acid sequence that is at least 98% homologous to the amino acid sequence set forth in SEQ ID NO:1. The isolated SIRPα fusion protein comprises the amino acid sequence set forth in SEQ ID NO:1 and the amino acid sequence set forth in SEQ ID NO:2. In one examples, the isolated SIRPα fusion protein comprises an antigen binding region having an amino acid sequence of SEQ ID NO:1 wherein the fusion protein has at least two high affinity binding domains to CD47.

An SIRPα fusion protein, encoded by a synthetic mRNA, comprises an amino acid sequence that is at least 95% homologous to the amino acid sequence of SEQ ID NO: 2 or a sequence comprising amino acids 23 to 494 of SEQ ID NO: 3, wherein the fusion protein comprises a tetravalent structure having multiple binding domain to CD47. For example, the SIRPα fusion protein comprises ten or fewer amino acid substitutions within residues 1 and 116 relative to a wild-type SIRPα IgV sequence (e.g., five or fewer amino acid substitutions within residues 1 and 116 relative to a wild-type SIRPα IgV sequence, e.g., 1, 2, or 3 amino acid substitutions within residues 1 and 116 relative to a wild-type SIRPα IgV sequence, etc.). In another example, the SIRPα fusion protein comprises an amino acid sequence that is at least 98% homologous to the amino acid sequence set forth in SEQ ID NO:2. The human wild-type SIRPα IgV sequence is listed in SEQ ID NO: 23.

For example, an SIRPα fusion protein, encoded by a synthetic mRNA, comprising an amino acid sequence that is homologous to the amino acid sequence of SEQ ID NO: 2 or a sequence comprising amino acids 23 to 494 of SEQ ID NO: 3, wherein the fusion protein is part of a tetravalent structure having four or more binding domains to CD47. An exemplary isolated tetravalent SIRPα fusion protein comprises an amino acid sequence homologous with the amino acid sequence set forth in SEQ ID NO: 2.

For example, described herein are SIRPα fusion proteins comprising an amino acid sequence that is at least 95% homologous to the amino acid sequence of SEQ ID NO: 2 or a sequence comprising amino acids 23 to 494 of SEQ ID NO: 3, encoding two or more CD47 binding domains. In some examples the SIRPα fusion protein forms a tetravalent dimer having four CD47 binding domains. In other examples, the SIRPα fusion protein forms a hexavalent dimer having six CD47 binding domains. In another example, the SIRPα fusion protein forms an octavalent dimer having eight CD47 binding domains. The SIRPα fusion protein comprises ten or fewer amino acid substitutions relative to SEQ ID NO: 23. The SIRPα fusion protein comprises five or fewer amino acid substitutions relative to SEQ ID NO: 23. The amino acid sequence is at least 98% homologous to the amino acid sequence of SEQ ID NO: 2 or SEQ ID NO: 3. In some examples, the amino acid sequence comprises the amino acid sequence of SEQ ID NO: 2 or a sequence comprising amino acids 23 to 494 of SEQ ID NO: 3.

Any of the disclosed SIRPα multi-valent fusion proteins may further comprise at least one anti-C-C chemokine receptor type 4 (CCR4) binding domains (also referred to herein as “SIRPα/CCR4-binding fusion proteins”). In some embodiments, the SIRPα/CCR4-binding fusion protein comprises an amino acid sequence that is at least 85% homologous, at least 90% homologous or at least 95% homologous to the amino acid sequence of SEQ ID NO: 14, SEQ ID NO: 15, SEQ ID NO: 16, SEQ ID NO: 17, SEQ ID NO: 18, SEQ ID NO: 19, SEQ ID NO: 20 or SEQ ID NO: 21. In other embodiments, the SIRPα/CCR4-binding fusion protein comprises the amino acid sequence of SEQ ID NO: 14, SEQ ID NO: 15, SEQ ID NO: 16, SEQ ID NO: 17, SEQ ID NO: 18, SEQ ID NO: 19, SEQ ID NO: 20 or SEQ ID NO: 21.

In other embodiments, the SIRPα/CCR4-binding fusion protein comprises an amino acid sequence that is at least 85% homologous, at least 90% homologous or at least 95% homologous to amino acids 23 to 372 of SEQ ID NO: 29, amino acids 23 to 601 of SEQ ID NO: 30, amino acids 23 to 727 of SEQ ID NO: 31, amino acids 23 to 377 of SEQ ID NO: 32, amino acids 23 to 502 of SEQ ID NO: 33, amino acids 23 to 497 of SEQ ID NO: 34, or amino acids 23 to 492 of SEQ ID NO: 36. In other embodiments, the SIRPα/CCR4-binding fusion protein comprises amino acids 23 to 372 of SEQ ID NO: 29, amino acids 23 to 601 of SEQ ID NO: 30, amino acids 23 to 727 of SEQ ID NO: 31, amino acids 23 to 377 of SEQ ID NO: 32, amino acids 23 to 502 of SEQ ID NO: 33, amino acids 23 to 497 of SEQ ID NO: 34, or amino acids 23 to 492 of SEQ ID NO: 36.

The disclosure also provides for polynucleotide sequence encoding the SIRPα/CCR4-binding fusion protein, such as mRNA, comprising the nucleotide sequence of SEQ ID NO: 52, SEQ ID NO: 53, SEQ ID NO: 51, SEQ ID NO: 49, SEQ ID NO: 57 or SEQ ID NO: 50. The disclosure also provides for polynucleotides, such as mRNA, comprising a nucleotide sequence encoding the fusion proteins having a signal sequence such as the nucleotide sequences of SEQ ID NO: 42, SEQ ID NO: 43, SEQ ID NO: 41, SEQ ID NO: 39, SEQ ID NO: 47 or SEQ ID NO: 40 and inclusion of the signal sequence facilitates secretion of the fusion protein.

Any of the SIRPα/CCR4-binding fusion proteins disclosed herein may be homodimers or heterodimers. In some embodiments, the heterodimer fusion proteins comprise the knob and/or hole domain to facilitate heterodimerization.

In any of the SIRPα/CCR4-binding fusion proteins disclosed herein, the SIRPα domains are fused to the N-terminal ends of anti-CCR4 LC domains. In another embodiment, the SIRPα domains are fused to (i) the N-terminal ends of anti-CCR4 LC domains, and (ii) the C-terminal ends of anti-CCR4 HC domains.

In addition, in any of the SIRPα/CCR4-binding fusion proteins disclosed herein, i) a SIRPα domain fused to the N-terminal end of an anti-CCR4 LC domain, (ii) a SIRPα domain fused to the C-terminal end of an anti-CCR4 HC domain, and (iii) SIRPα domains fused to both the N and C-terminal ends of an anti-CCR4 HC domain, wherein the Fc domains comprises the knob and/or hole domain to facilitate heterodimerization.

The disclosure also provides for SIRPα/CCR4-binding fusion proteins wherein the SIRPα domains are fused to the N-terminal ends of anti-CCR4 HC domains. In addition, the disclosure provides for SIRPα/CCR4-binding fusion proteins wherein the SIRPα domains are fused to (i) the N-terminal ends of anti-CCR4 HC domains, and (ii) the C-terminal ends of anti-CCR4 LC domains.

In another embodiment, the disclosure provides for SIRPα/CCR4-binding fusion proteins wherein the SIRPα domains are fused to both the N- and C-terminal ends of anti-CCR4 HC domains. In addition, the disclosure provides for SIRPα/CCR4-binding fusion proteins wherein the SIRPα domains fused to (i) both the N- and C-terminal ends of anti-CCR4 HC domains, and (ii) the C-terminal ends of anti-CCR4 LC domains.

The disclosure further provides for fusion proteins wherein the SIRPα domains are fused to (i) the N-terminal ends of anti-CCR4 HC domains, and (ii) the C-terminal ends of anti-CCR4 LC domains. In addition, the disclosure provides for SIRPα/CCR4-binding fusion proteins wherein the SIRPα domains fused to (i) both the N- and C-terminal ends of anti-CCR4 HC domains, and (ii) the C-terminal ends of anti-CCR4 LC IRPα/CCR4-binding fusion proteins domains.

In an exemplary embodiment, the disclosure provides for SIRPα/CCR4-binding fusion proteins wherein the anti-CCR4 scFv domains are fused to the N-terminal ends of the light chains. In another embodiment, the disclosure provides for SIRPα/CCR4-binding fusion proteins wherein anti-CCR4 scFv domains are fused to the N-terminal ends of the light chains, and SIRPα domains fused to the N-terminals of the heavy chains. In addition, the disclosed SIRPα/CCR4-binding fusion proteins comprise anti-CCR4 scFv domains fused to the N-terminal ends of the light chains, and SIRPα domains fused to both the N- and C-terminals of the heavy chains.

For example, human SIRPα and a C-C chemokine receptor type 4 (CCR4) binding fusion proteins (“SIRPα/CCR4-binding fusion proteins”), encoded by synthetic mRNAs, comprising i) an amino acid sequence that is at least 95% homologous to the amino acid sequence of SEQ ID NO: 14 and ii) the amino acid sequence of either SEQ ID NO. 22 or SEQ ID NO. 23. For example, these SIRPα/CCR4-binding fusion proteins are a single-chain variable fragments (scFv), e.g., fusion proteins of the variable regions of the heavy, VH, and light chains, VL, portions of these CCR4 immunoglobulins, connected with a short linker peptide. Thus, any of these SIRPα/CCR4-binding fusion proteins may further comprise an amino acid sequence that is at least 95% homologous to the amino acid sequence of SEQ ID NO:17.

For example, the amino acid sequence of the SIRPα/CCR4-binding fusion proteins comprise i) an amino acids sequence that is at least 99% homologous to the amino acid sequence of SEQ ID NO: 14 and ii) the amino acid sequence of either SEQ ID NO. 22 or SEQ ID NO. 23. For example, the SIRPα/CCR4-binding fusion protein comprises i) the amino acid sequence comprises of SEQ ID NO: 14 and ii) the amino acid sequence of either SEQ ID NO. 22 or SEQ ID NO. 23.

In some aspects, the SIRPα/CCR4-binding fusion protein, encoded by a synthetic mRNA, comprises an amino acid sequence that is at least 95% homologous to the amino acid sequence of SEQ ID NO: 15. In another case, the SIRPα/CCR4-binding fusion protein may comprise an amino acid sequence that is at least 95% homologous to the amino acid sequence of SEQ ID NO: 16. Also provided is the SIRPα/CCR4-binding fusion protein comprising an amino acid sequence that is at least 95% homologous to the amino acid sequence of SEQ ID NO: 20. In addition, provided is the SIRPα/CCR4-binding fusion protein comprising an amino acid sequence that is at least 95% homologous to the amino acid sequence of SEQ ID NO: 21. Any of these SIRPα/CCR4-binding fusion protein may further comprising an amino acid sequence that is at least 95% homologous to the amino acid sequence of SEQ ID NO:17 (e.g., the VL portion).

Also described herein are heterodimers comprising a human SIRPα binding site and a C-C chemokine receptor type 4 (CCR4) binding site, the heterodimer comprising first fusion protein dimerized to a second fusion protein, further wherein the first fusion protein comprises an amino acid sequence that is at least 95% homologous to the amino acid sequence of one of SEQ ID NO: 8, 11 or 20, and wherein the second fusion protein comprises an amino acid sequence that is at least 95% homologous to the amino acid sequence of one of SEQ ID NO: 9, 10, 19, or 21. For example, the first fusion protein comprises a hole modification and the second fusion protein comprises a knob modification. In another case, the first fusion protein comprises an amino acid sequence that is at least 99% homologous to the amino acid sequence of one of SEQ ID NO: 8, 11 or 20, and wherein the second fusion protein comprises an amino acid sequence that is at least 99% homologous to the amino acid sequence of one of SEQ ID NO: 9, 10, 19, or 21. In addition, the first fusion protein comprises an amino acid sequence of one of SEQ ID NO: 8, 11 or 20, and wherein the second fusion protein comprises an amino acid sequence of one of SEQ ID NO: 9, 10, 19, or 21. In some aspects, the heterodimer comprises a first fusion protein dimerized to a second fusion protein, wherein the first fusion protein comprises a hole modification and the second fusion protein comprises a knob modification.

Also described herein are heterodimers comprising a human SIRPα binding site and a C-C chemokine receptor type 4 (CCR4) binding site, wherein at least one of first and second fusion proteins is a SIRPα/CCR4-binding fusion protein, wherein the first fusion protein is dimerized to the second fusion protein, further wherein the first fusion protein comprises an amino acid sequence that is at least 95% homologous to the amino acid sequence of SEQ ID NO: 8, 11 or 20, and wherein the second fusion protein comprises an amino acid sequence that is at least 95% homologous to the amino acid sequence of one of SEQ ID NO: 9, 10, 19, or 21. The first fusion protein comprises an amino acid sequence that is at least 99% homologous to the amino acid sequence of SEQ ID NO: 8, 11 or 20, and wherein the second fusion protein comprises an amino acid sequence that is at least 98% homologous to the amino acid sequence of one of SEQ ID NO: 9, 10, 19, or 21. For example, the first fusion protein comprises the amino acid sequence of SEQ ID NO: 8, 11 or 20, and wherein the second fusion protein comprises the amino acid sequence of one of SEQ ID NO: 9, 10, 19, or 21.

The disclosure also provides for a heterodimer comprising a human SIRPα binding site fusion protein and a C-C chemokine receptor type 4 (CCR4) binding fusion protein in which a first fusion protein dimerized to a second fusion protein, further wherein the first fusion protein comprises an amino acid sequence that is at least 95% homologous to the amino acid sequence of one of SEQ ID NO: 11, and wherein the second fusion protein comprises an amino acid sequence that is at least 95% homologous to the amino acid sequence of one of SEQ ID NO: 9, 10, 19, or 21. For example, the first fusion protein comprises an amino acid sequence that is at least 99% homologous to the amino acid sequence of SEQ ID NO: 11, and wherein the second fusion protein comprises an amino acid sequence that is at least 98% homologous to the amino acid sequence of one of SEQ ID NO: 9, 10, 19, or 21. In some aspects, the first fusion protein comprises the amino acid sequence of SEQ ID NO: 11, and wherein the second fusion protein comprises the amino acid sequence of one of SEQ ID NO: 9, 10, 19, or 21.

The disclosure provides a heterodimer comprising a human SIRPα binding site fusion protein and a C-C chemokine receptor type 4 (CCR4) binding fusion protein, wherein the heterodimer comprises a first fusion protein dimerized to a second fusion protein, further wherein the first fusion protein comprises an amino acid sequence that is at least 95% homologous to the amino acid sequence of one of SEQ ID NO: 20, and wherein the second fusion protein comprises an amino acid sequence that is at least 95% homologous to the amino acid sequence of one of SEQ ID NO: 9, 10, 19, or 21. For example, the first fusion protein comprises an amino acid sequence that is at least 99% homologous to the amino acid sequence of SEQ ID NO: 20, and wherein the second fusion protein comprises an amino acid sequence that is at least 98% homologous to the amino acid sequence of one of SEQ ID NO: 9, 10, 19, or 21. In other examples, the first fusion protein comprises the amino acid sequence of SEQ ID NO: 20, and wherein the second fusion protein comprises the amino acid sequence of one of SEQ ID NO: 9, 10, 19, or 21.

Also described herein are fusion proteins forming dimers including one or more copies of a human SIRPα region as well as a C-C chemokine receptor type 4 (CCR4) binding (e.g., anti-CCR4) region. CCR4 is expressed by CD4+ T cells from only a subset of non-intestinal tissues, and expressed at the highest, most functional levels only by skin-infiltrating CD4⁺ cells. CCR4 binding polypeptides include anti-CCR4 antibodies such as Mogamulizumab; Mogamulizumab is a humanized monoclonal antibody (mAb) directed against CC chemokine receptor 4 (CCR4) for the treatment of Mycosis Fungoides (MF) and Sézary Syndrome (SS), the most common subtypes of cutaneous T-cell lymphoma. It includes both a heavy chain and light chain.

Described herein are SIRPα fusion proteins that are bispecific and may include a CCR4 scFv (“aCCR4 scFv”) fusion protein for improved localization and specificity to tumor cells. For example, FIGS. 11A-11F schematically illustrate examples of fusion proteins (forming both homodimers and heterodimers) of SIRPα IgV and aCCR4 Fab (FIGS. 3E-3F) or aCCR4 scFv (FIGS. 3A-3D). SEQ IDS NO: 9, 10, 14-18, and 20-21 illustrate examples of amino acid sequences that may form the structures illustrated in FIGS. 3A-3F. In any of these examples, the heterodimer may include a first fusion protein that may be dimerized to a second fusion protein. Thus, in general, the fusion proteins described herein may be modified to promote heterodimerization (preferentially over homodimerization). For example, any of these fusion proteins may include a heteromultimerization domain. A “heteromultimerization domain” may refer to alterations or additions to a biological molecule so as to promote heteromultimer formation and hinder homomultimer formation. Any heterodimerization domain having a strong preference for forming heterodimers over homodimers is within the scope of the invention. Illustrative examples include but are not limited to, for example, US Patent Application 20030078385 (describing knob into holes); WO2007147901 (describing ionic interactions); WO 2009089004 (describing electrostatic steering effects), etc. For example, dimerization (via the Fc domains, for example) may be modified to bias heterodimerization using a knob and hole modification to the fusion proteins.

One exemplary fusion protein comprises an antigen binding domain comprising an amino acid sequence at least 95% homologous to the amino acid sequence of SEQ ID NO:7, or comprises an antigen binding domain comprising an amino acid sequence of SEQ ID NO: 7.

In some examples, the any of the disclosed fusion protein is part of a bispecific (e.g., bivalent) molecule for binding to CD47 from two regions without interfering with each other. According to some examples described herein, the fusion protein is bivalent with two high affinity binding domains to CD47. The amino acid sequence of these fusion proteins have ten or fewer amino acid substitutions relative to a wild-type sequence of SIRPα of SEQ ID NO: 23; five or fewer amino acid substitutions relative to a wild-type sequence of SIRPα of SEQ ID NO: 23; or 7, 2, or 3 amino acid substitutions relative to a wild-type sequence of SIRPα of SEQ ID NO: 23. For example, the amino acid sequence set forth in SEQ ID NO:1 is 95% homologous with a wild-type sequence of SIRPα of SEQ ID NO: 23. In addition, the amino acid sequence is at least 98% homologous to the amino acid sequence set forth in SEQ ID NO:1. The amino acid sequence set forth in SEQ ID NO:1 may be combined in an amino acid sequence set forth in SEQ ID NO:2. For example, the fusion protein comprises at least four binding domains wherein at least one of the four binding domains has higher binding affinity to CD47 than wild type SIRPα.

In some examples, the fusion protein comprises an amino acid sequence that is at least 95% homologous to the amino acid sequence set forth in SEQ ID NO:2 wherein the fusion protein comprises at least one binding domain to CD47 (and preferably four). In some aspects, the binding domain has a high affinity binding domain. The disclosure also provides for tetravalent fusion proteins comprising at least four binding domains to CD47 (including high affinity binding domains). For example, the disclosed fusion proteins comprise a modified amino acid sequence of SEQ ID NO: 2 or a sequence comprising amino acids 23 to 494 of SEQ ID NO: 3 wherein the amino acid sequence comprises ten or fewer amino acid substitutions relative to a wild-type sequence of SIRPα of SEQ ID NO: 23. In addition, the disclosed fusion proteins comprise a modified amino acid sequence of SEQ ID NO: 2 or a sequence comprising amino acids 23 to 494 of SEQ ID NO: 3 wherein the amino acid sequence comprises five or fewer amino acid substitutions relative to a wild-type sequence of SIRPα of SEQ ID NO: 23. In addition, the fusion protein comprises a modified amino acid sequence of SEQ ID NO: 2 or a sequence comprising amino acids 23 to 494 of SEQ ID NO: 3 wherein the amino acid sequence comprises 1, 2, or 3 amino acid substitutions relative to a wild-type sequence of SIRPα of SEQ ID NO: 23.

In some embodiments, the disclosure provides fusion proteins comprising a SIRPαdomain. The fusion protein comprises an amino acid sequence of SEQ ID NO: 2 or a sequence comprising amino acids 23 to 494 of SEQ ID NO: 3 which is at least 95% homologous to the wild-type sequence of SIRPα or SEQ ID NO: 23. For example, the amino acid sequence is at least 98% homologous to the amino acid sequence set forth in SEQ ID NO: 2 or a sequence comprising amino acids 23 to 494 of SEQ ID NO: 3. In some aspects, the amino acid sequence set forth in SEQ ID NO: 2 or 3 is combined with all or a functional segment of an amino acid sequence set forth in SEQ ID NO:1.

The SIRPα domain of the fusion protein is encoded by a modified SIRPα mRNA. For example, the modified SIRPα mRNA comprises a nucleotide sequence having five or fewer amino acid substitutions relative to a wild-type SIRPα mRNA sequence, and wherein the modified SIRPα mRNA encodes a CD47 binding domain that has a specific binding affinity to CD47 greater than that of a SIRPα wild type CD47 domain.

In some embodiments, the fusion protein comprises an amino acid sequence of homologous with the amino acid sequence set forth in SEQ ID NO:1 or SEQ ID NO: 2. For example, the amino acid sequence is at least 95% homologous to the amino acid sequence set forth in SEQ ID NO:1 or SEQ ID NO:2. In some aspects, the modified SIRPα binding region comprises at least two amino acid substitutions in a linker region between a SIRPα antigen binding fragment and a crystallizable fragment region.

The disclosure provides for a tetravalent fusion protein comprising at least one CD47 binding domain with an amino acid sequence homologous with the amino acid sequence set forth in SEQ ID NO:2. For example, the amino acid sequence is at least 90% homologous to the amino acid sequence set forth in SEQ ID NO:2. The at least one CD47 binding domain may be derived from SIRPα and engineered to have a higher binding affinity than wild type SIRPα. In another case, the amino acid sequence is a fusion of two amino acid sequences homologous with the amino acid sequence set forth in SEQ ID NO:1. The least one CD47 binding domain has a CD47 binding domain having a high affinity domain than the CD47 binding domain of wild type SIRPα. In some aspects, the tetravalent fusion protein comprising an amino acid sequence having at least two amino acid substitutions in a linker region between a binding fragment and a crystallizable fragment region.

Any of the disclosed fusion proteins are high affinity fusion proteins comprising one or more variable antigen binding domains with an amino acid sequence homologous to the amino acid sequence set forth in SEQ ID NO:1 or SEQ ID NO:2. In some aspects, the one or more variable antigen binding domains have a higher binding affinity to CD47 than a binding affinity of a wild-type SIRPα.

For example, the high affinity fusion protein has two high affinity antigen binding domains each having a higher binding affinity to CD47 than a binding affinity of WT SIRPα, or the high affinity fusion protein has three high affinity antigen binding domains each of which may have a higher binding affinity to CD47 than a binding affinity of WT SIRPα, or the high affinity fusion protein may have four high affinity antigen binding domains each of which may have a higher binding affinity to CD47 than a binding affinity of WT SIRPα.

Exemplary fusion proteins include fusion proteins comprising a) a dimer of amino acid sequence of SEQ ID NO: 2, b) a dimer of amino acid sequence of SEQ ID NO: 12, c) a dimer of amino acid sequence comprising amino acids 23 to 627 of SEQ ID NO: 35, d) a dimer of amino acid sequence of SEQ ID NO: 13, e) the amino acid sequences of SEQ ID NO: 10 and SEQ ID NO: 11, f) the amino acid sequences of SEQ ID NO: 26 and SEQ ID NO: 10, g) the amino acid sequences of SEQ ID NO: 16 and SEQ ID NO: 18, h) the amino acid sequences of SEQ ID NO: 20, SEQ ID NO: 18 and SEQ ID NO: 19, i) the amino acid sequences of SEQ ID NO: 16 and SEQ ID NO: 17, j) the amino acid sequences of SEQ ID NO: 14 and SEQ ID NO: 18, k) the amino acid sequences of SEQ ID NO: 17, SEQ ID NO: 19 and SEQ ID NO: 20, l) the amino acid sequences of SEQ ID NO: 17, amino acids 23 to 492 of SEQ ID NO: 36 and SEQ ID NO: 21, m) the amino acid sequences of SEQ ID NO: 19, SEQ ID NO: 20 and SEQ ID NO: 18, n) the amino acid sequences of SEQ ID NO: 18, amino acids 23 to 492 of SEQ ID NO: 36 and SEQ ID NO: 21, o) the amino acid sequences of SEQ ID NO: 14 and amino acids 23 to 372 of SEQ ID NO: 29, p) the amino acid sequence of SEQ ID NO: 16 and amino acids 23 to 372 of SEQ ID NO: 29, q) the amino acid sequence of SEQ ID NO: 19, SEQ ID NO: 20 and amino acids 23 to 372 of amino acids 23 to 372 of SEQ ID NO: 29, r) the amino acid sequence of SEQ ID NO: 21, amino acids 23 to 492 of SEQ ID NO: 36, and amino acids 23 to 372 of SEQ ID NO: 29, s) the amino acid sequence of SEQ ID NO: 17 and amino acids 23 to 601 of SEQ ID NO: 30, t) the amino acid sequence of SEQ ID NO: 17 and the amino acids 23 to 727 of SEQ ID NO: 3, u) a sequence comprising amino acids 23 to 727 of SEQ ID NO: 31 and amino acids 23 to 372 of SEQ ID NO: 29, v) a sequence comprising amino acids 23 to 601 of SEQ ID NO: 30 and amino acids 23 to 377 of SEQ ID NO: 32, w) as sequence comprising amino acids 23 to 601 of SEQ ID NO: 30 and amino acids 23 to 502 of SEQ ID NO: 33, x) a sequence comprising amino acids 23 to 727 of SEQ ID NO: 31 and amino acids 23 to 502 of SEQ ID NO: 33, y) a sequence comprising amino acids 23 to 601 of SEQ ID NO: 30 and amino acids 23 to 497 of SEQ ID NO: 34, or z) a sequence comprising amino acids 23 to 727 of SEQ ID NO: 31 and amino acids 23 to 497 of SEQ ID NO: 34. In any of the disclosed heterodimeric fusion proteins, the IgG Fc receptor comprises the knob and/or hole domain. In any of the disclosed fusion proteins, the amino acid sequences may be enjoined using any linker known in the art, such as a peptide comprising two consecutive glycines (G-G) or a peptide comprising proline and serine (P-S).

The disclosure provides for polynucleotide sequences, such as mRNA sequences, encoding the amino acid sequence of any of the disclosed SIRPα fusion proteins, SIRPα/CCR4-binding fusion protein, high affinity fusion proteins or heterodimers including bivalent and tetravalent fusion proteins and hexavalent and octovalent fusion proteins. The disclosed polynucleotides may comprise the mature amino acid sequence of the disclosed fusion protein. In some embodiments, the disclosure provides polynucleotides encoding the amino acid sequence comprising the 22 amino acid signal sequence (MDMRVPAQLLGLLLLWLRGARC; SEQ ID NO: 66), which facilitates the secretion of the fusion protein. Exemplary mature fusion proteins are provided in SEQ ID NOS: 29-36.

The disclosure also provides for plasmids comprising at least one of the disclosed polynucleotide sequences, such a mRNA sequence that is at least 95% identical to any of the disclosed nucleotide sequences of SEQ ID NOS: 39-58, and 61-64, In addition, the disclosure also provides for plasmids comprising at least one of the disclosed polynucleotide sequences, such as the disclosed mRNA comprising the nucleotide sequence of SEQ ID NOS: 39-58, and 61-64. In some embodiments, the disclosure provides plasmids comprising at least one of the disclosed mRNA sequences of SEQ ID NOS: 39-58, and 61-64 wherein the mRNA sequences further comprise the 5′ untranslated region (UTR) sequence of SEQ ID NO: 37 and/or the 3′ UTR sequence of SEQ ID NO: 38.

The disclosure also provides for therapeutic composition comprising at least two of the polynucleotides encoding a fusion protein disclosed herein and a delivery vehicle molecule comprising an amino-lipidated peptoid. In any of the disclosed therapeutic compositions, the composition further comprises a mRNA encoding a tumor-specific antigen.

mRNA encoding any of the disclosed fusion proteins, such as SIRPα fusion proteins, SIRPα/CCR4-binding fusion protein and/or heterodimers, may be delivered in a delivery vehicle composition and the delivery vehicle comprises a cationic peptoid. In some embodiments, the cationic peptoid is a hydroxyethyl-capped tertiary amino lipidated cationic peptoid. In some embodiments, the delivery vehicle comprises a compound having Formula (I) or Formula (II). In some embodiments, the compound of Formula (I) is any one of Compounds 1-35, as set out in Table 1. In some implementations, the compound of Formula (I) is compound 1, 6, 21, or 30. In some cases, the compound of Formula (I) is compound 1. In some cases, the compound of Formula (I) is compound 6. In some cases, the compound of Formula (I) is compound 21. In some cases, the compound of Formula (I) is compound 30. In other embodiments, the compound of Formula (II) is any one of Compounds 140, 146, 151, 152, 160, 161, 162, 140, 146, 151, 152, 160, 161, or 162. In some implementations, the compound of Formula (II) is compound 140.

Also described herein are methods of using any of the disclosed fusion proteins, such as SIRPα fusion proteins, SIRPα/CCR4-binding fusion protein and/or heterodimers described herein. For example, a method comprises a step of injecting an mRNA nanoparticle or therapeutic compositions comprising a first mRNA encoding a tumor-specific antigen; a second mRNA encoding any of the disclosed fusion proteins, such as SIRPα fusion proteins, SIRPα/CCR4-binding fusion protein and/or heterodimers or fragments thereof, and a delivery vehicle molecule encapsulating the first mRNA, the second mRNA or both the first and the second mRNA. Injecting may include injecting into a tumor, and/or systemic injection. The tumor-specific antigen may be a viral antigen. For example, the viral antigen may be associated with Human papillomavirus (HPV), Kaposi© sarcoma-associated herpesvirus (KSHV), Epstein-Barr virus (EBV), Merkel cell polyomavirus, Human cytomegalovirus (CMV), or any combination thereof. In some examples, the tumor-specific antigen comprises a neo-epitope, a patient-specific antigen, and/or a shared tumor antigen. The tumor-specific antigen may comprise a plurality of patient-specific antigens. In some aspects, the delivery vehicle molecule comprises an amino-lipidated peptoid. The first mRNA encoding the tumor-specific antigen and the second mRNA is on a single mRNA strand, or multiple mRNA strands. The first mRNA encoding the tumor-specific antigen and the second mRNA are co-packaged in the same delivery vehicle molecule or are on different delivery vehicle molecules. Alternatively, the first mRNA and the second mRNA are packaged in different copies of the delivery vehicle molecule.

The nanoparticle or the therapeutic composition may further comprise a third mRNA encoding a second immunomodulatory agent. For example, the mRNA nanoparticle may further comprise an immunomodulatory siRNA. The immunomodulatory agent may encode one or more of: a checkpoint inhibitor, an immunosuppression antagonist, a pro-inflammatory agent, or any combination thereof. The immunomodulatory agent may encode a pro-inflammatory cytokine, such as one or more of: (IL), IL-1, IL-2, IL-12, IL-17, IL-18, IFN-γ, and TNF-α. The pro-inflammatory cytokine may be, for example, interleukin-12 (IL-12).

In any of the therapeutic composition or lipid nanoparticles disclosed herein, the wherein the polynucleotides comprise a) a first mRNA encoding the amino acid sequence of SEQ ID NO: 8 and a second mRNA encoding the amino acid sequence of SEQ ID NO: 9, b) a first mRNA encoding the amino acid sequence of SEQ ID NO: 10 and a second mRNA encoding the amino acid sequence of SEQ ID NO: 11, c) a first mRNA encoding the amino acid sequence of SEQ ID NO: 10 and a second mRNA encoding the amino acid sequence of SEQ ID NO: 26, d) a first mRNA encoding the amino acid sequence of SEQ ID NO: 14 and a second mRNA encoding the amino acid sequence of SEQ ID NO: 18, e) a first mRNA encoding the amino acid sequence of SEQ ID NO: 16 and a second mRNA encoding the amino acid sequence of SEQ ID NO: 18, f) a first mRNA encoding the amino acid sequence of SEQ ID NO: 18, a second mRNA encoding the amino acid sequence of SEQ ID NO: 19, and a third mRNA encoding the amino acid sequence of SEQ ID NO: 20, g) a first mRNA encoding the amino acid sequence of SEQ ID NO: 16 and a second mRNA encoding the amino acid sequence of SEQ ID NO: 17, h) a first mRNA encoding the amino acid sequence of SEQ ID NO: 17, a second mRNA encoding the amino acid sequence of SEQ ID NO: 19, and a third mRNA encoding the amino acid sequence of SEQ ID NO: 20, i) a first mRNA encoding the amino acid sequence of SEQ ID NO: 17, a second mRNA encoding the amino acid sequence of SEQ ID NO: 36, and a third mRNA encoding the amino acid sequence of SEQ ID NO: 21, j) a first mRNA encoding the amino acid sequence of SEQ ID NO: 18, a second mRNA encoding the amino acid sequence of SEQ ID NO: 19, and a third mRNA encoding the amino acid sequence of SEQ ID NO: 20, k) a first mRNA encoding the amino acid sequence of SEQ ID NO: 18, a second mRNA encoding a sequence comprising amino acids 23 to 492 of SEQ ID NO: 36, and a third mRNA encoding the amino acid sequence of SEQ ID NO: 21, l) a first mRNA encoding the amino acid sequence of SEQ ID NO: 14 and a second mRNA encoding a sequence comprising amino acids 23 to 372 of SEQ ID NO: 29, m) a first mRNA encoding the amino acid sequence of SEQ ID NO: 16 and a second mRNA encoding a sequence comprising amino acids 23 to 372 of SEQ ID NO: 29, n) a first mRNA encoding the amino acid sequence of SEQ ID NO: 19, a second mRNA encoding the amino acid sequence of SEQ ID NO: 20, and a third mRNA encoding a sequence comprising amino acids 23 to 372 of SEQ ID NO: 29, o) a first mRNA encoding a sequence comprising amino acids 23 to 492 of SEQ ID NO: SEQ ID NO: 36, a second mRNA encoding the amino acid sequence of SEQ ID NO: 21, and a third mRNA encoding a sequence comprising amino acids 23 to 372 of SEQ ID NO: 29, p) a first mRNA encoding a sequence comprising amino acids 23 to 601 of SEQ ID NO: 30, and a second mRNA encoding a sequence comprising amino acids 23 to 372 of SEQ ID NO: 29, q) a first mRNA encoding a sequence comprising amino acids 23 to 727 of SEQ ID NO: 31, and a second mRNA encoding the amino acid sequence of SEQ ID NO: 17, r) a first mRNA encoding a sequence comprising amino acids 23 to 727 of SEQ ID NO: 31, and a second mRNA encoding a sequence comprising amino acids 23 to 372 of SEQ ID NO: 29, s) a first mRNA encoding a sequence comprising amino acids 23 to 601 of SEQ ID NO: 30, and a second mRNA encoding a sequence comprising amino acids 23 to 377 of SEQ ID NO: 32, t) a first mRNA encoding a sequence comprising amino acids 23 to 727 of SEQ ID NO: 31, and a second mRNA encoding a sequence comprising amino acids 23 to 377 of SEQ ID NO: 32, u) a first mRNA encoding a sequence comprising amino acids 23 to 601 of SEQ ID NO: 30, and a second mRNA encoding a sequence comprising amino acids 23 to 502 of SEQ ID NO: 33, v) a first mRNA encoding a sequence comprising amino acids 23 to 727 of SEQ ID NO: 31 encodes the amino acid sequence of SEQ ID NO: 31, and a second mRNA encoding a sequence comprising amino acids 23 to 502 of SEQ ID NO: 33, w) a first mRNA encoding the amino acid sequence of SEQ ID NO: 14, and a second mRNA encoding a sequence comprising amino acids 23 to 497 of SEQ ID NO: 34, x) a first mRNA encoding a sequence comprising amino acids 23 to 601 of SEQ ID NO: 30, and a second mRNA encoding a sequence comprising amino acids 23 to 497 of SEQ ID NO: 34, or y) a first mRNA a sequence comprising amino acids 23 to 727 of SEQ ID NO: 31, and a second mRNA encoding a sequence comprising amino acids 23 to 497 of SEQ ID NO: 34.

The method may be a method of treating a patient having lymphoma. The method may be a method of treating a patient having cervical cancer.

Also described herein are therapeutic compositions comprising at least two polynucleotides, such as mRNA encoding the amino acid sequence of any of the disclosed fusion proteins such as SIRPα fusion proteins, SIRPα/CCR4-binding fusion proteins, high affinity fusion proteins or heterodimers including bivalent, tetravalent, hexavalent or octovalent fusion proteins and a delivery vehicle agent comprising an amino-lipidated peptoid.

In certain aspects, the polynucleotides, mRNAs and/or compositions described herein are formulated with a vehicle, a delivery agent a delivery vehicle molecule, or a delivery vehicle composition to make delivery vehicle complexes or pharmaceutical formulations. Such polyanionic compounds, e.g., polynucleotides disclosed herein, may also be referred to as polyanionic cargo compounds or cargos of a delivery vehicle complex (also referred to as a multicomponent delivery system), which complex or system also includes delivery vehicle compositions.

In some embodiments, the delivery vehicle molecule or delivery vehicle composition comprises a peptoid, a lipoid, a liposome, a lipoplex, a lipid nanoparticle, a polymeric compound, or a conjugate.

In some embodiments, the delivery vehicle molecule or delivery vehicle composition comprises lipid nanoparticles (LNPs), such as cationic lipid nanoparticles. Exemplary cationic lipid nanoparticles are described, for example, in WO2020/219941 and WO2020/097548, each of which is incorporated herein by reference. In some embodiments, the delivery vehicle molecule or delivery vehicle composition comprises peptoids, such as tertiary amino lipidated and/or PEGylated cationic peptoids. Exemplary cationic peptoids are described, for example, in WO 2020/069442, WO 2020/069445, WO 2021/030218, WO 2022/32058, and WO2023/014931, each of which is incorporated herein by reference.

In some embodiments, the delivery vehicle molecule or delivery vehicle composition is suitable for use with systemic delivery, for example, where the delivery vehicle molecule or delivery vehicle composition is preferably taken up and/or expressed by the liver. Examples of delivery vehicles having such characteristics are known in the art.

In some embodiments, the delivery vehicle molecules or delivery vehicle compositions include tertiary amino lipidated cationic peptoids (see, e.g., Example 14 herein). For example, the delivery vehicle molecules or delivery vehicle compositions may comprise 2-aminopropane-1,3-diol-capped cationic peptoids, and complexes of the delivery vehicle compositions with polyanionic compounds, such as nucleic acids. In one aspect, such peptoids have a structure of Formula (I):

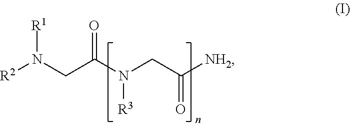

wherein n is 1, 2, 3, 4, 5, 6, 7, 8, 9, or 10; R¹ is H or C₂₋₅ alkyl optionally substituted with 1-3 OH; R² is C₂₋₅ alkylene-OH substituted with 1-3 additional OH; and each R³ independently is C₆₋₂₄ alkyl or C₆₋₂₄ alkenyl. In some cases, n is 3. In some cases, n is 4. In some cases, n is 6. In some cases, n is 8. In some cases, n is 9. In some cases, R¹ is H. In some cases, R² is C₂₋₅ alkylene-OH substituted with 1 additional OH. In some cases, C₂₋₅ alkylene is substituted with 2 or 3 additional OH. In some cases, R² is C₃₋₄ alkylene substituted with 1-3 additional OH. In some cases, each R³ independently is C₆₋₁₈ alkyl or C₆₋₁₈ alkenyl. In some cases, each R³ independently is C₈₋₁₈ alkyl or C₈₋₁₈ alkenyl. In some cases, each R³ independently is selected from the group consisting of

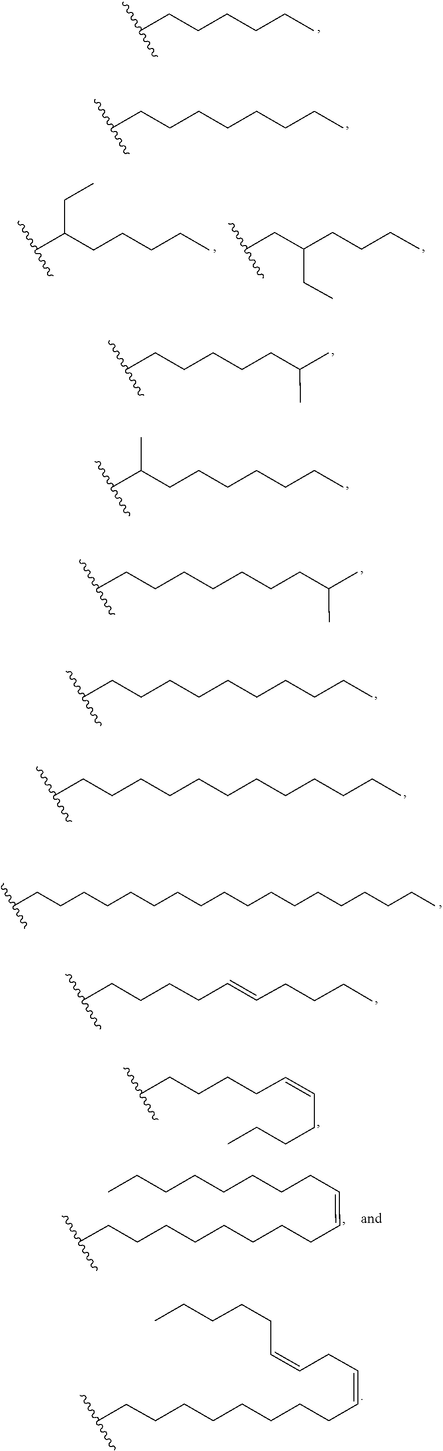

In some cases, each R³ independently is selected from the group consisting of

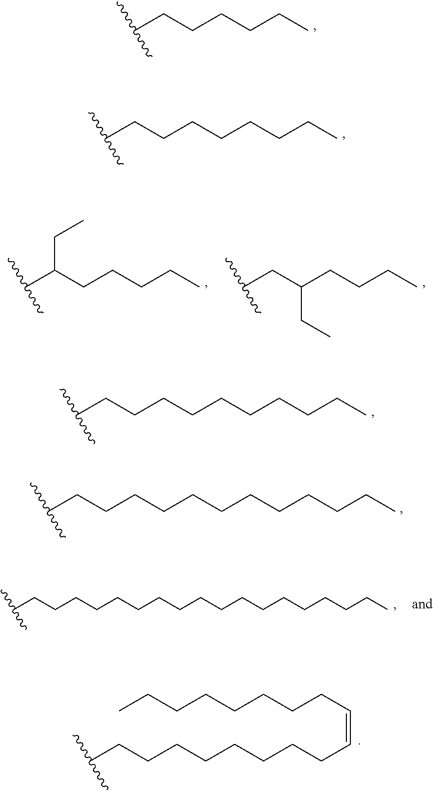

In some cases, at least one R³ is selected from the group consisting of

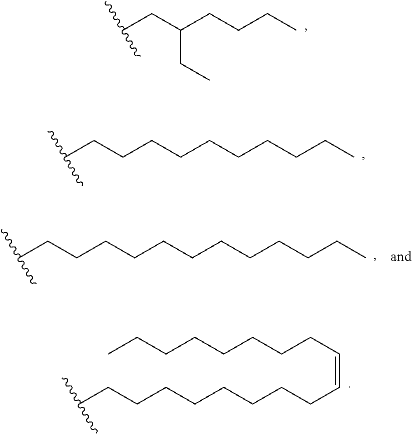

In some cases, at least one R³ is

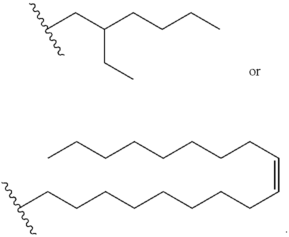

In some cases, the compound of Formula (I) has a structure selected from the group consisting of:

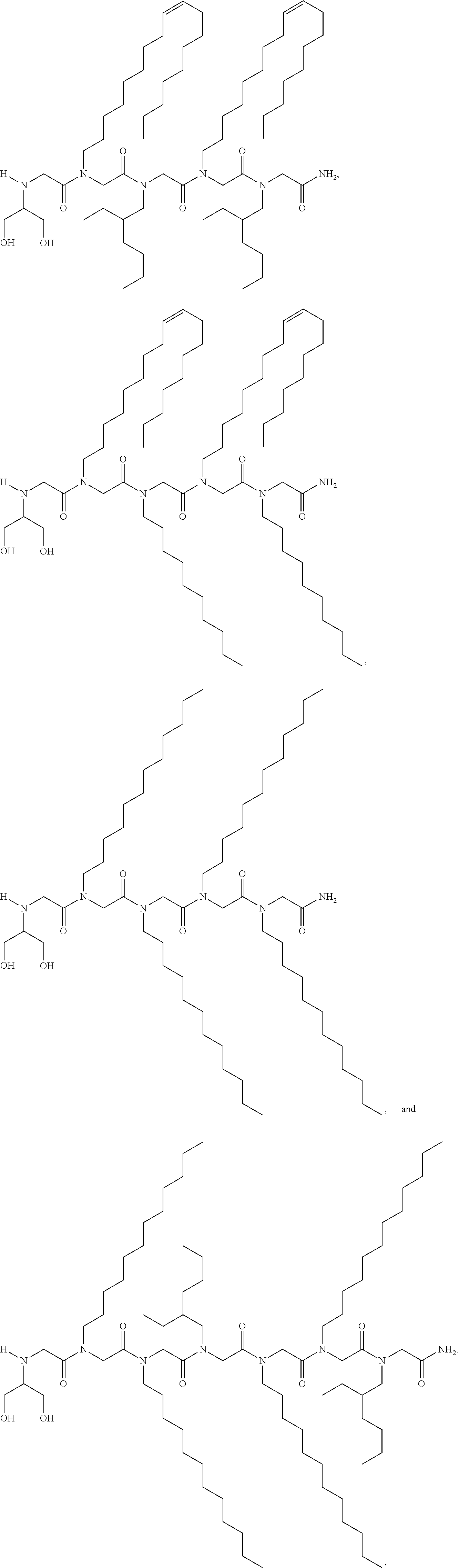

In some cases, the compound of Formula (I) has a structure:

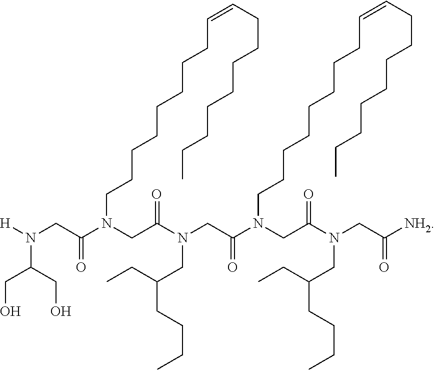 [SMILES: CCCCCCCC/C=C\CCCCCCCCN(CC(=O)N(CC(=O)N(CCCCCCCC/C=C\CCCCCCCC)CC(=O)N(CC(N)=O)CC(CC)CCCC)CC(CC)CCCC)C(=O)CNC(CO)CO]

In some cases, the compound of Formula (I) has a structure:

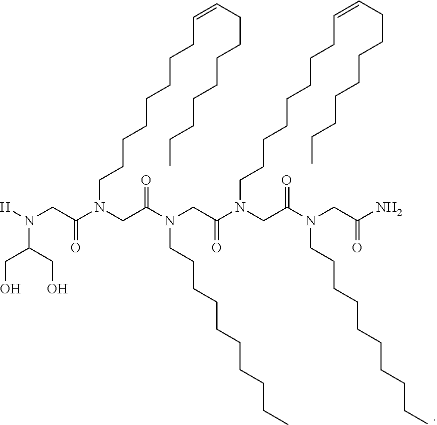 [SMILES: CCCCCCCC/C=C\CCCCCCCCN(CC(=O)N(CCCCCCCCCC)CC(=O)N(CCCCCCCC/C=C\CCCCCCCC)CC(=O)N(CCCCCCCCCC)CC(N)=O)C(=O)CNC(CO)CO]

In some cases, the compound of Formula (I) has a structure:

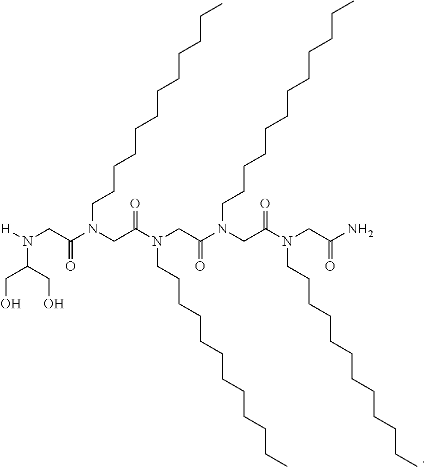 [SMILES: CCCCCCCCCCCCN(CC(N)=O)C(=O)CN(CCCCCCCCCCCC)C(=O)CN(CCCCCCCCCCCC)C(=O)CN(CCCCCCCCCCCC)C(=O)CNC(CO)CO]

In some cases, the compound of Formula (I) has a structure:

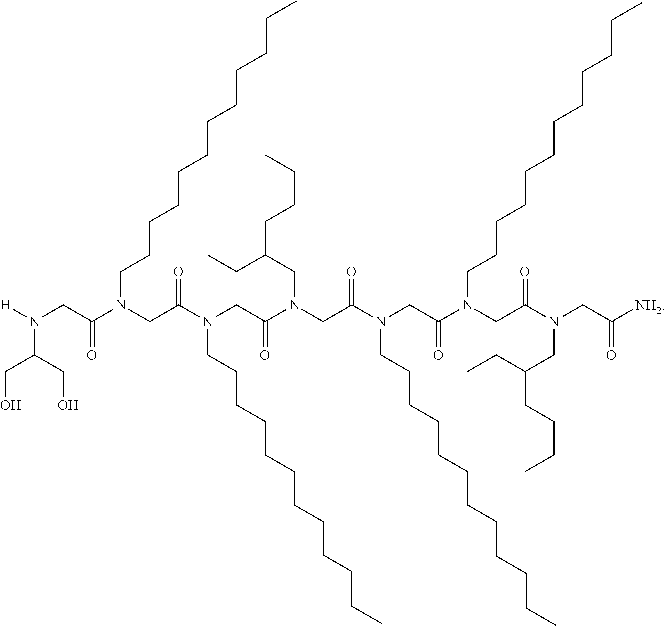 [SMILES: CCCCCCCCCCCCN(CC(=O)N(CCCCCCCCCCCC)CC(=O)N(CC(=O)N(CCCCCCCCCCCC)CC(=O)N(CCCCCCCCCCCC)CC(=O)N(CC(N)=O)CC(CC)CCCC)CC(CC)CCCC)C(=O)CNC(CO)CO]

Further disclosed herein are pharmaceutically acceptable salts of the compounds of Formula (I). For example, the delivery vehicle is any one of Compounds 1-35, as set out in Table 1. In some implementations, the compound of Formula (I) is compound 1, 6, 21, or 30. In some cases, the compound of Formula (I) is compound 1.

In some embodiments, such delivery vehicle compositions comprise the compounds disclosed herein or a pharmaceutically acceptable salt thereof. In some implementations, the composition further comprises one or more of a phospholipid, a sterol, and a PEGylated lipid. In some implementations, the compound or salt of Formula (I) is present in the delivery vehicle composition in an amount of about 30 mol % to about 60 mol %. In some implementations, the compound or salt of Formula (I) is present in the delivery vehicle composition in an amount of about 35 mol % to about 55 mol %. In various implementations, the compound or salt of Formula (I) is present in the delivery vehicle composition in an amount of about 30 mol % to about 45 mol %. In various implementations, the compound or salt of Formula (I) is present in the delivery vehicle composition in an amount of about 35 mol % to about 39 mol %. In some cases, the compound or salt of Formula (I) is present in the delivery vehicle composition in an amount of about 39 mol % to about 52 mol %. In various implementations, the compound or salt of Formula (I) is present in the delivery vehicle composition in an amount of about 30 mol % to about 35 mol %. In various implementations, the compound or salt of Formula (I) is present in the delivery vehicle composition in an amount of about 40 mol % to about 45 mol %. In various cases, the compound or salt of Formula (I) is present in an amount of about 42 mol % to about 49 mol %. In some implementations, the compound or salt of Formula (I) is present in an amount of about 50 mol % to about 52 mol %.

In various implementations, the composition comprises a phospholipid, a sterol, and a PEGylated lipid. In some cases, the composition consists essentially of a compound disclosed herein or a salt thereof, a phospholipid, a sterol, and a PEGylated lipid. In some aspects, the delivery vehicle composition consists essentially of a compound disclosed herein or a salt thereof, a phospholipid, a sterol, and a PEGylated lipid. In some aspects, the delivery vehicle composition comprises about 30 mol % to about 60 mol % of the compound of Formula (I); about 3 mol % to about 20 mol % of the phospholipid, about 25 mol % to about 60 mol % of the sterol, and about 1 mol % to about 5 mol % of the PEGylated lipid. In various aspects, the delivery vehicle composition comprises about 35 mol % to about 55 mol % of the compound or salt of Formula (I); about 5 mol % to about 15 mol % of the phospholipid, about 30 mol % to about 55 mol % of the sterol, and about 1 mol % to about 3 mol % of the PEGylated lipid. In some implementations, the delivery vehicle composition comprises about 38 mol % to about 52 mol % of the compound or salt of Formula (I); about 9 mol % to about 12 mol % of the phospholipid, about 35 mol % to about 50 mol % of the sterol, and about 1 mol % to about 2 mol % of the PEGylated lipid. In various implementations, the delivery vehicle composition comprises about 30 mol % to about 49 mol % of the compound of Formula (I); about 5 mol % to about 15 mol % of the phospholipid, about 30 mol % to about 55 mol % of the sterol, and about 1 mol % to about 3 mol % of the PEGylated lipid. In some aspects, the delivery vehicle composition comprises about 35 mol % to about 49 mol % of the compound or salt of Formula (I); about 7 mol % to about 12 mol % of the phospholipid, about 35 mol % to about 50 mol % of the sterol, and about 1 mol % to about 2 mol % of the PEGylated lipid. In some aspects, the delivery vehicle composition comprises about 30 mol % to about 45 mol % of the compound or salt of Formula (I); about 7 mol % to about 12 mol % of the phospholipid, about 40 mol % to about 55 mol % of the sterol, and about 1 mol % to about 3 mol % of the PEGylated lipid. In some aspects, the delivery vehicle composition comprises about 30 mol % to about 35 mol % of the compound or salt of Formula (I); about 7 mol % to about 12 mol % of the phospholipid, about 50 mol % to about 55 mol % of the sterol, and about 2 mol % to about 3 mol % of the PEGylated lipid. In some aspects, the delivery vehicle composition comprises about 40 mol % to about 45 mol % of the compound or salt of Formula (I); about 7 mol % to about 12 mol % of the phospholipid, about 40 mol % to about 45 mol % of the sterol, and about 1 mol % to about 2 mol % of the PEGylated lipid. In some aspects, the phospholipid is selected from the group consisting of 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C 16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanolamine (DOPE), 1,2-dipalmitoyl-sn-glycero-3-phosphoethanolamine (DPPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and combinations thereof. In some aspects, the phospholipid is DOPE, DSPC, or a combination thereof. In various aspects, the phospholipid is DSPC. In some implementations, the sterol is selected from the group consisting of cholesterol, fecosterol, sitosterol, ergosterol, campesterol, stigmasterol, brassicasterol, tomatidine, ursolic acid, alpha-tocopherol, and mixtures thereof. In some aspects, the sterol is cholesterol. In some implementations, the PEGylated lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, a PEG-modified sterol, and a PEG-modified phospholipid. In various implementations, the PEG-modified lipid is selected from the group consisting of PEG-modified cholesterol, N-octanoyl-sphingosine-1-{succinyl[methoxy(polyethylene glycol)]}, N-palmitoyl-sphingosine-1-{succinyl[methoxy(polyethylene glycol)]}, PEG-modified DMPE (DMPE-PEG), PEG-modified DSPE (DSPE-PEG), PEG-modified DPPE (DPPE-PEG), PEG-modified DOPE (DOPE-PEG), dimyristoylglycerol-polyethylene glycol (DMG-PEG), distearoylglycerol-polyethylene glycol (DSG-PEG), dipalmitoylglycerol-polyethylene glycol (DPG-PEG), dioleoylglycerol-polyethylene glycol (DOG-PEG), and a combination thereof. In some aspects, the PEG-modified lipid is dimyristoylglycerol-polyethylene glycol 2000 (DMG-PEG 2000). In various aspects, the delivery vehicle composition comprises about 38.2 mol % of Compound 140, about 11.8 mol % of DSPC, about 48.2 mol % of cholesterol, and about 1.9 mol % of DMG-PEG 2000.

In some implementations, the delivery vehicle composition comprises about 42.6 mol % of Compound 140, about 10.9 mol % of DSPC, about 44.7 mol % of cholesterol, and about 1.7 mol % of DMG-PEG 2000. In some implementations, the delivery vehicle composition comprises about 48.2 mol % of Compound 140, about 9.9 mol % of DSPC, about 40.4 mol % of cholesterol, and about 1.6 mol % of DMG-PEG 2000. In various aspects, the delivery vehicle composition comprises about 51.3 mol % of Compound 140, about 9.3 mol % of DSPC, about 38 mol % of cholesterol, and about 1.5 mol % of DMG-PEG 2000. In various aspects, the delivery vehicle composition comprises about 44.4 mol % of Compound 140, about 10.6 mol % of DSPC, about 43.3 mol % of cholesterol, and about 1.7 mol % of DMG-PEG 2000. In various aspects, the delivery vehicle composition comprises about 44.4 mol % of Compound 140, about 10.6 mol % of DSPC, about 43.4 mol % of cholesterol, and about 1.7 mol % of DMG-PEG 2000. In various aspects, the delivery vehicle composition comprises about 33.1 mol % of Compound 140, about 10.6 mol % of DSPC, about 53.8 mol % of cholesterol, and about 2.5 mol % of DMG-PEG 2000.

Further disclosed herein is a therapeutic formulation comprising one or more polyanionic compounds (e.g., mRNAs encoding polynucleotides described herein) with a delivery vehicle complex comprising the delivery vehicle composition described herein and a polyanionic compound. In some aspects, the compound of Formula (I) or salt thereof is complexed to the polyanionic compound. In various aspects, the compound or salt of Formula (I) and the polyanionic compound are present in a mass ratio of about 5:1 to about 25:1. In some implementations, the compound or salt of Formula (I) and the polyanionic compound are present in a mass ratio of about 7:1 to about 20:1. In various aspects, the compound or salt of Formula (I) and the polyanionic compound are present in a mass ratio of about 10:1 to about 17:1. In some aspects, the compound or salt of Formula (I) and the polyanionic compound are present in a mass ratio of about 19:1. In some aspects, the compound or salt of Formula (I) and the polyanionic compound are present in a mass ratio of about 20:1. In some aspects, the compound or salt of Formula (I) and the polyanionic compound are present in a mass ratio of about 10:1. In various aspects, the compound or salt of Formula (I) and the polyanionic compound are present in a mass ratio of about 12:1, the compound or salt of Formula (I) and the polyanionic compound are present in a mass ratio of about 13:1. In some implementations, the compound or salt of Formula (I) and the polyanionic compound are present in a mass ratio of about 15:1. In various implementations, the compound or salt of Formula (I) and the polyanionic compound are present in a mass ratio of about 17:1. In some aspects, the phospholipid and the polyanionic compound are present in a mass ratio of about 2:1 to about 10:1. In some aspects, the phospholipid and the polyanionic compound are present in a mass ratio of about 2:1 to about 4:1. In various aspects, the phospholipid and the polyanionic compound are present in a mass ratio of about 2:1 to about 3:1. In various aspects, the phospholipid and the polyanionic compound are present in a mass ratio of about 4.0:1. In various aspects, the phospholipid and the polyanionic compound are present in a mass ratio of about 2.7:1. In some implementations, the sterol and the polyanionic compound are present in a mass ratio of about 5:1 to about 8:1. In some implementations, the sterol and the polyanionic compound are present in a mass ratio of about 5:1 to about 6:1. In various implementations, the sterol and the polyanionic compound are present in a mass ratio of about 5.4:1. In some aspects, the sterol and the polyanionic compound are present in a mass ratio of about 8.1:1. In some aspects, the sterol and the polyanionic compound are present in a mass ratio of about 6.7:1. In some aspects, the PEGylated lipid and the polyanionic compound are present in a mass ratio of about 0.5:1 to about 2.5:1. In various aspects, the PEGylated lipid and the polyanionic compound are present in a mass ratio of about 1:1 to about 2:1. In some aspects, the phospholipid and the polyanionic compound are present in a mass ratio of about 2.1:1. In some aspects, the phospholipid and the polyanionic compound are present in a mass ratio of about 1.4:1. In various aspects the delivery vehicle complex comprises Compound 140 having about a 10:1 mass ratio to the polyanionic compound, DSPC having about a 2.7:1 mass ratio to the polyanionic compound, cholesterol having about a 5.4:1 mass ratio to the polyanionic compound, and DMG-PEG 2000 having about a 1.4:1 mass ratio to the polyanionic compound. In various aspects, the delivery vehicle complex comprises Compound 140 having about a 12:1 mass ratio to the polyanionic compound, DSPC having about a 2.7:1 mass ratio to the polyanionic compound, cholesterol having about a 5.4:1 mass ratio to the polyanionic compound, and DMG-PEG 2000 having about a 1.4:1 mass ratio to the polyanionic compound. In some aspects, the delivery vehicle complex comprises Compound 140 having about a 15:1 mass ratio to the polyanionic compound, DSPC having about a 2.7:1 mass ratio to the polyanionic compound, and cholesterol having about a 5.4:1 mass ratio to the polyanionic compound, and DMG-PEG 2000 having about a 1.4:1 mass ratio to the polyanionic compound. In various aspects, the delivery vehicle complex comprises Compound 140 having about a 17:1 mass ratio to the polyanionic compound, DSPC having about a 2.7:1 mass ratio to the polyanionic compound, cholesterol having about a 5.4:1 mass ratio to the polyanionic compound, and DMG-PEG 2000 having about a 1.4:1 mass ratio to the polyanionic compound. In various aspects, the delivery vehicle complex comprises Compound 140 having about a 13:1 mass ratio to the polyanionic compound, DSPC having about a 2.7:1 mass ratio to the polyanionic compound, cholesterol having about a 5.4:1 mass ratio to the polyanionic compound, and DMG-PEG 2000 having about a 1.4:1 mass ratio to the polyanionic compound. In various aspects, the delivery vehicle complex comprises Compound 140 having about a 19:1 mass ratio to the polyanionic compound, DSPC having about a 4.0:1 mass ratio to the polyanionic compound, cholesterol having about a 5.4:1 mass ratio to the polyanionic compound, and DMG-PEG 2000 having about a 2.1:1 mass ratio to the polyanionic compound. In various aspects, the delivery vehicle complex comprises Compound 140 having about a 9.7:1 mass ratio to the polyanionic compound, DSPC having about a 2.7:1 mass ratio to the polyanionic compound, cholesterol having about a 6.7:1 mass ratio to the polyanionic compound, and DMG-PEG 2000 having about a 2.1:1 mass ratio to the polyanionic compound.

In some cases, the complex exhibits a particle size of about 50 nm to about 200 nm and/or a polydispersity index (PDI) of less than 0.25. In various cases, the complex exhibits a particle size of about 60 nm to about 100 nm. In some implementations, the complex exhibits a particle size between about 60 nm to about 90 nm. In various implementations, the complex exhibits a particle size of about 105 nm to about 200 nm. In various cases, the complex exhibits a particle size of about 150 nm to about 200 nm. In some cases, the delivery vehicle complex exhibits a particle size of about 105 nm to about 200 nm. In some cases, the delivery vehicle complex exhibits a particle size of about 40 nm to about 115 nm, or about 55 nm to about 95 nm, or about 70 to about 80 nm, or about 75 nm. In various cases, the delivery vehicle complex exhibits a particle size of about 135 nm to about 225 nm, or about 155 nm to about 195 nm, or about 170 to about 180 nm, or about 175 nm. In various cases, at least 80% of the polyanionic compound is retained after storage at 4° C. for 48 days, or the delivery vehicle complex retains at least 80% of its original size after storage at 4° C. for 48 days, or both.

In some cases, the polyanionic compound comprises at least one nucleic acid. In various cases, the at least one nucleic acid comprises RNA, DNA, or a combination thereof. In various cases, the at least one nucleic acid comprises RNA. In some implementations, the RNA is mRNA encoding a peptide, a protein, or a functional fragment of the foregoing.

In some embodiments, the delivery vehicle molecule or delivery vehicle composition is a cationic peptoid.

In some embodiments, the cationic peptoid is a hydroxyethyl-capped tertiary amino lipidated cationic peptoid. In some embodiments, the cationic peptoid complexes with polyanionic compounds, such as nucleic acids, e.g., mRNAs polynucleotides (described herein) encoding polypeptides (described herein), and is particularly suitable for local delivery (versus systemic delivery). In some embodiments, the delivery vehicle comprises a compound having Formula (II)

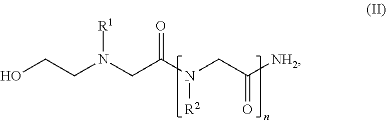

wherein n is 1, 2, 3, 4, 5, or 6; R1 is H, C1-3 alkyl, or hydroxyethyl; and each R2 independently is C8-24 alkyl or C8-24 alkenyl. In some aspects, n is 3. In various aspects, n is 4. In some implementations, R1 is H. In some aspects, R1 is ethyl or hydroxyethyl. In various aspects, R2 independently is C8-18 alkyl or C8-18 alkenyl. In some implementations, each R2 is selected from the group consisting of

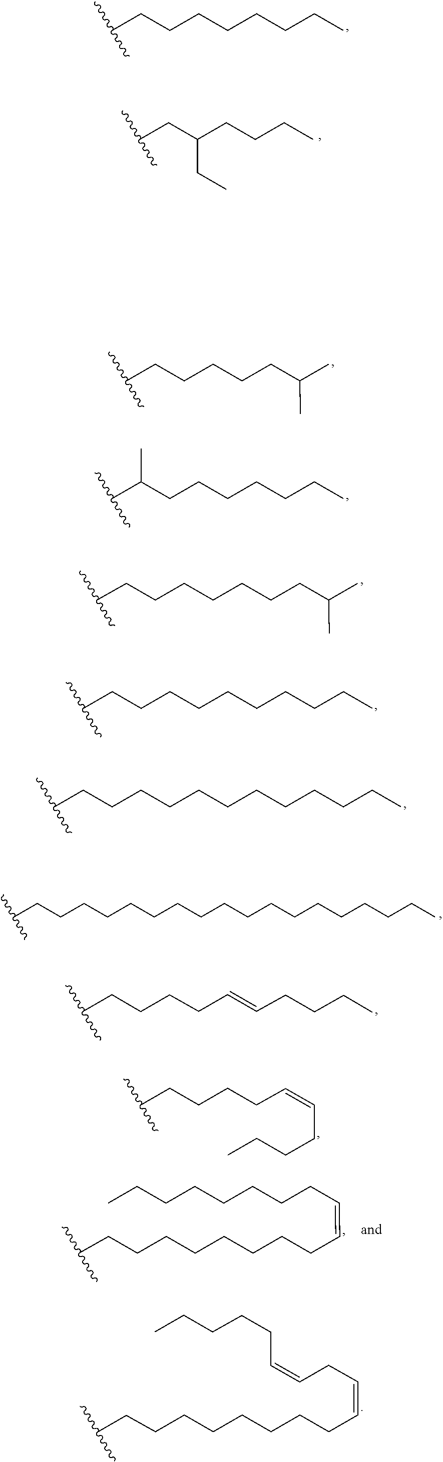

In various aspects, each R² independently is selected from the group consisting of

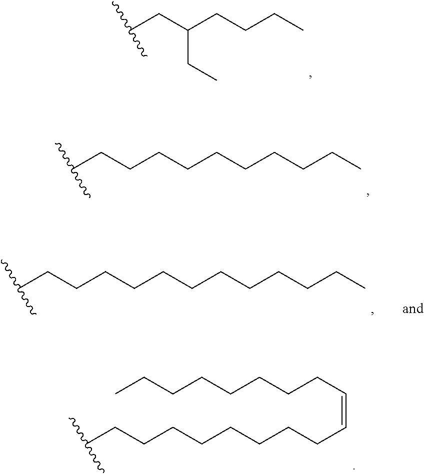

In some implementations, R² independently is selected from the group consisting of

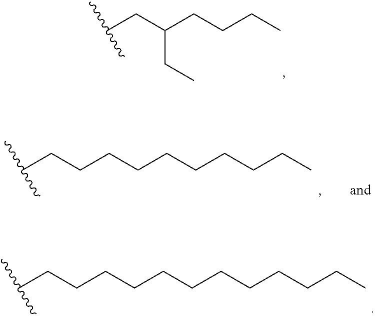

In various implementations, each R² is

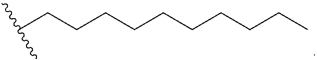 [SMILES: *CCCCCCCCCC]

In some aspects, the compound of Formula (II) has a structure selected from the group consisting of:

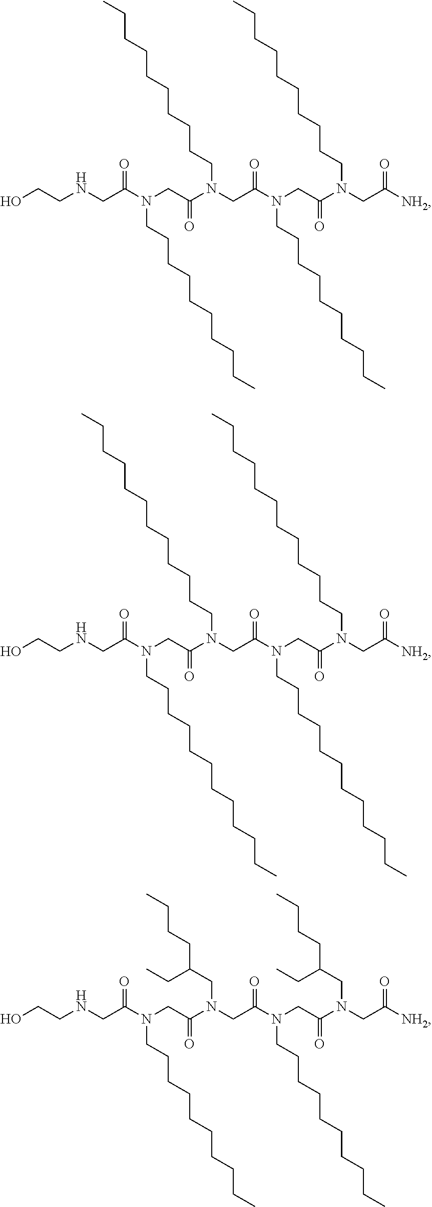

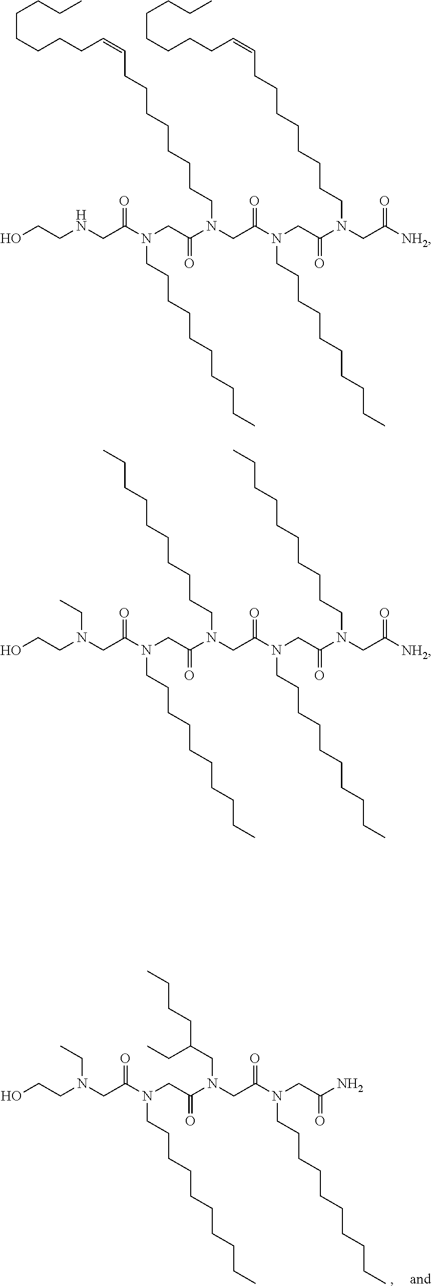

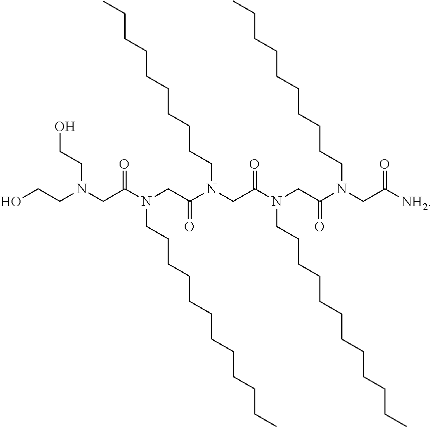 [SMILES: CCCCCCCCCCCCN(CC(=O)N(CCCCCCCCCC)CC(=O)N(CCCCCCCCCCCC)CC(=O)N(CCCCCCCCCC)CC(N)=O)C(=O)CN(CCO)CCO]

In various aspects, the compound of Formula (II) has a structure:

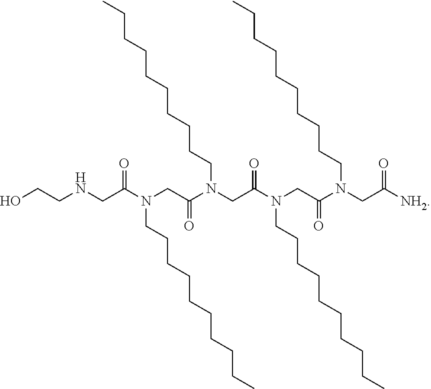 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CNCCO]

In some embodiments, the delivery vehicle molecule comprises pharmaceutically acceptable salts of the above compounds and/or the compounds of Formula (II).

Another aspect of the disclosure provides mRNA therapeutic formulations in delivery vehicle compositions comprising the compounds disclosed above or a pharmaceutically acceptable salt thereof. In some implementations, the delivery vehicle composition further comprises one or more of a phospholipid, a sterol, and a PEGylated lipid. In some implementations, the compound or salt of Formula (II) is present in the delivery vehicle composition in an amount of about 30 mol % to about 60 mol %. In some implementations, the compound or salt of Formula (II) is present in the delivery vehicle composition in an amount of about 35 mol % to about 55 mol %. In various implementations, the compound or salt of Formula (II) is present in the delivery vehicle composition in an amount of about 30 mol % to about 45 mol %. In various implementations, the compound or salt of Formula (II) is present in the delivery vehicle composition in an amount of about 35 mol % to about 39 mol %. In some aspects, the compound or salt of Formula (II) is present in the delivery vehicle composition in an amount of about 39 mol % to about 52 mol %. In various implementations, the compound or salt of Formula (II) is present in the delivery vehicle composition in an amount of about 30 mol % to about 35 mol %. In various implementations, the compound or salt of Formula (II) is present in the delivery vehicle composition in an amount of about 40 mol % to about 45 mol %. In various aspects, the compound or salt of Formula (II) is present in an amount of about 42 mol % to about 49 mol %. In some implementations, the compound or salt of Formula (II) is present in an amount of about 50 mol % to about 52 mol %.

In various implementations, the delivery vehicle composition comprises a phospholipid, a sterol, and a PEGylated lipid. In some aspects, the mRNA encodes for a SIRPα fusion protein comprising an amino acid sequence that is at least 95% homologous to the amino acid sequence of SEQ ID NO: 2 or SEQ ID NO: 3, wherein the mRNA is in a delivery vehicle composition and the delivery vehicle molecule comprises a cationic peptoid. In some embodiments, the cationic peptoid is a hydroxyethyl-capped tertiary amino lipidated cationic peptoid. In some embodiments, the delivery vehicle molecule comprises a compound having Formula (I) or Formula (II).

In some aspects, the mRNA encodes for a SIRPα fusion protein comprising an amino acid sequence of SEQ ID NO: 2 or SEQ ID NO: 3, wherein the mRNA is in a delivery vehicle composition and the delivery vehicle molecule comprises a cationic peptoid. In some embodiments, the cationic peptoid is a hydroxyethyl-capped tertiary amino lipidated cationic peptoid. In some embodiments, the delivery vehicle molecule comprises a compound having Formula (I) or Formula (II).

In some aspects, the mRNA encodes for a SIRPα fusion protein comprising an amino acid sequence that is at least 95% homologous to the amino acid sequence of SEQ ID NO: 2 or SEQ ID NO: 3, wherein the SIRPα fusion protein comprises a tetravalent structure and the amino acid sequence comprises ten or fewer amino acid substitutions relative to SEQ ID NO: 23, wherein the mRNA is in a delivery vehicle composition and the delivery vehicle molecule comprises a cationic peptoid. In some embodiments, the cationic peptoid is a hydroxyethyl-capped tertiary amino lipidated cationic peptoid. In some embodiments, the delivery vehicle molecule comprises a compound having Formula (I) or Formula (II).

In some aspects, the mRNA encodes for a SIRPα/CCR4-binding fusion protein comprising an amino acid sequence that is at least 95% homologous to the amino acid sequence of SEQ ID NO: 14 and an amino acid sequence that is at least 95% homologous to the amino acid sequence of SEQ ID NO: 22 or SEQ ID NO: 23, wherein the mRNA is in a delivery vehicle composition and the delivery vehicle molecule comprises a cationic peptoid. In some embodiments, the cationic peptoid is a hydroxyethyl-capped tertiary amino lipidated cationic peptoid. In some embodiments, the delivery vehicle molecule comprises a compound having Formula (I) or Formula (II) such as any one of Compounds 1-35, as set out in Table 1, or the Compounds 140, 146, 151, 152, 160, 161, 162, 140, 146, 151, 152, 160, 161, or 162 ion Table 2. In some implementations, the compound of Formula (I) is compound 1, 6, 21, or 30. In some cases, the compound of Formula (I) is compound 1. In some cases, the compound of Formula (I) is compound 6. In some cases, the compound of Formula (I) is compound 21. In some cases, the compound of Formula (I) is compound 30. In some implementations, the compound of Formula (II) is compound 140.

In some aspects, the mRNA encodes for a SIRPα/CCR4-binding fusion protein comprising an amino acid sequence of SEQ ID NO: 14 and either SEQ ID NO: 22 or SEQ ID NO: 23, wherein the mRNA is in a delivery vehicle composition and the delivery vehicle molecule comprises a cationic peptoid. In some embodiments, the cationic peptoid is a hydroxyethyl-capped tertiary amino lipidated cationic peptoid. In some embodiments, the delivery vehicle molecule comprises a compound having Formula (I) or Formula (II).

In some aspects, the mRNA encodes for a SIRPα fusion protein comprising an amino acid sequence that is at least 95% homologous to the amino acid sequence of SEQ ID NO: 15, SEQ ID NO: 16, SEQ ID NO: 17, SEQ ID NO: 20, or SEQ ID NO: 21, wherein the mRNA is in a delivery vehicle composition and the delivery vehicle molecule comprises a cationic peptoid. In some embodiments, the cationic peptoid is a hydroxyethyl-capped tertiary amino lipidated cationic peptoid. In some embodiments, the delivery vehicle molecule comprises a compound having Formula (I) or Formula (II) such as any one of Compounds 1-35, as set out in Table 1, or the Compounds 140, 146, 151, 152, 160, 161, 162, 140, 146, 151, 152, 160, 161, or 162 ion Table 2. In some implementations, the compound of Formula (I) is compound 1, 6, 21, or 30. In some cases, the compound of Formula (I) is compound 1. In some cases, the compound of Formula (I) is compound 6. In some cases, the compound of Formula (I) is compound 21. In some cases, the compound of Formula (I) is compound 30. In some implementations, the compound of Formula (II) is compound 140.

In some aspects, the mRNA encodes for a SIRPα/CCR4-binding fusion protein comprising an amino acid sequence of SEQ ID NO: 15, SEQ ID NO: 16, SEQ ID NO: 17, SEQ ID NO: 20, or SEQ ID NO: 21 wherein the mRNA is in a delivery vehicle composition and the delivery vehicle molecule comprises a cationic peptoid. In some embodiments, the cationic peptoid is a hydroxyethyl-capped tertiary amino lipidated cationic peptoid. In some embodiments, the delivery vehicle molecule comprises a compound having Formula (I) or Formula (II) such as any one of Compounds 1-35, as set out in Table 1, or the Compounds 140, 146, 151, 152, 160, 161, 162, 140, 146, 151, 152, 160, 161, or 162 ion Table 2. In some implementations, the compound of Formula (I) is compound 1, 6, 21, or 30. In some cases, the compound of Formula (I) is compound 1. In some cases, the compound of Formula (I) is compound 6. In some cases, the compound of Formula (I) is compound 21. In some cases, the compound of Formula (I) is compound 30. In some implementations, the compound of Formula (II) is compound 140.

Further disclosed herein is a pharmaceutical composition or formulation comprising the delivery vehicle complexes or multicomponent delivery systems described herein and a pharmaceutically acceptable excipient. In some aspects, the pharmaceutical composition is suitable for intramuscular (IM), intratumoral (IT), intravenous (IV), intraperitoneal, or subcutaneous (SQ), intradermal or mucosal delivery.

Also disclosed herein is a method of delivering one or more polyanionic compounds, e.g., one or more mRNA polynucleotides described herein, to a cell, comprising: contacting the cell with a pharmaceutical formulation or multicomponent delivery system comprising a delivery vehicle complex described herein. In some aspects, the cell is a muscle cell, an epithelial cell, a tumor cell, or a combination of two or all three. In some aspects, the cell is a cervical cancer cell, hepatocellular carcinoma cell, ovarian cancer cell, a colon cancer cell, a disseminated gastric tumor cell, B-cell lymphoma, T-cell lymphoma, prostate cancer cell, lung cancer cell. In some aspects, the polyanionic compound is an mRNA that encodes for a peptide, a protein, or a fragment of any of the foregoing, and the cell expresses the peptide, the protein, or the fragment after being contacted with the delivery vehicle complex.

Also disclosed herein is a method of forming the delivery vehicle complex disclosed herein, comprising contacting a compound or salt of Formula (I) or Formula (II) with the polyanionic compound (such as an mRNA). In some aspects, the method comprises admixing a solution comprising the compound or salt of Formula (I) or Formula (II), with a solution comprising the polyanionic compound (e.g., an mRNA).

Also disclosed herein are vaccines comprising a delivery vehicle complex disclosed herein or a pharmaceutical composition disclosed herein. Also disclosed are vaccines comprising a delivery vehicle complex disclosed herein or a pharmaceutical composition disclosed herein for use in the treatment or prevention of cancer.

Also disclosed are methods of treating or preventing cancer in a patient, comprising administering to the patient a delivery vehicle complex disclosed herein, or a pharmaceutical composition disclosed herein. In some aspects, the administering is to treat cervical cancer, hepatocellular carcinoma, ovarian cancer, colon cancer, a disseminated gastric tumor, B-cell lymphoma, T-cell lymphoma, prostate cancer, lung cancer, or a combination thereof.

Also described herein methods of inducing an immune response in a subject, the method comprising: administering a lipid nanoparticle, vaccine or therapeutic composition disclosed herein, wherein the lipid nanoparticle comprises i) a first mRNA encoding a tumor-specific antigen; ii) a second mRNA encoding any of the SIRPα fusion proteins, SIRPα/CCR4-binding fusion proteins, high affinity fusion proteins or heterodimers including bivalent and tetravalent fusion proteins disclosed herein and iii) a delivery vehicle molecule, lipid nanoparticle at least partially encapsulates the first mRNA, the second mRNA or both the first and the second mRNA.

Also described herein are methods of treating a subject, the method comprising: administering a lipid nanoparticle, vaccine or therapeutic composition disclosed herein, wherein the lipid nanoparticle comprises i) a first mRNA encoding a tumor-specific antigen; ii) a second mRNA encoding any of the fusion proteins, such as the SIRPα fusion proteins or SIRPα/CCR4-binding fusion proteins, high affinity fusion proteins and/or heterodimers including bivalent, tetravalent, hexavalent or octovalent fusion proteins disclosed herein disclosed herein and iii) a delivery vehicle molecule or a lipid nanoparticle at least partially encapsulates the first mRNA, the second mRNA or both the first and the second mRNA.

Also described herein are methods of treating or preventing cancer in a subject, the method comprising: administering a lipid nanoparticle, vaccine or therapeutic composition disclosed herein wherein the lipid nanoparticle comprises i) a first mRNA encoding a tumor-specific antigen; ii) a second mRNA encoding any of the fusion proteins, such as SIRPα fusion proteins, SIRPα/CCR4-binding fusion proteins, high affinity fusion proteins and/or heterodimers including bivalent, tetravalent, hexavalent or octovalent fusion proteins disclosed herein and iii) a delivery vehicle molecule, lipid nanoparticle at least partially encapsulates the first mRNA, the second mRNA or both the first and the second mRNA.

In any of the nanoparticles, therapeutic compositions or vaccines described herein, the tumor-specific antigen may comprise a viral antigen. The viral antigen may be with Human papillomavirus (HPV), Kaposi© sarcoma-associated herpesvirus (KSHV), Epstein-Barr virus (EBV), Merkel cell polyomavirus, Human cytomegalovirus (CMV), or any combination thereof. The tumor-specific antigen may comprise a neo-epitope. In some examples, the tumor-specific antigen comprises a patient-specific antigen. In some examples, the tumor-specific antigen comprises a plurality of patient-specific antigens. The tumor-specific antigen may comprise a shared tumor antigen. The delivery vehicle molecule may comprise an amino-lipidated peptoid. In some examples, the mRNA nanoparticle further comprises a third mRNA encoding a second immunomodulatory agent. The mRNA nanoparticle may further comprise an immunomodulatory siRNA. The immunomodulatory agent may encode one or more of: a checkpoint inhibitor, an immunosuppression antagonist, a pro-inflammatory agent, or any combination thereof. The immunomodulatory agent may encode a pro-inflammatory cytokine. For example, the pro-inflammatory cytokine may be one of: (IL), IL-1, IL-2, IL-12, IL-17, IL-18, IFN-γ, and TNF-α. The pro-inflammatory cytokine may be interleukin-12 (IL-12). The first mRNA encoding the tumor-specific antigen and the second mRNA may be on a single mRNA strand. The first mRNA encoding the tumor-specific antigen and the second mRNA may be co-packaged in the same delivery vehicle molecule. The first mRNA and the second mRNA may be packaged in different copies of the delivery vehicle molecule. The method may be a method of treating a subject having lymphoma or inducing an immune response in a subject having lymphoma. The method may be a method of treating a subject having cervical cancer or inducing an immune response in a subject having cervical cancer.

The disclosure also provides for compositions for delivering one or more polyanionic compounds, e.g., one or more mRNA polynucleotides described herein, to a cell, wherein the composition comprises a pharmaceutical formulation or multicomponent delivery system comprising a delivery vehicle complex described herein. In some aspects, the composition is delivered to a muscle cell, an epithelial cell, a tumor cell, or a combination of two or all three. In some aspects, the cell is a cervical cancer cell, hepatocellular carcinoma cell, ovarian cancer cell, a colon cancer cell, a disseminated gastric tumor cell, B-cell lymphoma, T-cell lymphoma, prostate cancer cell, lung cancer cell. In some aspects, the polyanionic compound is an mRNA that encodes for a peptide, a protein, or a fragment of any of the foregoing, and the cell expresses the peptide, the protein, or the fragment after being contacted with the delivery vehicle complex.

Also disclosed are compositions for treating or preventing cancer in a patient, wherein the composition comprises a delivery vehicle complex disclosed herein, or a pharmaceutical composition disclosed herein. In some aspects, the composition is for treating or preventing cervical cancer, hepatocellular carcinoma, ovarian cancer, a colon cancer, a disseminated gastric tumor, B-cell lymphoma, T-cell lymphoma, prostate cancer, lung cancer, or a combination thereof.

Also described herein are compositions for inducing an immune response in a subject, wherein the composition comprises a lipid nanoparticle, vaccine or therapeutic composition disclosed herein, wherein the lipid nanoparticle comprises i) a first mRNA encoding a tumor-specific antigen; ii) a second mRNA encoding any of the fusion proteins, such as SIRPα fusion proteins, SIRPα/CCR4-binding fusion proteins, high affinity fusion proteins or heterodimers including bivalent, tetravalent, hexavalent and octovalent fusion proteins disclosed herein and iii) a delivery vehicle molecule, lipid nanoparticle at least partially encapsulates the first mRNA, the second mRNA or both the first and the second mRNA.

Also described herein are compositions for treating a subject, wherein the composition comprises a lipid nanoparticle, vaccine or therapeutic composition disclosed herein wherein the lipid nanoparticle comprises i) a first mRNA encoding a tumor-specific antigen; ii) a second mRNA encoding any of the fusion proteins, such as SIRPα fusion proteins or SIRPα/CCR4-binding fusion proteins, high affinity fusion proteins and/or heterodimers including bivalent, tetravalent, hexavalent or octovalent fusion proteins disclosed herein and disclosed herein and iii) a delivery vehicle molecule, lipid nanoparticle at least partially encapsulates the first mRNA, the second mRNA or both the first and the second mRNA.

Also described herein are uses of a lipid nanoparticle, vaccine or therapeutic composition disclosed herein for the preparation of a medicament for treating or preventing cancer in a subject, wherein the lipid nanoparticle comprises i) a first mRNA encoding a tumor-specific antigen; ii) a second mRNA encoding any of the fusion proteins, such as SIRPα fusion proteins, SIRPα/CCR4-binding fusion proteins, high affinity fusion proteins or heterodimers including bivalent, tetravalent, hexavalent and octovalent fusion proteins disclosed herein and iii) a delivery vehicle molecule, lipid nanoparticle at least partially encapsulates the first mRNA, the second mRNA or both the first and the second mRNA.

Also disclosed herein are uses of a lipid nanoparticle, vaccine or therapeutic composition disclosed herein for the preparation of a medicament for delivering one or more polyanionic compounds, e.g., one or more mRNA polynucleotides described herein, to a cell, comprising: contacting the cell with a pharmaceutical formulation or multicomponent delivery system comprising a delivery vehicle complex described herein. In some aspects, the cell is a muscle cell, an epithelial cell, a tumor cell, or a combination of two or all three. In some aspects, the cell is a cervical cancer cell, hepatocellular carcinoma cell, ovarian cancer cell, a colon cancer cell, a disseminated gastric tumor cell, B-cell lymphoma, T-cell lymphoma, prostate cancer cell, lung cancer cell. In some aspects, the polyanionic compound is an mRNA that encodes for a peptide, a protein, or a fragment of any of the foregoing, and the cell expresses the peptide, the protein, or the fragment after being contacted with the delivery vehicle complex.

Also disclosed are uses of a delivery vehicle complex disclosed herein, or a pharmaceutical composition disclosed herein for the preparation of a medicament for methods of treating or preventing cancer in a patient. In some aspects, use is for treating or preventing cervical cancer, hepatocellular carcinoma, ovarian cancer, colon cancer, a disseminated gastric tumor, B-cell lymphoma, T-cell lymphoma, prostate cancer, lung cancer, or a combination thereof.

Also described herein are uses of a lipid nanoparticle, vaccine or therapeutic composition disclosed herein for the preparation of a medicament for methods of inducing an immune response in a subject, wherein the lipid nanoparticle comprises i) a first mRNA encoding a tumor-specific antigen; ii) a second mRNA encoding any of the fusion protein, such as SIRPα fusion proteins, SIRPα/CCR4-binding fusion proteins, high affinity fusion proteins or heterodimers including bivalent, tetravalent, hexavalent and octovalent fusion proteins disclosed herein and iii) a delivery vehicle molecule, lipid nanoparticle at least partially encapsulates the first mRNA, the second mRNA or both the first and the second mRNA.

Also described herein are uses of a lipid nanoparticle, vaccine or therapeutic composition disclosed herein for the preparation of a medicament for methods of treating a subject, the lipid nanoparticle wherein the lipid nanoparticle comprises i) a first mRNA encoding a tumor-specific antigen; ii) a second mRNA encoding any of the fusion proteins, such as SIRPα fusion proteins or SIRPα/CCR4-binding fusion proteins, high affinity fusion proteins and/or heterodimers including bivalent, tetravalent, hexavalent or octovalent fusion proteins disclosed herein and iii) a delivery vehicle molecule, lipid nanoparticle at least partially encapsulates the first mRNA, the second mRNA or both the first and the second mRNA.

Also described herein are uses of a lipid nanoparticle, vaccine or therapeutic composition disclosed herein for the preparation of a medicament for methods of treating or preventing cancer in a subject, wherein the lipid nanoparticle comprises i) a first mRNA encoding a tumor-specific antigen; ii) a second mRNA encoding any of the fusion proteins, such as SIRPα fusion proteins, SIRPα/CCR4-binding fusion proteins, high affinity fusion proteins or heterodimers including bivalent, tetravalent, hexavalent and octovalent fusion proteins disclosed herein and iii) a delivery vehicle molecule, lipid nanoparticle at least partially encapsulates the first mRNA, the second mRNA or both the first and the second mRNA.

All of the methods and apparatuses described herein, in any combination, are herein contemplated and can be used to achieve the benefits as described herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

A better understanding of the features and advantages of the methods and apparatuses described herein will be obtained by reference to the following detailed description that sets forth illustrative embodiments, and the accompanying drawings of which:

FIG. 1 is a portion of a SIRPα crystal structure of and a partial sequence of SIRPα (SEQ ID NO: 1) showing a configuration of a GG linker region between the SIRPα CD47 binding domain and the Fc domain of an antibody.

FIG. 2A is a graphical depiction of bivalent SIRPα CD47 binding domains fused with an antibody Fc domain (see, e.g., SEQ ID NO: 7).

FIG. 2B is a graphical depiction of a tetravalent engineered SIRPα fusion protein comprising antibody Fc domains (205) fused with SIRPα CD47 binding domains (201) as described herein (see, e.g., SEQ. ID NO: 2).

FIG. 2C is a graphical depiction of a hexavalent engineered SIRPα fusion protein comprising antibody Fc domains fused with SIRPα binding domains as described herein (see, e.g., SEQ. ID NO: 12, amino acids 23 to 627 of SEQ ID NO: 35, amino acids 23 to 617 of SEQ ID NO: 59).

FIG. 2D is a graphical depiction of an octavalent engineered SIRPα fusion protein comprising antibody Fc domains fused with SIRPα binding domains as described herein (see, e.g., SEQ. ID NO: 13, amino acids 23 to 742 of SEQ ID NO: 60).

FIGS. 3A-3F are graphical representations of exemplary SIRPα-anti-CCR4 bispecific fusion proteins formed around human IgG1 Fc (CH2, CH3) domains. The anti-CCR4 antibody portion may be either single chain or full length heavy and light chain. The SIRPα domain can be fused to either N-terminus or C-terminus of the Fc domains (which may be the Fc domain of an anti-CCR4 antibody). The structures shown in FIGS. 3A, 3B, 3C, and 3D include SIRPα domains and one or more anti-CCR4 scFv domains. The structures shown in FIGS. 3E and 3F include SIRPα domains and one or more anti-CCR4 Fab domains. The structure shown in FIG. 3A may be exemplified by SEQ ID NOs: 8 & 9; the structure shown in FIG. 3B may be exemplified by SEQ ID NOs: 10 & 11; the structure shown in FIG. 3C may be exemplified by SEQ ID NOs: 10 & 26; the structure shown in FIG. 3D may be exemplified by SEQ ID NO: 10; the structure shown in FIG. 3E may be exemplified by SEQ ID NOs: 16 & 18; the structure shown in FIG. 3F may be exemplified by SEQ ID NOs: 18, 19 & 20.

FIGS. 4A-4E are graphical depictions of an anti-CCR4 antibody, which may be modified into various exemplary fusion proteins as described herein. FIG. 4A is a graphical representation of an anti-CCR4 antibody. FIG. 4B is a graphical representation of an anti-CCR4 antibody with two high-affinity (HA) SIRPα domains fused at the C-terminus ends of the antibody heavy chains (HCs). FIG. 4C is a graphical representation of an anti-CCR4 antibody with two wild-type (WT) SIRPα domains fused at the C-terminus ends of the antibody HCs. FIG. 4D is a graphical representation of an anti-CCR4 antibody with two SIRPα domains fused at the C-terminus ends of the antibody light chains (LCs). FIG. 4E is a graphical representation of an anti-CCR4 antibody with four SIRPα domains, two fused at the C-terminus ends of the antibody LCs and two fused at the C-terminus ends of the antibody HCs. The structure shown in FIG. 4A may be exemplified by SEQ ID NOs: 14 & 17; the structure shown in FIG. 4B may be exemplified by SEQ ID NOs: 15 & 17; the structure shown in FIG. 4C may be exemplified by SEQ ID NOs: 16 & 17; the structure shown in FIG. 4D may be exemplified by SEQ ID NOs: 14 & 18; the structure shown in FIG. 4E may be exemplified by SEQ ID NOs: 16 & 18.

FIGS. 5A-5D are graphical depictions of exemplary SIRPα-anti-CCR4 bispecific heterodimeric fusion proteins that utilize “knob & hole” technology to facilitate formation of heterodimers (versus homodimers). FIG. 5A is a graphical representation of a fusion protein with three SIRPα domains and one anti-CCR4 domain, which in turn comprises anti-CCR4 LC and anti-CCR4 HC regions. FIG. 5B is a graphical representation of a fusion protein similar to that shown in FIG. 5A, but with the knob and hole domains switched. FIG. 5C is a graphical representation of a fusion protein with four SIRPα domains and one anti-CCR4 domain, as illustrated. FIG. 5D is a graphical representation of a fusion protein similar to that shown in FIG. 5C, but with the knob and hole domains switched. The fusion protein shown in FIG. 5A may be exemplified by SEQ ID NOs: 17, 19 & 20; the fusion protein shown in FIG. 5B may be exemplified by SEQ ID NOs: 17, amino acids 23 to 492 of SEQ ID NO: 36 & SEQ ID NO: 21; the fusion protein shown in FIG. 5C may be exemplified by SEQ ID NOs: 18, 19 & 20; the fusion protein shown in FIG. 5D may be exemplified by SEQ ID NOs: 18, SEQ ID NO: 21, & amino acids 23 to 492 of SEQ ID NO: 36.

FIGS. 6A and 6B are graphical depictions of exemplary SIRPα-anti-CCR4 bispecific homodimeric fusion proteins. FIG. 6A is a graphical representation of a fusion protein with SIRPα domains fused to the N-terminal ends of anti-CCR4 LC domains. FIG. 6B is a graphical representation of a fusion protein with SIRPα domains fused to (i) the N-terminal ends of anti-CCR4 LC domains, and (ii) the C-terminal ends of anti-CCR4 HC domains. The fusion protein shown in FIG. 6A may be exemplified by SEQ ID NOs: 14 & 29; the fusion protein shown in FIG. 6B may be exemplified by SEQ ID NOs: 16 & 29.

FIGS. 6C and 6D are graphical depictions of exemplary SIRPα-anti-CCR4 bispecific heterodimeric fusion proteins that utilize “knob & hole” technology to facilitate formation of heterodimers (versus homodimers). FIG. 6C is a graphical representation of a fusion protein with (i) a SIRPα domain fused to the N-terminal end of an anti-CCR4 LC domain, (ii) a SIRPα domain fused to the C-terminal end of an anti-CCR4 HC domain, and (iii) SIRPα domains fused to both the N and C-terminal ends of an anti-CCR4 HC domain. FIG. 6D is a graphical representation of a fusion protein similar to that shown in FIG. 6C, but with the knob and hole domains switched. The fusion protein shown in FIG. 6C may be exemplified by SEQ ID NO: 19, SEQ ID NO: 20 & amino acids 23 to 372 of SEQ ID NO: 29; the fusion protein shown in FIG. 6D may be exemplified by SEQ ID NO: 36, SEQ ID NO: 21 & amino acids 23 to 372 of SEQ ID NO: 29.

FIGS. 7A and 7B are graphical depictions of exemplary SIRPα-anti-CCR4 bispecific homodimeric fusion proteins. FIG. 7A is a graphical representation of a fusion protein with SIRPα domains fused to the N-terminal ends of anti-CCR4 HC domains. FIG. 7B is a graphical representation of a fusion protein with SIRPα domains fused to (i) the N-terminal ends of anti-CCR4 HC domains, and (ii) the C-terminal ends of anti-CCR4 LC domains. The fusion protein shown in FIG. 7A may be exemplified by SEQ ID NO: 17 & amino acids 23 to 601 of SEQ ID NO: 30; the fusion protein shown in FIG. 7B may be exemplified by amino acids 23 to 601 of SEQ ID NO: 30 & amino acids 23 to 372 of SEQ ID NO: 29.

FIGS. 7C and 7D are graphical depictions of exemplary SIRPα-anti-CCR4 bispecific homodimeric fusion proteins. FIG. 7C is a graphical representation of a fusion protein with SIRPα domains fused to both the N- and C-terminal ends of anti-CCR4 HC domains. FIG. 7D is a graphical representation of a fusion protein with SIRPα domains fused to (i) both the N- and C-terminal ends of anti-CCR4 HC domains, and (ii) the C-terminal ends of anti-CCR4 LC domains. The fusion protein shown in FIG. 7C may be exemplified by SEQ ID NO: 17 & amino acids 23 to 727 of SEQ ID NO: 31; the fusion protein shown in FIG. 7D may be exemplified by amino acids 23 to 727 of SEQ ID NO: 31 & amino acids 23 to 372 of SEQ ID NO: 29.

FIGS. 8A and 8B are graphical depictions of exemplary SIRPα-anti-CCR4 bispecific homodimeric fusion proteins, with longer linkers connecting domains of the fusion protein. FIG. 8A is a graphical representation of a fusion protein with SIRPα domains fused to (i) the N-terminal ends of anti-CCR4 HC domains, and (ii) the C-terminal ends of anti-CCR4 LC domains. FIG. 8B is a graphical representation of a fusion protein with SIRPα domains fused to (i) both the N- and C-terminal ends of anti-CCR4 HC domains, and (ii) the C-terminal ends of anti-CCR4 LC domains. The fusion protein shown in FIG. 8A may be exemplified by amino acids 23 to 377 of SEQ ID NO: 32 & amino acids 23 to 601 of SEQ ID NO: 30; the fusion protein shown in FIG. 8B may be exemplified by amino acids 23 to 727 of SEQ ID NO: 31 & amino acids 23 to 377 of SEQ ID NO: 32.

FIGS. 9A, 9B and 9C are graphical depictions of exemplary SIRPα-anti-CCR4 bispecific homodimeric fusion proteins, combining both anti-CCR4 Fab portions and anti-CCR4 scFv portions in a single fusion protein. FIG. 9A is a graphical representation of a fusion protein comprising an anti-CCR4 antibody with anti-CCR4 scFv domains fused to the C-terminal ends of the light chains. FIG. 9B is a graphical representation of a fusion protein comprising an anti-CCR4 antibody with anti-CCR4 scFv domains fused to the C-terminal ends of the light chains, and SIRPα domains fused to the N-terminals of the heavy chains. FIG. 9C is a graphical representation of a fusion protein comprising an anti-CCR4 antibody with anti-CCR4 scFv domains fused to the C-terminal ends of the light chains, and SIRPα domains fused to both the N- and C-terminals of the heavy chains. The fusion protein shown in FIG. 9A may be exemplified by SEQ ID NO: 14 & amino acids 23 to 502 of SEQ ID NO: 33; the fusion protein shown in FIG. 9B may be exemplified by amino acids 23 to 601 of SEQ ID NO: 30 & amino acids 23 to 502 of SEQ ID NO: 33; the fusion protein shown in FIG. 9C may be exemplified by amino acids 23 to 727 of SEQ ID NO: 31 & amino acids 23 to 502 of SEQ ID NO: 33.

FIGS. 10A, 10B and 10C are graphical depictions of exemplary SIRPα-anti-CCR4 bispecific homodimeric fusion proteins, combining both anti-CCR4 Fab portions and anti-CCR4 scFv domains in a single fusion protein. FIG. 10A is a graphical representation of a fusion protein comprising an anti-CCR4 antibody with anti-CCR4 scFv domains fused to the N-terminal ends of the light chains. FIG. JOB is a graphical representation of a fusion protein comprising an anti-CCR4 antibody with anti-CCR4 scFv domains fused to the N-terminal ends of the light chains, and SIRPα domains fused to the N-terminals of the heavy chains. FIG. 10C is a graphical representation of a fusion protein comprising an anti-CCR4 antibody with anti-CCR4 scFv domains fused to the N-terminal ends of the light chains, and SIRPα domains fused to both the N- and C-terminals of the heavy chains. The fusion protein shown in FIG. 10A may be exemplified by SEQ ID NOs: 14 & amino acids 23 to 497 of SEQ ID NO: 34; the fusion protein shown in FIG. 10B may be exemplified by amino acids 23 to 601 of SEQ ID NO: 30 & amino acids 23 to 497 of SEQ ID NO: 34; the fusion protein shown in FIG. 10C may be exemplified by amino acids 23 to 727 of SEQ ID NO: 31 & amino acid 23 to 497 of SEQ ID NO: 34.

FIGS. 11A and 11B show data from binding assays indicating binding concentrations of select molecules including an example of an engineered high affinity bivalent SIRPα construct and an example of an engineered tetravalent SIRPα construct as described herein.

FIGS. 12A-12E are data from size exclusion-high-performance liquid chromatography assays of examples of engineered SIRPα molecules as described herein, fusion proteins 1052, 1053, 1063, 1052 and 1054 as described herein.

FIG. 13 shows results from mass spectroscopy confirmation of purity and identification of examples of engineered SIRPα z from an antibody-dependent cell-mediated phagocytosis (ADCP) assays of examples of engineered SIRPα molecules as described herein.

FIGS. 14A-14C are charts summarizing results from associated purification methods of examples of engineered SIRPα molecules as described herein.

FIGS. 15A and 15B show results from an antibody-dependent cell-mediated phagocytosis (ADCP) reporter cell (hCD32 expressing NFAT-Lucia Jurkat line) assay reading CD32 activation associated with activity of examples of engineered SIRPα molecules as described herein. FIG. 15A shows data obtained using supernatants from mRNA transfected HEK cells, while FIG. 15B shows data obtained using purified proteins.

FIG. 16A shows results from an antibody-dependent cell-mediated cytotoxicity (ADCC) assay measuring NK cells killing of Raji tumor cells in the presence of increasing concentrations of engineered SIRPα molecules as described herein.

FIG. 16B shows results from an ADCC assay measuring NK cells killing of H9 tumor cells in the presence of increasing concentrations of engineered SIRPα molecules as described herein.

FIG. 16C shows the binding (or lack thereof) of different constructs, including a tetravalent SIRPα-Fc construct as described herein, to human red blood cells (RBCs) in vitro.

FIG. 16D shows results from an ADCC assay measuring NK cells killing of H9 tumor cells in the presence of increasing concentrations of engineered fusion proteins comprising SIRPα and anti-CCR4 domains as described herein.

FIG. 17A shows the degree of binding of different constructs, including a tetravalent SIRPα-Fc construct as described herein, to red blood cells (RBCs) from five healthy human donors, in vitro.

FIG. 17B shows serum expression of tetravalent SIRPα-Fc in mice in vivo.

FIG. 18 shows superior suppression of tumor growth in a Raji xenograft model by a tetravalent SIRPα-Fc fusion protein as described herein.

### DETAILED DESCRIPTION

### Definitions

In order that the present disclosure can be more readily understood, certain terms are first defined. As used in this application, except as otherwise expressly provided herein, each of the following terms shall have the meaning set forth below. Additional definitions are set forth throughout the application.

Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Where a value is explicitly recited, it is to be understood that values which are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

Nucleotides are referred to by their commonly accepted single-letter codes. Unless otherwise indicated, nucleic acids are written left to right in 5′ to 3′ orientation. Nucleotides are referred to herein by their commonly known one-letter symbols recommended by the IUPAC-TUB Biochemical Nomenclature Commission. Accordingly, A represents adenine, C represents cytosine, G represents guanine, T represents thymine, and U represents uracil.

Amino acids are referred to herein by either their commonly known three letter symbols or by the one-letter symbols recommended by the IUPAC-IUB Biochemical Nomenclature Commission. Unless otherwise indicated, amino acid sequences are written left to right in amino to carboxy orientation.

The term “about” as used in connection with a numerical value throughout the specification and the claims denotes an interval of accuracy, familiar and acceptable to a person skilled in the art. In general, such interval of accuracy is +/−10%.

Where ranges are given, endpoints are included. Furthermore, unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

The term “amino acid substitution” refers to replacing an amino acid residue present in a parent or reference sequence (e.g., a wild type sequence) with another amino acid residue. An amino acid can be substituted in a parent or reference sequence (e.g., a wild type polypeptide sequence), for example, via recombinant methods known in the art. Accordingly, a reference to a “substitution at position X” refers to the substitution of an amino acid present at position X with an alternative amino acid residue. In some aspects, substitution patterns can be described according to the schema AnY, wherein A is the single letter code corresponding to the amino acid naturally or originally present at position n, and Y is the substituting amino acid residue. In other aspects, substitution patterns can be described according to the schema An(YZ), wherein A is the single letter code corresponding to the amino acid residue substituting the amino acid naturally or originally present at position X, and Y and Z are alternative substituting amino acid residue.

In the context of the present disclosure, substitutions (even when they referred to as amino acid substitution) are conducted at the nucleic acid level, i.e., substituting an amino acid residue with an alternative amino acid residue is conducted by substituting the codon encoding the first amino acid with a codon encoding the second amino acid.

As used herein with respect to a disease, the term “associated with” means that the symptom, measurement, characteristic, or status in question is linked to the diagnosis, development, presence, or progression of that disease. As association may, but need not, be causatively linked to the disease.

Nucleotide Sequence Optimization refers to a process or series of processes by which nucleotides in a reference nucleic acid sequence are replaced with alternative nucleotides, resulting in a nucleic acid sequence with improved properties, e.g., improved protein expression or immunogenicity.

As used herein the term “CD47 binding domain” refers to a region of the SIRPα molecule that is capable of binding a CD47 polypeptide. SEQ ID NO: 23 is an example of the wild type (WT) SIRPα binding site (e.g., CD47 binding domain). The term “CD47 binding domain” refers to peptide that binds to CD47 having an amino acid sequence that is at least 90% homologous to the sequence of SEQ I NO: 23 while retaining a Gln in position 37 of SEQ ID NO: 23.

The terms “codon substitution” or “codon replacement” in the context of nucleotide sequence optimization refer to replacing a codon present in a reference nucleic acid sequence with another codon. Accordingly, references to a “substitution” or “replacement” at a certain location in a nucleic acid sequence (e.g., an mRNA) or within a certain region or subsequence of a nucleic acid sequence (e.g., an mRNA) refer to the substitution of a codon at such location or region with an alternative codon.

As used herein, the terms “coding region” and “region encoding” and grammatical variants thereof, refer to an open reading frame (ORF) in a polynucleotide that upon expression yields a polypeptide or protein.

A “conservative amino acid substitution” is one in which the amino acid residue is replaced with an amino acid residue having a similar side chain. Families of amino acid residues having similar side chains have been defined in the art, including basic side chains (e.g., lysine, arginine, or histidine), acidic side chains (e.g., aspartic acid or glutamic acid), uncharged polar side chains (e.g., glycine, asparagine, glutamine, serine, threonine, tyrosine, or cysteine), nonpolar side chains (e.g., alanine, valine, leucine, isoleucine, proline, phenylalanine, methionine, or tryptophan), beta-branched side chains (e.g., threonine, valine, isoleucine) and aromatic side chains (e.g., tyrosine, phenylalanine, tryptophan, or histidine). Thus, if an amino acid in a polypeptide is replaced with another amino acid from the same side chain family, the amino acid substitution is considered to be conservative. In another aspect, a string of amino acids can be conservatively replaced with a structurally similar string that differs in order and/or composition of side chain family members.

Non-conservative amino acid substitutions include those in which (i) a residue having an electropositive side chain (e.g., Arg, His or Lys) is substituted for, or by, an electronegative residue (e.g., Glu or Asp), (ii) a hydrophilic residue (e.g., Ser or Thr) is substituted for, or by, a hydrophobic residue (e.g., Ala, Leu, Ile, Phe or Val), (iii) a cysteine or proline is substituted for, or by, any other residue, or (iv) a residue having a bulky hydrophobic or aromatic side chain (e.g., Val, His, Ile or Trp) is substituted for, or by, one having a smaller side chain (e.g., Ala or Ser) or no side chain (e.g., Gly).

As used herein, the term “conserved” refers to nucleotides or amino acid residues of a polynucleotide sequence or polypeptide sequence, respectively, that are those that occur unaltered in the same position of two or more sequences being compared. Nucleotides or amino acids that are relatively conserved are those that are conserved amongst more related sequences than nucleotides or amino acids appearing elsewhere in the sequences.

In general, a “delivery vehicle molecule” is a molecule used to delivery mRNA to a cell, in which the molecule protects the mRNA(s) from RNase degradation, and thereby may increase their cellular uptake, and may facilitate endosome escape, thereby expressing functional proteins in the cytosol. Referring to something as a delivery vehicle molecule does not mean that it may not also have therapeutic effects.

As used herein, when referring to polypeptides, the term “domain” refers to a portion of a polypeptide having one or more identifiable structural or functional characteristics or properties (e.g., binding capacity, serving as a site for protein-protein interactions).

As used herein, the term “encapsulate” means to enclose, surround or encase.

As used herein, the term “homology” refers to the overall relatedness between polymeric molecules, e.g., between peptides of polypeptide molecules. In the context of the present disclosure, the term homology encompasses both identity and similarity (e.g., percent homologous).

An “immune checkpoint inhibitor” or simply “checkpoint inhibitor” refers to a molecule that prevents immune cells from being downregulated or inhibited by cancer cells. As used herein, the term checkpoint inhibitor refers to polypeptides (e.g., fusion proteins) that neutralize or inhibit inhibitory checkpoint molecules such as cytotoxic T-lymphocyte-associated protein 4 (CTLA-4), programmed death 1 receptor (PD-1), or PD-1 ligand 1 (PD-L1).

The term “inflammatory cytokine” refers to cytokines that are elevated in an inflammatory response. Examples of inflammatory cytokines include interleukin-6 (IL-6), CXCL1 (chemokine (C-X-C motif) ligand 1; also known as GROc, interferon-γ (IFNγ), tumor necrosis factor α (TNFα), interferon 7-induced protein 10 (IP-10), or granulocyte-colony stimulating factor (G-CSF). The term inflammatory cytokines also include other cytokines associated with inflammatory responses known in the art, e.g., interleukin-1 (IL-1), interleukin-8 (IL-8), interleukin-12 (L-12), interleukin-13 (IL-13), interferon α (IFN-α), etc.

A polynucleotide, vector, mRNA, polypeptide, cell, or any composition disclosed herein which is “isolated” is a polynucleotide, vector, mRNA, polypeptide, cell, or composition which is in a form not found in nature. Isolated polynucleotides, vectors, polypeptides, or compositions include those which have been purified to a degree that they are no longer in a form in which they are found in nature. In some aspects, a polynucleotide, vector, polypeptide, or composition which is isolated is substantially pure.

“Knob and Holes” or “Knob-into-Holes” terms are used interchangeably herein. A CH3 domains of an Fc-region of can be altered by the “knob-into-holes” technology which is described in detail with several examples in e.g., U.S. Pat. No. 8,679,785, WO 96/027011, Ridgway, J. B., et al., Protein Eng. 9 (1996) 617-621; and Merchant, A. M., et al., Nat. Biotechnol. 16 (1998) 677-681. In this method the interaction surfaces of the two CH3 domains are altered to increase the heterodimerization of both heavy chains containing these two CH3 domains. Each of the two CH3 domains (of the two heavy chains) can be the “knob”, while the other is the “hole”. The introduction of a disulfide bridge may further stabilize the heterodimers (Merchant, A. M., et al., Nature Biotech. 16 (1998) 677-681; Atwell, S., et al., J. Mol. Biol. 270 (1997) 26-35) and increases the yield. For example, the mutation T366W in the CH3 domain of an antibody heavy chain is denoted as “knob mutation” and the mutations T366S, L368A, Y407V in the CH3 domain of an antibody heavy chain are denoted as “mutations hole” (numbering according to Kabat EU index). An additional interchain disulfide bridge between the CH3 domains can also be used (Merchant, A. M., et al., Nature Biotech. 16 (1998) 677-681) e.g. by introducing a S354C mutation into the CH3 domain of the heavy chain with the “knob mutation” (denotes as “knob-cys mutations” or “mutations knob-cys”) and by introducing a Y349C mutation into the CH3 domain of the heavy chain with the “hole mutations” (denotes as “hole-cys mutations” or “mutations hole-cys”) (numbering according to Kabat EU index) or vice versa.

As used herein, “naturally occurring” means existing in nature without artificial aid.

As used herein, a “part” or “region” of a polynucleotide is defined as any portion of the polynucleotide that is less than the entire length of the polynucleotide.

The term “synthetic” means produced, prepared, and/or manufactured by the hand of man. Synthesis of polynucleotides or other molecules of the present disclosure can be chemical or enzymatic.

RNA is the usual abbreviation for ribonucleic acid. It is a nucleic acid molecule, i.e. a polymer consisting of nucleotide monomers. These nucleotides are usually adenosine monophosphate (AMP), uridine monophosphate (UMP), guanosine monophosphate (GMP) and cytidine monophosphate (CMP) monomers or analogues thereof, which are connected to each other along a so-called backbone. The backbone is formed by phosphodiester bonds between the sugar, i.e., ribose, of a first and a phosphate moiety of a second, adjacent monomer. The specific order of the monomers, i.e., the order of the bases linked to the sugar/phosphate-backbone, is called the RNA sequence. Usually, RNA may be obtainable by transcription of a DNA sequence, e.g., inside a cell. In eukaryotic cells, transcription is typically performed inside the nucleus or the mitochondria. In vivo, transcription of DNA usually results in the so-called premature RNA (also called pre-mRNA, precursor mRNA or heterogeneous nuclear RNA) which has to be processed into so-called messenger RNA, usually abbreviated as mRNA. Processing of the premature RNA, e.g., in eukaryotic organisms, comprises a variety of different posttranscriptional modifications such as splicing, 5 Lapping, polyadenylation, export from the nucleus or the mitochondria and the like. The sum of these processes is also called maturation of RNA. The mature messenger RNA usually provides the nucleotide sequence that may be translated into an amino acid sequence of a particular peptide or protein. Typically, a mature mRNA comprises a 5 kap, optionally a 5′ untranslated region (“5 UTR”), an open reading frame, optionally a 3′ untranslated region (“3 UTR”) and a poly(A) tail.

In addition to messenger RNA (mRNA), several non-coding types of RNA exist which may be involved in regulation of transcription and/or translation, and immunostimulation. Within the present disclosure the term “RNA” further encompasses any type of single stranded (ssRNA) or double stranded RNA (dsRNA) molecule known in the art, such as viral RNA, retroviral RNA and replicon RNA, small interfering RNA (siRNA), antisense RNA (asRNA), circular RNA (circRNA), ribozymes, aptamers, riboswitches, immunostimulating/immunostimulatory RNA, transfer RNA (tRNA), ribosomal RNA (rRNA), small nuclear RNA (snRNA), small nucleolar RNA (snoRNA), microRNA (miRNA), and Piwi-interacting RNA (piRNA).

5-CAP-Structure: A 5-CAP is typically a modified nucleotide (CAP analogue), particularly a guanine nucleotide, added to the 5 end of an mRNA molecule. In certain implementations, the 5-CAP is added using a 5-5-triphosphate linkage (also named m7GpppN). Further examples of 5-CAP structures include glyceryl, inverted deoxy abasic residue (moiety), 4,5 methylene nucleotide, 1-(beta-D-erythrofuranosyl) nucleotide, 4-thio nucleotide, carbocyclic nucleotide, 1,5-anhydrohexitol nucleotide, L-nucleotides, alpha-nucleotide, modified base nucleotide, threo-pentofuranosyl nucleotide, acyclic 3,4-seco nucleotide, acyclic 3,4-dihydroxybutyl nucleotide, acyclic 3,5 dihydroxypentyl nucleotide, 3-3-inverted nucleotide moiety, 3-3 inverted abasic moiety, 3-2 inverted nucleotide moiety, 3-2-inverted abasic moiety, 1,4-butanediol phosphate, 3-phosphoramidate, hexylphosphate, aminohexyl phosphate, 3-phosphate, 3 Phosphorothioate, phosphorodithioate, or bridging or non-bridging methylphosphonate moiety. These modified 5-CAP structures may be used in the context of the present disclosure to modify the RNA sequence of the present disclosure. Further modified 50 CAP structures which may be used in the context of the present disclosure are CAP1 (additional methylation of the ribose of the adjacent nucleotide of m7GpppN), CAP2 (additional methylation of the ribose of the 2nd nucleotide downstream of the m7GpppN), CAP3 (additional methylation of the ribose of the 3rd nucleotide downstream of the m7GpppN), CAP4 (additional methylation of the ribose of the 4th nucleotide downstream of the m7GpppN), ARCA (anti-reverse CAP analogue), modified ARCA (e.g. phosphothioate modified ARCA), inosine, N1-methyl-guanosine, 2-fluoro-guanosine, 7-deaza-guanosine, 8-oxo-guanosine, 2-amino-guanosine, LNA-guanosine, and 2-azido-guanosine.

In the context of the present disclosure, a 5 cap structure may also be formed in chemical RNA synthesis or RNA in vitro transcription (co-transcriptional capping) using cap analogues, or a cap structure may be formed in vitro using capping enzymes (e.g., commercially available capping kits).

A cap analogue refers to a non-polymerizable di-nucleotide that has cap functionality in that it facilitates translation or localization, and/or prevents degradation of the RNA molecule when incorporated at the 5 end of the RNA molecule. Non-polymerizable means that the cap analogue will be incorporated only at the 5 terminus because it does not have a 5 triphosphate and therefore cannot be extended in the 3 direction by a template-dependent RNA polymerase.

Cap analogues include, but are not limited to, a chemical structure selected from the group consisting of m7GpppG, m7GpppA, m7GpppC; unmethylated cap analogues (e.g., GpppG); dimethylated cap analogue (e.g., m2,7GpppG), trimethylated cap analogue (e.g., m2,2,7GpppG), dimethylated symmetrical cap analogues (e.g., m7Gpppm7G), or anti reverse cap analogues (e.g., ARCA; m7,2OmeGpppG, m7,2dGpppG, m7,3 OmeGpppG, m7,3 dGpppG and their tetraphosphate derivatives). The synthesis of N7-(4-chlorophenoxyethyl) substituted dinucleotide cap analogues has been described recently.

A poly(A) tail also called “3-poly(A) tail” or “Poly(A) sequence” is typically a long homopolymeric sequence of adenosine nucleotides of up to about 400 adenosine nucleotides, e.g. from about 25 to about 400, from about 50 to about 400, from about 50 to about 300, from about 50 to about 250, or from about 60 to about 250 adenosine nucleotides, added to the 3 end of an mRNA. In certain implementations of the present disclosure, the poly(A) tail of an mRNA or srRNA is derived from a DNA template by RNA in vitro transcription. Alternatively, the poly(A) sequence may also be obtained in vitro by common methods of chemical synthesis without being necessarily transcribed from a DNA-progenitor. Moreover, poly(A) sequences, or poly(A) tails may be generated by enzymatic polyadenylation of the RNA.

A stabilized nucleic acid, typically, exhibits a modification increasing resistance to in vivo degradation (e.g., degradation by an exo- or endo-nuclease) and/or ex vivo degradation (e.g., by the manufacturing process prior to composition administration, e.g., in the course of the preparation of the composition to be administered). Stabilization of RNA can, e.g., be achieved by providing a 5-CAP-Structure, a poly(A) tail, or any other UTR-modification. Stabilization can also be achieved by backbone-modification (e.g., use of synthetic backbones such as phosphorothioate) or modification of the G/C-content or the C-content of the nucleic acid. Various other methods are known in the art and conceivable in the context of the disclosure, to stabilize or otherwise improve the function of the nucleic acid. Provided herein, therefore, are polynucleotides which have been designed to improve one or more of the stability and/or clearance in tissues, receptor uptake and/or kinetics, cellular access, engagement with translational machinery, RNA half-life, translation efficiency, immune evasion, immune induction (for vaccines), protein production capacity, secretion efficiency (when applicable), accessibility to circulation, protein half-life and/or modulation of a cells status, function and/or activity.

A 5-UTR is typically understood to be a particular section of RNA. It is located 5 of the open reading frame of the mRNA. In the case of srRNA, the open reading frame encodes the viral non-structural proteins while the sequence of interest is encoded in the subgenomic fragment of the viral RNA. Thus, the 5′UTR is upstream of nsP1 open reading frame. In addition, the subgenomic RNA of the srRNA has a 5′UTR. Thus, the subgenomic RNA containing a sequence of interest encoding a protein of interest contains a 5′UTR. Typically, the LTR starts with the transcriptional start site and ends one nucleotide before the start codon of the open reading frame. The 5-UTR may comprise elements for controlling gene expression, also called regulatory elements. Such regulatory elements may be, for example, ribosomal binding sites or a 5-Terminal Oligopyrimidine Tract. The 5-UTR may be posttranscriptionally modified, for example by addition of a 5-CAP. In the context of the present disclosure, a 5 UTR corresponds to the sequence of a mature mRNA or srRNA which is located between the 5-CAP and the start codon. In one implementations, the 5-UTR corresponds to the sequence which extends from a nucleotide located 3 to the 5-CAP, and in certain implementations from the nucleotide located immediately 3 to the 5-CAP, to a nucleotide located 5 to the start codon of the protein coding region and in some aspects to the nucleotide located immediately 5 to the start codon of the protein coding region. The nucleotide located immediately 3 to the 5-CAP of a mature mRNA or srRNA typically corresponds to the transcriptional start site. The term “corresponds to” means that the 5 UTR sequence may be an RNA sequence, such as in the mRNA sequence used for defining the 5 L-UTR sequence, or a DNA sequence which corresponds to such RNA sequence. In the context of the present disclosure, the term “a 5-UTR of a gene”, such as “a 5-UTR of a NYESO1 gene”, is the sequence which corresponds to the 5-UTR of the mature mRNA derived from this gene, i.e., the mRNA obtained by transcription of the gene and maturation of the pre-mature mRNA. The term “5-UTR of a gene” encompasses the DNA sequence and the RNA sequence of the 5-UTR. An exemplary 5′ UTR is provided herein as SEQ TD NO: 37.

Generally, the term “3-UTR” refers to a part of the nucleic acid molecule which is located 3 (i.e., “downstream”) of an open reading frame and which is not translated into protein. Typically, a 3 UTR is the part of an RNA which is located between the protein coding region (open reading frame (ORF) or coding sequence (CDS)) and the poly(A) sequence of the mRNA. In the context of the present disclosure, the term 3 UTR may also comprise elements, which are not encoded in the template, from which an RNA is transcribed, but which are added after transcription during maturation, e.g., a poly(A) sequence. A 3-UTR of the RNA is not translated into an amino acid sequence. An exemplary 3′ UTR is provided herein as SEQ ID NO: 38.

With respect to srRNA, the 3-UTR sequence is generally encoded by the viral genomic RNA, which is transcribed into the respective mRNA during the gene expression process. The genomic sequence is first transcribed into pre-mature mRNA. The pre-mature mRNA is then further processed into mature mRNA in a maturation process. This maturation process comprises 5 capping. In the context of the present disclosure, a 3-UTR corresponds to the sequence of a mature mRNA or srRNA (and the srRNA subgenomic RNA), which is located between the stop codon of the protein coding region, preferably immediately 3 to the stop codon of the protein coding region for the sequence of interest, and the poly(A) sequence of the mRNA. The term “corresponds to” means that the 3 UTR sequence may be an RNA sequence, such as in the mRNA sequence used for defining the 3 UTR sequence, or a DNA sequence, which corresponds to such RNA sequence. In the context of the present disclosure, the term “a 3-UTR of a gene”, is the sequence, which corresponds to the 3-UTR of the mature mRNA derived from this gene, i.e., the mRNA obtained by transcription of the gene and maturation of the pre-mature mRNA. The term “3-UTR of a gene” encompasses the DNA sequence and the RNA sequence (both sense and antisense strand and both mature and immature) of the 3-UTR.

According to certain implementations of the disclosure, the RNAs for use in the delivery vehicle complexes herein comprise an RNA comprising at least one region encoding a peptide (e.g., a polypeptide), or protein, or functional fragment of the foregoing. As used herein, “functional fragment” refers to a fragment of a peptide, (e.g., a polypeptide), or protein that retains the ability to induce an immune response.

### Polynucleotide Synthesis

Methods of making polynucleotides of a predetermined sequence are well-known. Solid-phase synthesis methods are known for both polyribonucleotides and polydeoxyribonucleotides (the well-known methods of synthesizing DNA are also useful for synthesizing RNA). Polyribonucleotides can also be prepared enzymatically. Non-naturally occurring nucleobases can be incorporated into the polynucleotide, as well.

Any method known in the art for making RNA is contemplated herein for making the RNAs. Illustrative methods for making RNA include but are not limited to, chemical synthesis and in vitro transcription.

In certain implementations, the RNA for use in the methods herein is chemically synthesized. Chemical synthesis of relatively short fragments of oligonucleotides with defined chemical structure provides a rapid and inexpensive access to custom-made oligonucleotides of any desired sequence. Whereas enzymes synthesize DNA and RNA only in the 5 to 3 direction chemical oligonucleotide synthesis does not have this limitation, although it is most often carried out in the opposite, i.e., the 3 to 5 direction. In certain implementations, the process is implemented as solid-phase synthesis using the phosphoramidite method and phosphoramidite building blocks derived from protected nucleosides (A, C, G, and U), or chemically modified nucleosides.

In some implementations, modifications are included in the modified nucleic acid or in one or more individual nucleoside or nucleotide. For example, modifications to a nucleoside may include one or more modifications to the nucleobase, the sugar, and/or the internucleoside linkage. In some implementations having at least one modification, the polynucleotide includes a backbone moiety containing the nucleobase, sugar, and internucleoside linkage of: pseudouridine-alpha-thio-MP, 1-methyl-pseudouridine-alpha-thio-MP, 1-ethyl-pseudouridine-MP, 1-propyl-pseudouridine-MP, 1-(2,2,2-trifluoroethyl)-pseudouridine-MP, 2-amino-adenine-MP, xanthosine-MP, 5-bromo-cytidine-MP, 5-aminoallyl-cytidine-MP, or 2-aminopurine-riboside-MP.

In other implementations having at least one modification, the polynucleotide includes a backbone moiety containing the nucleobase, sugar, and internucleoside linkage of: pseudouridine-alpha-thio-MP, 1-methyl-pseudouridine-alpha-thio-MP, or 5-bromo-cytidine-MP. Nucleoside and nucleotide modifications contemplated for use in the present disclosure are known in the art.

To obtain the desired oligonucleotide, the building blocks are sequentially coupled to the growing oligonucleotide chain on a solid phase in the order required by the sequence of the product in a fully automated process. Upon the completion of the chain assembly, the product is released from the solid phase to the solution, deprotected, and collected. The occurrence of side reactions sets practical limits for the length of synthetic oligonucleotides (up to about 200 nucleotide residues), because the number of errors increases with the length of the oligonucleotide being synthesized. Products are often isolated by HPLC to obtain the desired oligonucleotides in high purity.

In certain implementations, RNA is made using in vitro transcription. The terms “RNA in vitro transcription” or “in vitro transcription” relate to a process wherein RNA is synthesized in a cell-free system (in vitro). DNA, particularly plasmid DNA, is used as template for the generation of RNA transcripts. RNA may be obtained by DNA-dependent in vitro transcription of an appropriate DNA template, which in certain implementations is a linearized plasmid DNA template. The promoter for controlling in vitro transcription can be any promoter for any DNA-dependent RNA polymerase. Particular examples of DNA-dependent RNA polymerases are the T7, T3, and SP6 RNA polymerases. A DNA template for in vitro RNA transcription may be obtained by cloning of a nucleic acid, in particular cDNA corresponding to the respective RNA to be in vitro transcribed, and introducing it into an appropriate vector for in vitro transcription, for example into plasmid DNA. In one implementation of the present disclosure, the DNA template is linearized with a suitable restriction enzyme, before it is transcribed in vitro. The cDNA may be obtained by reverse transcription of mRNA or chemical synthesis. Moreover, the DNA template for in vitro RNA synthesis may also be obtained by gene synthesis.

Methods for in vitro transcription are known in the art. Reagents used in the methods typically include: 1) a linearized DNA template with a promoter sequence that has a high binding affinity for its respective RNA polymerase such as bacteriophage-encoded RNA polymerases; 2) ribonucleoside triphosphates (NTPs) for the four bases (adenine, cytosine, guanine and uracil); 3) in some aspects, a cap analogue as defined above (e.g. m7G(5 ppp(5 G (m7G)); 4) a DNA-dependent RNA polymerase capable of binding to the promoter sequence within the linearized DNA template (e.g. T7, T3 or SP6 RNA polymerase); 5) optionally a ribonuclease (RNase) inhibitor to inactivate any contaminating RNase; 6) optionally a pyrophosphatase to degrade pyrophosphate, which may inhibit transcription; 7) MgCl2, which supplies Mg2+ ions as a co-factor for the polymerase; 8) a buffer to maintain a suitable pH value, which can also contain antioxidants (e.g. DTT), and/or polyamines such as spermidine at optimal concentrations.

Methods of making polynucleotides of a predetermined sequence are well-known. Solid-phase synthesis methods are known for both polyribonucleotides and polydeoxyribonucleotides (the well-known methods of synthesizing DNA are also useful for synthesizing RNA). Polyribonucleotides can also be prepared enzymatically. Non-naturally occurring nucleobases can be incorporated into the polynucleotide, as well.

Any method known in the art for making RNA is contemplated herein for making the RNAs. Illustrative methods for making RNA include but are not limited to, chemical synthesis and in vitro transcription.

In certain implementations, the RNA for use in the methods herein is chemically synthesized. Chemical synthesis of relatively short fragments of oligonucleotides with defined chemical structure provides rapid and inexpensive access to custom-made oligonucleotides of any desired sequence. Whereas enzymes synthesize DNA and RNA only in the 5 to 3 direction, chemical oligonucleotide synthesis does not have this limitation, although it is most often carried out in the opposite, i.e., the 3 to 5 direction. In certain implementations, the process is implemented as solid-phase synthesis using the phosphoramidite method and phosphoramidite building blocks derived from protected nucleosides (A, C, G, and U), or chemically modified nucleosides.

In some implementations, modifications are included in the modified nucleic acid or in one or more individual nucleoside or nucleotide. For example, modifications to a nucleoside may include one or more modifications to the nucleobase, the sugar, and/or the internucleoside linkage. In some implementations having at least one modification, the polynucleotide includes a backbone moiety containing the nucleobase, sugar, and internucleoside linkage of: pseudouridine-alpha-thio-MP, 1-methyl-pseudouridine-alpha-thio-MP, 1-ethyl-pseudouridine-MP, 1-propyl-pseudouridine-MP, 1-(2,2,2-trifluoroethyl)-pseudouridine-MP, 2-amino-adenine-MP, xanthosine-MP, 5-bromo-cytidine-MP, 5-aminoallyl-cytidine-MP, or 2-aminopurine-riboside-MP.

In other implementations having at least one modification, the polynucleotide includes a backbone moiety containing the nucleobase, sugar, and internucleoside linkage of: pseudouridine-alpha-thio-MP, 1-methyl-pseudouridine-alpha-thio-MP, or 5-bromo-cytidine-MP. Nucleoside and nucleotide modifications contemplated for use in the present disclosure are known in the art.

To obtain the desired oligonucleotide, the building blocks are sequentially coupled to the growing oligonucleotide chain on a solid phase in the order required by the sequence of the product in a fully automated process. Upon the completion of the chain assembly, the product is released from the solid phase to the solution, deprotected, and collected. The occurrence of side reactions sets practical limits for the length of synthetic oligonucleotides (up to about 200 nucleotide residues), because the number of errors increases with the length of the oligonucleotide being synthesized. Products are often isolated by HPLC to obtain the desired oligonucleotides in high purity.

In certain implementations, RNA is made using in vitro transcription. The terms “RNA in vitro transcription” or “in vitro transcription” relate to a process wherein RNA is synthesized in a cell-free system (in vitro). DNA, particularly plasmid DNA, is used as template for the generation of RNA transcripts. RNA may be obtained by DNA-dependent in vitro transcription of an appropriate DNA template, which in certain implementations is a linearized plasmid DNA template. The promoter for controlling in vitro transcription can be any promoter for any DNA-dependent RNA polymerase. Particular examples of DNA-dependent RNA polymerases are the T7, T3, and SP6 RNA polymerases. A DNA template for in vitro RNA transcription may be obtained by cloning of a nucleic acid, in particular cDNA corresponding to the respective RNA to be in vitro transcribed, and introducing it into an appropriate vector for in vitro transcription, for example into plasmid DNA. In one implementation of the present disclosure, the DNA template is linearized with a suitable restriction enzyme, before it is transcribed in vitro. The cDNA may be obtained by reverse transcription of mRNA or chemical synthesis. Moreover, the DNA template for in vitro RNA synthesis may also be obtained by gene synthesis.

Methods for in vitro transcription are known in the art. Reagents used in the methods typically include: 1) a linearized DNA template with a promoter sequence that has a high binding affinity for its respective RNA polymerase such as bacteriophage-encoded RNA polymerases; 2) ribonucleoside triphosphates (NTPs) for the four bases (adenine, cytosine, guanine and uracil); 3) in some aspects, a cap analogue as defined above (e.g. m7G(5 ppp(5)G (m7G)): 4) a DNA-dependent RNA polymerase capable of binding to the promoter sequence within the linearized DNA template (e.g. T7, T3 or SP6 RNA polymerase); 5) optionally a ribonuclease (RNase) inhibitor to inactivate any contaminating RNase; 6) optionally a pyrophosphatase to degrade pyrophosphate, which may inhibit transcription; 7) MgCl2, which supplies Mg²⁺ ions as a co-factor for the polymerase; 8) a buffer to maintain a suitable pH value, which can also contain antioxidants (e.g. DTT), and/or polyamines such as spermidine at optimal concentrations.

### SIRPα Fusion Proteins

Described herein are SIRPα fusion proteins encoded by synthetic mRNAs that have increased binding affinity for to CD47 as multi-valent molecules, including (but not limited to) tetramers, hexamers, octamers, etc. of the CD47 binding domain. The CD47 binding domain may be wild type (WT), e.g., WT human SIRPα binding site, or it may be a modified version of the CD47 binding domain having similar or better binding activity and/or affinity.

The SIRPα fusion proteins described herein may include multiple copies of a SIRPα CD47 binding domain (or engineered variant having significant CD47 binding), and/or it may include one or more regions or domains binding C-C chemokine receptor type 4 (CCR4). CCR4. CCR4 is a protein that in humans is encoded by the CCR4 gene and may also be referred to as CD194 (cluster of differentiation 194). The protein encoded by CCR4 belongs to the G protein-coupled receptor family. Described herein are SIRPα fusion proteins that may be fused to one or more CCR4 binding polypeptide. The CCR4 binding polypeptide may be referred to herein as anti-CCR4 or as a CCR4 binding polypeptide. The CCR4 binding polypeptide may be an antibody or antibody fragment binding CCR4, such as a human or humanized antibody or antibody fragment binding to CCR4.

Synthetic mRNA molecules encoding SIRPα fusion proteins described herein may be used (and may be configured, compounded or otherwise prepared for) use as, or as part of, a therapeutic composition for treating an animal, including a human. Thus, any of these synthetic mRNA-encoded SIRPα fusion proteins may be used in combination with mRNAs encoding a target antigen, such as a tumor-specific antigen for treatment of a subject. The SIRPα fusion protein mRNA may be at least partially encapsuled by a delivery vehicle molecule, to form an mRNA nanoparticle. For example, the delivery vehicle molecule may be (but is not limited to) a hydroxyethyl-capped cationic peptoid, including, for example, hydroxyethyl-capped tertiary amino lipidated cationic peptoid. The SIRPα fusion protein mRNA may be encapsulated with or separately from a target antigen. Also described herein are methods of using these SIRPα fusion proteins to treat a subject, including injecting an mRNA nanoparticle comprising any of these SIRPα fusion proteins.

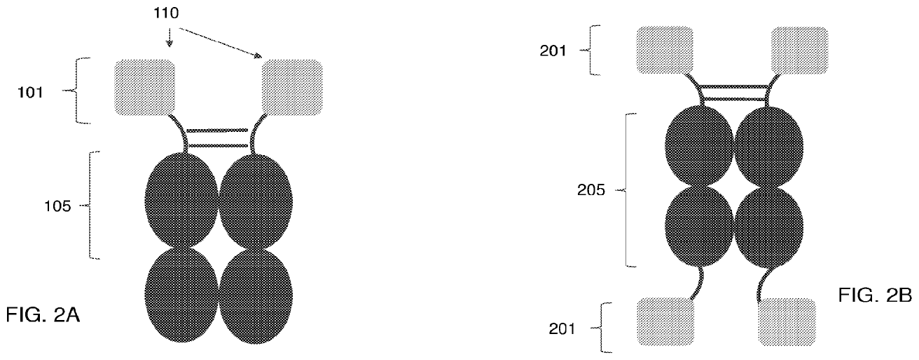

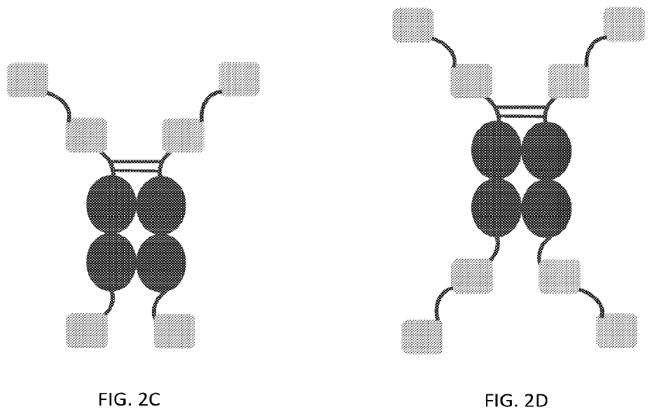

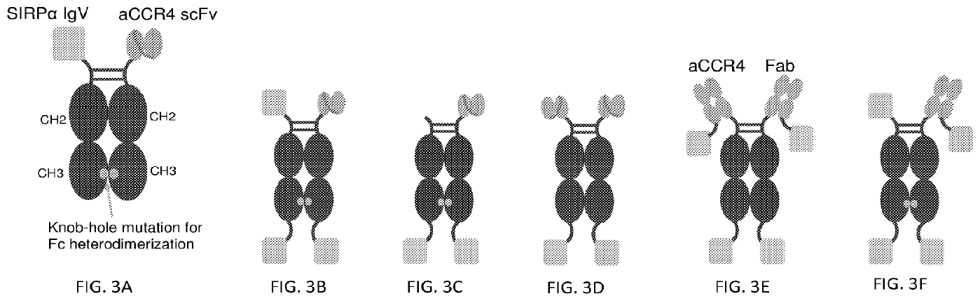

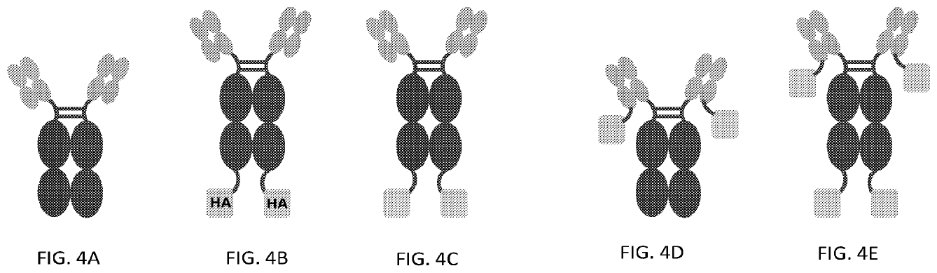

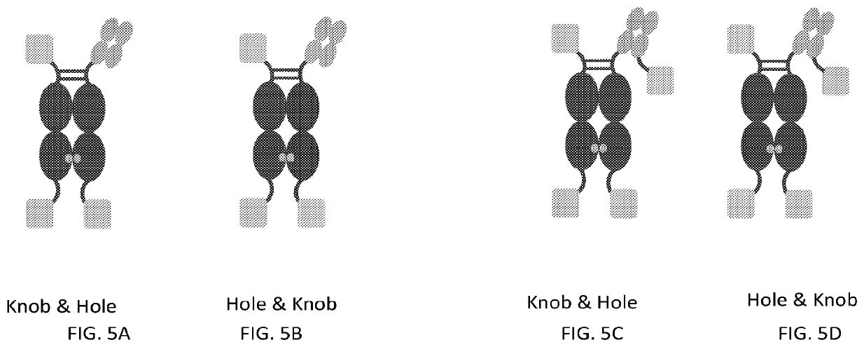

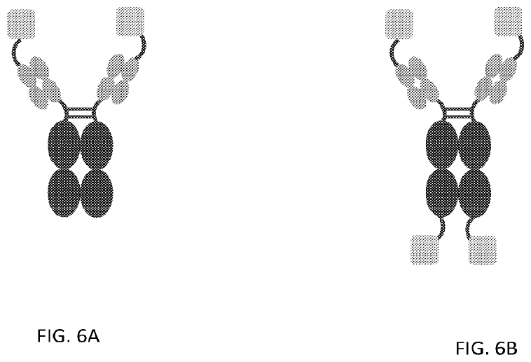

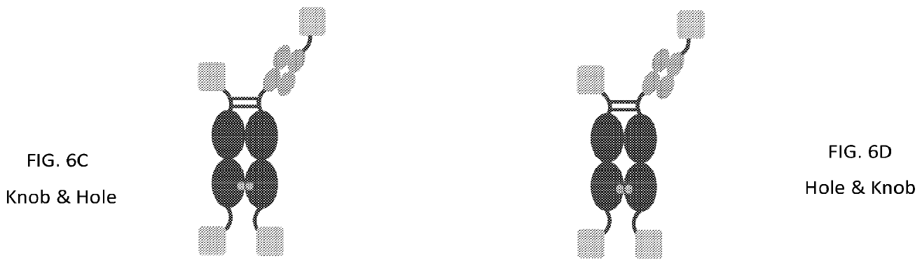

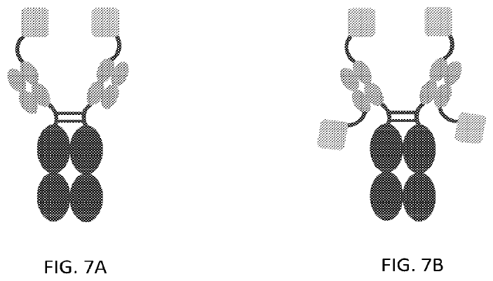

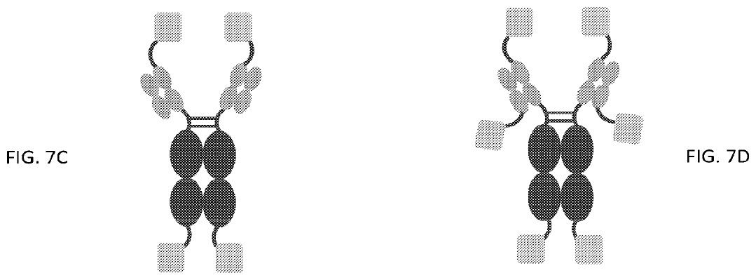

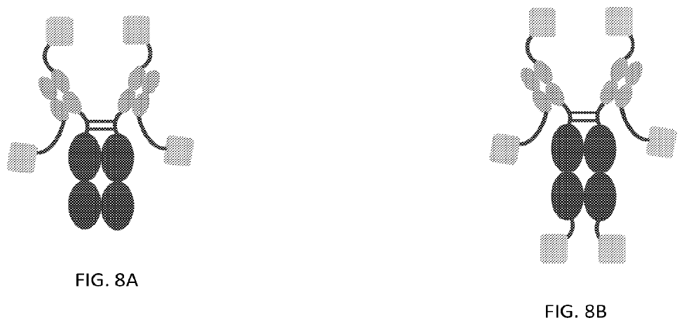

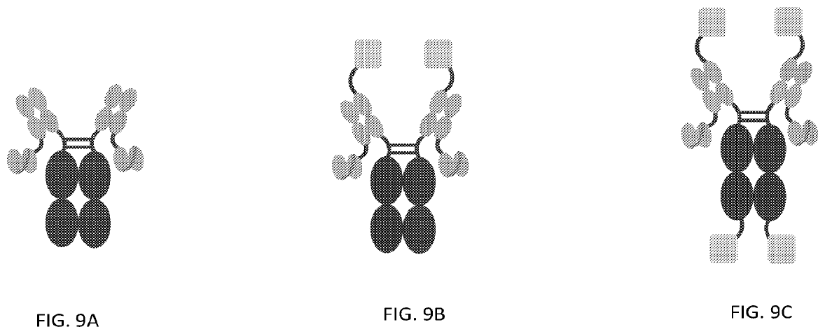

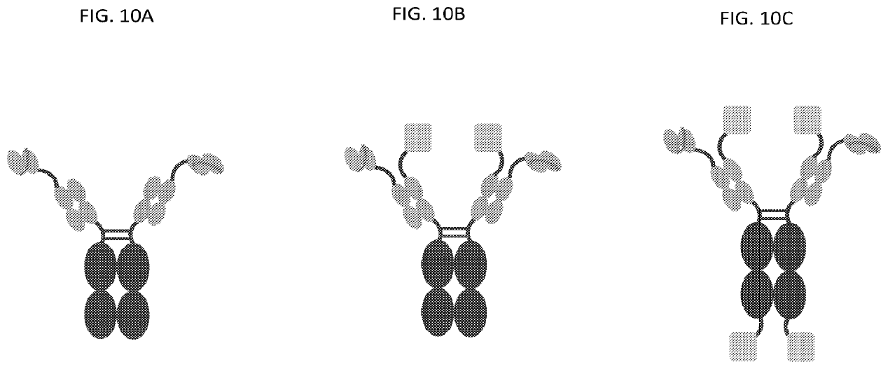

FIG. 2B illustrates structural representation of multivalent fusion proteins having a crystallizable fragment (Fc) 205 and an antigen binding fragment (Fab) 201. Both the Fc and Fab regions may have a constant domain, variable domains, or a combination thereof. SIRPα proteins may have an extracellular domain (ECD) with a Fab comprising multiple immunoglobulins. In some examples, wild type (WT) genes encoding human SIRPα can be polymorphic. Two common variants have been identified (e.g., SIRPV1—Allele 1; and SIRPαV2—Allele 2) with changes in surface amino acids that generally do not modify binding activity of WT SIRPα to CD47.

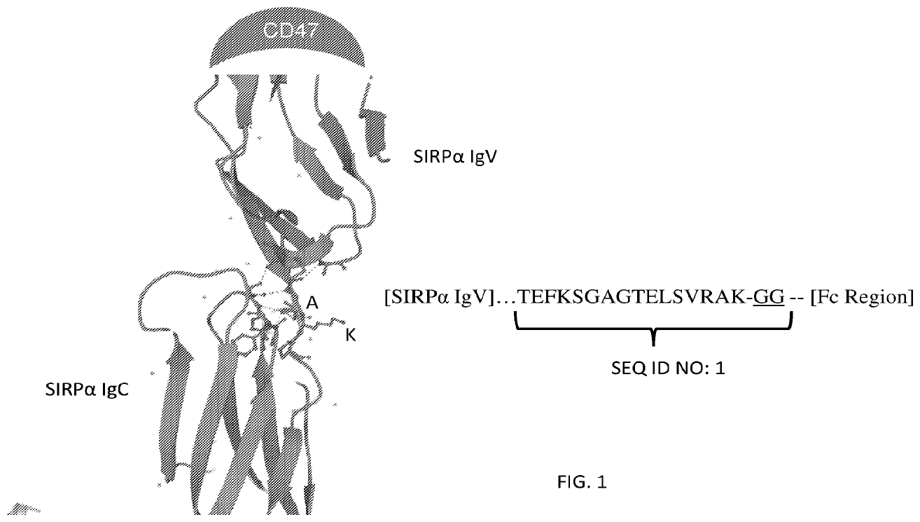

As described herein, an mRNA fusion protein may include engineered RNA sequences derived from or based on WT SIRPα antigen binding domain region coupled with a crystallizable fragment (Fc region). The engineered mRNA sequence may encode functional elements of the molecular structure with binding targets and related characteristics based on one or more signal regulatory proteins (e.g., SIRPα). The binding target or ligand can specifically be CD47. FIGS. 1 and 2 schematically illustrate examples engineered fusion proteins described herein. FIG. 2B illustrates a tetravalent example of an engineered SIRPα fusion protein with the antigen binding fragment 201, 201′ coupled to the Fc 205. The tetravalent antigen binding construct provides four antigen binding sites engineered with improved affinity to their target ligand (e.g., CD47). In first SIRPα CD47 binding region 201 is linked to the Fc region by a linker 209, such as, e.g., a GG linker. The second SIRPα CD47 binding region 201′ may be linked with the same or a different linker 209′ (e.g., a (GGGGS)ₙ linker).

FIG. 2A illustrates a bivalent example of an engineered SIRPα fusion protein with the antigen binding fragment 201 coupled to the Fc 205. In FIG. 2, the bivalent antigen binding construct provides two antigen binding sites 210, which may be engineered to improve affinity to their target ligand (e.g., CD47), e.g., high affinity (HA) engineered constructs.

Binding affinity of SIRPα WT to CD47 can be appropriately measured in micromolar (μM) affinity with dissociation contacts (Kd) in the order of 10⁻⁴ to 10⁻⁶. High affinity engineered variations of SIRPα, as described herein, can be appropriately measured in picomolar (pM) affinity to CD47 with Kd values substantially lower than WT (e.g., Kd values in the order of 10-10 to 10-12).

In some examples, the Fc region can be a heterogenous or homogenous combination of immunoglobulins (Ig). For example, an Fc region of the molecules including the amino acid sequences described here, may include 2 CH2, 2 CH3 (IgA, IgD, IgG). Considering FIG. 2A and the bivalent design of a molecule, the antigen binding sites may be derived from one or more of the different WT variants of SIRPα proteins. In the examples described in greater detail below, labeled molecules “1052” and “1053” are shown through the various assays to represent different allelic variants of WT SIRPα (e.g., allele 1, SEQ ID NO: 5, and allele 2, SEQ ID NO: 6).

The SIRPα fusion proteins described herein may be referred to by their amino acid sequences. Corresponding mRNA sequences may be readily determined by one of skill in the art from the amino acid sequences. One of skill in the art, without undue experimentation, may apply codon substitution to determine one or more mRNA sequences for any of these polypeptides described herein. Exemplary RNA sequences are provided herein as (e.g., SEQ. ID. NOs:39-59). In some examples, the SIRPα fusion proteins described herein may have an antigen binding fragment (e.g., and antigen binding site) based on an engineered sequence of mRNA relating to an RNA sequence of a native or wild-type SIRPα. For example, an engineered sequence of mRNA may be homologous to a wild-type SIRPα RNA sequence. The percentage of homology between the engineered mRNA and the WT RNA sequences may relate to the number of amino acid substitutions in the engineered mRNA sequence. In some examples, the engineered mRNA sequence may have at least one amino acid substitution relative to a WT SIRPα sequence. In some examples, the number of amino acid substitutions in the engineered SIRPα mRNA sequence may be two or more. In some examples, the number of amino acid substitutions in the engineered SIRPα mRNA sequence may be three or more. In some examples, the number of amino acid substitutions in the engineered SIRPα mRNA sequence may be fewer than fifteen.

In some examples, amino acid substitutions result in a variation or mutation to the antigen binding fragment of the protein encoded by the engineered mRNA. As an illustrative example, one or more amino acid substitutions encoded at specific locations of the engineered mRNA or polypeptide may result in an antigen binding site having improved activity compared to a WT variable Ig.

In some examples, the antigen binding site of the fusion proteins described herein may have fewer variable Ig domains compared to the complete extracellular domain (ECD) of a WT SIRPα protein.

The polypeptide of SEQ ID NO:7 is an example of an engineered sequence having amino acid substitutions promoting high binding affinity of the encoded SIRPα molecule. The number of amino acid substitutions may be fewer than fifteen substitutions at locations of the engineered sequence to reduce potential toxicity with an optimized high binding affinity for CD47. Fewer mutations or amino acid substitutions may retain WT SIRPα related structure and biophysical properties while reducing the risk of immunogenicity.

In this example, the high-affinity bivalent structure of SEQ ID NO: 7 includes a human light chain sequence (or a portion of a human light chain sequence, e.g., residues 1-22), coupled to a high-affinity sequence of SIRPα (e.g., residues 23-139). An optimized GG linker (amino acid residues 140-141) separates the high-affinity SIRPα sequence from a human Ig heavy chain region (amino acid residues 142-369).

An engineered mRNA molecule (e.g., encoding a SIRPα fusion protein) may encode a molecule forming a tetravalent structure having four distinct antigen binding sites (See FIG. 2B and SEQ ID NO:2). An mRNA encoding a tetravalent engineered structure (e.g., SEQ ID NOS: 44 and 54) may encode a fusion protein with increased binding affinity to a ligand (e.g., CD47) with fewer liabilities, e.g., decreased binding to red blood cells. For example, a tetravalent fusion protein including engineered SIRPα related antigen binding sites may have increased binding affinity as a result of an increase in avidity due to the tetravalent binding sites when compared to a bivalent construct with the same or similar mRNA encoding functional antigen binding sites. According to some examples provided herein, avidity refers to the measure of the total binding strength of a fusion protein at every binding site.

The amino acid sequence of SEQ ID NO:2 provides an example of an engineered polypeptide sequence encoding a tetravalent fusion protein targeting SIRPα. The example of an SIRPα sequence in the tetravalent fusion protein is preferably wild-type, but it may be a high-affinity SIRPα region. In the example shown in SEQ ID NO:2 the SIRPα portion (residues 1 and 116 and/or resides 354 to 470 of WT SIRPα) is homologous with a WT SIRPα. In general, the two SIRPα regions may be identical, or they may be different.

In some examples, one or more of the antigen binding sites of the tetravalent fusion protein encoded by an engineered mRNA sequence as described herein may be a modified sequence encoding a high affinity SIRPα construct having a higher binding affinity compared to a WT SIRPα protein. In some examples, all of the antigen binding sites of the tetravalent fusion protein are encoded by an engineered sequence described herein may be a modified polypeptide sequence encoding a high affinity SIRPα construct having a higher binding affinity compared to a WT SIRPα protein. SEQ ID NO: 3 illustrates another example of a tetravalent fusion protein including SIRPα.

In the amino acid sequence of SEQ ID NO: 2, the construct is configured to form a tetravalent fusion protein, and the amino acid sequence includes a portion of the SIRPα sequence configured to bind to CD47 (amino acid residues 1 to 116), coupled via a GG linker a human Ig heavy chain region (amino acid residues 119 to 344), and a second linker, a flexible linker, (GGGS)x (in this example x=2), couples the human Ig heavy chain region to a second portion of the SIRPα sequence configured to bind to CD47 (amino acid residues 354 to 470).

In some examples described herein, the engineered SIRPα-Fc constructs are made as single chain with flexible linker with dimerization post-translation. The linker region of the molecule may have one or more modifications to the amino acid sequence relative to a linker region of a WT SIRPα protein. As shown in FIG. 1, a representation of the protein structure of the SIRPα-Fc region can be seen. An example of a partial sequence highlights the linker region of the engineered sequence and underlines an example of amino acid substitutions specifically within the linker region of the engineered sequence. In this particular example, FIG. 1 provides a double glycine substitution as shown by the underline “GG”. In some examples, the linker region of a fusion protein described herein comprises a modified linker region between the antigen binding domains and the crystallizable fragment. Although a specific double glycine substitution is illustrated by FIG. 1 (part of SEQ IN NO: 1), and may be particularly beneficial, alternative amino acids may be substituted in accordance with the desired biophysiological impact of associated changes to the structure and function of the encoded linker region.

Any of the fusion proteins described herein may include one or more Fc modifications for half-life extension (which may allow less frequent dosing), and/or for enhanced effector function (which may improve tumor cell killing), and/or for reduced effector function (which may enhance improved safety) or may incorporate other IgG isotypes (such as IgG2, IgG3, or IgG4 Fc), which may provide different effector function properties. For example, two variants in the IgG1 Fc may be used with any of the fusion proteins described herein, e.g., S239D/I332E and/or S239D/A330L/I332E. Thus, the Fe domains of any of these fusion proteins may be engineered to contain one or more mutations to increase the affinity for FcRn, thereby further increasing the half-life in circulation. Such mutations include, but are not limited to, M252Y/S254T/T256E, M428L/N434S, T250Q/M428L, N434H, T307Q/N434A, T307Q/N434S, T307Q/E380A/N434S, and V308P/N434S. Fe modifications that may result be made (and may result in a change in effector function) or binding affinity: S298A/E333A/K334A, Afucosylation, S239D/I332E, S239D/A330L/I332E, G236A, G236A/S239D/1332E, G236A/A330L/I332E, G236A/S239D/A330L/1332E, F243L/R292P/Y300L/V305I/P396L, L235V/F243L/R292P/Y300L/P396L, One heavy chain: L234Y/L235Q/G236W/S239M/H268D/D270E/S298A and opposing heavy chain: D270E/K326D/A330M/K334E. (numbering according to Kabat EU index).

### Therapeutic Compositions

Any of the SIRPα fusion proteins described herein may be part of a therapeutic composition comprising an mRNA encoding the amino acid sequence of the SIRPα fusion protein and a delivery vehicle molecule. For example, any of the SIRPα fusion proteins described herein may be part of a method and/or composition (including kits). These methods may be, for example, methods for treating cancer and/or treating tumors.

The SIRPα fusion proteins described herein may be encoded by an mRNA nanoparticle and may be used as part of a patient treatment using these mRNA nanoparticles (e.g., including mRNA vaccines). In particular, described herein are mRNA therapies being administered to a patient in need thereof. The patient can be an animal, for example, a human. The therapies may be mRNA therapies involve administering (to a patient or a site of a patient) mRNA (treatment) nanoparticles, including mRNA encoding one or more patient-specific antigens and mRNA encoding mRNA encoding one or more SIRPα fusion proteins described herein, as well as apparatuses and methods for making patient-specific mRNA therapies including these.

The mRNA vaccines including any of the mRNA encoding SIRPα fusion proteins described herein may be used in a methods for treating cancer, including treating tumors that are not benign, or which can be benign or cancerous, using these mRNA treatments, and methods of forming these mRNA vaccines. For example, described herein are methods of that include the intratumoral injection of an mRNA treatment nanoparticle encoding a tumor-specific antigen, and an mRNA encoding a SIRPα fusion protein, wherein the mRNA encoding the tumor-specific antigen and the mRNA encoding the SIRPα fusion proteins are encapsulated together by a delivery vehicle molecule, for example, a hydroxyethyl-capped cationic peptoid, including, for example, hydroxyethyl-capped tertiary amino lipidated cationic peptoid. While the injection may be intratumoral, as provided in some examples herein, other routes of administration are also possible.

Any of the methods described herein may be methods of treatment, including methods of treating a cancer (e.g., to reduce, or in some instances eliminate, a tumor).

The methods described herein may include: injecting an mRNA treatment nanoparticle comprising: one or more mRNAs encoding a tumor-specific antigen and an mRNA encoding a SIRPα fusion protein; and a delivery vehicle molecule encapsulating the one or more mRNAs. For example, a method may include: one or more mRNAs encoding a tumor-specific antigen and an mRNA encoding a SIRPα fusion protein; and a delivery vehicle molecule encapsulating the one or more mRNAs. In some examples, a method may include intratumorally injecting an mRNA treatment nanoparticle comprising one or more mRNAs encoding a tumor-specific antigen and an mRNA encoding a SIRPα fusion protein as well at least one or more additional immunomodulatory agent, wherein the at least one or more immunomodulatory agent is a checkpoint inhibitor; and a delivery vehicle molecule encapsulating the one or more mRNAs. In some examples, the method may include: intratumorally injecting an mRNA treatment nanoparticle comprising: one or more mRNAs encoding a tumor-specific antigen, one or more mRNA encoding a SIRPα fusion protein; and a delivery vehicle molecule encapsulating the mRNAs, wherein the delivery vehicle molecule comprises a hydroxyethyl-capped cationic peptoid, including, for example, hydroxyethyl-capped tertiary amino lipidated cationic peptoid delivery vehicle.

For example, described herein are methods comprising: injecting an mRNA treatment nanoparticle comprising an mRNA encoding a tumor-specific antigen (e.g., an mRNA vaccine) and an mRNA encoding a SIRPα fusion protein, wherein the mRNA encoding the tumor-specific antigen and the mRNA encoding a SIRPα fusion protein are encapsulated with the same delivery vehicle molecule. For example, the method may include: intratumorally injecting an mRNA treatment nanoparticle comprising an mRNA encoding a tumor-specific antigen and an mRNA encoding a SIRPα fusion protein, wherein the mRNA vaccine and the immunomodulatory agent are encapsulated with the same nanoparticle delivery vehicle (or in some examples a different delivery vehicle).

A method may include: intratumorally injecting an mRNA treatment nanoparticle comprising an mRNA encoding a tumor-specific antigen (e.g., mRNA vaccine) and an mRNA encoding a SIRPα fusion protein and one or more additional immunomodulatory agents, wherein at least one of the two or more immunomodulatory agents is a checkpoint inhibitor, further wherein the mRNA encoding the tumor-specific antigen and the mRNAs encoding the immunomodulatory agents are encapsulated with the same delivery vehicle molecule (or in some examples a different delivery vehicle).

Any appropriate delivery vehicle molecule may be used. For example, the delivery vehicle molecule may be a lipid based vehicle (such as a lipid nanoparticle) or a polymer-based nanoparticle. In particular the delivery vehicle (DV) molecule may be an amino-lipidated peptoid delivery vehicle. In any of these methods the delivery vehicle molecule may comprise an amino-lipidated peptoid. In one example, the delivery vehicle molecule comprises at least one peptoid, which can be any type of peptoid described herein. In another example, the delivery vehicle molecule comprises more than one peptoid, which can be any type of peptoid described herein.

The mRNA encoding the tumor-specific antigen may comprise an mRNA encoding a patient-specific antigen (or multiple patient-specific antigens). For example, the mRNA treatment (e.g., mRNA vaccine) may comprise an mRNA encoding a plurality of patient-specific antigens. The mRNA encoding the tumor-specific antigen may include an mRNA encoding a shared tumor antigen.

An mRNA encoding an immunomodulatory agent may encode: a checkpoint inhibitor, an immunosuppression antagonist, a pro-inflammatory agent, or combinations thereof. For example, the mRNA encoding an immunomodulatory agent encodes anti-CTLA-4. The mRNA encoding an immunomodulatory agent may encode a TGF-beta antagonist. The mRNA encoding an immunomodulatory agent may encode a single chain interleukin-12 (IL-12).

Any of the mRNA therapies and methods of making or using them may include the addition of one or more adjuvant, which may be included with the mRNA treatment. In some variations the delivery vehicle molecules may be mixed with, include and/or encapsulate the additional adjuvant. In some variations the additional adjuvant may be combined with the solution including the nanoparticles encapsulating the therapeutic mRNAs (e.g., the tumor-specific mRNA and the mRNA encoding a SIRPα fusion protein). Any of these mRNA therapies may further include an immunostimulator. For example, the immunostimulator may comprise a CpG oligodeoxynucleotides.

In use, any of these methods may include repeating the injection treatment one or more times with a 1-7 day wait between treatments. As mentioned, the injection may be (but is not limited to) intratumoral. In some variations, the injection may be both intratumoral and/or by other means (subcutaneous, etc.).

The mRNA encoding the tumor-specific antigen and the mRNA encoding a SIRPα fusion protein may be on a single mRNA strand, or on separate mRNA strands.

Any appropriate cancer or tumor may be treated, as described herein. For example, and of these methods may be methods of treating Lymphoma. The method described herein may be methods of treating cervical cancer.

As mentioned above, any appropriate delivery vehicle molecule may be used. For example, the delivery vehicle molecule may comprise an amino-lipidated peptoid delivery vehicle.

The mRNA vaccines described herein may include an mRNA encoding one or more tumor-specific antigen and one or more mRNA encoding a SIRPα fusion protein that is co-formulated with the same delivery vehicle molecule. The delivery vehicle molecule may be, for example, a lipid nanoparticle (LNP). LNP formulations may be composed of an ionizable or cationic lipid or polymeric material, bearing tertiary or quaternary amines to encapsulate the polyanionic mRNA; a zwitterionic lipid (e.g., 1,2-dioleoyl-sn-glycero-3-phosphoethanolamine) that resembles the lipids in the cell membrane; cholesterol to stabilize the lipid bilayer of the LNP; and a polyethylene glycol (PEG)-lipid to lend the nanoparticle a hydrating layer, improve colloidal stability, and reduce protein absorption.

Multicomponent LNPs may be taken up by endocytosis and can electrostatically attach and fuse with the cell membrane using inverted non-bilayer lipid phases. Once inside the cell, LNPs may be routed into early endosomes, followed by late endosomes, and finally the lysosomes where the mRNA contents are enzymatically degraded.

One class of delivery vehicle molecules includes the cationic or ionizable lipids and lipid-like materials. Cationic lipids bear alkylated quaternary ammonium groups and retain their cationic nature in a pH-independent fashion, while ionizable lipids acquire positive charges by protonation of free amines as pH is lowered. Lipid-like materials bear more hydrophobic side chains than natural lipids. Cationic lipids, such as N-[1-(2,3-dioleoyloxy)propyl]-N,N,N-trimethylammonium chloride (DOTMA) may be used. Alternatively or additionally, pH-dependent ionizable materials may be used. The ionizable lipid named Dlin-MC3-DMA (MC3) can also be used to transfect mRNA in order to express therapeutic proteins.

Polymeric materials may be used for the delivery of therapeutic mRNA. For example, low-molecular-weight polyethyleneimine (PEI) modified with fatty chains may be used for siRNA and mRNA delivery to reduce toxicity of high-molecular weight PEI. Poly(glycoamidoamine) polymers modified with fatty chains (e.g., in some variations including a tartrate backbone) have been shown to deliver mRNA. Poly(β-amino)esters (PBAEs) are biodegradable polymers that may be used for nucleic acid delivery, including PBAEs formulated with PEG-lipid to increase their serum stability. Hyperbranched PBAEs may be used for mRNA delivery.

Polymethacrylates with amine-bearing side chains, polyaspartamides with oligoaminoethylene side chains, and polyacrylic acids amidated with tetramine with alternating ethyl-propyl-ethyl spacers have been reported to transfect mRNA and may be used as a delivery vehicle molecule. Self-immolative polycarbonate-block-poly(α-amino)esters may release mRNA upon rearrangement followed by degradation at pH 7.4, to facilitate endosomal escape. Biodegradable amino polyesters (APEs), may be synthesized with low dispersity from tertiary amino alcohols as initiators in ring-opening polymerization of various lactones, and may be capable of tissue-selective mRNA delivery. Other biodegradable polymers with biocompatible degradation products and enhanced endosomal escape capabilities may be used for mRNA delivery.

In some variations the delivery vehicle molecule may include dendrimers. For example, polyamidoamine (PAMAM) or polypropylenimine-based dendrimers have been extensively studied for gene delivery. Fatty chain-modified PAMAM dendrimers, and/or a modified PAMAM dendrimer co-formulated with poly(lactic-co-glycolic acid) (PLGA) and ceramide-PEG may be used as a delivery vehicle molecule.

Alternatively, in some variations cell-penetrating peptides (CPPs) may be used as delivery vehicle molecules. CPPs may promote clustering of the negatively charged glycosaminoglycans on the cell surface, which in turn triggers macropinocytosis and lateral diffusion or directly disrupts the lipid bilayer. A CPP with arginine-rich amphipathic RALA sequence repeats may be used. In some variations the delivery vehicle molecule may be a combination of cationic and zwitterionic lipids, reminiscent of cationic and helper lipids (“zwitterionic amino lipids” or ZALs).

In particular, the delivery vehicle molecules described herein may be amino-lipidated peptoid delivery vehicles. For example, these delivery vehicles may be a lipid-containing amphipathic delivery vehicle that provides packaging and protection of mRNA cargos during circulation, avoid immune recognition, and may facilitate cellular uptake and release. Examples of these delivery vehicles may be found in international patent application, PCT/US19/53661, titled “LIPID NANOPARTICLE FORMULATIONS COMPRISING LIPIDATED CATIONIC PEPTIDE COMPOUNDS FOR NUCLEIC ACID DELIVERY”, and filed on Sep. 27, 2019, and in international patent application PCT/US19/53655, titled “TERTIARY AMINO LIPIDATED CATIONIC PEPTIDES FOR NUCLEIC ACID DELIVERY” and filed on Sep. 27, 2019, each of which is herein incorporated by reference in its entirety. Multiple types of delivery vehicle molecules may be used, and each type may be encapsulated together with both the mRNA encoding one or more tumor-specific antigen and mRNA encoding one or more immunomodulatory agents as described herein.

### Delivery Vehicle Compositions

Disclosed herein are delivery vehicle compositions comprising hydroxyethyl-capped cationic peptoids, including, for example, hydroxyethyl-capped tertiary amino lipidated cationic peptoids. The delivery vehicle compositions of the disclosure can form an electrostatic interaction between the hydroxyethyl-capped tertiary amino lipidated cationic peptoids of the delivery vehicle composition and a polyanionic compound, such as a nucleic acid, to form a delivery vehicle complex, wherein the polyanionic compound functions as the cargo of the complex. The delivery vehicle complex is useful for the delivery of polyanionic compounds, such as nucleic acids (e.g., mRNA), into cells. Delivery vehicle complexes of the disclosure that include mRNA as the polyanionic cargo unexpectedly exhibit superior mRNA expression both in vitro and in vivo. When the mRNA of the delivery vehicle complex encodes, e.g., for a viral antigen, the delivery vehicle complexes can elicit humoral and cellular immune responses in vivo, thus functioning as a vaccine. The delivery vehicle complexes disclosed herein are further advantages in that they are stable, and demonstrate good tolerability and low toxicity.

As used herein, “peptoid” refers to a peptidomimetic compound in which one or more of the nitrogen atoms of the peptide backbone are substituted with side chains. As used herein, “lipidated peptoid” refers to a peptoid in which one or more of the side chains on the nitrogen atom comprises a lipid. As used herein, “polyanionic” refers to a compound having at least two negative charges, such as nucleic acids.

Some example delivery vehicle compositions of the disclosure comprise one or more hydroxyethyl-capped tertiary amino lipidated cationic peptoids. These positively charged peptoids can associate with a polyanionic compound, such as a nucleic acid, to form a delivery vehicle complex. In some implementations, the delivery vehicle compositions further comprise one or more of an anionic or zwitterionic component, such as a phospholipid; a neutral lipid, such as a sterol; and a shielding lipid, such as a PEGylated lipid. In various implementations, the delivery vehicle compositions further comprise an anionic or zwitterionic component (e.g., a phospholipid), a neutral lipid (e.g., a sterol), and a shielding lipid (e.g., a PEGylated lipid). In some aspects, the delivery vehicle compositions consist essentially of a hydroxyethyl-capped tertiary amino lipidated cationic peptoid, an anionic or zwitterionic component (e.g., a phospholipid), a neutral lipid (e.g., a sterol), and a shielding lipid (e.g., a PEGylated lipid).

### Hydroxyalkyl-Capped Tertiary Amino Lipidated Cationic Peptoid Component

The delivery vehicle compositions may comprise hydroxyalkylcapped cationic peptoids, including, for example, a 2-aminopropane-1,3-diol-capped cationic peptoid. The delivery vehicle compositions can form an electrostatic interaction between the 2-aminopropane-1,3-diol-capped cationic peptoids of the delivery vehicle composition and a polyanioniccompound, such as a nucleic acid, to form a delivery vehicle complex, wherein the polyanionic compound functions as the cargo of the complex.

In some implementations, the hydroxyalkyl-capped cationic peptoids comprise a compound of Formula (I):

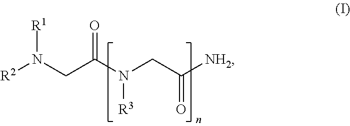

wherein n is n is 1, 2, 3, 4, 5, 6, 7, 8, 9, or 10; R1 is H or C2-5 alkyl optionally substituted with 1-3 OH; R2 is H or C2-5 alkylene-OH substituted with 1-3 additional OH; and each R3 independently is C6-24 alkyl or C6-24 alkenyl. As used herein, “alkyl” refers to straight chained and branched saturated hydrocarbon groups containing one to thirty carbon atoms, for example, one to twenty four carbon atoms (e.g., 1, 2, 3, 4, 5, 6, 7, 8, 9, 10, 11, 12, 13, 14, 15, 16, 17, 18, 19, 20, 21, 22, 23, or 24 carbon atoms). The term Cn means the alkyl group has “n” carbon atoms. For example, C3 alkyl refers to an alkyl group that has 3 carbon atoms. C1-24 alkyl refers to an alkyl group having a number of carbon atoms encompassing the entire range (i.e., 1 to 24 carbon atoms), as well as all subgroups (e.g., 1-2, 1-3, 1-4, 1-5, 1-6, 1-7, 1-8, 1-9, 1-10, 1-11, 1-12, 1-13, 1-14, 1-15, 1-16, 1-17, 1-18, 1-19, 1-20, 1-21, 1-22, 1-23, 1-24, 2-3,2-4,2-5,2-6,2-7,2-8,2-9,2-10,2-11,2-12,2-13,2-14,2-15, 2-16, 2-17, 2-18, 2-19, 2-20, 2-21,2-22,2-23,2-24,3-4, 3-5,3-6, 3-7,3-8,3-9, 3-10, 3-11, 3-12, 3-13, 3-14, 3-15, 3-16, 3-17, 3-18, 3-19, 3-20, 3-21, 3-22, 3-23, 3-24, 4-5, 4-6, 4-7, 4-8, 4-9, 4-10, 4-11, 4-12, 4-13, 4-14, 4-15, 4-16, 4-17, 4-18, 4-19, 4-20, 4-21, 4-22, 4-23, 4-24, 5-6, 5-7, 5-8, 5-9, 5-10, 5-11, 5-12, 5-13, 5-14, 5-15, 5-16, 5-17, 5-18, 5-19, 5-20, 5-21, 5-22, 5-23, 5-24, 6-7, 6-8,6-9, 6-10, 6-11,6-12, 6-13,6-14, 6-15, 6-16,6-17,6-18, 6-19, 6-20, 6-21,6-22,6-23,6-24,7-8,7-9,7-10,7-11,7-12,7-13,7-14,7-15,7-16,7-17,7-18,7-19, 7-20, 7-21, 7-22, 7-23, 7-24, 8-9, 8-10, 8-11, 8-12, 8-13, 8-14, 8-15, 8-16, 8-17, 8-18, 8-19, 8-20, 8-21, 8-22, 8-23, 8-24, 9-10, 9-11, 9-12, 9-13, 9-14, 9-15, 9-16, 9-17, 9-18, 9-19, 9-20, 9-21, 9-22, 9-23, 9-24, 10-11, 10-12, 10-13, 10-14, 10-15, 10-16, 10-17, 10-18, 10-19, 10-20, 10-21, 10-22, 10-23, 10-24, 11-12, 11-13, 11-14, 11-15, 11-16, 11-17, 11-18, 11-19, 11-20, 11-21, 11-22, 11-23, 11-24, 12-13, 12-14, 12-15, 12-16, 12-17, 12-18, 12-19, 12-20, 12-21, 12-22, 12-23, 12-24, 13-14, 13-15, 13-16, 13-17, 13-18, 13-19, 13-20, 13-21, 13-22, 13-23, 13-24, 14-15, 14-16, 14-17, 14-18, 14-19, 14-20, 14-21, 14-22, 14-23, 14-24, 15-16, 15-17, 15-18, 15-19, 15-20, 15-21, 15-22, 15-23, 15-24, 16-17, 16-18, 16-19, 16-20, 16-21, 16-22, 16-23, 16-24, 17-18, 17-19, 17-20, 17-21, 17-22, 17-23, 17-24, 18-19, 18-20, 18-21, 18-22, 18-23, 18-24, 19-20, 19-21, 19-22, 19-23, 19-24, 20-21, 20-22, 20-23, 20-24, 21-22, 21-23, 21-24, 22-23, 22-24, 23-24, 1, 2, 3, 4, 5, 6, 7, 8, 9, 10, 11, 12, 13, 14, 15, 16, 17, 18, 19, 20, 21, 22, 23, or 24 carbon atoms). Nonlimiting examples of alkyl groups include, methyl, ethyl, n-propyl, isopropyl, n-butyl, sec-butyl (2-methylpropyl), and t-butyl (1,1-dimethylethyl). Unless otherwise indicated, an alkyl group can be an unsubstituted alkyl group or a substituted alkyl group. As used herein, “alkenyl” refers to straight chained and branched hydrocarbon groups having a double bond and containing two to thirty carbon atoms, for example, two to twenty four carbon atoms (e.g., 1, 2, 3, 4, 5, 6, 7, 8, 9, 10, 11, 12, 13, 14, 15, 16, 17, 18, 19, 20, 21, 22, 23, or 24 carbon atoms). The term Cn means the alkenyl group has “n” carbon atoms. For example, C3 alkenyl refers to an alkenyl group that has 3 carbon atoms. C2-C24 alkenyl refers to an alkenyl group having a number of carbon atoms encompassing the entire range (i.e., 2 to 24 carbon atoms), as well as all subgroups (e.g., 2-3, 2-4, 2-5, 2-6, 2-7, 2-8, 2-9, 2-10, 2-11, 2-12, 2-13, 2-14, 2-15, 2-16, 2-17, 2-18, 2-19, 2-20, 2-21, 2-22, 2-23, 2-24, 3-4, 3-5, 3-6, 3-7, 3-8, 3-9, 3-10, 3-11, 3-12, 3-13, 3-14, 3-15, 3-16, 3-17, 3-18, 3-19, 3-20, 3-21, 3-22, 3-23, 3-24, 4-5, 4-6, 4-7, 4-8, 4-9, 4-10, 4-11, 4-12, 4-13, 4-14, 4-15, 4-16, 4-17, 4-18, 4-19, 4-20, 4-21, 4-22, 4-23, 4-24, 5-6, 5-7, 5-8, 5-9, 5-10, 5-11, 5-12, 5-13, 5-14, 5-15, 5-16, 5-17, 5-18, 5-19, 5-20, 5-21, 5-22, 5-23, 5-24, 6-7,6-8, 6-9,6-10, 6-11, 6-12, 6-13,6-14, 6-15, 6-16, 6-17, 6-18, 6-19, 6-20, 6-21, 6-22, 6-23, 6-24, 7-8, 7-9, 7-10, 7-11, 7-12, 7-13, 7-14, 7-15, 7-16, 7-17, 7-18, 7-19, 7-20, 7-21, 7-22, 7-23, 7-24, 8-9, 8-10, 8-11, 8-12, 8-13, 8-14, 8-15, 8-16, 8-17, 8-18, 8-19, 8-20, 8-21, 8-22, 8-23, 8-24, 9-10, 9-11, 9-12, 9-13, 9-14, 9-15, 9-16, 9-17, 9-18, 9-19, 9-20, 9-21, 9-22, 9-23, 9-24, 10-11, 10-12, 10-13, 10-14, 10-15, 10-16, 10-17, 10-18, 10-19, 10-20, 10-21, 10-22, 10-23, 10-24, 11-12, 11-13, 11-14, 11-15, 11-16, 11-17, 11-18, 11-19, 11-20, 11-21, 11-22, 11-23, 11-24, 12-13, 12-14, 12-15, 12-16, 12-17, 12-18, 12-19, 12-20, 12-21, 12-22, 12-23, 12-24, 13-14, 13-15, 13-16, 13-17, 13-18, 13-19, 13-20, 13-21, 13-22, 13-23, 13-24, 14-15, 14-16, 14-17, 14-18, 14-19, 14-20, 14-21, 14-22, 14-23, 14-24, 15-16, 15-17, 15-18, 15-19, 15-20, 15-21, 15-22, 15-23, 15-24, 16-17, 16-18, 16-19, 16-20, 16-21, 16-22, 16-23, 16-24, 17-18, 17-19, 17-20, 17-21, 17-22, 17-23, 17-24, 18-19, 18-20, 18-21, 18-22, 18-23, 18-24, 19-20, 19-21, 19-22, 19-23, 19-24, 20-21, 20-22, 20-23, 20-24, 21-22, 21-23, 21-24, 22-23, 22-24, 23-24, 1, 2, 3, 4, 5, 6, 7, 8, 9, 10, 11, 12, 13, 14, 15, 16, 17, 18, 19, 20, 21, 22, 23, or 24 carbon atoms). Nonlimiting examples of alkenyl groups include ethenyl, propenyl, butenyl, geranyl, and oleyl. Unless otherwise indicated, an alkenyl group can be an unsubstituted alkenyl group or a substituted alkenyl group.

In some implementations, n is 2 to 5. In various implementations, n is 3 to 4. In some implementations, n is 1. In various implementations, n is 2. In some cases, n is 3. In various cases, n is 4. In some implementations n is 5. In various implementations, n is 6. In various cases, n is 7. In various cases, n is 8. In various cases, n is 9. In various cases, n is 10.

In some implementations, R1 is H. In various implementations, R1 is C2-5 alkyl optionally substituted with 1-3 OH. In some cases, R1 is methyl or ethyl. In some implementations, R1 is ethyl. In various implementations, R1 is C2-5 alkylene-OH substituted with 0-2 additional OH. In some cases, R1 is

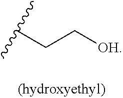 [SMILES: *CCO]

In various cases, R1 is ethyl or hydroxyethyl. In some cases, C2-5 alkyl is substituted with 1 OH. In some cases, C2-5 alkyl is substituted with 2 OH. In some cases, C2-5 alkyl is substituted with 3 OH.

In some implementations, R2 is C2-5 alkylene-OH substituted with 1-3 additional OH. In some cases, R2 is C2 alkylene-OH substituted with 1-3 additional OH. In some cases, R2 is C3 alkylene-OH substituted with 1-3 additional OH. In some cases, R2 is C4 alkylene-OH substituted with 1-3 additional OH. In some cases, R2 is C5 alkylene-OH substituted with 1-3 additional OH. In some cases, C2-5 alkylene-OH is substituted with 1 additional OH. In some cases, C2-5 alkylene-OH is substituted with 2 additional OH. In some cases, C2-5 alkylene-OH is substituted with 3 additional OH. In some cases, R2 is propyl-1,3-diol. In some cases, R2 is

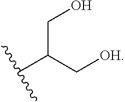 [SMILES: *C(CO)CO]

In some implementations, each R3 independently is C8-18 alkyl or C8-18 alkenyl. In various implementations, each R3 independently is C8-16 alkyl or C10-18 alkenyl. In some cases, each R3 independently is C6-18 alkyl or C6-18 alkenyl. In some cases, each R3 independently is C10-12 alkyl or C10-18 alkenyl. In some implementations, each R3 independently is: C8-18 alkyl, or C8-16 alkyl, or C8-14 alkyl, or C8-12 alkyl. In various implementations, each R3 independently is selected from the group consisting of

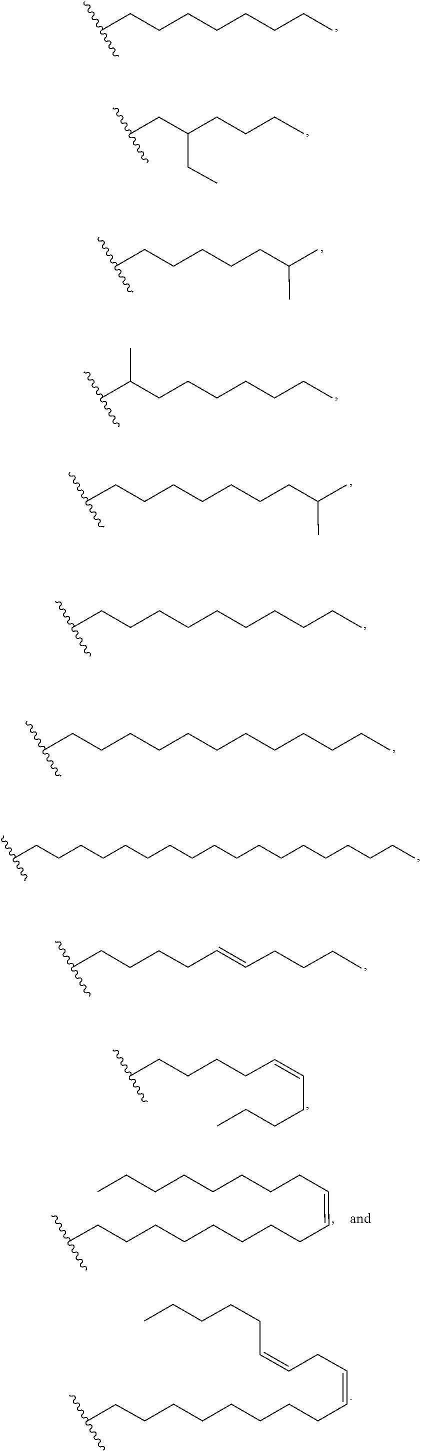

In some cases, each R3 independently is selected from the group consisting of

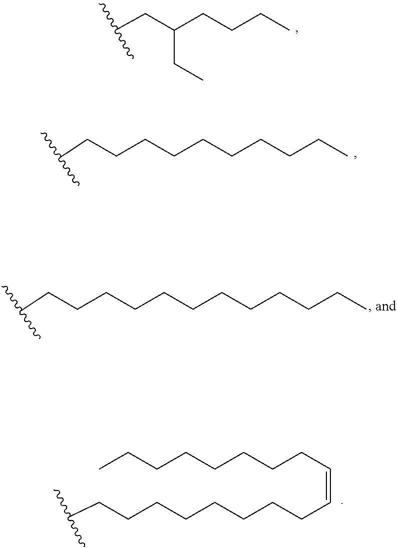

In various cases, each R3 independently is selected from the group consisting of

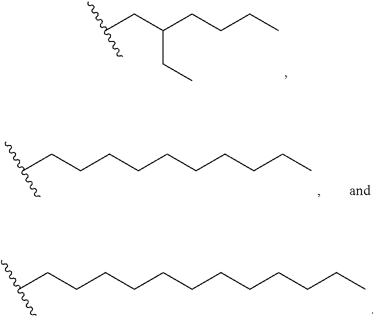

In some implementations, each R3 independently is

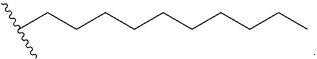 [SMILES: *CCCCCCCCCC]

Contemplated compounds of Formula (I) include, but are not limited to, the compounds listed in Table 1.

TABLE 1
Examples of hydroxyalkyl-capped cationic peptoids.

| Com- | |
|---|---|
| pound | Structure |
| 1 | 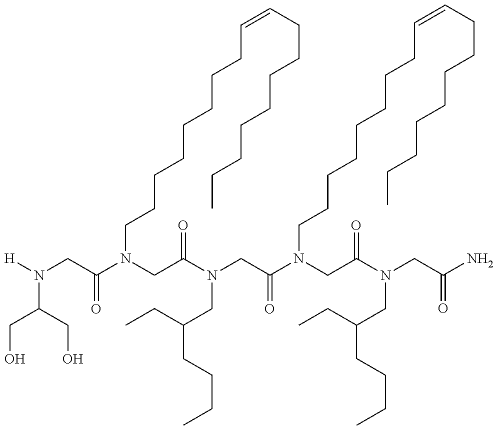 [SMILES: CCCCCCCC/C=C\CCCCCCCCN(CC(=O)N(CC(=O)N(CCCCCCCC/C=C\CCCCCCCC)CC(=O)N(CC(N)=O)CC(CC)CCCC)CC(CC)CCCC)C(=O)CNC(CO)CO] |
| 2 | 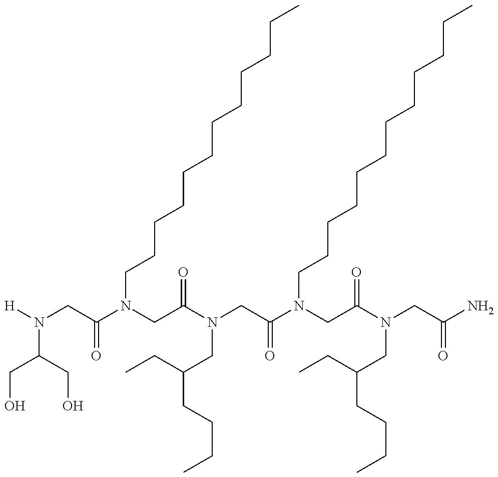 [SMILES: CCCCCCCCCCCCN(CC(=O)N(CC(=O)N(CCCCCCCCCCCC)CC(=O)N(CC(N)=O)CC(CC)CCCC)CC(CC)CCCC)C(=O)CNC(CO)CO] |
| 3 | 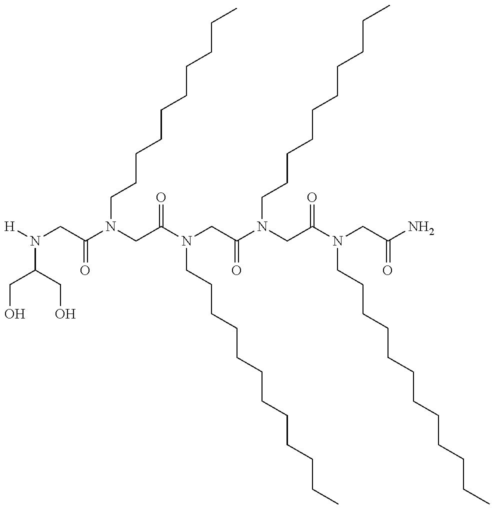 [SMILES: CCCCCCCCCCCCN(CC(N)=O)C(=O)CN(CCCCCCCCCC)C(=O)CN(CCCCCCCCCCCC)C(=O)CN(CCCCCCCCCC)C(=O)CNC(CO)CO] |
| 4 | 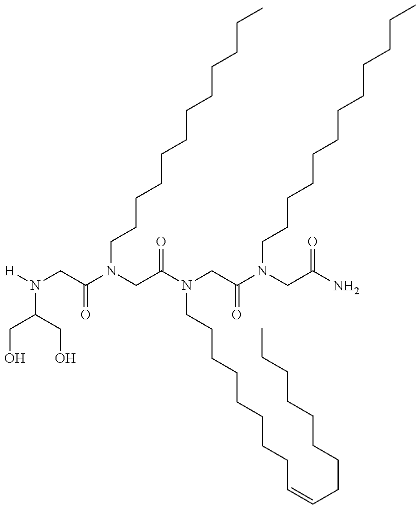 [SMILES: CCCCCCCC/C=C\CCCCCCCCN(CC(=O)N(CCCCCCCCCCCC)CC(N)=O)C(=O)CN(CCCCCCCCCCCC)C(=O)CNC(CO)CO] |
| 5 | 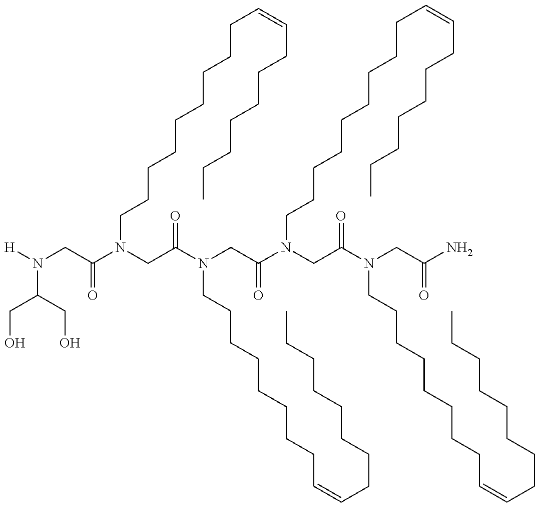 [SMILES: CCCCCCC/C=C\CCCCCCCCCN(CC(=O)N(CCCCCCCC/C=C\CCCCCCCC)CC(=O)N(CCCCCCCCC/C=C\CCCCCCC)CC(=O)N(CCCCCCCC/C=C\CCCCCCCC)CC(N)=O)C(=O)CNC(CO)CO] |
| 6 | 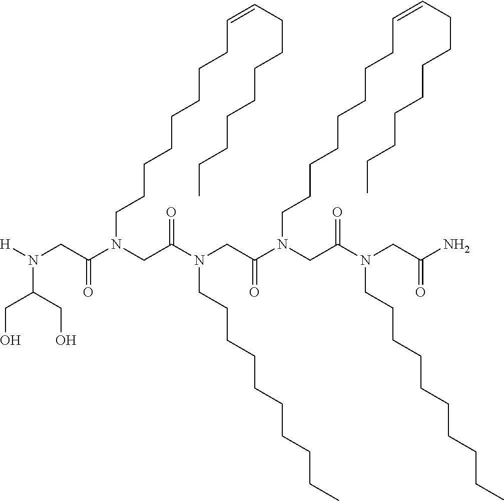 [SMILES: CCCCCCCC/C=C\CCCCCCCCN(CC(=O)N(CCCCCCCCCC)CC(=O)N(CCCCCCCC/C=C\CCCCCCCC)CC(=O)N(CCCCCCCCCC)CC(N)=O)C(=O)CNC(CO)CO] |
| 7 | 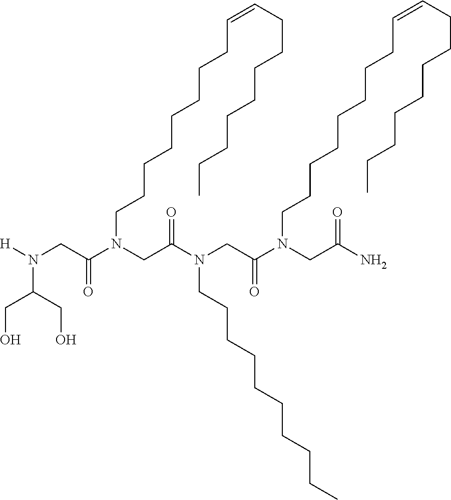 [SMILES: CCCCCCCC/C=C\CCCCCCCCN(CC(N)=O)C(=O)CN(CCCCCCCCCC)C(=O)CN(CCCCCCCC/C=C\CCCCCCCC)C(=O)CNC(CO)CO] |
| 8 | 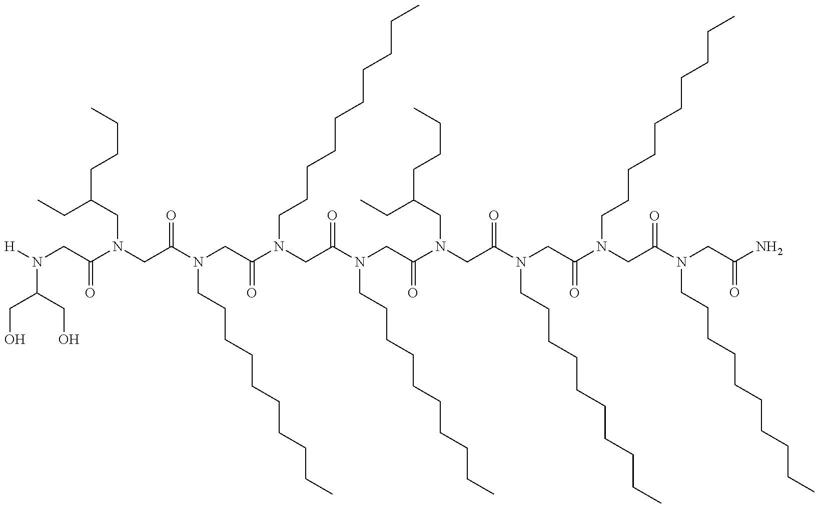 [SMILES: CCCCCCCCCCN(CC(N)=O)C(=O)CN(CCCCCCCCCC)C(=O)CN(CCCCCCCCCC)C(=O)CN(CC(CC)CCCC)C(=O)CN(CCCCCCCCCC)C(=O)CN(CCCCCCCCCC)C(=O)CN(CCCCCCCCCC)C(=O)CN(CC(CC)CCCC)C(=O)CNC(CO)CO] |
| 9 | 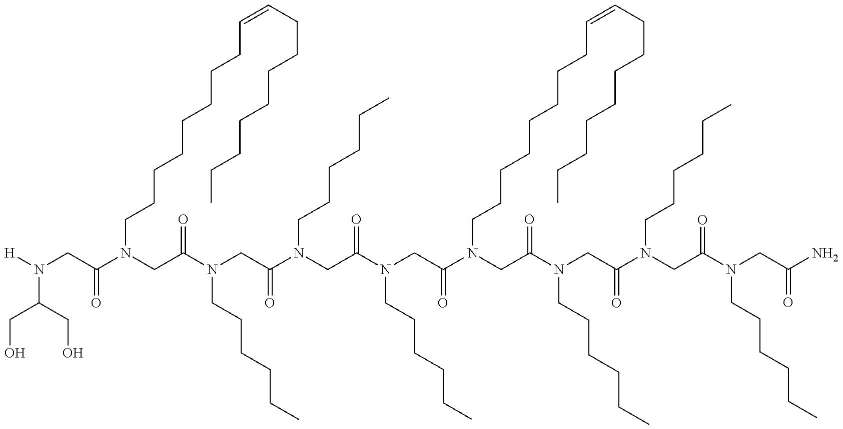 [SMILES: CCCCCCCC/C=C\CCCCCCCCN(CC(=O)N(CCCCCC)CC(=O)N(CCCCCC)CC(=O)N(CCCCCC)CC(=O)N(CCCCCCCC/C=C\CCCCCCCC)CC(=O)N(CCCCCC)CC(=O)N(CCCCCC)CC(=O)N(CCCCCC)CC(N)=O)C(=O)CNC(CO)CO] |
| 10 | 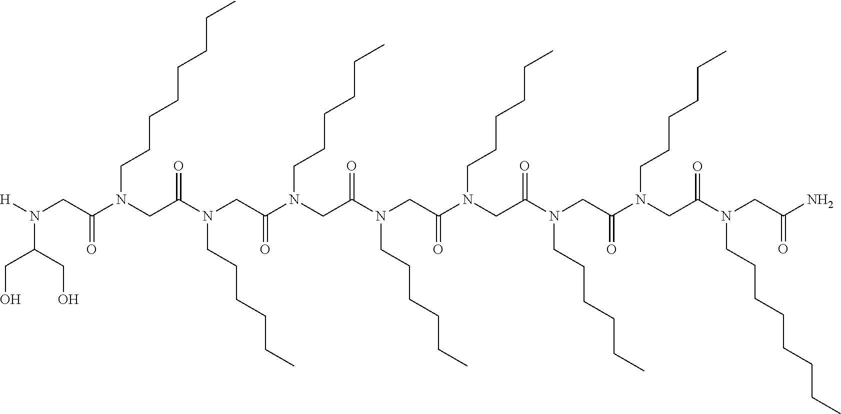 [SMILES: CCCCCCCCN(CC(N)=O)C(=O)CN(CCCCCC)C(=O)CN(CCCCCC)C(=O)CN(CCCCCC)C(=O)CN(CCCCCC)C(=O)CN(CCCCCC)C(=O)CN(CCCCCC)C(=O)CN(CCCCCCCC)C(=O)CNC(CO)CO] |
| 11 | 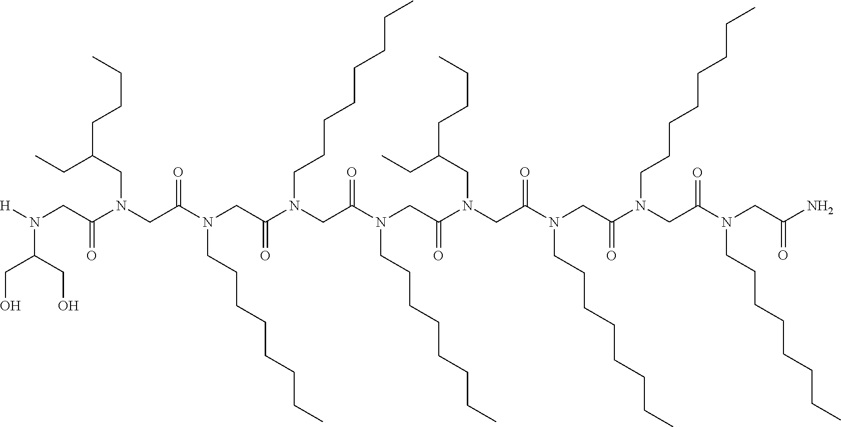 [SMILES: CCCCCCCCN(CC(N)=O)C(=O)CN(CCCCCCCC)C(=O)CN(CCCCCCCC)C(=O)CN(CC(CC)CCCC)C(=O)CN(CCCCCCCC)C(=O)CN(CCCCCCCC)C(=O)CN(CCCCCCCC)C(=O)CN(CC(CC)CCCC)C(=O)CNC(CO)CO] |
| 12 | 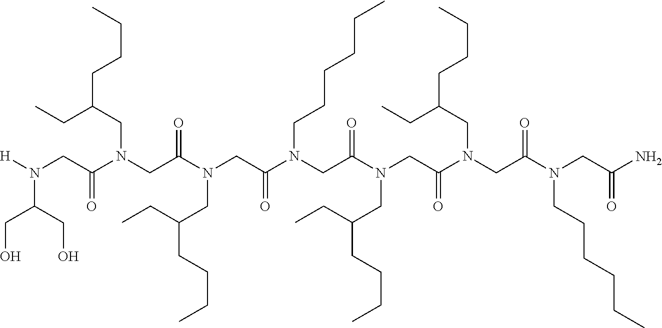 [SMILES: CCCCCCN(CC(N)=O)C(=O)CN(CC(CC)CCCC)C(=O)CN(CC(CC)CCCC)C(=O)CN(CCCCCC)C(=O)CN(CC(CC)CCCC)C(=O)CN(CC(CC)CCCC)C(=O)CNC(CO)CO] |
| 13 | 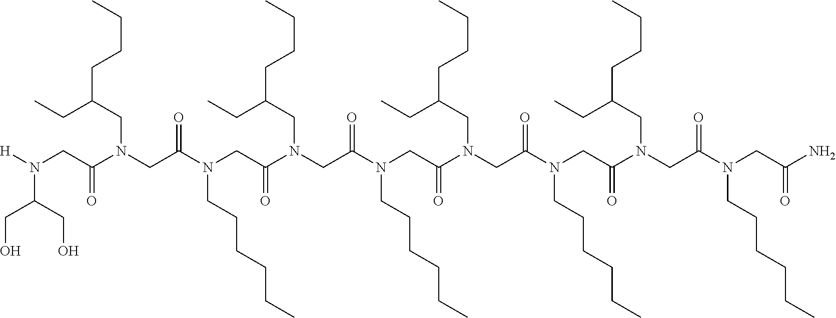 [SMILES: CCCCCCN(CC(N)=O)C(=O)CN(CC(CC)CCCC)C(=O)CN(CCCCCC)C(=O)CN(CC(CC)CCCC)C(=O)CN(CCCCCC)C(=O)CN(CC(CC)CCCC)C(=O)CN(CCCCCC)C(=O)CN(CC(CC)CCCC)C(=O)CNC(CO)CO] |
| 14 | 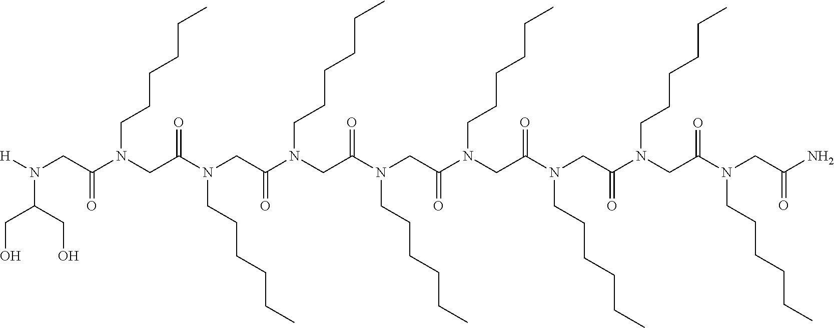 [SMILES: CCCCCCN(CC(N)=O)C(=O)CN(CCCCCC)C(=O)CN(CCCCCC)C(=O)CN(CCCCCC)C(=O)CN(CCCCCC)C(=O)CN(CCCCCC)C(=O)CN(CCCCCC)C(=O)CN(CCCCCC)C(=O)CNC(CO)CO] |
| 15 | 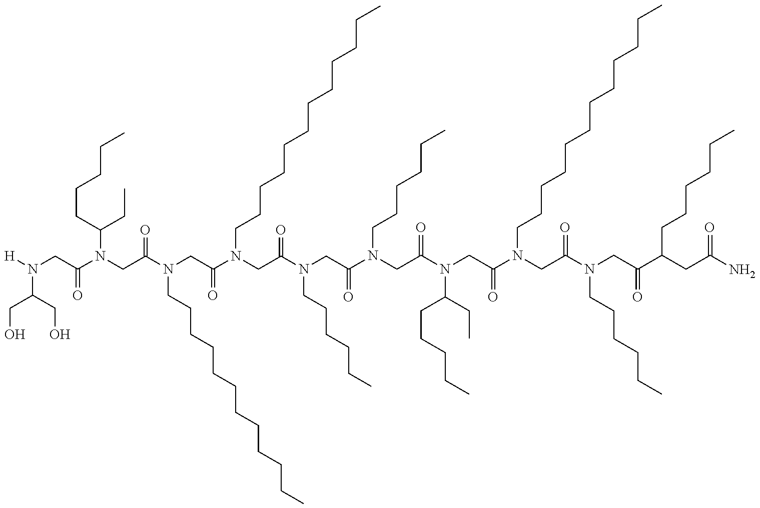 [SMILES: CCCCCCCCCCCCN(CC(=O)N(CCCCCC)CC(=O)N(CCCCCC)CC(=O)N(CC(=O)N(CCCCCCCCCCCC)CC(=O)N(CCCCCC)CC(=O)C(CCCCCC)CC(N)=O)C(CC)CCCCC)C(=O)CN(CCCCCCCCCCCC)C(=O)CN(C(=O)CNC(CO)CO)C(CC)CCCCC] |
| 16 | 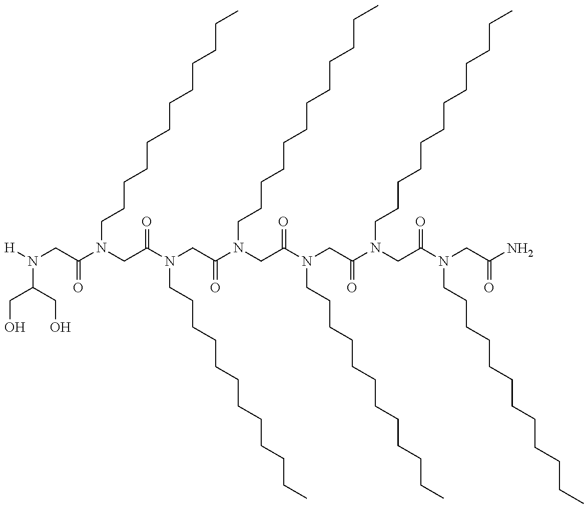 [SMILES: CCCCCCCCCCCCN(CC(N)=O)C(=O)CN(CCCCCCCCCCCC)C(=O)CN(CCCCCCCCCCCC)C(=O)CN(CCCCCCCCCCCC)C(=O)CN(CCCCCCCCCCCC)C(=O)CN(CCCCCCCCCCCC)C(=O)CNC(CO)CO] |
| 17 | 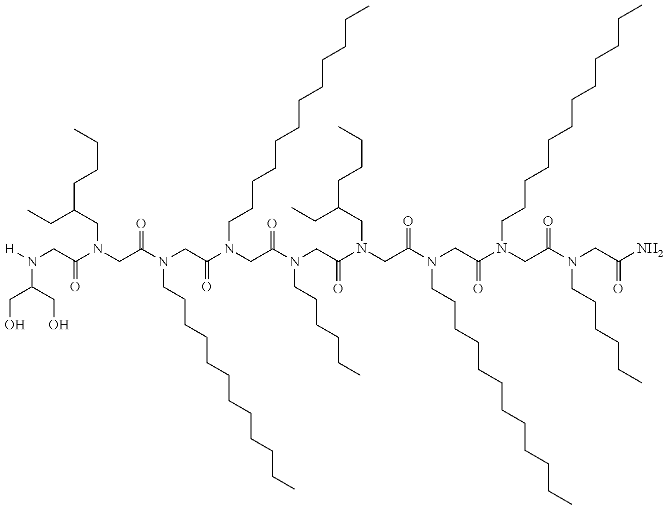 [SMILES: CCCCCCCCCCCCN(CC(=O)N(CCCCCC)CC(N)=O)C(=O)CN(CCCCCCCCCCCC)C(=O)CN(CC(CC)CCCC)C(=O)CN(CCCCCC)C(=O)CN(CCCCCCCCCCCC)C(=O)CN(CCCCCCCCCCCC)C(=O)CN(CC(CC)CCCC)C(=O)CNC(CO)CO] |
| 18 | 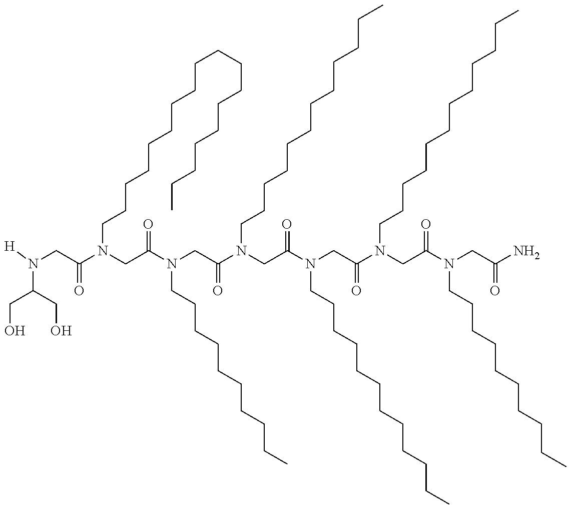 [SMILES: CCCCCCCCCCCCCCCCCCN(CC(=O)N(CCCCCCCCCC)CC(=O)N(CCCCCCCCCCCC)CC(=O)N(CCCCCCCCCCCC)CC(=O)N(CCCCCCCCCCCC)CC(=O)N(CCCCCCCCCC)CC(N)=O)C(=O)CNC(CO)CO] |
| 19 | 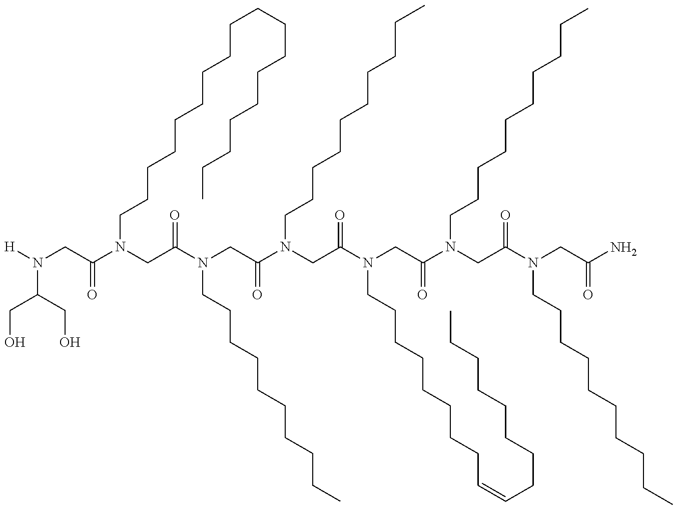 [SMILES: CCCCCCCC/C=C\CCCCCCCCN(CC(=O)N(CCCCCCCCCC)CC(=O)N(CCCCCCCCCC)CC(N)=O)C(=O)CN(CCCCCCCCCC)C(=O)CN(CCCCCCCCCC)C(=O)CN(CCCCCCCCCCCCCCCCCC)C(=O)CNC(CO)CO] |
| 20 | 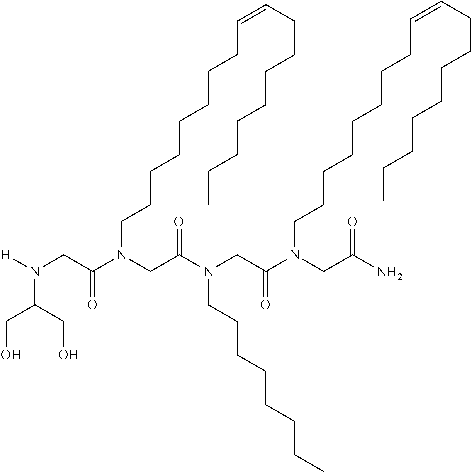 [SMILES: CCCCCCCC/C=C\CCCCCCCCN(CC(N)=O)C(=O)CN(CCCCCCCC)C(=O)CN(CCCCCCCC/C=C\CCCCCCCC)C(=O)CNC(CO)CO] |
| 21 | 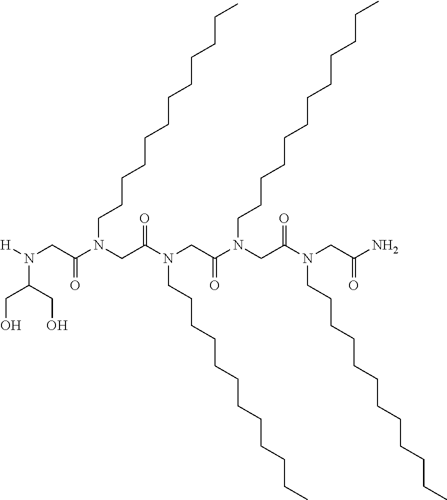 [SMILES: CCCCCCCCCCCCN(CC(N)=O)C(=O)CN(CCCCCCCCCCCC)C(=O)CN(CCCCCCCCCCCC)C(=O)CN(CCCCCCCCCCCC)C(=O)CNC(CO)CO] |
| 22 | 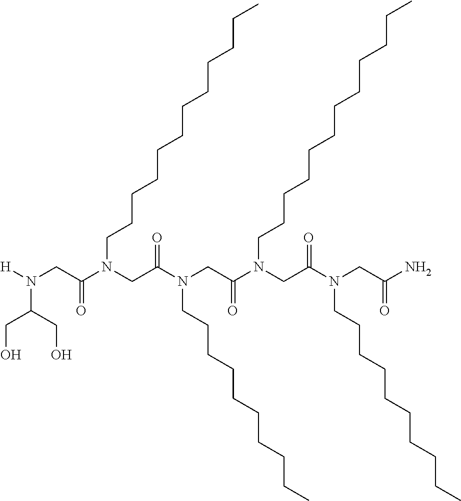 [SMILES: CCCCCCCCCCCCN(CC(=O)N(CCCCCCCCCC)CC(=O)N(CCCCCCCCCCCC)CC(=O)N(CCCCCCCCCC)CC(N)=O)C(=O)CNC(CO)CO] |
| 23 | 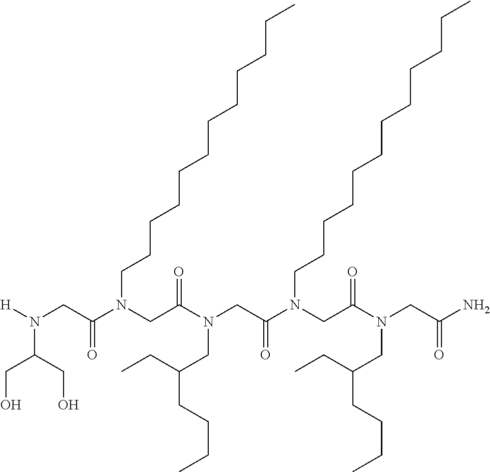 [SMILES: CCCCCCCCCCCCN(CC(=O)N(CC(=O)N(CCCCCCCCCCCC)CC(=O)N(CC(N)=O)CC(CC)CCCC)CC(CC)CCCC)C(=O)CNC(CO)CO] |
| 24 | 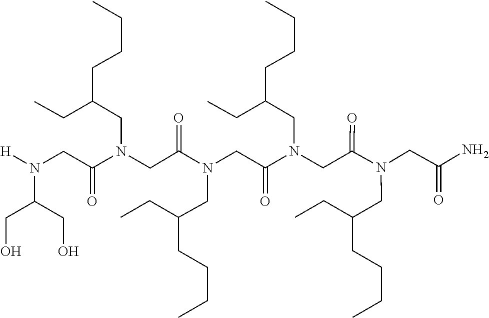 [SMILES: CCCCC(CC)CN(CC(N)=O)C(=O)CN(CC(CC)CCCC)C(=O)CN(CC(CC)CCCC)C(=O)CN(CC(CC)CCCC)C(=O)CNC(CO)CO] |
| 25 | 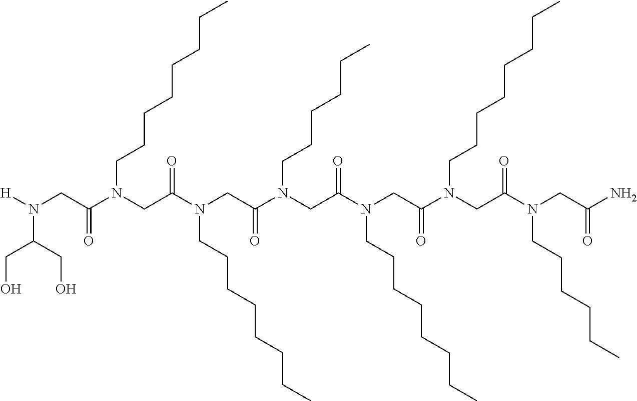 [SMILES: CCCCCCCCN(CC(=O)N(CCCCCCCC)CC(=O)N(CCCCCC)CC(=O)N(CCCCCCCC)CC(=O)N(CCCCCCCC)CC(=O)N(CCCCCC)CC(N)=O)C(=O)CNC(CO)CO] |
| 26 | 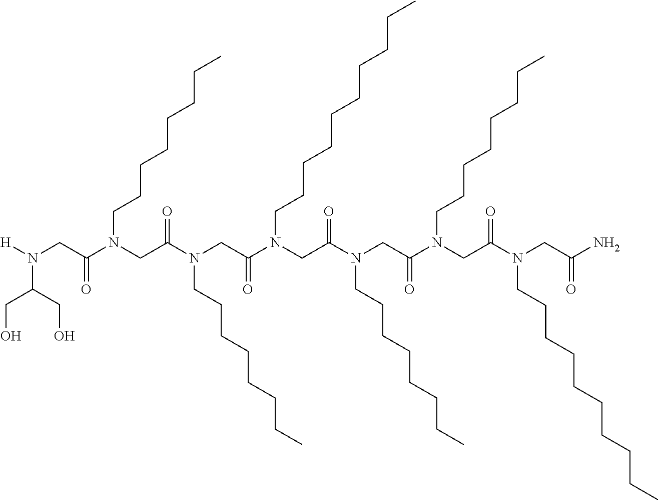 [SMILES: CCCCCCCCCCN(CC(N)=O)C(=O)CN(CCCCCCCC)C(=O)CN(CCCCCCCC)C(=O)CN(CCCCCCCCCC)C(=O)CN(CCCCCCCC)C(=O)CN(CCCCCCCC)C(=O)CNC(CO)CO] |
| 27 | 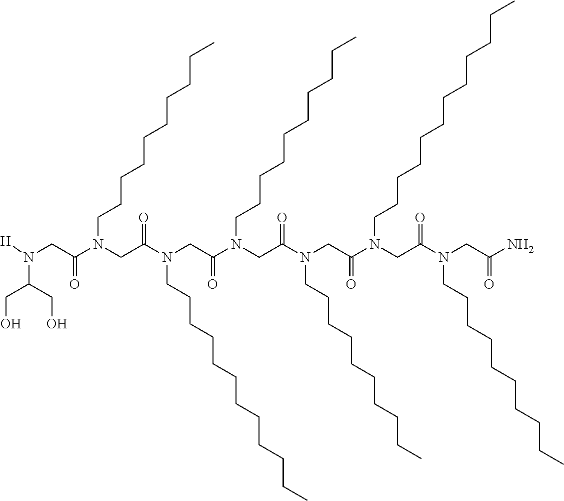 [SMILES: CCCCCCCCCCCCN(CC(=O)N(CCCCCCCCCC)CC(N)=O)C(=O)CN(CCCCCCCCCC)C(=O)CN(CCCCCCCCCC)C(=O)CN(CCCCCCCCCCCC)C(=O)CN(CCCCCCCCCC)C(=O)CNC(CO)CO] |
| 28 | 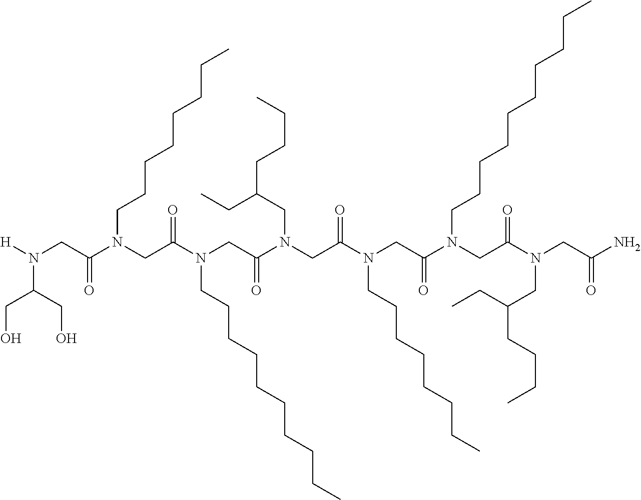 [SMILES: CCCCCCCCCCN(CC(=O)N(CC(N)=O)CC(CC)CCCC)C(=O)CN(CCCCCCCC)C(=O)CN(CC(CC)CCCC)C(=O)CN(CCCCCCCCCC)C(=O)CN(CCCCCCCC)C(=O)CNC(CO)CO] |
| 29 | 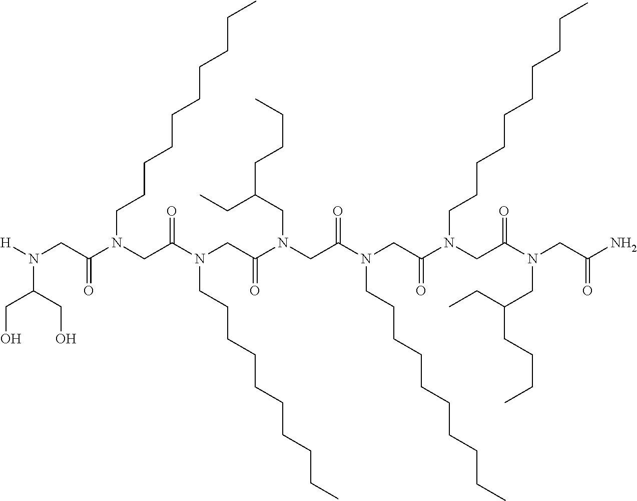 [SMILES: CCCCCCCCCCN(CC(=O)N(CCCCCCCCCC)CC(=O)N(CC(=O)N(CCCCCCCCCC)CC(=O)N(CCCCCCCCCC)CC(=O)N(CC(N)=O)CC(CC)CCCC)CC(CC)CCCC)C(=O)CNC(CO)CO] |
| 30 | 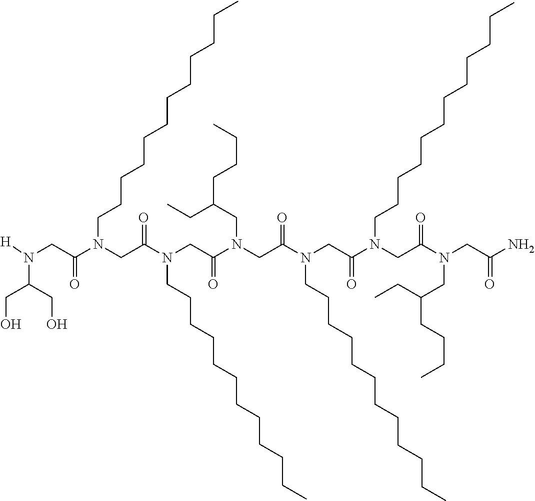 [SMILES: CCCCCCCCCCCCN(CC(=O)N(CCCCCCCCCCCC)CC(=O)N(CC(=O)N(CCCCCCCCCCCC)CC(=O)N(CCCCCCCCCCCC)CC(=O)N(CC(N)=O)CC(CC)CCCC)CC(CC)CCCC)C(=O)CNC(CO)CO] |
| 31 | 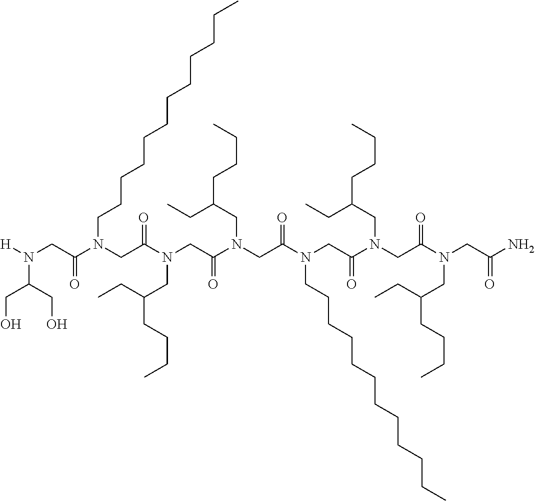 [SMILES: CCCCCCCCCCCCN(CC(=O)N(CC(=O)N(CC(=O)N(CCCCCCCCCCCC)CC(=O)N(CC(=O)N(CC(N)=O)CC(CC)CCCC)CC(CC)CCCC)CC(CC)CCCC)CC(CC)CCCC)C(=O)CNC(CO)CO] |
| 32 | 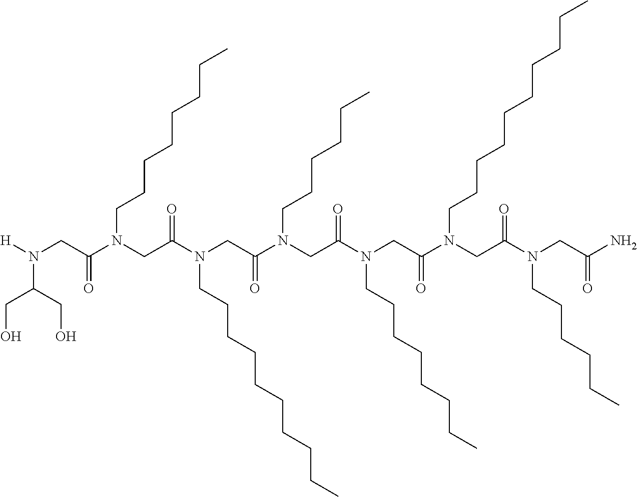 [SMILES: CCCCCCCCCCN(CC(=O)N(CCCCCC)CC(N)=O)C(=O)CN(CCCCCCCC)C(=O)CN(CCCCCC)C(=O)CN(CCCCCCCCCC)C(=O)CN(CCCCCCCC)C(=O)CNC(CO)CO] |
| 33 | 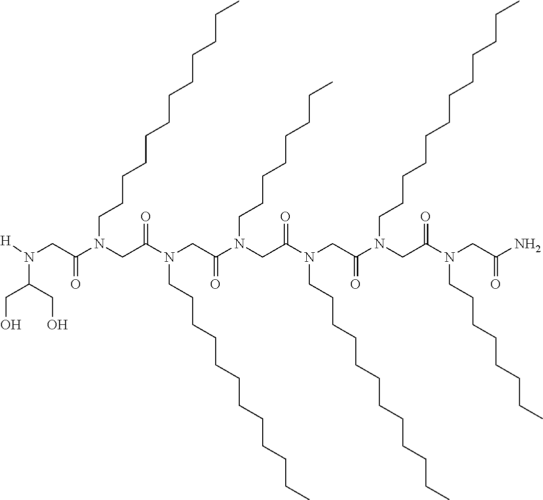 [SMILES: CCCCCCCCCCCCN(CC(=O)N(CCCCCCCCCCCC)CC(=O)N(CCCCCCCC)CC(=O)N(CCCCCCCCCCCC)CC(=O)N(CCCCCCCCCCCC)CC(=O)N(CCCCCCCC)CC(N)=O)C(=O)CNC(CO)CO] |
| 34 | 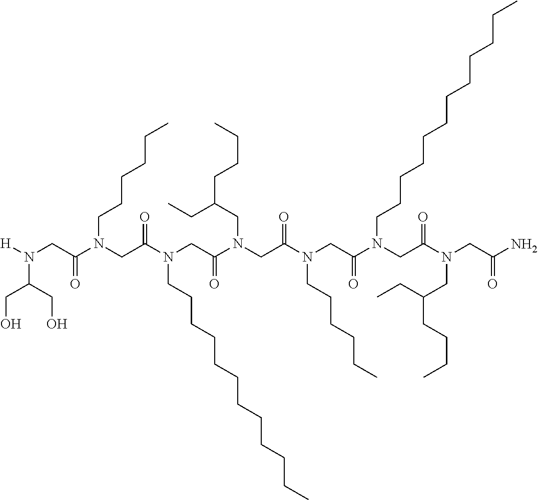 [SMILES: CCCCCCCCCCCCN(CC(=O)N(CC(N)=O)CC(CC)CCCC)C(=O)CN(CCCCCC)C(=O)CN(CC(CC)CCCC)C(=O)CN(CCCCCCCCCCCC)C(=O)CN(CCCCCC)C(=O)CNC(CO)CO] |
| 35 | 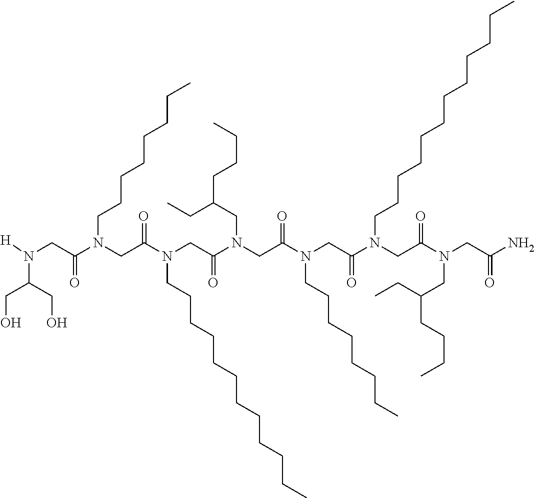 [SMILES: CCCCCCCCCCCCN(CC(=O)N(CC(N)=O)CC(CC)CCCC)C(=O)CN(CCCCCCCC)C(=O)CN(CC(CC)CCCC)C(=O)CN(CCCCCCCCCCCC)C(=O)CN(CCCCCCCC)C(=O)CNC(CO)CO] |

In some implementations the compound of Formula (I) is compound 1, 6, 21, or 30. In some cases, the compound of Formula (I) is compound 1. In some cases, the compound of Formula (I) is compound 6. In some cases, the compound of Formula (I) is compound 21. In some cases, the compound of Formula (I) is compound 30.

### Hydroxyethyl-Capped Tertiary Amino Lipidated Cationic Peptoid Component

The delivery vehicle compositions may comprise a hydroxyethyl-capped tertiary amino lipidated cationic peptoid (“cationic component”, sometimes referred to as an “ionizable lipid”). In some implementations, the hydroxyethyl-capped tertiary amino lipidated cationic peptoids comprise a compound of Formula (II):

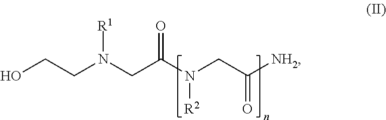

wherein n is 1, 2, 3, 4, 5, or 6; R1 is H, C1-3 alkyl, or C2-3 hydroxyalkyl; and each R2 independently is C8-24 alkyl or C8-24 alkenyl. As used herein, “alkyl” refers to straight chained and branched saturated hydrocarbon groups containing one to thirty carbon atoms, for example, one to four carbon atoms (e.g., 1, 2, 3, or 4). The term Cn means the alkyl group has “n” carbon atoms. For example, C3 alkyl refers to an alkyl group that has 3 carbon atoms. C1-4 alkyl refers to an alkyl group having a number of carbon atoms encompassing the entire range (i.e., 1 to 4 carbon atoms), as well as all subgroups (e.g., 1-2, 1-3, 2-3, 2-4, 1, 2, 3, and 4 carbon atoms). Nonlimiting examples of alkyl groups include, methyl, ethyl, n-propyl, isopropyl, n-butyl, sec-butyl (2-methylpropyl), and t-butyl (1,1-dimethylethyl). Unless otherwise indicated, an alkyl group can be an unsubstituted alkyl group or a substituted alkyl group. As used herein, “hydroxyalkyl” refers to an alkyl group, as defined herein, that is substituted with a hydroxyl group. For example, “C2 hydroxyalkyl” or “hydroxyethyl” has a structure:

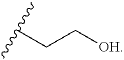 [SMILES: *CCO]

As used herein, “alkenyl” refers to straight chained and branched hydrocarbon groups having a double bond and containing two to thirty carbon atoms, for example, two to four carbon atoms (e.g., 2, 3, or 4). The term Cn means the alkenyl group has “n” carbon atoms. For example, C3 alkenyl refers to an alkenyl group that has 3 carbon atoms. C2-C4 alkenyl refers to an alkenyl group having a number of carbon atoms encompassing the entire range (i.e., 2 to 4 carbon atoms), as well as all subgroups (e.g., 2-3, 2-4, 2, 3, and 4 carbon atoms). Nonlimiting examples of alkenyl groups include, ethenyl, propenyl, and butenyl. Unless otherwise indicated, an alkenyl group can be an unsubstituted alkenyl group or a substituted alkenyl group.

In some implementations, n is 2 to 5. In various implementations, n is 3 to 4. In some implementations, n is 1. In various implementations, n is 2. In some aspects, n is 3. In various aspects, n is 4. In some implementations n is 5. In various implementations, n is 6.

In some implementations, R1 is H. In various implementations, R1 is C1-3 alkyl. In some aspects, R1 is methyl or ethyl. In some implementations, R1 is ethyl. In various implementations, R1 is C2-3 hydroxyalkyl. In some aspects, R1 is

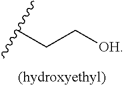 [SMILES: *CCO]

In various aspects, R1 is ethyl or hydroxyethyl.

In some implementations, each R2 independently is C8-18 alkyl or C8-18 alkenyl. In various implementations, each R2 independently is C8-16 alkyl or C10-18 alkenyl. In some aspects, each R2 independently is C10-12 alkyl or C10-18 alkenyl. In some implementations, each R2 independently is: C8-18 alkyl, or C8-16 alkyl, or C8-14 alkyl, or C8-12 alkyl. In various implementations, each R2 independently is selected from the group consisting of

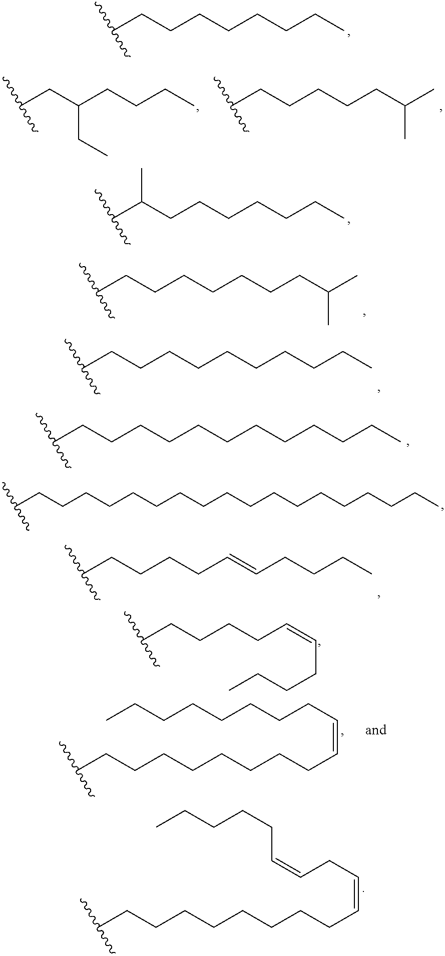

In some aspects, each R2 independently is selected from the group consisting of

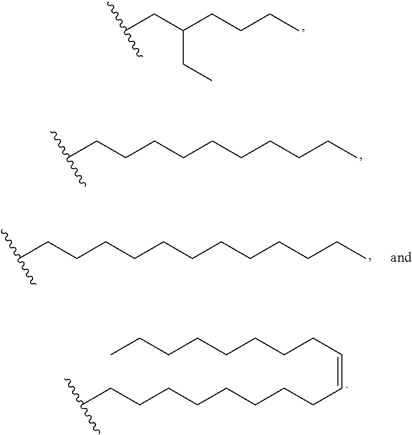

In various aspects, each R2 independently is selected from the group consisting of

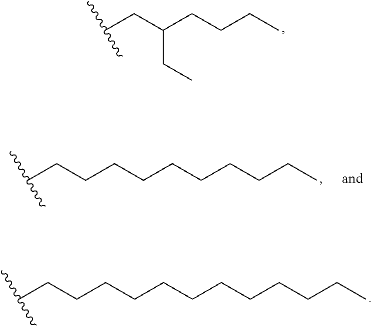

In some implementations, each R2 independently is

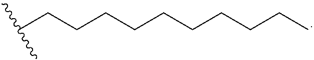 [SMILES: *CCCCCCCCCC]

In some implementations, n is 4, R1 is H, and each R2 is

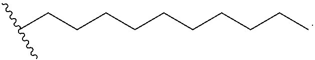 [SMILES: *CCCCCCCCCC]

Contemplated compounds of Formula (II) include, but are not limited to, the compounds listed in Table 2.

TABLE 2
Examples of hydroxyethyl-capped tertiary ammino lipidated cationic peptoids.

| | | Found Mass |
|---|---|---|
| Compound | Structure | (LC-MS) |
| 140 | 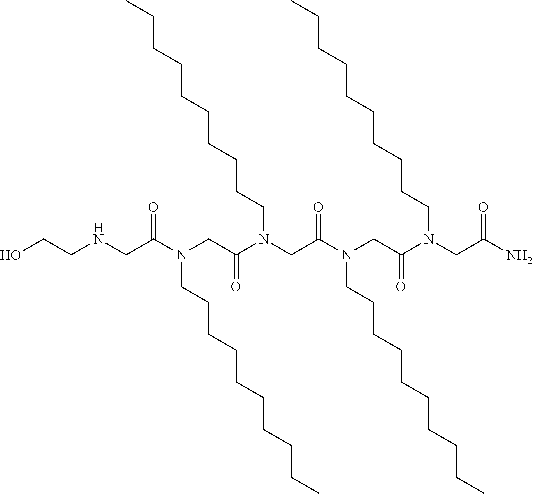 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CNCCO] | 907.9 |
| 146 | 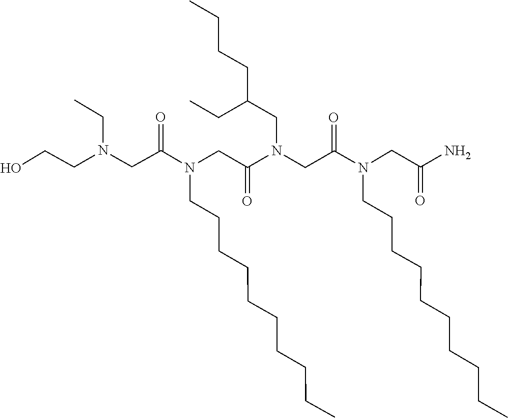 [SMILES: CCCCCCCCCCN(CC(N)=O)C(=O)CN(CC(CC)CCCC)C(=O)CN(CCCCCCCCCC)C(=O)CN(CC)CCO] | 711.4 |
| 151 | 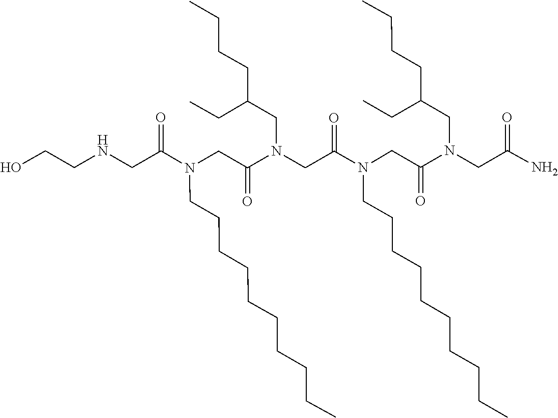 [SMILES: CCCCCCCCCCN(CC(=O)N(CC(=O)N(CCCCCCCCCC)CC(=O)N(CC(N)=O)CC(CC)CCCC)CC(CC)CCCC)C(=O)CNCCO] | 852.8 |
| 152 | 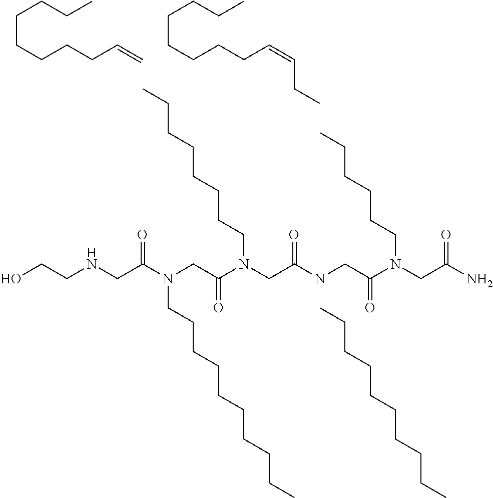 | 1128.0 |
| 160 | 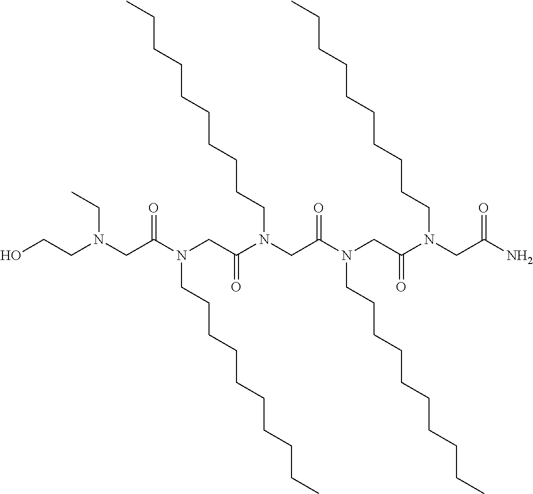 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(CC)CCO] | 935.8 |
| 161 | 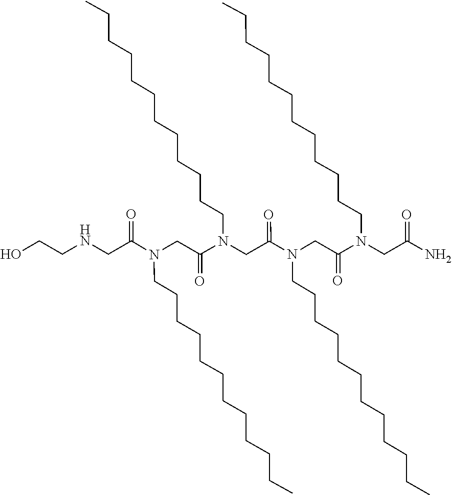 [SMILES: CCCCCCCCCCCCN(CC(N)=O)C(=O)CN(CCCCCCCCCCCC)C(=O)CN(CCCCCCCCCCCC)C(=O)CN(CCCCCCCCCCCC)C(=O)CNCCO] | 1019.9 |
| 162 | 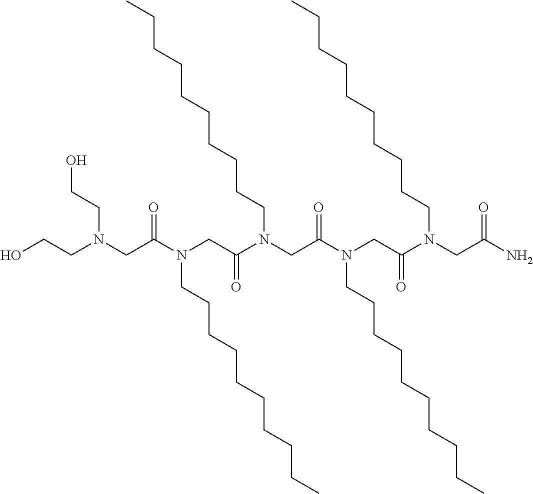 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(CCO)CCO] | 951.8 |
| 140 | 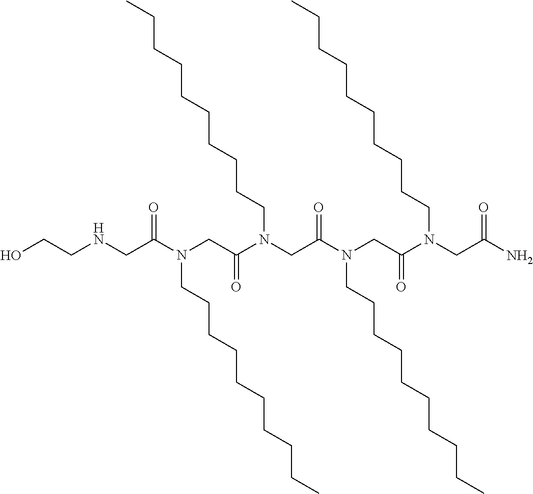 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CNCCO] | 907.9 |
| 146 | 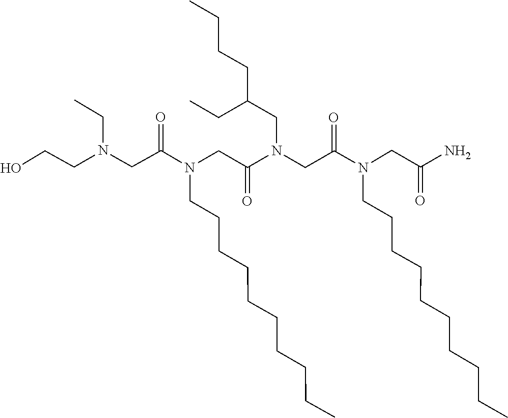 [SMILES: CCCCCCCCCCN(CC(N)=O)C(=O)CN(CC(CC)CCCC)C(=O)CN(CCCCCCCCCC)C(=O)CN(CC)CCO] | 711.4 |
| 151 | 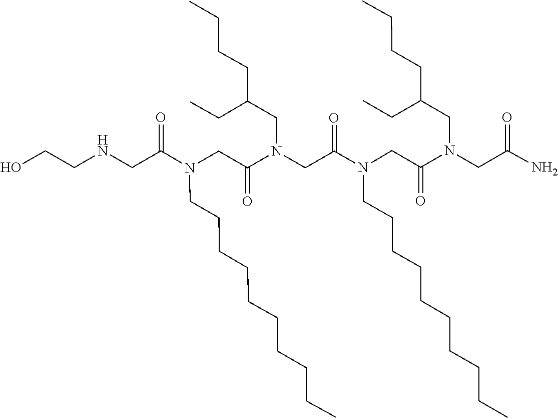 [SMILES: CCCCCCCCCCN(CC(=O)N(CC(=O)N(CCCCCCCCCC)CC(=O)N(CC(N)=O)CC(CC)CCCC)CC(CC)CCCC)C(=O)CNCCO] | 852.8 |
| 152 | 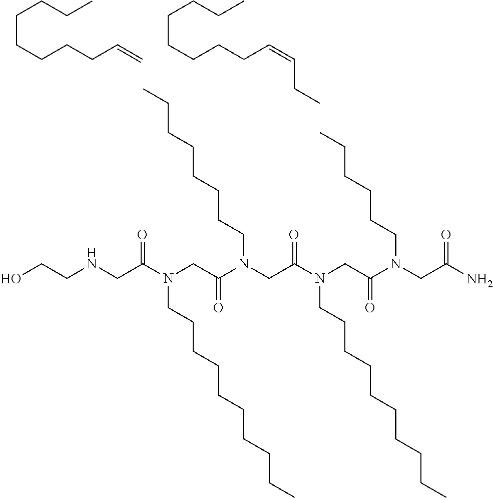 | 1128.0 |
| 160 | 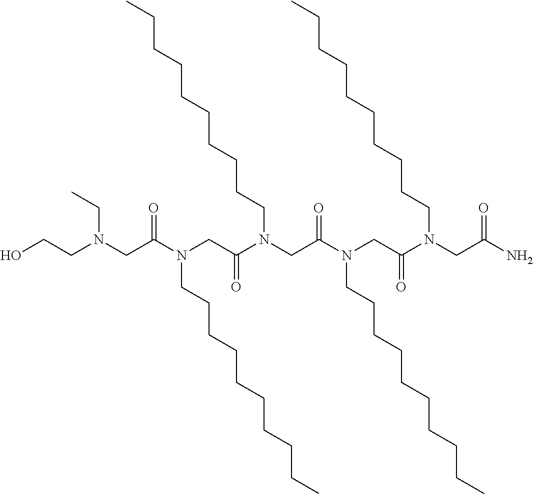 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(CC)CCO] | 935.8 |
| 161 | 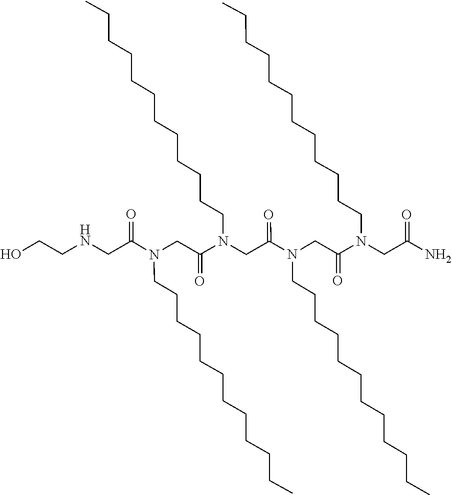 [SMILES: CCCCCCCCCCCCN(CC(N)=O)C(=O)CN(CCCCCCCCCCCC)C(=O)CN(CCCCCCCCCCCC)C(=O)CN(CCCCCCCCCCCC)C(=O)CNCCO] | 1019.9 |
| 162 | 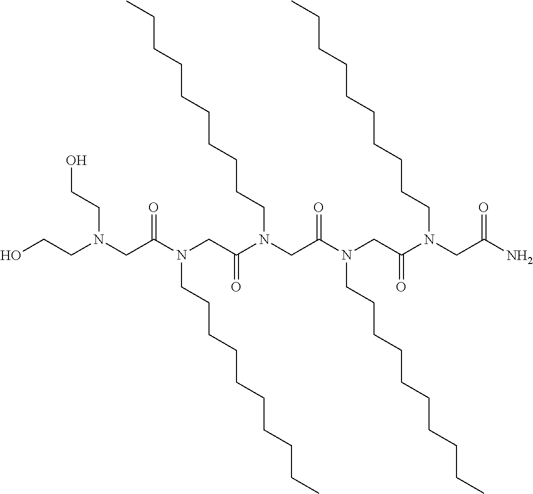 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(CCO)CCO] | 951.8 |

In some implementations the compound of Formula (II) is Compound 140.

### Anionic/Zwitterionic Component

In some implementations, the delivery vehicle composition further includes a component that is anionic or zwitterionic (“anionic/zwitterionic component”). The anionic/zwitterionic component can buffer the zeta potential of a particle or a delivery vehicle complex formed from the delivery vehicle composition, without affecting the ratio of the cargo and/or contributing to particle or delivery vehicle endosomal escape through protonation at low pH in the endosome. Zwitterionic components can serve a further function of holding particles together by interacting with both the hydroxyethyl-capped tertiary amino lipidated cationic peptoid and the polyanionic cargo compounds. Anionic components can also allow for the formation of a core-shell structure of the particle or delivery vehicle, where first a net positive zeta potential particle is made (e.g., by mixing the hydroxyethyl-capped tertiary amino lipidated cationic peptoid and the cargo at a positive+/−charge ratio), which is then coated with the anionic components. These negatively charged multicomponent system particles would avoid reticuloendothelial system (RES) clearance better than positively charged ones.

Examples of suitable anionic and zwitterionic components of the delivery vehicle composition are described in WO2020/069442 and WO2020/069445, each of which is incorporated herein by reference in its entirety. In some implementations, the zwitterionic component comprises one or more phospholipids. Phospholipids can provide further stabilization to complexes in solution, as well as facilitate cell endocytosis, by virtue of their amphipathic character and ability to disrupt the cell membrane.

In some implementations, the one or more phospholipids are selected from the group consisting of 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C 16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanolamine (DOPE), 1,2-dipalmitoyl-sn-glycero-3-phosphoethanolamine (DPPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof. In some aspects, the phospholipid is DSPC, DOPE, or a combination thereof. In various implementations, the phospholipid is DSPC. In various aspects, the phospholipid is DOPE.

In implementations, the delivery vehicle composition comprises between about 1 mol % to about 40 mol % of the phospholipid (e.g., DSPC or DOPE), based on the total number of moles of components in the delivery vehicle composition. In some aspects, the delivery vehicle composition comprises between about 3 mol % to about 30 mol %, or about 5 mol % to about 15 mol %, or about 5 mol % to about 10 mol %, or about 10 mol % to about 15 mol %, or about 9 mol % to about 12 mol %, or about 7 mol % to about 11 mol %, or about 7 mol % to about 12 mol %, or about 10 mol % to about 14 mol %, or about 9 mol %, or about 12 mol % of the phospholipid (e.g., DSPC or DOPE), based on the total number of moles of components in the delivery vehicle composition. In some aspects, the delivery vehicle composition comprises between about 10 mol % to about 11 mol % of the phospholipid (e.g., DSPC or DOPE), based on the total number of moles of components in the delivery vehicle composition. In some aspects, the delivery vehicle composition comprises about 10.0 mol %, about 10.1 mol %, about 10.2 mol %, about 10.3 mol %, about 10.4 mol %, about 10.5 mol %, about 10.6 mol %, about 10.7 mol %, about 10.8 mol %, about 10.9 mol %, or about 11.0 mol % of the phospholipid (e.g., DSPC or DOPE), based on the total number of moles of components in the delivery vehicle composition.

### Neutral Lipid Component

In some implementations, the delivery vehicle composition further includes a component that is a neutral lipid (“neutral lipid component”). The neutral lipid component can be designed to degrade or hydrolyze to facilitate in vivo clearance of the multicomponent delivery system. Contemplated neutral lipid components include, for example, naturally occurring lipids and lipidated peptoids comprising lipid moieties at the N-position of the peptoid. Further examples of lipidated peptoids are described in WO2020/069442 and WO2020/069445, each of which is incorporated herein by reference in its entirety.

In some aspects, the neutral lipid component of the delivery vehicle composition comprises one or more sterols. In some implementations, the one more sterols are selected from the group consisting of cholesterol, fecosterol, sitosterol, ergosterol, campesterol, stigmasterol, brassicasterol, tomatidine, ursolic acid, alpha-tocopherol, and mixtures thereof. In some aspects, the sterol comprises cholesterol. In implementations, the delivery vehicle composition comprises between about 10 mol % to about 80 mol % of the sterol (e.g., cholesterol), based on the total number of moles of components in the delivery vehicle composition. In some aspects, the delivery vehicle composition comprises between about 20 mol % to about 70 mol %, or about 25 mol % to about 60 mol %, or about 30 mol % to about 55 mol %, or about 35 mol % to about 50 mol %, or about 25 mol % to about 45 mol %, or about 40 mol % to about 60 mol %, or about 30 mol % to about 40 mol %, or about 45 mol % to about 55 mol %, or about 35 mol %, or about 50 mol % of the sterol (e.g., cholesterol), based on the total number of moles of components in the delivery vehicle composition. In some aspects, the delivery vehicle composition comprises between about 40 mol % to about 55 mol %, or about 40 mol % to about 45 mol %, or about 50 mol % to about 55 mol % of the sterol (e.g., cholesterol), based on the total number of moles of components in the delivery vehicle composition. In some aspects, the delivery vehicle composition comprises about 40 mol %, about 41 mol %, about 42 mol %, about 43 mol %, about 44 mol %, about 45 mol %, about 46 mol %, about 47 mol %, about 48 mol %, about 49 mol %, about 50 mol %, about 51 mol %, about 52 mol %, about 53 mol %, about 54 mol %, or about 55 mol % of the sterol (e.g., cholesterol), based on the total number of moles of components in the delivery vehicle composition.

### Shielding Component

In some implementations, the delivery vehicle composition further comprises a shielding component. The shielding component can increase the stability of the particle or delivery vehicle in vivo by serving as a steric barrier, thus improving circulation half-life. Examples of suitable shielding components are described in WO2020/069442 and WO2020/069445, each of which is incorporated herein by reference in its entirety.

In some implementations, the shielding component comprises one or more PEGylated lipids. As used herein, a “PEGylated lipid” includes any lipid or lipid-like compound covalently bound to a polyethylene glycol moiety. Suitable lipid moieties for the PEGylated lipid can include, for example, branched or straight chain aliphatic moieties that can be unsubstituted or substituted, or moieties derived from natural lipid compounds, including fatty acids, sterols, and isoprenoids, that either be unsubstituted or substituted.

In some implementations, the lipid moieties may include branched or straight chain aliphatic moieties having from about 6 to about 50 carbon atoms or from about 10 to about 50 carbon atoms. The aliphatic moieties can comprise, in some implementations, one or more heteroatoms, and/or one or more double or triple bonds (i.e., saturated or mono- or poly-unsaturated). In some aspects, the lipid moieties may include aliphatic, straight chain or branched moieties, each hydrophobic tail independently having from about 8 to about 30 carbon atoms or from about 6 to about 30 carbon atoms, wherein the aliphatic moieties can be unsubstituted or substituted. In various implementations, the lipid moieties may include, for example, aliphatic carbon chains derived from fatty acids and fatty alcohols. In some implementations, each lipid moiety is independently C₈-C₂₄-alkyl or C₈-C₂₄-alkenyl, wherein the C₈-C₂₄-alkenyl can be, in some aspects, mono- or poly-unsaturated.

Natural lipid moieties employed in the practice of the present disclosure can be derived from, for example, phospholipids, glycerides (such as di- or tri-glycerides), glycosylglycerides, sphingolipids, ceramides, and saturated and unsaturated sterols, isoprenoids, and other like natural lipids.

Other suitable lipid moieties may include lipophilic aromatic groups such as optionally substituted aryl or arylalkyl moieties, including for example naphthalenyl or ethylbenzyl, or lipids comprising ester functional groups including, for example, sterol esters and wax esters.

In some implementations, the one or more PEGylated lipids are selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and any combinations thereof. In some implementations, the PEGylated lipids comprise a PEG-modified sterol. In various implementations, the PEGylated lipids comprise PEG-modified cholesterol. In some implementations, the PEGylated lipid is a PEG-modified ceramide. In some aspects, the PEG-modified ceramine is selected from the group consisting of N-octanoyl-sphingosine-1-{succinyl[methoxy(polyethylene glycol)]} and N-palmitoyl-sphingosine-1-{succinyl[methoxy(polyethylene glycol)]}, and any combination thereof.

In some implementations, the PEGylated lipids are PEG-modified phospholipids, wherein the phospholipid is selected from the group consisting of 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18.0 Diether PC), 1-oleoyl-2-cholesterylhemisuccinoyl-sn-glycero-3-phosphocholine (OChemsPC), 1-hexadecyl-sn-glycero-3-phosphocholine (C 16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanolamine (DOPE), 1,2-dipalmitoyl-sn-glycero-3-phosphoethanolamine (DPPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof. In various implementations, the phospholipid is DOPE.

In some implementations, the one or more PEGylated lipids comprise a PEG-modified phosphatidylethanol. In some implementations, the PEGylated lipid is a PEG-modified phosphatidylethanol selected from the group consisting of PEG-modified DMPE (DMPE-PEG), PEG-modified DSPE (DSPE-PEG), PEG-modified DPPE (DPPE-PEG), and PEG-modified DOPE (DOPE-PEG).

In various implementations, the PEGylated lipid is selected from the group consisting of dimyristoylglycerol-polyethylene glycol (DMG-PEG), distearoylglycerol-polyethylene glycol (DSG-PEG), dipalmitoylglycerol-polyethylene glycol (DPG-PEG), and dioleoylglycerol-polyethylene glycol (DOG-PEG). In some implementations, the PEG lipid is DMG-PEG.

The molecular weights of the PEG chain in the foregoing PEGylated lipids can be tuned, as desired, to optimize the properties of the delivery vehicle compositions. In some implementations, the PEG chain has a molecular weight between 350 and 6,000 g/mol, between 1,000 and 5,000 g/mol, or between 2,000 and 5,000 g/mol, or between about 1,000 and 3,000 g/mol, or between abut 1,500 and 4,000 g/mol. In some aspects, the PEG chain of the PEG lipid has a molecular weight of about 350 g/mol, 500 g/mol, 600 g/mol, 750 g/mol, 1,000 g/mol, 2,000 g/mol, 3,000 g/mol, 5,000 g/mol, or 10,000 g/mol. In some implementations, the PEG chain of the PEGylated lipid has a molecular weight of about 500 g/mol, 750 g/mol, 1,000 g/mol, 2,000 g/mol or 5,000 g/mol. The PEG chain can be branched or linear. In some aspects, the PEGylated lipid is dimyristoylglycerol-polyethylene glycol 2000 (DMG-PEG 2000).

In implementations, the delivery vehicle composition comprises between about 1 mol % to about 5 mol % of the PEGylated lipid (e.g., DMG-PEG 2000), based on the total number of moles of components in the delivery vehicle composition. In some aspects, the delivery vehicle composition comprises between about 1 mol % to about 3 mol %, or about 1 mol % to about 2 mol %, or about 2 mol % to about 5 mol %, or about 0.5 mol % to about 1.5 mol %, or about 1.5 mol % to about 2.5 mol %, or about 1.5 mol % to about 2.0 mol %, or about 2.0 mol % to about 2.5 mol %, or about 1 mol %, or about 1.5 mol %, or about 2 mol %, or about 2.5 mol %, or about 3 mol %, or about 3.5 mol %, or about 4 mol %, or about 5 mol % of the PEGylated lipid (e.g., DMG-PEG 2000), based on the total number of moles of components in the delivery vehicle composition. In some aspects, the delivery vehicle composition comprises between about 1 mol % to about 3 mol %, or about 1 mol % to about 2 mol %, or about 2 mol % to about 5 mol %, or about 0.5 mol % to about 1.5 mol %, or about 1.5 mol % to about 2.5 mol %, or about 1 mol %, or about 1.5 mol %, or about 2 mol %, or about 2.5 mol %, or about 3 mol %, or about 3.5 mol %, or about 4 mol %, or about 5 mol % of the PEGylated lipid (e.g., DMG-PEG 2000), based on the total number of moles of components in the delivery vehicle composition. In some aspects, the delivery vehicle composition comprises about 1.5 mol %, 1.6 mol %, 1.7 mol %, 1.8 mol %, 1.9 mol %, 2.0 mol %, 2.1 mol %, 2.2 mol %, 2.3 mol %, 2.4 mol %, or about 2.5 mol % of the PEGylated lipid (e.g., DMG-PEG 2000), based on the total number of moles of components in the delivery vehicle composition.

### Representative Examples

Non-limiting delivery vehicle combinations are described below. As previously described, the unit “mol %” or “molar percentage” refers to the number of moles of a particular component of the delivery vehicle composition divided by the total number of moles of all components in the delivery vehicle composition, times 100%.

In some implementations, the delivery vehicle composition comprises at least 99 mol % the cationic component and less than about 1 mol % shielding component (e.g., Formula F1A in Table 3). In some aspects, the delivery vehicle composition comprises less than about 20 mol % of the cationic component, less than about 5 mol % of a shielding component, and more than about 75 mol % of a mixture of the anionic/zwitterionic component and the neutral lipid component (e.g., Formula F2A and Formula F4A in Table 3). In some aspects, the delivery vehicle composition comprises about 30 to about 45 mol % of the cationic component, about 50 to about 70 mol % of a mixture of the anionic/zwitterionic component and the neutral lipid component, and about 1.5 to about 4.5 mol % of the shielding component (e.g., Formula F3A and Formula F5A in Table 3). In various aspects, the delivery vehicle composition comprises about 15 to about 35 mol % of the cationic component, about 60 to about 80 mol % of a mixture of the anionic/zwitterionic component and the neutral lipid component, and about 1.5 to about 3.0 mol % of a shielding component (e.g., Formula F2A and Formula F3A in Table 3). In some implementations, the delivery vehicle composition comprises about 15 to about 35 mol % of the cationic component, about 10-about 20 mol % of an anionic/zwitterionic component, about 50 to about 65 mol % of a neutral lipid component, and about 1.5 to about 3.0 mol % of a shielding component (e.g., Formula F2A and Formula F3A in Table 3). In various implementations, the delivery vehicle composition comprises about 10 to about 20 mol % of the cationic component, about 75 to about 89 mol % of a lipid component, and about 1 to about 5 mol % of a shielding component (e.g., Formula F4A in Table 3). In some aspects, the delivery vehicle composition comprises about 40 to about 50 mol % of the cationic component, about 50 to about 59 mol % of an anionic/zwitterionic component, and about 1 to about 5 mol % shielding component (e.g., Formula F5A in Table 3). In various aspects, the delivery vehicle composition comprises about 30 to about 50 mol % of the cationic component, about 50 to about 70 mol % of a neutral lipid component, and about 1 to about 5 mol % shielding component (e.g., Formula F6A in Table 3). In various aspects, the delivery vehicle composition comprises about 40 to about 45 mol % of the cationic component, about 50 to about 60 mol % of a mixture of the anionic/zwitterionic component and the neutral lipid component, and about 1.5 to about 2.0 mol % of a shielding component (e.g., Formula F6.1 and Formula F6.2 in Table 3). In some implementations, the delivery vehicle composition comprises about 40 to about 45 mol % of the cationic component, about 10 to about 15 mol % of an anionic/zwitterionic component, about 40 to about 45 mol % of a neutral lipid component, and about 1.5 to about 2.0 mol % of a shielding component (e.g., Formula F6.1 and Formula F6.2 in Table 3). In various aspects, the delivery vehicle composition comprises about 30 to about 35 mol % of the cationic component, about 60 to about 70 mol % of a mixture of the anionic/zwitterionic component and the neutral lipid component, and about 2.0 to about 3.0 mol % of a shielding component (e.g., Formula F6.3 in Table 3). In some implementations, the delivery vehicle composition comprises about 30 to about 35 mol % of the cationic component, about 10 to about 15 mol % of an anionic/zwitterionic component, about 50 to about 55 mol % of a neutral lipid component, and about 2.0 to about 3.0 mol % of a shielding component (e.g., Formula F6.3 in Table 3). The cationic component can be any cationic component described herein, such as any of the compounds of Formula (I) or Formula (II) (e.g., the compounds listed in Table 1, such as Compounds 1-35 or the compounds listed in Table 2, such as compound 140, 146, 151, 152, 160, 161, and 162). In some implementations, the cationic compound is Compound 1, Compound 6, Compound 21, Compound 30 or Compound 140. The anionic/zwitterionic component can be any anionic/zwitterionic component described herein (e.g., a phospholipid). In some implementations, the anionic/zwitterionic component is DSPC or DOPE. The neutral lipid component can be any neutral lipid described herein (e.g., a sterol). In some implementations, the neutral lipid component is cholesterol. The shielding component can be any shielding component described herein (e.g., PEGylated lipids). In some implementations, the shielding component is DMG-PEG2000.

In some implementations, the delivery vehicle composition comprises about 30 mol % to about 60 mol % (e.g., about 35 mol % to about 39 mol %, or about 39 mol % to about 52 mol %, or about 42 mol % to about 49 mol %, or about 50 mol % to about 52 mol %) of the cationic component; about 3 mol % to about 20 mol % of the anionic/zwitterionic component, about 25 mol % to about 60 mol % of the neutral lipid compound, and about 1 mol % to about 5 mol % of the shielding component. In various implementations, the delivery vehicle composition comprises about 35 to about 55 mol % of the cationic component; about 5 mol % to about 15 mol % of the anionic/zwitterionic component, about 30 mol % to about 55 mol % of the neutral lipid compound, and about 1 mol % to about 3 mol % of the shielding component. In various implementations, the delivery vehicle composition comprises about 38 to about 52 mol % of the cationic component; about 9-about 12 mol % of the anionic/zwitterionic component, about 35 mol % to about 50 mol % of the neutral lipid compound, and about 1 mol % to about 2 mol % of the shielding component. In some aspects, the delivery vehicle composition comprises about 30 mol % to about 49 mol % of the compound of Formula (I) or Formula (II); about 5 mol % to about 15 mol % of the phospholipid, about 30 mol % to about 55 mol % of the sterol, and about 1 mol % to about 3 mol % of the PEGylated lipid. In some aspects, the composition comprises about 35 mol % to about 49 mol % of the compound or salt of Formula (II); about 7 mol % to about 12 mol % of the phospholipid, about 35 mol % to about 50 mol % of the sterol, and about 1 mol % to about 2 mol % of the PEGylated lipid. The cationic component can be any cationic component described herein, such as any of the compounds of Formula (I) or Formula (II) (e.g., the compounds listed in Table 1 such as Compounds 1-35 or the compounds listed in or Table 2, such as compound 1, 6, 21, 30, 140, 146, 151, 152, 160, 161, and 162). In some implementations, the cationic compound is Compound 140. The anionic/zwitterionic component can be any anionic/zwitterionic component described herein (e.g., a phospholipid). In some implementations, the anionic/zwitterionic component is DSPC or DOPE. The neutral lipid component can be any neutral lipid described herein (e.g., a sterol). In some implementations, the neutral lipid component is cholesterol. The shielding component can be any shielding component described herein (e.g., PEGylated lipids). In some implementations, the shielding component is DMG-PEG2000.

In some implementations, the delivery vehicle composition comprises about 30 mol % to about 45 mol % of the cationic component; about 5 mol % to about 15 mol % of the anionic/zwitterionic component, about 40 mol % to about 60 mol % of the neutral lipid compound, and about 1 mol % to about 5 mol % of the shielding component. In various implementations, the delivery vehicle composition comprises about 35 mol % to about 40 mol % of the cationic component; about 8 mol % to about 12 mol % of the anionic/zwitterionic component, about 45 mol % to about 50 mol % of the neutral lipid compound, and about 1 mol % to about 3 mol % of the shielding component. In various implementations, the delivery vehicle composition comprises about 38.2 mol % of the cationic component; about 11.8 mol % of the anionic/zwitterionic component, about 48.2 mol % of the neutral lipid compound, and about 1.9 mol % of the shielding component (“Form F2”). The cationic component can be any cationic component described herein, such as any of the compounds of Formula (I) or Formula (II) (e.g., the compounds listed in Table 1 such as Compounds 1-35 or the compounds listed in Table 2, such as compound 140, 146, 151, 152, 160, 161, and 162). In some implementations, the cationic compound is Compound 140. The anionic/zwitterionic component can be any anionic/zwitterionic component described herein (e.g., a phospholipid). In some implementations, the anionic/zwitterionic component is DSPC or DOPE. The neutral lipid component can be any neutral lipid described herein (e.g., a sterol). In some implementations, the neutral lipid component is cholesterol. The shielding component can be any shielding component described herein (e.g., PEGylated lipids). In some implementations, the shielding component is DMG-PEG-2000. In some implementations, the delivery vehicle composition comprises Form F2, as shown in Table 3, below. In some implementations, the delivery vehicle composition comprises about 38.2 mol % of Compound 140, about 11.8 mol % of DSPC, about 48.2 mol % of cholesterol, and about 1.9 mol % of DMG-PEG-2000 (“DV-140-F2”).

In some implementations, the delivery vehicle composition comprises about 45 to about 55 mol % of the cationic component; about 5 mol % to about 15 mol % of the anionic/zwitterionic component, about 35 mol % to about 55 mol % of the neutral lipid compound, and about 1 mol % to about 5 mol % of the shielding component. In various implementations, the delivery vehicle composition comprises about 48 mol % to about 52 mol % of the cationic component; about 5 mol % to about 12 mol % of the anionic/zwitterionic component, about 38 mol % to about 42 mol % of the neutral lipid compound, and about 1 mol % to about 3 mol % of the shielding component. In various implementations, the delivery vehicle composition comprises about 51.3 mol % of the cationic component; about 9.3 mol % of the anionic/zwitterionic component, about 38.0 mol % of the neutral lipid compound, and about 1.5 mol % of the shielding component (“Form F6/17”). The cationic component can be any cationic component described herein, such as any of the compounds of Formula (I) or Formula (II) (e.g., the compounds listed in Table 1 such as Compounds 1-35 or the compounds listed in or Table 2, such as compound 140, 146, 151, 152, 160, 161, and 162). In some implementations, the cationic compound is Compound 140. The anionic/zwitterionic component can be any anionic/zwitterionic component described herein (e.g., a phospholipid). In some implementations, the anionic/zwitterionic component is DSPC or DOPE. The neutral lipid component can be any neutral lipid described herein (e.g., a sterol). In some implementations, the neutral lipid component is cholesterol. The shielding component can be any shielding component described herein (e.g., PEGylated lipids). In some implementations, the shielding component is DMG-PEG 2000. In some implementations, the delivery vehicle composition comprises Form F6/17, as shown in Table 3, below. In some implementations, the delivery vehicle composition comprises about 51.3 mol % of Compound 140, about 9.3 mol % of DSPC, about 38.0 mol % of cholesterol, and about 1.5 mol % of DMG-PEG 2000 (“DV-140-F6/17”).

In some implementations, the delivery vehicle composition comprises about 30 mol % to about 49 mol % of the cationic component; about 5 mol % to about 15 mol % of the anionic/zwitterionic component, about 30 mol % to about 55 mol % of the neutral lipid compound, and about 1 mol % to about 3 mol % of the shielding component. In various implementations, the delivery vehicle composition comprises about 48 mol % to about 52 mol % of the cationic component; about 5 mol % to about 12 mol % of the anionic/zwitterionic component, about 38 mol % to about 42 mol % of the neutral lipid compound, and about 1 mol % to about 3 mol % of the shielding component. In various implementations, the delivery vehicle composition comprises about 42.6 mol % of the cationic component; about 10.0 mol % of the anionic/zwitterionic component, about 44.7 mol % of the neutral lipid compound, and about 1.7 mol % of the shielding component. The cationic component can be any cationic component described herein, such as any of the compounds of Formula (I) or Formula (II) (e.g., the compounds listed in Table 1 or Table 2, such as compound 1, 6, 21, 30, 140, 146, 151, 152, 160, 161, and 162). In some implementations, the cationic compound is Compound 140. The anionic/zwitterionic component can be any anionic/zwitterionic component described herein (e.g., a phospholipid). In some implementations, the anionic/zwitterionic component is DSPC or DOPE. The neutral lipid component can be any neutral lipid described herein (e.g., a sterol). In some implementations, the neutral lipid component is cholesterol. The shielding component can be any shielding component described herein (e.g., PEGylated lipids). In some implementations, the shielding component is DMG-PEG 2000. In some implementations, the delivery vehicle composition comprises Form F6/12 or Form F6/15, as shown in Table 3, below. In some implementations, the delivery vehicle composition comprises about 42.6 mol % of Compound 140, about 10.9 mol % of DSPC, about 44.7 mol % of cholesterol, and about 1.7 mol % of DMG-PEG 2000 (“DV-140-F6/12”). In some implementations, the delivery vehicle composition comprises about 48.1 mol % of Compound 140, about 9.9 mol % of DSPC, about 40.4 mol % of cholesterol, and about 1.6 mol % of DMG-PEG 2000 (“DV-140-F6/15”).

In some implementations, the delivery vehicle composition comprises about 40 mol % to about 49 mol % of the cationic component; about 5 mol % to about 15 mol % of the anionic/zwitterionic component, about 30 mol % to about 55 mol % of the neutral lipid compound, and about 1 mol % to about 3 mol % of the shielding component. In various implementations, the delivery vehicle composition comprises about 42 mol % to about 46 mol % of the cationic component; about 7 mol % to about 12 mol % of the anionic/zwitterionic component, about 41 mol % to about 45 mol % of the neutral lipid compound, and about 1 mol % to about 2 mol % of the shielding component. In various implementations, the delivery vehicle composition comprises about 44.4 mol % of the cationic component; about 10.6 mol % of the anionic/zwitterionic component, about 43.3 mol % of the neutral lipid compound, and about 1.7 mol % of the shielding component. In various implementations, the delivery vehicle composition comprises about 44.4 mol % of the cationic component; about 10.6 mol % of the anionic/zwitterionic component, about 43.4 mol % of the neutral lipid compound, and about 1.7 mol % of the shielding component. The cationic component can be any cationic component described herein, such as any of the compounds of Formula (I) or Formula (II) (e.g., the compounds listed in Table 1 or Table 2, such as compound 1, 6, 21, 30, 140, 146, 151, 152, 160, 161, and 162). In some implementations, the cationic compound is Compound 140. The anionic/zwitterionic component can be any anionic/zwitterionic component described herein (e.g., a phospholipid). In some implementations, the anionic/zwitterionic component is DSPC or DOPE. The neutral lipid component can be any neutral lipid described herein (e.g., a sterol). In some implementations, the neutral lipid component is cholesterol. The shielding component can be any shielding component described herein (e.g., PEGylated lipids). In some implementations, the shielding component is DMG-PEG 2000. In some implementations, the delivery vehicle composition comprises F6.1 or F6.2, as shown in Table 3, below. In some implementations, the delivery vehicle composition comprises about 44.4 mol % of Compound 140, about 10.6 mol % of DSPC, about 43.3 mol % of cholesterol, and about 1.7 mol % of DMG-PEG 2000 (“DV-140-F6.1”). In some implementations, the delivery vehicle composition comprises about 44.4 mol % of Compound 140, about 10.6 mol % of DSPC, about 43.4 mol % of cholesterol, and about 1.7 mol % of DMG-PEG 2000 (“DV-140-F6.2”).

In some implementations, the delivery vehicle composition comprises about 30 mol % to about 39 mol % of the cationic component; about 5 mol % to about 15 mol % of the anionic/zwitterionic component, about 30 mol % to about 55 mol % of the neutral lipid compound, and about 1 mol % to about 3 mol % of the shielding component. In various implementations, the delivery vehicle composition comprises about 30 mol % to about 35 mol % of the cationic component; about 7 mol % to about 12 mol % of the anionic/zwitterionic component, about 50 mol % to about 55 mol % of the neutral lipid compound, and about 2 mol % to about 3 mol % of the shielding component. In various implementations, the delivery vehicle composition comprises about 33.1 mol % of the cationic component; about 10.5 mol % of the anionic/zwitterionic component, about 53.8 mol % of the neutral lipid compound, and about 2.5 mol % of the shielding component. The cationic component can be any cationic component described herein, such as any of the compounds of Formula (I) or Formula (II) (e.g., the compounds listed in Table 1 or Table 2, such as compound 1, 6, 21, 30, 140, 146, 151, 152, 160, 161, and 162). In some implementations, the cationic compound is Compound 140. The anionic/zwitterionic component can be any anionic/zwitterionic component described herein (e.g., a phospholipid). In some implementations, the anionic/zwitterionic component is DSPC or DOPE. The neutral lipid component can be any neutral lipid described herein (e.g., a sterol). In some implementations, the neutral lipid component is cholesterol. The shielding component can be any shielding component described herein (e.g., PEGylated lipids). In some implementations, the shielding component is DMG-PEG 2000. In some implementations, the delivery vehicle composition comprises Form F6/12 or Form F6/15, as shown in Table 32, below. In some implementations, the delivery vehicle composition comprises about 42.6 mol % of Compound 140, about 10.9 mol % of DSPC, about 44.7 mol % of cholesterol, and about 1.7 mol % of DMG-PEG 2000 (“DV-140-F6/12”). In some implementations, the delivery vehicle composition comprises about 48.1 mol % of Compound 140, about 9.9 mol % of DSPC, about 40.4 mol % of cholesterol, and about 1.6 mol % of DMG-PEG 2000 (“DV-140-F6/15”).

In some implementations, the delivery vehicle composition comprises about 40 mol % to about 49 mol % of the cationic component; about 5 mol % to about 15 mol % of the anionic/zwitterionic component, about 30 mol % to about 55 mol % of the neutral lipid compound, and about 1 mol % to about 3 mol % of the shielding component. In various implementations, the delivery vehicle composition comprises about 42 mol % to about 46 mol % of the cationic component; about 7 mol % to about 12 mol % of the anionic/zwitterionic component, about 41 mol % to about 45 mol % of the neutral lipid compound, and about 1 mol % to about 2 mol % of the shielding component. In various implementations, the delivery vehicle composition comprises about 44.4 mol % of the cationic component; about 10.6 mol % of the anionic/zwitterionic component, about 43.3 mol % of the neutral lipid compound, and about 1.7 mol % of the shielding component. In various implementations, the delivery vehicle composition comprises about 44.4 mol % of the cationic component; about 10.6 mol % of the anionic/zwitterionic component, about 43.4 mol % of the neutral lipid compound, and about 1.7 mol % of the shielding component. The cationic component can be any cationic component described herein, such as any of the compounds of Formula (I) or Formula (II) (e.g., the compounds listed in Table 1 or Table 2, such as compound 140, 146, 151, 152, 160, 161, and 162). In some implementations, the cationic compound is Compound 140. The anionic/zwitterionic component can be any anionic/zwitterionic component described herein (e.g., a phospholipid). In some implementations, the anionic/zwitterionic component is DSPC or DOPE. The neutral lipid component can be any neutral lipid described herein (e.g., a sterol). In some implementations, the neutral lipid component is cholesterol. The shielding component can be any shielding component described herein (e.g., PEGylated lipids). In some implementations, the shielding component is DMG-PEG 2000. In some implementations, the delivery vehicle composition comprises F6.1 or F6.2, as shown in Table 3, below. In some implementations, the delivery vehicle composition comprises about 44.4 mol % of Compound 140, about 10.6 mol % of DSPC, about 43.3 mol % of cholesterol, and about 1.7 mol % of DMG-PEG 2000 (“DV-140-F6.1”). In some implementations, the delivery vehicle composition comprises about 44.4 mol % of Compound 140, about 10.6 mol % of DSPC, about 43.4 mol % of cholesterol, and about 1.7 mol % of DMG-PEG 2000 (“DV-140-F6.2”).

In some implementations, the delivery vehicle composition comprises about 30 mol % to about 39 mol % of the cationic component; about 5 mol % to about 15 mol % of the anionic/zwitterionic component, about 30 mol % to about 55 mol % of the neutral lipid compound, and about 1 mol % to about 3 mol % of the shielding component. In various implementations, the delivery vehicle composition comprises about 30 mol % to about 35 mol % of the cationic component; about 7 mol % to about 12 mol % of the anionic/zwitterionic component, about 50 mol % to about 55 mol % of the neutral lipid compound, and about 2 mol % to about 3 mol % of the shielding component. In various implementations, the delivery vehicle composition comprises about 33.1 mol % of the cationic component; about 10.5 mol % of the anionic/zwitterionic component, about 53.8 mol % of the neutral lipid compound, and about 2.5 mol % of the shielding component. The cationic component can be any cationic component described herein, such as any of the compounds of Formula (I) or Formula (II) (e.g., the compounds listed in Table 1 or Table 2, such as compound 140, 146, 151, 152, 160, 161, and 162). In some implementations, the cationic compound is Compound 140. The anionic/zwitterionic component can be any anionic/zwitterionic component described herein (e.g., a phospholipid). In some implementations, the anionic/zwitterionic component is DSPC or DOPE. The neutral lipid component can be any neutral lipid described herein (e.g., a sterol). In some implementations, the neutral lipid component is cholesterol. The shielding component can be any shielding component described herein (e.g., PEGylated lipids). In some implementations, the shielding component is DMG-PEG 2000. In some implementations, the delivery vehicle composition comprises F6.3, as shown in Table 3, below. In some implementations, the delivery vehicle composition comprises about 33.1 mol % of Compound 140, about 10.5 mol % of DSPC, about 53.8 mol % of cholesterol, and about 2.5 mol % of DMG-PEG 2000 (“DV-140-F6.1”).

Non-limiting examples delivery vehicle compositions of the disclosure based on Compound 140 as the cationic component (characterized by mol %) can be found in Table 2, below.

TABLE 3
Delivery Vehicle Compositions

| Molecular | | Anionic or | Non-cationic | |
|---|---|---|---|---|
| Percentages | Cationic | Zwitterionic | lipid | Shielding |
| (mol %) | component | component | component | component |
| F1A | 99.1 | 0 | 0 | 0.9 |
| F2A | 17.9 | 16.4 | 62.9 | 2.8 |
| F3A | 32.9 | 13.4 | 51.7 | 2.0 |
| F4A | 17.1 | 0 | 80.2 | 2.7 |
| F5A | 42.3 | 53.3 | 0 | 4.4 |
| F6A | 38.0 | 0 | 59.7 | 2.3 |
| F1 | 21.4 | 15.7 | 60.3 | 2.7 |
| F2 | 38.2 | 11.8 | 48.2 | 1.9 |
| F3 | 36.0 | 10.0 | 51.7 | 2.4 |
| F4 | 30.2 | 29.6 | 39.5 | 0.7 |
| F5 | 32.0 | 16.7 | 48.7 | 2.6 |
| F6/12 | 42.6 | 10.9 | 44.7 | 1.7 |
| F6/15 | 48.1 | 9.9 | 40.4 | 1.6 |
| F6/17 | 51.3 | 9.3 | 38.0 | 1.5 |
| F6.1 | 44.4 | 10.6 | 43.3 | 1.7 |
| F6.2 | 44.4 | 10.6 | 43.4 | 1.7 |
| F6.3 | 33.1 | 10.5 | 53.8 | 2.5 |

In some aspects, the delivery vehicle composition is F6.1, F6.2, or F6.3. In some aspects, the delivery vehicle composition is F1A, F72A, F73A, F74A, F5A, F76A, F1, F2, F3, F4, F5, F6/12, F6/15, or F6/17.

### Delivery Vehicle Complexes (DV)

The delivery vehicle compositions disclosed herein can form complexes with one or more polyanionic compounds (e.g., nucleic acids) through an electrostatic interaction between the cationic component of the delivery vehicle composition and the polyanionic compound. Thus, a delivery vehicle complex refers to a mixture comprising a delivery vehicle composition, as disclosed herein, and a polyanionic compound. The complexes, in some instances, permit a high amount of cargo encapsulation, are stable, and demonstrate excellent efficiency and tolerability in vivo. The delivery vehicle complexes, therefore, are useful as delivery vehicles for the transportation of the polyanionic cargo encapsulated therein to a target cell. Additionally or alternatively, the delivery vehicle complexes can include a non-anionic cargo. Accordingly, another aspect of the disclosure relates to a delivery vehicle complex comprising: (1) a delivery vehicle composition, as previously described herein, and (2) a polyanionic compound (or cargo). In some implementations, the delivery vehicle composition complexes with one polyanionic compound (e.g., one RNA). In various implementations, the delivery vehicle composition complexes with two different polyanionic compound (e.g., two different RNAs or an RNA and a DNA). In some implementations, the delivery vehicle composition complexes with three or more different polyanionic compounds (e.g., 3, 4, or 5 different RNAs).

The delivery vehicle complexes described herein may be characterized by the relative mass ratio of one of the components of the delivery vehicle composition to the cargo (e.g., a polyanionic compound) in the complex. Mass ratios of the components in the delivery vehicle complex can be readily calculated based upon the known concentrations and volumes of stock solutions of each component used in preparing the complex. Moreover, if non-anionic cargoes are present in the delivery vehicle complex, mass ratios may provide a more accurate representation of the relative amounts of delivery vehicle components to the overall cargo than cation:anion charge ratios, which do not account for non-anionic material. Specifically, the mass ratio of a component refers to the ratio of the mass of this particular component in the system to the mass of the “cargo” in the system. “Cargo” may refer to the total polyanionic compound(s) present in the system. In one example, the polyanionic compound(s) may refer to nucleic acid(s). In one example, the polyanionic compound(s) refer to mRNA(s) encoding at least one protein.

In some implementations, the cationic component and the polyanionic compound of the delivery vehicle complex have a mass ratio between about 0.5:1 and about 20:1, between about 0.5:1 and about 10:1, between about 0.5:1 and about 5:1, between about 1:1 and about 20:1, between about 1:1 and about 10:1, between about 1:1 and about 5:1, between about 2:1 and about 20:1, between about 2:1 and about 10:1, or between about 2:1 and about 5:1. In some implementations, the cationic component and the polyanionic compound of the delivery vehicle complex have a mass ratio between about 2:1 and about 5:1. In still yet other implementations, the cationic component and the polyanionic compound of the delivery vehicle complex have a mass ratio of about 3:1. In other implementations, the cationic component and the polyanionic compound of the delivery vehicle complex have a mass ratio of about 19:1. In other implementations, the cationic component and the polyanionic compound of the delivery vehicle complex have a mass ratio of about 20:1. In other implementations, the cationic component and the polyanionic compound of the delivery vehicle complex have a mass ratio of about 13:1. In other implementations, the cationic component and the polyanionic compound of the delivery vehicle complex have a mass ratio of about 10:1. In some implementations, the cationic component can be a compound of Formula (I) or Formula (II), such as a compound listed in Table 1 or Table 2.

In certain implementations wherein the delivery vehicle complex comprises a nucleic acid as the polyanionic compound, or cargo, the mass ratio of the cationic component and the nucleic acid is between about 0.5:1 and about 20:1, or between about 0.5:1 and about 10:1, or between about 0.5:1 and about 5:1, or between about 1:1 and about 20:1, or between about 1:1 and about 10:1, or between about 1:1 and about 5:1, or between about 2:1 and about 20:1, or between about 2:1 and about 10:1, or between about 2:1 and about 5:1. In certain implementations, the mass ratio of the cationic component and the nucleic acid is between about 2:1 and about 5:1. In still yet other implementations, the mass ratio of the cationic component and the nucleic acid is about 3:1. In other implementations, the mass ratio of the cationic component and the nucleic acid is about 19:1. In other implementations, the mass ratio of the cationic component and the nucleic acid is about 20:1. In other implementations, the mass ratio of the cationic component and the nucleic acid is about 13:1. In other implementations, the mass ratio of the cationic component and the nucleic acid is about 10:1. In some implementations, the cationic component can be a compound of Formula (I) or Formula (II), such as a compound listed in Table 1 or Table 2.

In some implementations, the mass ratio of the cationic component and the nucleic acid is between about 5:1 to about 25:1, or about 7:1 to about 20:1, or about 10:1 to about 17:1, or about 9.5:1 to about 10.5:1, or about 11:1 to about 17:1. In various implementations, the mass ratio of the cationic component and the nucleic acid is about 20:1. In various implementations, the mass ratio of the cationic component and the nucleic acid is about 19:1. In some implementations, the mass ratio of the cationic component and the nucleic acid is about 17:1. In various implementations, the mass ratio of the cationic component and the nucleic acid is about 15:1. In various implementations, the mass ratio of the cationic component and the nucleic acid is about 13:1. In various implementations, the mass ratio of the cationic component and the nucleic acid is about 12:1. In various implementations, the mass ratio of the cationic component and the nucleic acid is about 10:1. In some implementations, the cationic component can be a compound of Formula (I) or Formula (II), as previously described herein, such as a compound listed in Table 1 or Table 2. In various implementations, the cationic component is Compound 140. In some implementations, the polyanionic cargo is a nucleic acid, such as RNA.

In some aspects, the mass ratio of the anionic/zwitterionic component and the polyanionic compound is about 2:1 to about 10:1, or about 2:1 to about 3:1, or about 2:1 to about 4:1, or about 5:1 to about 10:1. In some aspects, the mass ratio of the anionic/zwitterionic component and the polyanionic compound is about 2:1 to about 10:1, or about 2:1 to about 3:1, or about 5:1 to about 10:1. In some implementations, the mass ratio of the anionic/zwitterionic component and the polyanionic compound is about 4:1. In some implementations, the mass ratio of the anionic/zwitterionic component and the polyanionic compound is about 2.7:1. In some implementations, the anionic/zwitterionic component can be a phospholipid, as previously described herein. In various implementations, the anionic/zwitterionic component is DOPE, DSPC, or a combination thereof. In some aspects, the anionic/zwitterionic component is DSPC. In some implementations, the polyanionic cargo is a nucleic acid, such as RNA.

In some aspects, the mass ratio of the neutral lipid component and the polyanionic compound is between about 5:1 to about 8:1, or about 4:1 to about 7:1, or about 5:1 to about 6:1, or about 1:1 to about 5:1. In some aspects, the mass ratio of the neutral lipid component and the polyanionic compound is between about 4:1 to about 7:1, or about 5:1 to about 6:1, or about 1:1 to about 5:1. In some implementations, the mass ratio of the neutral lipid component and the polyanionic compound is about 5.4:1. In some implementations, the mass ratio of the neutral lipid component and the polyanionic compound is about 8.1:1. In some implementations, the mass ratio of the neutral lipid component and the polyanionic compound is about 6.7:1. In some implementations, the neutral lipid component can be a sterol, as previously described herein. In various implementations, the neutral lipid component is cholesterol. In some implementations, the polyanionic cargo is a nucleic acid, such as RNA.

In some aspects, the mass ratio of the shielding component and the polyanionic compound is between about 0.5:1 to about 2.5:1, or about 1:1 to about 2:1, or about 2:1 to about 3:1. In some implementations, the mass ratio of the neutral lipid component and the polyanionic compound is about 2.1:1. In some implementations, the mass ratio of the neutral lipid component and the polyanionic compound is about 1.4:1. In some implementations, the shielding component can be a PEGylated lipid, as previously described herein. In various implementations, the shielding component is DMG-PEG 2000. In some implementations, the polyanionic cargo is a nucleic acid, such as RNA.

In some implementations, the delivery vehicle complex comprises the cationic component and the polyanionic cargo at a mass ratio of about 10:1, the anionic/zwitterionic component and the polyanionic cargo at a mass ratio of about 2.7:1, the neutral lipid component and the polyanionic cargo at a mass ratio of about 5.4:1, and the shielding component and the polyanionic cargo at a mass ratio of about 1.4:1 (“Form F2”). In some implementations, the cationic component is a compound of Formula (I) or Formula (II), the anionic/zwitterionic component is a phospholipid, the neutral lipid component is cholesterol, and the shielding component is a PEGylated lipid. In some implementations, the polyanionic compound is a nucleic acid, such as RNA. In various implementations, the delivery vehicle complex comprises Compound 1, Compound 6, Compound 21, Compound 30 or Compound 140 at a mass ratio of about 10:1 with the nucleic acid, DSPC at a mass ratio of about 2.7:1 with the nucleic acid, cholesterol at a mass ratio of about 5.4:1 with the nucleic acid, and DMG-PEG 2000 at a mass ratio of about 1.4 with the nucleic acid (“DV-140-F2”).

In some implementations, the delivery vehicle complex comprises the cationic component and the polyanionic cargo at a mass ratio of about 17:1, the anionic/zwitterionic component and the polyanionic cargo at a mass ratio of about 2.7:1, the neutral lipid component and the polyanionic cargo at a mass ratio of about 5.4:1, and the shielding component and the polyanionic cargo at a mass ratio of about 1.4:1 (“Form F6/17”). In some implementations, the cationic component is a compound of Formula (I) or Formula (II), the anionic/zwitterionic component is a phospholipid, the neutral lipid component is cholesterol, and the shielding component is a PEGylated lipid. In some implementations, the polyanionic cargo is a nucleic acid, such as RNA. In various implementations, the delivery vehicle complex comprises Compound 1, Compound 6, Compound 21, Compound 30 or Compound 140 at a mass ratio of about 17:1 with the nucleic acid, DSPC at a mass ratio of about 2.7:1 with the nucleic acid, cholesterol at a mass ratio of about 5.4:1 with the nucleic acid, and DMG-PEG 2000 at a mass ratio of about 1.4 with the nucleic acid (“DV-140-F6/17”).

In some implementations, the delivery vehicle complex comprises the cationic component and the polyanionic cargo at a mass ratio of about 12:1, the anionic/zwitterionic component and the polyanionic cargo at a mass ratio of about 2.7:1, the neutral lipid component and the polyanionic cargo at a mass ratio of about 5.4:1, and the shielding component and the polyanionic cargo at a mass ratio of about 1.4:1 (“Form F6/12”). In some implementations, the cationic component is a compound of Formula (I) or Formula (II), the anionic/zwitterionic component is a phospholipid, the neutral lipid component is cholesterol, and the shielding component is a PEGylated lipid. In some implementations, the polyanionic cargo is a nucleic acid, such as RNA. In various implementations, the delivery vehicle complex comprises Compound 1, Compound 6, Compound 21, Compound 30 or Compound 140 at a mass ratio of about 12:1 with the nucleic acid, DSPC at a mass ratio of about 2.7:1 with the nucleic acid, cholesterol at a mass ratio of about 5.4:1 with the nucleic acid, and DMG-PEG 2000 at a mass ratio of about 1.4 with the nucleic acid (“DV-140-F6/12”).

In some implementations, the delivery vehicle complex comprises the cationic component and the polyanionic cargo at a mass ratio of about 15:1, the anionic/zwitterionic component and the polyanionic cargo at a mass ratio of about 2.7:1, the neutral lipid component and the polyanionic cargo at a mass ratio of about 5.4:1, and the shielding component and the polyanionic cargo at a mass ratio of about 1.4:1 (“Form F6/15”). In some implementations, the cationic component is a compound of Formula (I) or Formula (II), the anionic/zwitterionic component is a phospholipid, the neutral lipid component is cholesterol, and the shielding component is a PEGylated lipid. In some implementations, the polyanionic cargo is a nucleic acid, such as RNA. In various implementations, the delivery vehicle complex comprises Compound 1, Compound 6, Compound 21, Compound 30 or Compound 140 at a mass ratio of about 15:1 with the nucleic acid, DSPC at a mass ratio of about 2.7:1 with the nucleic acid, cholesterol at a mass ratio of about 5.4:1 with the nucleic acid, and DMG-PEG 2000 at a mass ratio of about 1.4 with the nucleic acid (“DV-140-F6/15”).

In some implementations, the delivery vehicle complex comprises the cationic component and the polyanionic cargo at a mass ratio of about 13:1, the anionic/zwitterionic component and the polyanionic cargo at a mass ratio of about 2.7:1, the neutral lipid component and the polyanionic cargo at a mass ratio of about 5.4:1, and the shielding component and the polyanionic cargo at a mass ratio of about 1.4:1 (“F6.1”). In some implementations, the cationic component is a compound of Formula (I) or Formula (II), the anionic/zwitterionic component is a phospholipid, the neutral lipid component is cholesterol, and the shielding component is a PEGylated lipid. In some implementations, the polyanionic cargo is a nucleic acid, such as RNA. In various implementations, the delivery vehicle complex comprises Compound 1, Compound 6, Compound 21, Compound 30 or Compound 140 at a mass ratio of about 13:1 with the nucleic acid, DSPC at a mass ratio of about 2.7:1 with the nucleic acid, cholesterol at a mass ratio of about 5.4:1 with the nucleic acid, and DMG-PEG 2000 at a mass ratio of about 1.4 with the nucleic acid (“DV-140-F6.1”).

In some implementations, the delivery vehicle complex comprises the cationic component and the polyanionic cargo at a mass ratio of about 19:1, the anionic/zwitterionic component and the polyanionic cargo at a mass ratio of about 4.0:1, the neutral lipid component and the polyanionic cargo at a mass ratio of about 8.1:1, and the shielding component and the polyanionic cargo at a mass ratio of about 2.1:1 (“F6.2”). In some implementations, the cationic component is a compound of Formula (I) or Formula (II), the anionic/zwitterionic component is a phospholipid, the neutral lipid component is cholesterol, and the shielding component is a PEGylated lipid. In some implementations, the polyanionic cargo is a nucleic acid, such as RNA. In various implementations, the delivery vehicle complex comprises Compound 1, Compound 6, Compound 21, Compound 30 or Compound 140 at a mass ratio of about 19:1 with the nucleic acid, DSPC at a mass ratio of about 4.0:1 with the nucleic acid, cholesterol at a mass ratio of about 8.1:1 with the nucleic acid, and DMG-PEG 2000 at a mass ratio of about 2.1 with the nucleic acid (“DV-140-F6.2”).

In some implementations, the delivery vehicle complex comprises the cationic component and the polyanionic cargo at a mass ratio of about 9.7, the anionic/zwitterionic component and the polyanionic cargo at a mass ratio of about 2.7:1, the neutral lipid component and the polyanionic cargo at a mass ratio of about 6.7:1, and the shielding component and the polyanionic cargo at a mass ratio of about 2.1:1 (“F6.3”). In some implementations, the cationic component is a compound of Formula (I) or Formula (II), the anionic/zwitterionic component is a phospholipid, the neutral lipid component is cholesterol, and the shielding component is a PEGylated lipid. In some implementations, the polyanionic cargo is a nucleic acid, such as RNA. In various implementations, the delivery vehicle complex comprises Compound 1, Compound 6, Compound 21, Compound 30 or Compound 140 at a mass ratio of about 9.7:1 with the nucleic acid, DSPC at a mass ratio of about 2.7:1 with the nucleic acid, cholesterol at a mass ratio of about 6.7:1 with the nucleic acid, and DMG-PEG 2000 at a mass ratio of about 2.1 with the nucleic acid (“DV-140-F6.3”).

In still other implementations, the amount of polyanionic cargo present in the delivery vehicle complexes may be characterized by a mass ratio of delivery vehicle composition (e.g., hydroxyethyl capped lipidated cationic peptoids, phospholipid, cholesterol, and/or the shielding component in total) to the one or more polyanionic cargo compounds. In some implementations, the mass ratio of the delivery vehicle composition to the one or more polyanionic cargo compounds is between about 0.5:1 and about 20:1, between about 0.5:1 and about 10:1, between about 0.5:1 and about 5:1, between about 1:1 and about 20:1, between about 1:1 and about 10:1, between about 1:1 and about 5:1, between about 2:1 and about 20:1, between about 2:1 and about 10:1, or between about 2:1 and about 5:1. In certain implementations, the mass ratio of the delivery vehicle composition to the one or more polyanionic cargo compounds is between about 5:1 and about 8:1 or between about 6:1 and about 7:1.

### Methods of Making the Delivery Vehicle Complex

Components of the delivery vehicle complex can be prepared through a variety of physical and/or chemical methods to modulate their physical, chemical, and biological properties. These may involve rapid combination of the hydroxyethyl-capped tertiary amino lipidated cationic peptoids in water or a water-miscible organic solvent with the desired polyanionic cargo compound (e.g., oligonucleotides or nucleic acids) in water or an aqueous buffer solution. These methods can include simple mixing of the components by pipetting, or microfluidic mixing processes such as those involving T-mixers, vortex mixers, or other chaotic mixing structures. In some implementations, the multicomponent delivery system is prepared on a microfluidic platform.

It is to be understood that the particular process conditions for preparing the delivery vehicle complexes described herein may be adjusted or selected accordingly to provide the desired physical properties of the complexes. For example, parameters for mixing the components of the delivery system complex that may influence the final compositions may include, but are not limited to, order of mixing, temperature of mixing, mixing speed/rate, flow rate, physical dimensions of the mixing structure, concentrations of starting solutions, molar ratio of components, and solvents used.

Formulation of the delivery vehicle complexes can be accomplished in many ways. In some aspects, all components can be pre-mixed prior to addition of the nucleic acid cargo, which can result in a uniform distribution of components throughout the delivery particle.

Exemplary mixing methods are detailed in, e.g., U.S. Pat. Nos. 11,278,895 and 11,325,122, incorporated herein by reference.

In other aspects, the components can be added sequentially to produce a core-shell type structure. For example, a cationic component could be added first to begin particle condensation, followed by a lipid component to allow the particles surface to associate with target cells, followed by a shielding component to prevent particle aggregation. For example, the hydroxyethyl-capped tertiary amino lipidated cationic peptoid can be premixed with the nucleic acid cargo to form a core structure. Then, the lipid components (such as lipid components comprising phospholipids and cholesterol) can be added to influence cell/endosomal membrane association. Because the shielding component is primarily useful on the outside of the multicomponent delivery system, this component can be introduced last, so that it does not disrupt the internal structure of the system, but rather provides a coating of the system after it is formed.

Additional components in the complexes and composition, such as the additional components of polymers, surface-active agents, targeting moieties, and/or excipients, may be admixed and combined with the rest of the components before, during, or after the principal components of the nucleic acid cargo, the cationic component, the lipid component and the shielding component have been combined.

Accordingly, also provided herein is a method of forming the delivery vehicle complex disclosed herein, comprising contacting the compound or salt of Formula (I) or Formula (II) with the polyanionic compound. In some implementations, the method comprises admixing a solution comprising the compound or salt of Formula (I) or Formula (II) with a solution comprising the polyanionic compound.

### Pharmaceutical Formulations and Modes of Administration

Also provided herein are pharmaceutical compositions that include the delivery vehicle complexes of the disclosure, and an effective amount of one or more pharmaceutically acceptable excipients. An “effective amount” includes a “therapeutically effective amount” and a “prophylactically effective amount.” The term “therapeutically effective amount” refers to an amount effective in treating and/or ameliorating a disease or condition in a subject. The term “prophylactically effective amount” refers to an amount effective in preventing and/or substantially lessening the chances of a disease or condition in a subject. As used herein, the terms “patient” and “subject” may be used interchangeably and mean human patients or subjects. The terms “patient” and “subject” include males and females. As used herein, the term “excipient” means any pharmaceutically acceptable additive, carrier, diluent, adjuvant, or other ingredient, other than the active pharmaceutical ingredient (API) (and typically in addition to components of the delivery vehicle compositions), suitably selected with respect to the intended form of administration, and consistent with conventional pharmaceutical practices.

The complexes of the disclosure can be administered to a subject or patient in a therapeutically effective amount. The complexes can be administered alone or as part of a pharmaceutically acceptable composition or formulation. In addition, the complexes can be administered all at once, as for example, by a bolus injection, multiple times, or delivered substantially uniformly over a period of time. It is also noted that the dose of the compound can be varied over time.

The delivery vehicle complexes disclosed herein and the pharmaceutically active compounds described herein can be administered to a subject or patient by any suitable route, for example, by parenteral injection(s), e.g., intramuscularly (IM), intratumorally (IT), intravenous (IV), or by intraperitoneal delivery, subcutaneous (SQ) delivery, intradermal delivery or mucosal delivery. All methods that can be used by those skilled in the art to administer a pharmaceutically active agent are contemplated.

Compositions suitable for parenteral injection may comprise physiologically acceptable sterile aqueous or nonaqueous solutions, dispersions, suspensions, or emulsions, and sterile powders for reconstitution into sterile injectable solutions or dispersions. Examples of suitable aqueous and nonaqueous carriers, diluents, solvents, or vehicles include water, buffers, ethanol, polyols (propylene glycol, polyethylene glycol, glycerol, and the like), and others known in the art. Proper fluidity can be maintained, for example, by the use of a coating such as lecithin, by the maintenance of the required particle size in the case of dispersions, and by the use of surfactants.

These compositions may also contain adjuvants such as preserving, wetting, emulsifying, and dispersing agents. Microorganism contamination can be prevented by adding various antibacterial and antifungal agents, for example, parabens, chlorobutanol, phenol, sorbic acid, and the like. It may also be desirable to include isotonic agents, for example, sugars, sodium chloride, and the like. Prolonged absorption of injectable pharmaceutical compositions can be brought about by the use of agents delaying absorption, for example, aluminum monostearate and gelatin.

The pharmaceutical compositions may be in the form of a sterile injectable, an aqueous suspension or an oleaginous suspension. This suspension may be formulated according to the known art using those suitable dispersing or wetting agents and suspending agents which have been mentioned above. The sterile injectable preparation may also be sterile injectable solution or suspension in a non-toxic parentally acceptable diluent or solvent. Examples of acceptable vehicles and solvents that may be employed are water, a buffer, Ringer's solution and isotonic sodium chloride solution. Exemplary buffers include citrate, succinate, acetate, malate, succinate, histidine. In addition, stabilizers such as sucrose, may be included. For example, the pharmaceutical compositions may be suspended in a sucrose-containing citrate buffer at a pH between pH 5 and pH 6, e.g., at about pH 5.5.

Compositions for parenteral administration are administered in a sterile medium. Depending on the vehicle used and concentration the concentration of the drug in the formulation, the parenteral formulation can either be a suspension or a solution containing dissolved drug. Adjuvants such as local anesthetics, preservatives and buffering agents can also be added to parenteral compositions.

Vaccine compositions may further comprise one or more immunologic adjuvants. As used herein, the term “immunologic adjuvant” refers to a compound or a mixture of compounds that acts to accelerate, prolong, enhance or modify immune responses when used in conjugation with an immunogen (e.g., neoantigens). Adjuvant may be non-immunogenic when administered to a host alone, but that augments the hosts immune response to another antigen when administered conjointly with that antigen. Specifically, the terms “adjuvant” and “immunologic adjuvant” are used interchangeably in the present disclosure. Adjuvant-mediated enhancement and/or extension of the duration of the immune response can be assessed by any method known in the art including without limitation one or more of the following: (i) an increase in the number of antibodies produced in response to immunization with the adjuvant/antigen combination versus those produced in response to immunization with the antigen alone; (ii) an increase in the number of T cells recognizing the antigen or the adjuvant; and (iii) an increase in the level of one or more cytokines. Adjuvants may be aluminum based adjuvants including but not limiting to aluminum hydroxide and aluminum phosphate; saponins such as steroid saponins and triterpenoid saponins; bacterial flagellin and some cytokines such as GM-CSF. Adjuvants selection may depend on antigens, vaccines, and routes of administrations.

In some implementations, adjuvants improve the adaptive immune response to a vaccine antigen by modulating innate immunity or facilitating transport and presentation. Adjuvants act directly or indirectly on antigen presenting cells (APCs) including dendritic cells (DCs). Adjuvants may be ligands for toll-like receptors (TLRs) and can directly affect DCs to alter the strength, potency, speed, duration, bias, breadth, and scope of adaptive immunity. In other instances, adjuvants may signal via proinflammatory pathways and promote immune cell infiltration, antigen presentation, and effector cell maturation. This class of adjuvants includes mineral salts, oil emulsions, nanoparticles, and polyelectrolytes and comprises colloids and molecular assemblies exhibiting complex, heterogeneous structures. In one example, the composition further comprises pidotimod as an adjuvant. In another example, the composition further comprises CpG as an adjuvant.

The compounds of the disclosure can be administered to a subject or patient at dosage levels in the range of about 0.1 to about 100 mg per day. For weight-based dosing a normal adult human having a body weight of about 70 kg, a dosage in the range of about 0.001 to about 1 mg per kilogram body weight is contemplated to be sufficient. The specific dosage and dosage range that will be used can potentially depend on a number of factors, including the requirements of the subject or patient, the severity of the condition or disease being treated, and the pharmacological activity of the compound being administered. The determination of dosage ranges and optimal dosages for a particular subject or patient is within the ordinary skill in the art.

### Methods of Use

The delivery vehicle complexes disclosed herein can be used to deliver the polyanionic compound of the complex (or cargo) to a cell. Accordingly, disclosed herein are methods of delivering a polyanionic compound, such as a nucleic acid (e.g., RNA) encoding any of the SIRPα fusion proteins, SIRPα/CCR4-binding fusion proteins, high affinity fusion proteins or heterodimers including bivalent and tetravalent fusion proteins disclosed herein to a cell comprising contacting the cell with the delivery vehicle complex or pharmaceutical composition disclosed herein. In some implementations, the cell is obtained from a subject. In some aspects, the cell is a tumor cell. In some aspects, the cell is a muscle cell.

In some implementations, the one or more polyanionic cargo compounds may be delivered for therapeutic uses. Non-limiting therapeutic uses include CIN and cancer, e.g., as a vaccine.

### Vaccines.

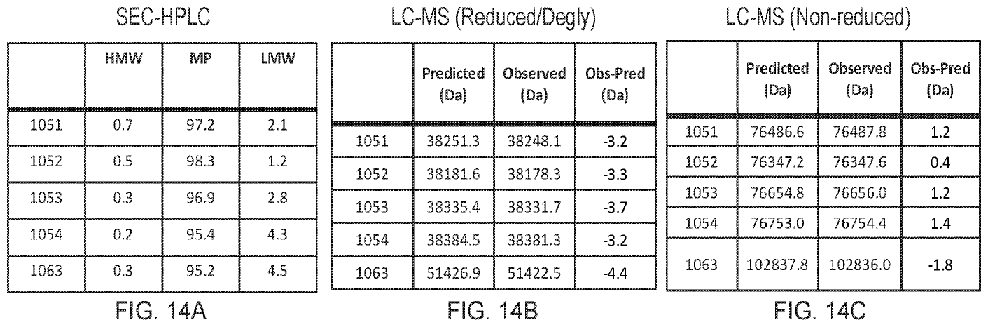

The delivery vehicle complexes of the disclosure are useful as vaccines, in which the polyanionic compound is an RNA encoding any of the SIRPα fusion proteins, SIRPα/CCR4-binding fusion proteins, high affinity fusion proteins or heterodimers including bivalent and tetravalent fusion proteins disclosed herein. The immune system of a host provides the means for quickly and specifically mounting a protective response to pathogenic microorganisms and also for contributing to rejection of malignant tumors. Immune responses have been generally described as including humoral responses, in which antibodies specific for antigens are produced by differentiated B lymphocytes, and cell mediated responses, in which various types of T lymphocytes eliminate antigens by a variety of mechanisms. For example, CD4 (also called CD4+) helper T cells that are capable of recognizing specific antigens may respond by releasing soluble mediators such as cytokines to recruit additional cells of the immune system to participate in an immune response. CD8 (also called CD8+) cytotoxic T cells are also capable of recognizing specific antigens and may bind to and destroy or damage an antigen-bearing cell or particle. In particular, cell mediated immune responses that include a cytotoxic T lymphocyte (CTL) response can be important for elimination of tumor cells and cells infected by a microorganism, such as virus, bacteria, or parasite. The delivery vehicle complexes of the disclosure have been found to induce immune responses when one or more of the polyanionic compound of the complex encodes a viral peptide (e.g., a viral polypeptide), a viral protein, or functional fragment of the foregoing. For example, delivery vehicle complexes comprising either DV-140-F2 or DV-140-F6/17 complexed with mRNA encoding the HPV E6/E7 (e.g., from HPV 16 and/or HPV 18) oncogene elicited strong humoral and cellular immune responses. See, e.g., Example 14 and FIGS. 14A and 14B.

Thus, the disclosure includes methods for inducing an immune response in a subject in need thereof, comprising administering to the subject an effective amount of the delivery vehicle complex (e.g., formulated as an antigenic composition) of the disclosure. Also disclosed herein is a method of treating CIN, squamous cell carcinoma and adenocarcinoma, for example cancers of the cervix, vulva, vagina, penis, anus, oropharynx and head & neck, in a subject in need thereof, comprising administering to the subject an effective amount of the delivery vehicle complex of the disclosure. In some implementations, the administering is by intramuscular, intratumoral, intravenous, intraperitoneal, or subcutaneous delivery.

In various implementations, administering the delivery vehicle complexes of the disclosure (e.g., formulated as a composition, pharmaceutical formulation, or antigenic composition) to a subject can result in an increase in the amount of antibodies (e.g., neutralizing antibodies) against the viral antigen that is produced in the subject relative to the amount of antibodies that is produced in a subject who was not administered the delivery vehicle complex. In some implementations, the increase is a 2-fold increase, a 5-fold increase, a 10-fold increase, a 50-fold increase, a 100-fold increase, a 200-fold increase, a 500-fold increase, a 700-fold increase, or a 1000-fold increase.

The immune response raised by the methods of the present disclosure generally includes an antibody response, preferably a neutralizing antibody response, maturation and memory of T and B cells, antibody dependent cell-mediated cytotoxicity (ADCC), antibody cell-mediated phagocytosis (ADCP), complement dependent cytotoxicity (CDC), and T cell-mediated response such as CD4+, CD8+. The immune response generated by the delivery vehicle complexes comprising RNA that encodes a viral antigen as disclosed herein generates an immune response that recognizes, and preferably ameliorates and/or neutralizes, a viral infection as described herein. Methods for assessing antibody responses after administration of an antigenic composition (immunization or vaccination) are known in the art and/or described herein. In some implementations, the immune response comprises a T cell-mediated response (e.g., peptide-specific response such as a proliferative response or a cytokine response). In some implementations, the immune response comprises both a B cell and a T cell response. Antigenic compositions can be administered in a number of suitable ways, such as intramuscular injection, intratumoral injection, subcutaneous injection, intradermal administration and mucosal administration such as oral or intranasal. Additional modes of administration include but are not limited to intravenous, intraperitoneal, intranasal administration, intra-vaginal, intra-rectal, and oral administration. A combination of different routes of administration in the immunized subject, for example intramuscular and intranasal administration at the same time, is also contemplated by the disclosure.

### Cancer

Various cancers (e.g., lymphoma, cervical cancer, hepatocellular carcinoma, ovarian tumor, a colon tumor or a disseminated gastric tumor) may be treated with the polyanionic cargo compounds delivered by the delivery vehicle complexes of the present disclosure. As shown in Example 14, DV-140-F2 complexed to an mRNA encoding for HPV E6/E7 (from HPV 16 and/or HPV 18) eliciting both strong cellular and humoral immune responses, illustrating the ability of the delivery vehicle complexes of the disclosure to treat cancer. As used herein, the term “cancer” refers to any of various malignant neoplasms characterized by the proliferation of anaplastic cells that tend to invade surrounding tissue and metastasize to new body sites and also refers to the pathological condition characterized by such malignant neoplastic growths. Cancers may be tumors or hematological malignancies, and include but are not limited to, all types of lymphomas/leukemias, carcinomas and sarcomas, such as those cancers or tumors found in the anus, bladder, bile duct, bone, brain, breast, cervix, colon/rectum, endometrium, esophagus, eye, gallbladder, head and neck, liver, kidney, larynx, lung, mediastinum (chest), mouth, ovaries, pancreas, penis, prostate, skin, small intestine, stomach, spinal marrow, tailbone, testicles, thyroid, uterus, vagina, and vulva.

As a non-limiting example, the carcinoma which may be treated may be Acute granulocytic leukemia, Acute lymphocytic leukemia, Acute myelogenous leukemia, Adenocarcinoma, Adenosarcoma, Adrenal cancer, Adrenocortical carcinoma, Anal cancer, Anaplastic astrocytoma, Angiosarcoma, Appendix cancer, Astrocytoma, Basal cell carcinoma, B-Cell lymphoma), Bile duct cancer, Bladder cancer, Bone cancer, Bowel cancer, Brain cancer, Brain stem glioma, Brain tumor, Breast cancer, Carcinoid tumors, Cervical cancer, Cholangiocarcinoma, Chondrosarcoma, Chronic lymphocytic leukemia, Chronic myelogenous leukemia, Colon cancer, Colorectal cancer, Craniopharyngioma, Cutaneous lymphoma, Cutaneous melanoma, Diffuse astrocytoma, Ductal carcinoma in situ, Endometrial cancer, Ependymoma, Epithelioid sarcoma, Esophageal cancer, Ewing sarcoma, Extrahepatic bile duct cancer, Eye cancer, Fallopian tube cancer, Fibrosarcoma, Gallbladder cancer, Gastric cancer, Gastrointestinal cancer, Gastrointestinal carcinoid cancer, Gastrointestinal stromal tumors, General, Germ cell tumor, Glioblastoma multiforme, Glioma, Hairy cell leukemia, Head and neck cancer, Hemangioendothelioma, Hodgkin lymphoma, Hodgkins disease, Hodgkins lymphoma, Hypopharyngeal cancer, Infiltrating ductal carcinoma, Infiltrating lobular carcinoma, Inflammatory breast cancer, Intestinal Cancer, Intrahepatic bile duct cancer, Invasive/infiltrating breast cancer, Islet cell cancer, Jaw cancer, Kaposi sarcoma, Kidney cancer, Laryngeal cancer, Leiomyosarcoma, Leptomeningeal metastases, Leukemia, Lip cancer, Liposarcoma, Liver cancer, Lobular carcinoma in situ, Low-grade astrocytoma, Lung cancer, Lymph node cancer, Lymphoma, Male breast cancer, Medullary carcinoma, Medulloblastoma, Melanoma, Meningioma, Merkel cell carcinoma, Mesenchymal chondrosarcoma, Mesenchymous, Mesothelioma, Metastatic breast cancer, Metastatic melanoma, Metastatic squamous neck cancer, Mixed gliomas, Mouth cancer, Mucinous carcinoma, Mucosal melanoma, Multiple myeloma, Nasal cavity cancer, Nasopharyngeal cancer, Neck cancer, Neuroblastoma, Neuroendocrine tumors, Non-Hodgkin lymphoma, Non-Hodgkins lymphoma, Non-small cell lung cancer, Oat cell cancer, Ocular cancer, Ocular melanoma, Oligodendroglioma, Oral cancer, Oral cavity cancer, Oropharyngeal cancer, Osteogenic sarcoma, Osteosarcoma, Ovarian cancer, Ovarian epithelial cancer, Ovarian germ cell tumor, Ovarian primary peritoneal carcinoma, Ovarian sex cord stromal tumor, Pagets disease, Pancreatic cancer, Papillary carcinoma, Paranasal sinus cancer, Parathyroid cancer, Pelvic cancer, Penile cancer, Peripheral nerve cancer, Peritoneal cancer, Pharyngeal cancer, Pheochromocytoma, Pilocytic astrocytoma, Pineal region tumor, Pineoblastoma, Pituitary gland cancer, Primary central nervous system lymphoma, Prostate cancer, Rectal cancer, Renal cell cancer, Renal pelvis cancer, Rhabdomyosarcoma, Salivary gland cancer, Sarcoma, Sarcoma, bone, Sarcoma, soft tissue, Sarcoma, uterine, Sinus cancer, Skin cancer, Small cell lung cancer, Small intestine cancer, Soft tissue sarcoma, Spinal cancer, Spinal column cancer, Spinal cord cancer, Spinal tumor, Squamous cell carcinoma, Stomach cancer, Synovial sarcoma, T-cell lymphoma), Testicular cancer, Throat cancer, Thymoma/thymic carcinoma, Thyroid cancer, Tongue cancer, Tonsil cancer, Transitional cell cancer, Transitional cell cancer, Transitional cell cancer, Triple-negative breast cancer, Tubal cancer, Tubular carcinoma, Ureteral cancer, Ureteral cancer, Urethral cancer, Uterine adenocarcinoma, Uterine cancer, Uterine sarcoma, Vaginal cancer, and Vulvar cancer.

In some implementations, the delivery vehicle complexes of the disclosure are used to treat a cancer is selected from the group consisting of cervical cancer, head and neck cancer, B-cell lymphoma, T-cell lymphoma, prostate cancer, and lung cancer.

### EXAMPLES

### Materials & Methods

Cell Culture—Human tumor lines (Raji cells (Cat #CCL-86), H9 cells (Cat #HTB-176) and HEK293 cells were purchased from American Type Culture Collection and cultured as recommended by the vendor. Raji cells express CD47, while H9 cells express both CD47 and CCR4.

RBC Binding Assay—50 μl of 1000× diluted whole blood from 5 different donors were incubated with indicated concentration of SIRPα-FC protein in cell staining buffer (Biolegend CAT #420201) for 20 min at room temperature (RT). After washing with cell staining buffer (Biolegend CAT #420201), the cells were incubated with Phycoerythrin (PE) labeled anti-human-IgG-FC (Invitrogen CAT #12-4998-82) for 20 min at RT and then the cells were washed and analyzed by flow cytometry.

### Example 1—Cell-Based Ligand-Binding Assay for SIRPα-CD47

About 200,000 Raji cells were stained with indicated concentration of SIRPα-FC protein in cell staining buffer (Biolegend CAT #420201) for 20 min at RT. After washing cell staining buffer, the cells were incubated with PE labeled anti-human-IgG-FC for 20 min at RT, and then the cells were washed and analyzed by flow cytometry.

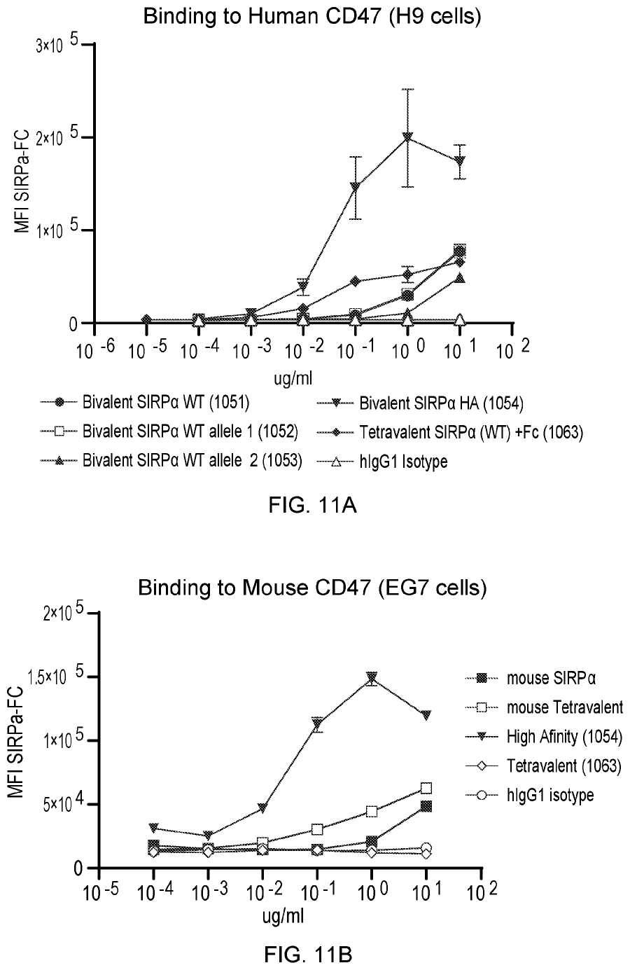

FIGS. 11A and 11B show data generated using molecules including molecules encoded by the synthesized mRNA sequences described herein, confirming expression of SIRPα. To generate the data shown in FIG. 11A, the indicated mRNA molecules were introduced to human embryonic kidney (HEK) for 20 hours after which the supernatants were collected for expression testing. Binding to human CD47 (on H9 cells) was assessed at various dilutions of the subject SIRPα molecules and plotted. Molecules assessed included 1051 (SEQ ID NO: 4; bivalent SIRPα WT), 1052 (SEQ ID NO: 5; SIRPα allele 1), 1053 (SEQ ID NO: 6; LL1 HC, SIRPα allele 2), 1054 (SEQ ID NO: 7; a high affinity SIRPα construct (1054)) and a tetravalent SIRPα fusion protein of the present invention (1063; SEQ ID NO: 2), based on an engineered mRNA sequence described herein having antigen binding sites that were wild-type (WT), i.e., not high affinity (HA). Note the high level of binding to CD47 in the Raji cells with the tetravalent design (1063) that does not include a high affinity binding site. The data show that the tetravalent construct shows a higher degree of binding relative to other WT bivalent constructs, but not as high as the HA construct.

Analogous experiments were conducted in EG7 cells expressing mouse CD47, using mouse SIRPα WT constructs, as well as the human HA construct (1054), which cross-reacts with mouse. Those data are shown in FIG. 11B.

### Example 2—Size Exclusion High Performance Liquid Chromatography (SE-HPLC)

Analytical Size Exclusion HPLC was performed using Agilent Bio SEC-55 um 300 A 7.8 mm×300 mm, with mobile phase of 2×PBS, and at the flow rate of 1 ml/min. ChemStation software was used to integrate and analyze the peaks. Insets show expanded view around the main peaks. The solvent peak, from the protein samples, between 11 and 12 min were excluded in the integration.

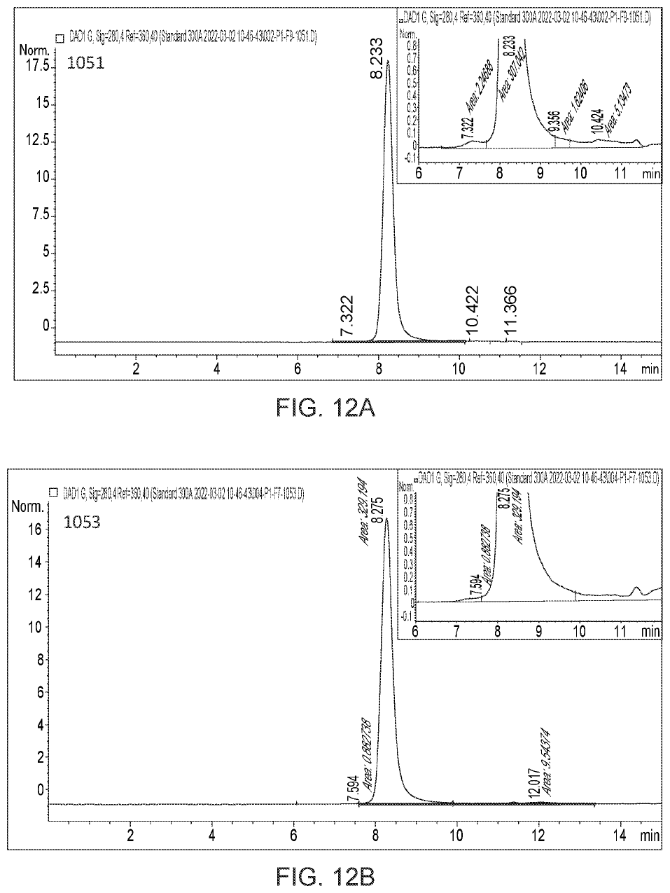

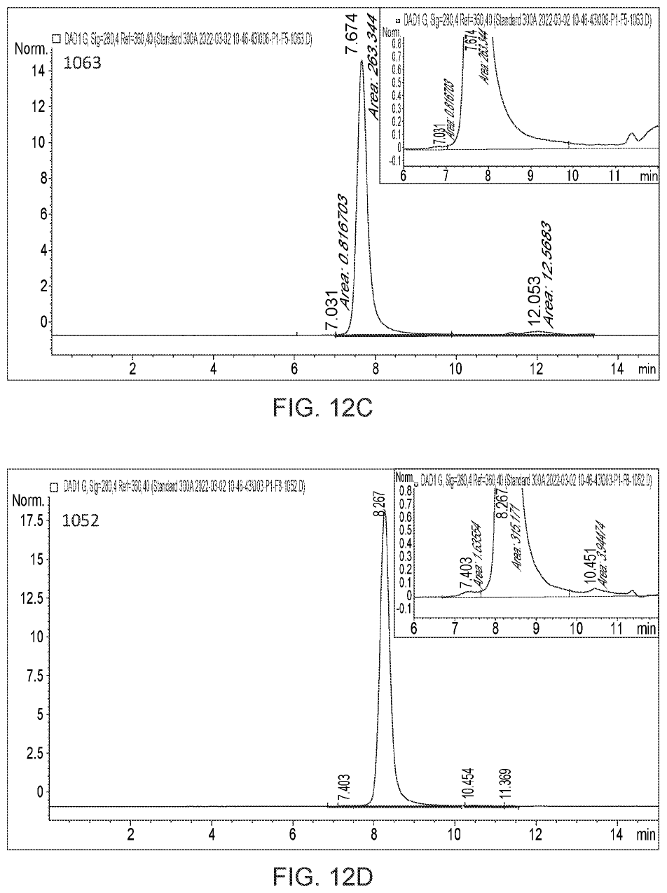

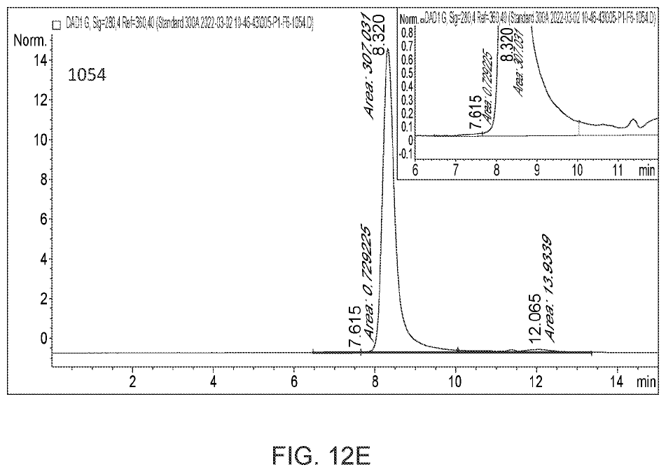

Fusion proteins were synthesized from mRNAs encoding the polypeptide sequences provided. The SEC was performed at 300 A and PBS as mobile phase. FIG. 14A provides a summary of the results from the SEC-HPLC analysis. The fusion proteins described above were well expressed. For example, FIGS. 12A-12E show examples of data from size exclusion-high-performance liquid chromatography assays of the engineered SIRPα molecules. In FIG. 12A, the fusion protein 1051 corresponds to SEQ ID NO: 4. FIG. 12B shows the results for fusion protein 1053 corresponding to SEQ ID NO: 6. FIG. 12C shows the results for fusion protein 1063 corresponds to SEQ ID NO: 2 (tetramer). FIG. 12D shows the results for fusion protein 1052 corresponding to SEQ ID NO: 5. FIG. 12E shows the results for fusion protein 1054 corresponding to SEQ ID NO: 7. The data are tabulated in FIG. 14A.

### Example 3—Mass Spectroscopy (LC-MS)

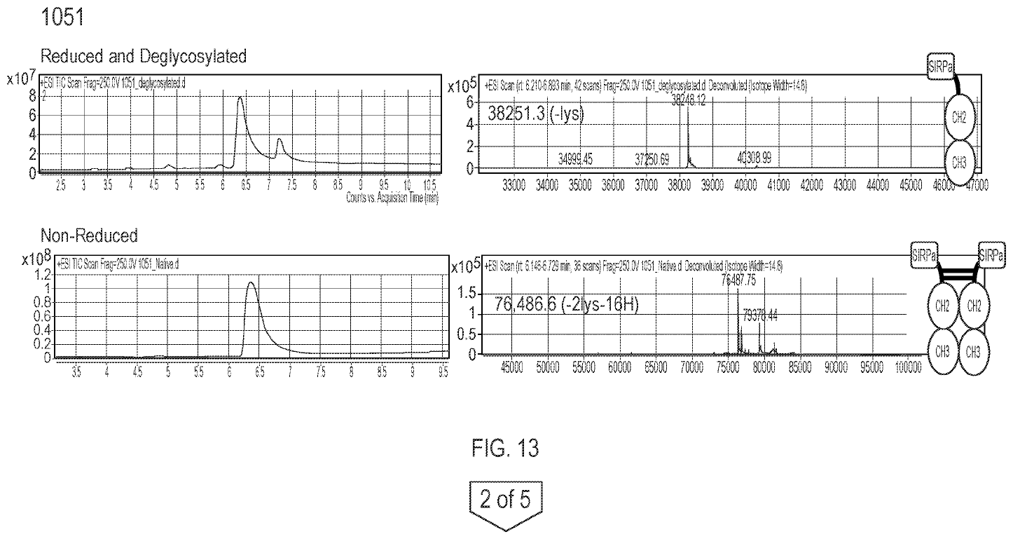

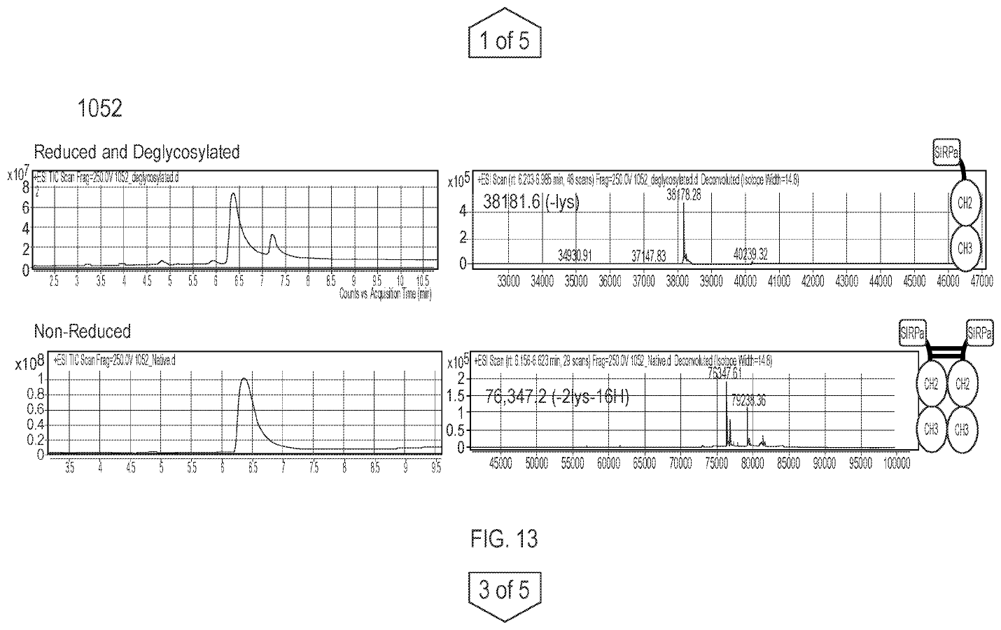

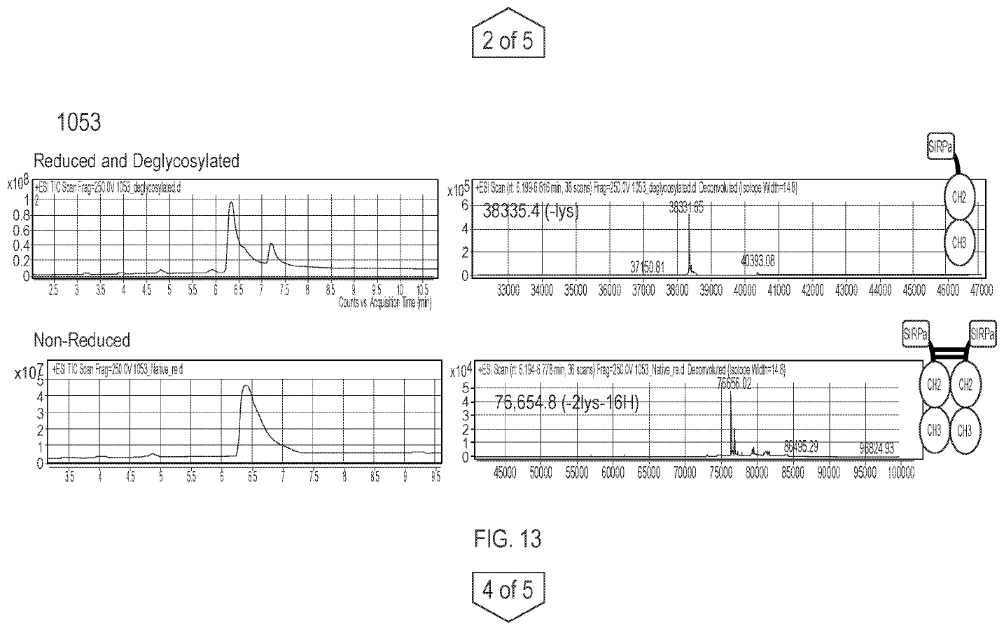

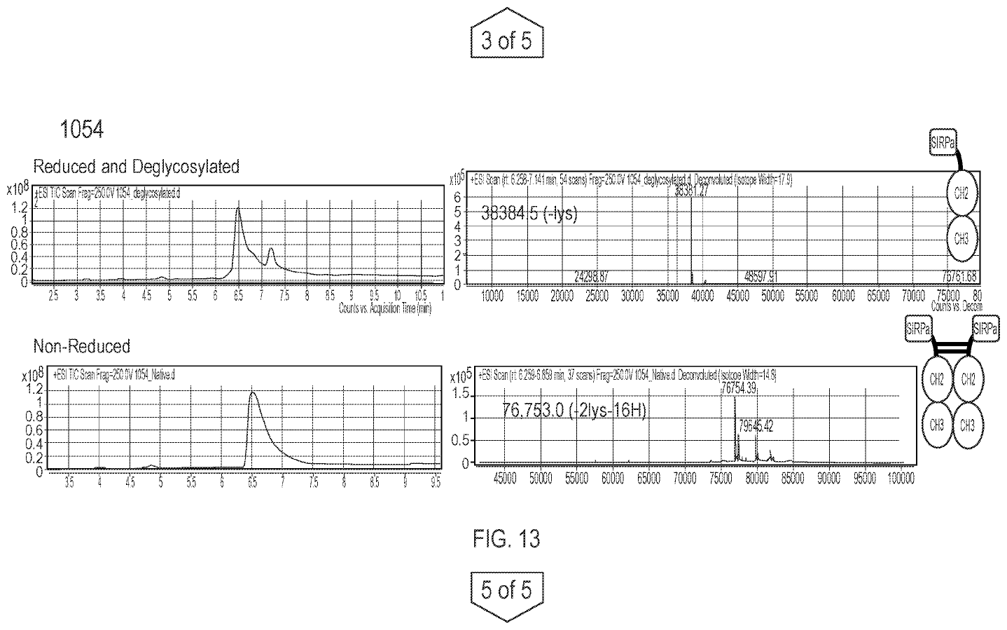

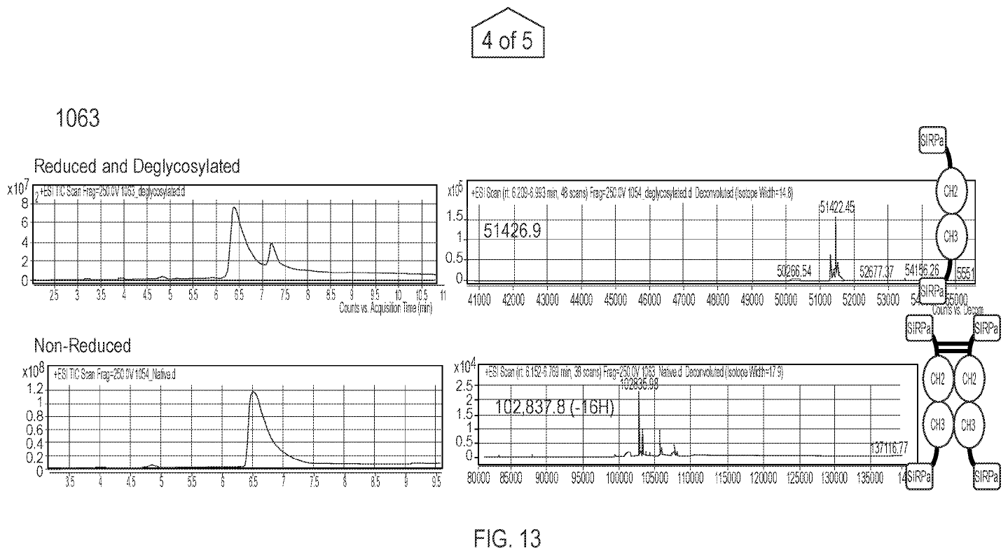

LC-MS was performed using the PLRP-S reverse phase column (1000 A; Sum; 50 mm×2.1 mm; using formic acid and acetonitrile mobile phase system), coupled to the Agilent Quadrupole time-of-flight (Q-TOF) MS system. Protein samples were all deglycosylated and then either treated with reducing agent (Left panel; FIG. 13) or untreated (Right panel; FIG. 13), before queued for LC-MS analysis. Masses were deconvoluted from the main peaks from the total ion chromatograms (TIC) as shown in first columns of the panels. Deconvoluted spectra are shown in second columns of the panels. Fusion protein 1051 (top) corresponds to SEQ ID NO: 4; fusion protein 1052 (second from top) corresponds to SEQ ID NO: 5; fusion protein 1053 (third from top) corresponds to SEQ ID NO: 6; fusion protein 1054 (fourth from top) corresponds to SEQ ID NO: 7; and fusion protein 1063 (bottom) corresponds to SEQ ID NO: 2 (tetramer). Significant purity was seen for all of these fusion proteins.

Identification and purity analysis included mass spectrometry showed that the fusion proteins described herein could be synthesized and were characterized, including testing for efficacy. Even with only a single column purification step, the molecules encoded by the engineered sequences described herein were relatively pure.

FIGS. 14B and 14C are charts tabulating the Labchip mass spectroscopy data described above in FIG. 13.

### Example 4—Antibody-Dependent Cell-Mediated Phagocytosis (ADCP) Assay

SIRPα fusion proteins described herein were assessed for physiological activity using an antibody-dependent cell-mediated phagocytosis (ADCP) reporter assay (Jurkat-Lucia™ NFAT-CD32 Cells; InvivoGen; https://www.invivogen.com/sites/defualt/files/invivogen/products/files/jurkat-lucia_nfat-ed32_tds.pdf) substantially according to the manufacturer's protocol.

ADCP is triggered by the cross-linking between antigen-bound antigen binding site and the Fc receptor CD32A at the surface of myeloid cells (e.g., monocytes, macrophages, and dendritic cells). CD32A receptor cross linking induces NFAT transcription factor translocation into the nucleus where it activates the downstream genes involved in phagocytosis. These interactions induce the phagocytosis of the target cells.

Supernatants from mRNA transfected HEK cells were quantified for SIRPα-FC content using human IgG1 ELISA per the manufacturer's protocol (Thermo CTA #BMS2092). About 100,000 reporter cells (hCD32 NFAT Lucia Jurkat cells, InvivoGen) were added to each well and the cells were cocultured for 18 h. Coculture supernatants were then harvested and serial dilution of the supernatants was performed into complete media.

About 50,000 Raji tumor cells were plated in a round bottom 96 well plate and incubated for 20 min with SIRPα-FC serial dilution to assess Lucia expression using QuantiLuc (InvivoGen) substrate and a luminometer.

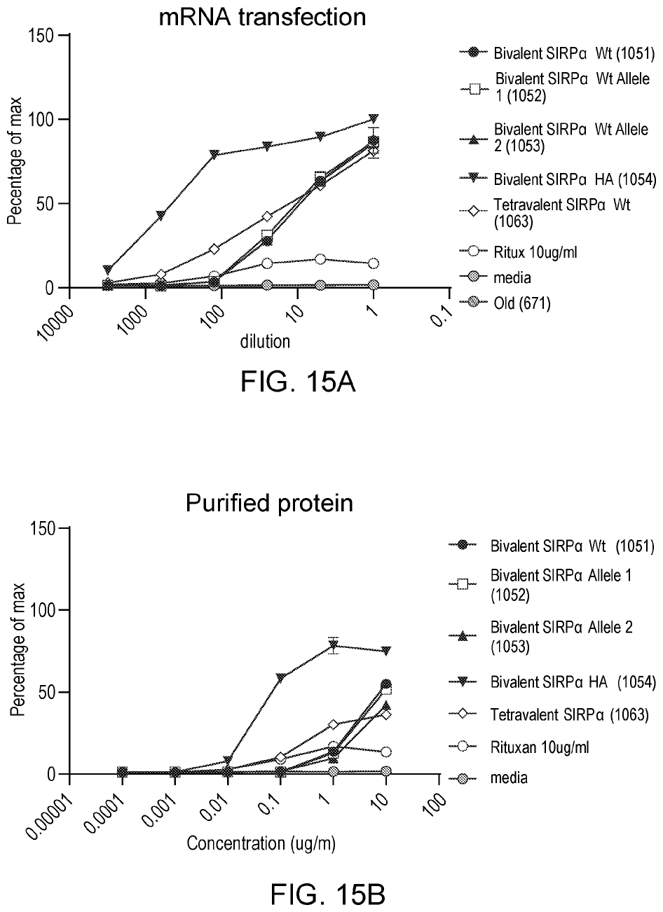

CD32 activation was associated with activity of examples of engineered SIRPα molecules as described herein. FIG. 15A shows data obtained using supernatants from mRNA transfected HEK cells, while FIG. 15B shows data obtained using purified proteins. The data are consistent with the idea that engineered fusion proteins (encoded by mRNAs) described herein prevent the CD47-mediated anti-phagocytotic “don't eat me” signaling to macrophages. The SIRPα fusion proteins resulting from the engineered mRNA provide significant inhibition against CD47 defense for cells that should otherwise be destroyed. The SIRPα fusion proteins resulting from the engineered mRNA provide significant CD32A crosslinking for inducing phagocytosis pathway. For example, the tetravalent WT SIRPα fusion protein showed greater effect at higher dilution than other subject molecules for the transfected study design supporting consideration that the tetravalent SIRPα fusion protein may perform better than bivalent high affinity fusion proteins for lower concentrations.

### Example 5—Antibody-Dependent Cell-Mediated Cytotoxicity (ADCC) Assay

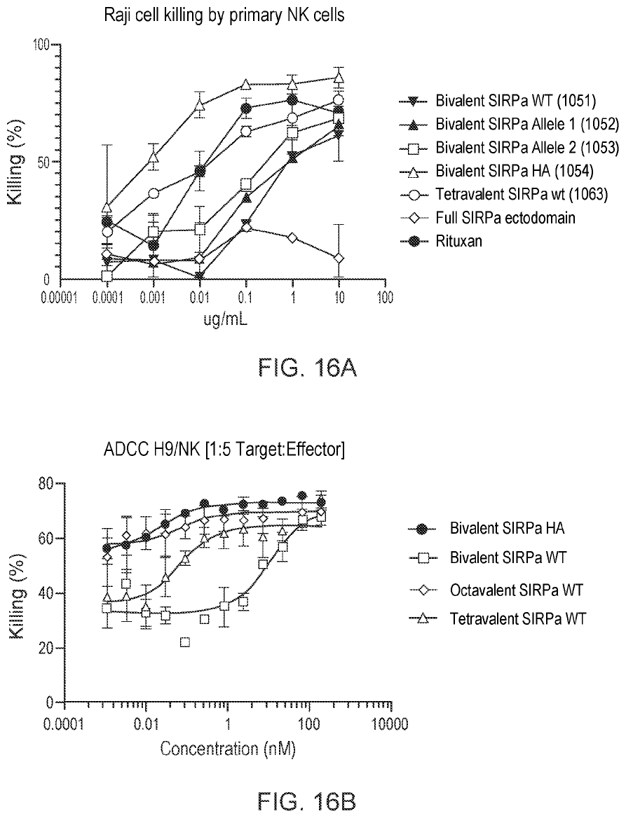

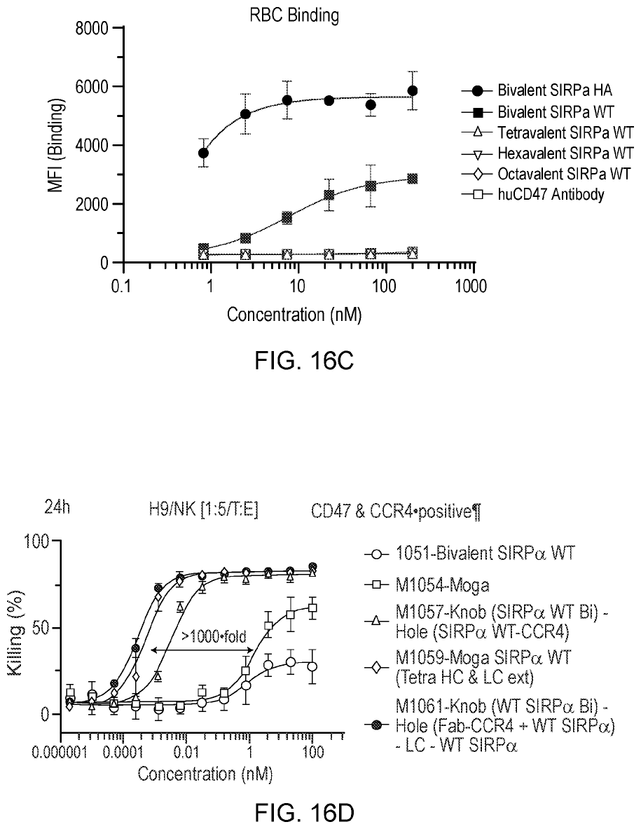

Raji or H9 tumor cells (as indicated) were labeled with CellTrace Violet (Cat #C34557; ThermoFisher) according to the suppliers instructions and seeded in a round bottom 96-well plate at the density of approximately 30,000 cells per well. The cells were then incubated with SIRPα-FC (serially diluted) fusion proteins. Human NK cells were isolated from healthy human PBMCs (STEMCELL Technologies) by negative selection (Cat #17955; StemCell) and cocultured with labeled Raji (FIG. 16A) or H9 (FIG. 16B) tumor cells at a 1:5 target to effector ratio for 24 hours. Cells were then stained with Zombi NIR viability dye (Cat #423106; BioLegend) and tumor cell death was measured by flow cytometry using Cytek. Specific cell lysis was calculated by using the equation: Specific killing (%)=(Observed killing-Baseline killing)/(100-Baseline killing)*100.

About 100,000 reporter cells (hCD16 NFAT Lucia Jurkat cells, InvivoGen) were added to each well and the cells were cocultured for 18 h. Coculture supernatants were then harvested to assess Lucia expression using QuantiLuc (InvivoGen) substrate and a luminometer.

FIGS. 16A and 16B show results of the above-described experiments. FIG. 16A shows data using Raji cells and demonstrates that % killing with wild-type tetravalent fusion protein 1063 (SEQ ID NO: 2) is nearly as effective as that obtained using the high affinity (HA) construct 1054 (SEQ ID NO: 7). As seen in FIG. 16B, experiments conducted using H9 cells showed that (i) the tetravalent design 1063; SEQ ID NO: 2) leads to higher activity than that of the bivalent WT SIRPα (exemplified by 1051; SEQ ID NO: 4), and (ii) the octavalent design leads to activity similar to that of the affinity enhanced SIRPα (HA) (SEQ ID NO: 7; 1054).

However, unlike the bivalent SIRPα HA, the multivalent fusion proteins described herein, including tetravalent, hexavalent and octavalent fusion proteins, show little to no binding to the RBC (FIG. 16C). Binding to RBS is undesirable because it can lead to RBC toxicity and anemia. Therefore, the therapeutic window of the multivalent SIRPα fusion proteins detailed herein is considerably wider than that of, e.g., SIRPα high affinity (HA) constructs.

FIG. 16D shows a comparison of tumor cell killing efficiency between 1051 (Bivalent SIRPα WT) and Mogamulizumab (which selectively binds to and blocks the activity of CCR4) on the one hand, and bivalent aCCR4-SIRPα fusion proteins of the invention on the other. Note that the SIRPα-aCCR4 bispecific fusion proteins of the present invention show substantially improved (>1000 fold) activity in the ADCC killing assay as compared with either 1051 (Bivalent SIRPα WT) or Mogamulizumab (M1054).

### Example 6—Binding to Human RBC In Vitro

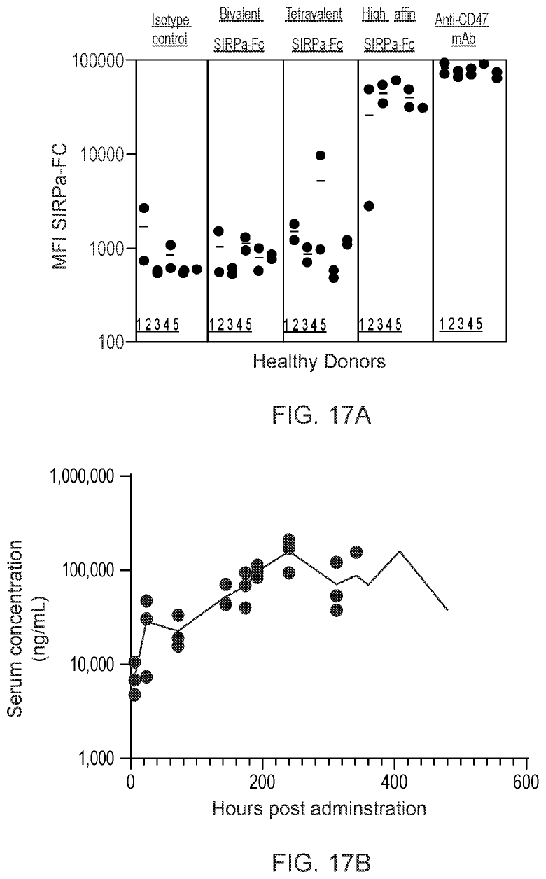

Human red blood cells (Innovative Research) were incubated with supernatants from HEK293 cells that had been transfected with formulated mRNA encoding wildtype tetravalent SIRPα-Fc (1063; SEQ ID NO: 2), wildtype-bivalent SIRPα-Fc (1051; SEQ ID NO: 4), high affinity bivalent SIRPα-Fc (1054; SEQ ID NO: 7) or with anti-CD47 antibody or an isotype control antibody. Red blood cells (RBC) were stained using a goat anti-Human IgG Fc secondary antibody (Cat #12-4998-82; Invitrogen) and RBC binding quantified by flow cytometry using Cytek. The data, shown in FIG. 17A, illustrates that tetravalent SIRPα-Fc retains low binding to healthy cells with lower target density.

### Example 7—Serum Expression of Tetravalent SIRPα-Fa In Vivo

Balb/c mice (n=3/timepoint, Charles River Labs) were dosed intravenously three times one week apart with 15 μg 197ND2-formulated mRNA encoding wildtype tetravalent SIRPα-Fc. Blood was collected by either cheek bleed or cardiac puncture at 6 h, 24 h, 72 h and 7 d post-each dose and processed for serum. Levels of tetravalent SIRPα-Fc in serum were quantified using human IgG ELISA kit (Invitrogen) according to the instructions, using in-house purified tetravalent SIRPα-Fc protein as a standard. The data, shown in FIG. 17B, illustrate high levels of tetravalent SIRPα-Fc protein in serum from IV-delivered mRNA formulated in an amino-lipidated peptoid.

### Example 8—Raii Xenograft Model

The Raji cell line was obtained from ATCC and experiments were performed largely as described in Lapalombella, et al., (2008) Clin Cancer Res. 14(2):569-78. Specifically, the cells were cultured with RPMI-1640 with 10% fetal bovine serum (FBS), maintained in a 37° C. incubator with 5% CO₂. CB17 SCID mice were procured from Charles River (female, 6 to 8 weeks old). The mice were allowed to acclimatize in animal facility for 3 to 7 days. While the animals were acclimatizing, the mice were ear tagged and their body weights will be recorded. Hair at the implantation site was shaved and depilated.

Burkitts lymphoma, Raji cells were cultured with RPMI-1640 with 10% fetal bovine serum (FBS). The cell culture was maintained in a 37° C. incubator with 5% CO₂. On the day of implantation, Raji cells were suspended at a final concentration of 100 million cells/ml in cold HBSS with 50% Matrigel in preparation for implantation.

Each mouse was injected subcutaneously with 10 million Raji cells in the lower right flank. When the average tumor size reached −100 mm³, animals were stratified into 5 groups (8 mice/group) according to tumor volume.

Treatment was initiated once tumors reached a volume of −100 mm³ and administered with 100 μg of proteins in 100 μl volume intravenously (IV) via lateral tail vein every three day (Q3D) for 7 doses for each non-vehicle arm. Anti huCD47 antibody was used as a positive control. All molecules had huFc IgG1 isotype. PBS was used for the Vehicle control. Body weights and tumor measurements were measured at least twice a week and be followed up to 45 days or the tumor volume exceeding 2,000 mm³, whichever was earlier.

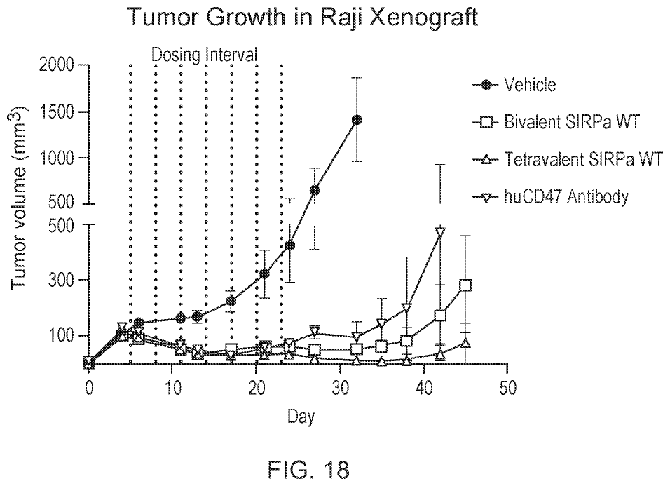

Data from the experiments are shown in FIG. 18. Tetravalent SIRPα demonstrated efficacy in the Raji xenograft mouse tumor model.

### Example 11—General Synthesis of Tertiary Amino Lipidated Cationic Peptoids

General protocols for synthesizing tertiary amino lipidated cationic peptoids disclosed herein can be found in WO2020/069442 and WO2020/069445, each of which is incorporated herein by reference in its entirety. The following example describes the general protocol for synthesis of the tertiary amino lipidated cationic peptoids

All polymers were synthesized using bromoacetic acid and primary amines. An Fmoc-Rink amide resin was used as the solid support. The Fmoc group on the resin was deprotected with 20% (v/v) piperidine-dimethylformamide (DMF). The amino resin was then amidated with bromoacetic acid. The amidation was followed by amination of the α-carbon by nucleophilic displacement of the bromide with a primary amine. The two steps were successively repeated to produce the desired cationic peptide sequence.

All reactions and washings were performed at room temperature unless otherwise noted. Washing of the resin refers to the addition of a wash solvent (usually DMF or dimethylsulfoxide (DMSO)) to the resin, agitating the resin so that a uniform slurry was obtained, followed by thorough draining of the solvent from the resin. Solvents were removed by vacuum filtration through the fritted bottom of the reaction vessel until the resin appeared dry. In all the syntheses, resin slurries were agitated via bubbling argon up through the bottom of the fritted vessel.

Initial Resin Deprotection. A fritted reaction vessel was charged with Fmoc-Rink amide resin. DMF was added to the resin and this solution was agitated to swell the resin. The DMF was then drained. The Fmoc group was removed by adding 20% piperidine in DMF to the resin, agitating the resin, and draining the resin. 20% piperidine in DMF was added to the resin and agitated for 15 minutes and then drained. The resin was then washed with DMF, six times.

Acylation Amidation. The deblocked amine was then acylated by adding bromoacetic acid in DMF to the resin followed by N,N-diisoprooplycarbodiimide (DIC) in DMF. This solution is agitated for 30 minutes at room temperature and then drained. This step was repeated a second time. The resin was then washed with DMF twice and DMSO once. This was one completed reaction cycle.

Nucleophilic Displacement Amination. The acylated resin was treated with the desired primary or secondary amine to undergo nucleophilic displacement at the bromine leaving group on the α-carbon. This acylation/displacement cycle was repeated until the desired peptide sequence was obtained.

Peptide Cleavage from Resin. The dried resin was placed in a glass scintillation vial containing a teflon-coated micro stir bar, and 95% trifluoroacetic acid (TFA) in water is added. The solution was stirred for 20 minutes and then filtered through solid-phase extraction (SPE) column fitted with a polyethylene frit into a polypropylene conical centrifuge tube. The resin was washed with 1 mL 95% TFA. The combined filtrates were then lyophilized three times from 1:1 acetonitrile:water. The lyophilized peptide was redissolved to a concentration of 5 mM in 5% acetonitrile in water.

Purification and Characterization. The redissolved crude peptide was purified by preparative HPLC. The purified peptide was characterized by LC-MS analysis.

### Example 12—Synthesis of Hydroxyethyl-Capped Tertiary Amino Lipidated Cationic Pentoids

Hydroxyethyl-capped lipidated peptoids were synthesized by the submonomer method described in Example 1 with bromoacetic acid and N,N′-diisopropylcarbodiimide (DIC). Polystyrene-supported MBHA Fmoc-protected Rink amide (200 mg representative scale, 0.64 mmol/g loading, Protein Technologies) resin was used as a solid support. For bromoacetylation, resin was combined with a 1:1 mixture of 0.8 M bromoacetic acid and 0.8 M N,N′-diisopropylcarbodiimide (DIC) for 15 minutes. Amine displacement was carried out using a 1M solution of amine in DMF for 45 minutes. Following synthesis, crude peptoids were cleaved from resin using 5 mL of a mixture of 95:2.5:2.5 trifluoroacetic acid (TFA):water:triisopropylsilane for 40 minutes at room temperature. Resin was removed by filtration and the filtrate concentrated using a vacuum centrifuge. The crude peptoids were further purified by reverse-phase flash chromatography (Biotage Selekt) using a C4 column and a gradient from 60-95% ACN/H2O+0.1% TFA. Purity and identity were assayed with a Waters Acquity UPLC system with Acquity Diode Array UV detector and Waters SQD2 mass spectrometer on a Waters Acquity UPLC Peptide BEH C4 Column over a 5-95% gradient. Select peptoids were further purified by preparative Waters Prep150LC system with Waters 2489 UV/Visible Detector on a Waters XBridge BEH300 Prep C4 column using a 40-85% acetonitrile in water with 0.1% TFA gradient over 30 minutes.

### Example 13—General Synthesis of Tertiary Amino Lipidated Cationic Peptoids

General protocols for synthesizing cationic peptoids disclosed herein can be found in WO2020/069442 and WO2020/069445, each of which is incorporated herein by reference in its entirety. The following example describes the general protocol for synthesis of the cationic peptoids

All polymers are synthesized using bromoacetic acid and primary amines. An Fmoc-Rink amide resin is used as the solid support. The Fmoc group on the resin is deprotected with 20% (v/v) piperidine-dimethylformamide (DMF). The amino resin is then amidated with bromoacetic acid. The amidation is followed by amination of the α-carbon by nucleophilic displacement of the bromide with a primary amine. The two steps are successively repeated to produce the desired cationic peptide sequence.

All reactions and washings are performed at room temperature unless otherwise noted. Washing of the resin refers to the addition of a wash solvent (usually DMF or dimethylsulfoxide (DMSO)) to the resin, agitating the resin so that a uniform slurry was obtained, followed by thorough draining of the solvent from the resin. Solvents are removed by vacuum filtration through the fritted bottom of the reaction vessel until the resin appears dry. In all the syntheses, resin slurries are agitated via bubbling argon up through the bottom of the fritted vessel.

Initial Resin Deprotection. A fritted reaction vessel is charged with Fmoc-Rink amide resin. DMF is added to the resin and this solution is agitated to swell the resin. The DMF is then drained. The Fmoc group is removed by adding 20% piperidine in DMF to the resin, agitating the resin, and draining the resin. 20% piperidine in DMF is added to the resin and agitated for 15 minutes and then drained. The resin is then washed with DMF, six times.

Acylation Amidation. The deblocked amine is then acylated by adding bromoacetic acid in DMF to the resin followed by N,N-diisoprooplycarbodiimide (DIC) in DMF. This solution is agitated for 30 minutes at room temperature and then drained. This step is repeated a second time. The resin is then washed with DMF twice and DMSO once. This is one completed reaction cycle.

Nucleophilic Displacement Amination. The acylated resin is treated with the desired primary or secondary amine to undergo nucleophilic displacement at the bromine leaving group on the α-carbon. This acylation/displacement cycle is repeated until the desired peptide sequence is obtained.

Peptide Cleavage from Resin. The dried resin is placed in a glass scintillation vial containing a teflon-coated micro stir bar, and 95% trifluoroacetic acid (TFA) in water is added. The solution is stirred for 20 minutes and then filtered through solid-phase extraction (SPE) column fitted with a polyethylene frit into a polypropylene conical centrifuge tube. The resin is washed with 1 mL 95% TFA. The combined filtrates are then lyophilized three times from 1:1 acetonitrile:water. The lyophilized peptide is redissolved to a concentration of 5 mM in 5% acetonitrile in water.

Purification and Characterization. The redissolved crude peptide is purified by preparative HPLC. The purified peptide is characterized by LC-MS analysis.

### Example 14—Synthesis of Delivery Vehicle Complexes

Synthesis. The hydroxyethyl-capped tertiary amino lipidated peptoids can be combined with polyanionic compounds, such as the mRNA polynucleotides described herein, to form delivery vehicle complexes that can be administered for therapeutic and/or prophylactic purposes in vitro or in vivo. Without being bound to any particular theory, the cationic portion(s) of the amino-lipidated peptoids binds to the negatively-charged phosphodiester backbone of the polyanionic cargo (e.g., nucleic acid cargo) through primarily electrostatic interactions, forming a mixed coacervate complex. Hydrophobic interactions between lipid chains on the hydroxyethyl-capped tertiary amino lipidated peptoids can act to stabilize particle formation and assist with membrane association.

Delivery vehicle complexes can be prepared through any physical and/or chemical methods known in the art to modulate their physical, chemical, and biological properties. These methods typically involve rapid combination of the hydroxyethyl-capped tertiary amino lipidated peptoid in water, or a water-miscible organic solvent, with the oligonucleotide in water or an aqueous buffer solution. These methods can include simple mixing of the components by pipetting, or microfluidic mixing processes such as those involving T-mixers, vortex mixers, or other chaotic mixing structures. Exemplary mixing methods are detailed in, e.g., U.S. Pat. Nos. 11,278,895 and 11,325,122, incorporated herein by reference.

In standard formulations, the hydroxyethyl-capped tertiary amino lipidated peptoid and additional lipids are dissolved in anhydrous ethanol at a concentration of 10 mg/mL to result in solutions that are stable at room temperature. In some implementations, the solutions are stored at −20° C. The nucleic acid cargo is dissolved in DNAse or RNAse-free water at a final concentration of 1-2 mg/mL. These solutions can be stored at −20° C. or −78° C. for extended time periods.

To prepare the delivery vehicle compositions disclosed herein, hydroxyethyl-capped tertiary amino lipidated peptoid and additional lipid components were first pre-mixed in an ethanol phase at the required mass ratios. Nucleic acid cargo(s) were diluted in ethanol and acidic buffer (e.g., 10 mM phosphate/citrate, pH 5.0). Ethanol and aqueous phases were mixed at a 3:1 volume ratio, and then immediately diluted with a 1:1 volume ratio of PBS, resulting in a final mRNA concentration of 0.1 μg/μL. Non-liming exemplary delivery vehicle compositions prepared by the aforementioned method include the compositions listed in Table 3, above (e.g., compositions F2, F6/17, F6/12, and F6/15).

The delivery vehicle compositions were combined with a polyanionic compound, such as the E6/E7 oncogene (e.g., from HPV16, HPV18, a functional fragment thereof, and/or a variant thereof) at the ratios indicated in Table 4 to form delivery vehicle complexes. The w/w in Table 4 is the ratio of the indicated component to the mRNA by mass.

TABLE 4
Delivery Vehicle System Components to Polyanionic Cargo

| | | Anionic or | | |
|---|---|---|---|---|
| | Cationic | Zwitterionic | Neutral Lipid | Shielding |
| Mass Ratio | Component | Component | Component | Component |
| F1A | 5.0 | 0 | 0 | 0.1 |
| F2A | 5.0 | 3.0 | 6.0 | 1.8 |
| F3A | 10 | 2.7 | 5.4 | 1.4 |
| F4A | 5.0 | 0 | 8.0 | 1.8 |
| F5A | 8.5 | 7.0 | 0 | 2.0 |
| F6A | 10 | 0 | 5.4 | 1.4 |
| F1 | 5.0 | 3.0 | 6.0 | 1.8 |
| F2 | 10 | 2.7 | 5.4 | 1.4 |
| F3 | 10 | 2.4 | 6.1 | 1.8 |
| F4 | 10 | 8.0 | 5.6 | 0.69 |
| F5 | 10 | 4.3 | 6.5 | 2.3 |
| F6/12 | 12 | 2.7 | 5.4 | 1.4 |
| F6/15 | 15 | 2.7 | 5.4 | 1.4 |
| F6/17 | 17 | 2.7 | 5.4 | 1.4 |
| F6.1 | 13 | 2.7 | 5.4 | 1.4 |
| F6.2 | 19 | 4.0 | 8.1 | 2.1 |
| F6.3 | 9.7 | 2.7 | 6.7 | 2.1 |

TABLE 5
Efficacy and Toxicology Studies

| | Therapeutic dose in | Therapeutic dose scaled | |
|---|---|---|---|
| Assessments | mouse | for rat | High dose in rat |
| Regimen | 3 doses 7 days apart; | 4 doses in 2 weeks; 30 μg | 4 doses in 2 weeks; 100 μg |
| | ~1-2 μg RNA IT or IM | RNA IM | RNA IM |
| Body weight | No changes compared to | No changes compared to | Reduced weight gain in |
| | control | control | some animals |
| Injection site | None observed | None observed | None observed |
| reactions or other | | | |
| clinical | | | |
| observations | | | |
| Gross organ | Not assessed | None observed | None observed |
| pathology | | | |
| Hematology | Not assessed | No changes compared to | Some transient changes |
| parameters | | control | in hematology; return to |
| | | | baseline by D 30 |
| Clinical | Not assessed | No meaningful changes in | No meaningful changes in |
| chemistries | | serum chemistries | serum chemistries |
| | | compared to control; | compared to control; |
| | | majority remain within | majority remain within |
| | | normal range | normal range |
| Serum cytokines | Not assessed | No changes compared to | Elevated levels in subset |
| | | control | of cytokines; return to |
| | | | baseline by D 30 |

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

SEQUENCES

| SEQ | | | | |
|---|---|---|---|---|
| ID | NTX No. | Fig. | Description | Sequence |
| 1 | | 1 | SIRPα fragment | TEFKSGAGTELSVRAKGG |
| | | | with GG linker | |
| 2 | 1063 | 2B | Tetravalent | EEELQVIQPDKSVSVAAGESAILHCTVTSLIPVGPIQWFRGAGPARELIYNQKEGHFPRVTTVSESTKRENM |
| | | | SIRPα sequence | DFSISISNITPADAGTYYCVKFRKGSPDTEFKSGAGTELSVRAKGGDKTHTCPPCPAPELLGGPSVFLFPPKPK |
| | | | (SIRPα +Fc) WT | DTLMISRTPEVTCVVVDVSHEDPEVKFNWYVDGVEVHNAKTKPREEQYNSTYRVVSVLTVLHQDWLNGK |
| | | | | EYKCKVSNKALPAPIEKTISKAKGQPREPQVYTLPPSREEMTKNQVSLTCLVKGFYPSDIAVEWESNGQPEN |
| | | | | NYKTTPPVLDSDGSFFLYSKLTVDKSRWQQGNVFSCSVMHEALHNHYTQKSLSLSPGGGGGSGGGGSEEE |
| | | | | LQVIQPDKSVSVAAGESAILHCTVTSLIPVGPIQWFRGAGPARELIYNQKEGHFPRVTTVSESTKRENMDFSI |
| | | | | SISNITPADAGTYYCVKFRKGSPDTEFKSGAGTELSVRAK |
| 3 | 1064 | 2B | Tetravalent | MDMRVPAQLLGLLLLWLRGARCEEELQIIQPDKSVLVAAGETATLRCTITSLFPVGPIQWFRGAGPGRVLIY |
| | | | SIRPα_high | NQRQGPFPRVTTVSDTTKRNNMDFSIRIGNITPADAGTYYCIKFRKGSPDDVEFKSGAGTELSVRAKGGDK |
| | | | affinity | THTCPPCPAPELLGGPSVFLFPPKPKDTLMISRTPEVTCVVVDVSHEDPEVKFNWYVDGVEVHNAKTKPRE |
| | | | | EQYNSTYRVVSVLTVLHQDWLNGKEYKCKVSNKALPAPIEKTISKAKGQPREPQVYTLPPSREEMTKNQVS |
| | | | | LTCLVKGFYPSDIAVEWESNGQPENNYKTTPPVLDSDGSFFLYSKLTVDKSRWQQGNVFSCSVMHEALHN |
| | | | | HYTQKSLSLSPGGGGGSGGGGSEEELQIIQPDKSVLVAAGETATLRCTITSLFPVGPIQWFRGAGPGRVLIY |
| | | | | NQRQGPFPRVTTVSDTTKRNNMDFSIRIGNITPADAGTYYCIKFRKGSPDDVEFKSGAGTELSVRAK |
| 4 | 1051 | 2A | Bivalent SIRPα | EEELQVIQPDKSVSVAAGESAILHCTVTSLIPVGPIQWFRGAGPARELIYNQKEGHFPRVTTVSESTKRENM |
| | | | Wt allele 1 | DFSISISNITPADAGTYYCVKFRKGSPDTEFKSGAGTELSVRAKPSDKTHTCPPCPAPELLGGPSVFLFPPKPK |
| | | | | DTLMISRTPEVTCVVVDVSHEDPEVKFNWYVDGVEVHNAKTKPREEQYNSTYRVVSVLTVLHQDWLNGK |
| | | | | EYKCKVSNKALPAPIEKTISKAKGQPREPQVYTLPPSREEMTKNQVSLTCLVKGFYPSDIAVEWESNGQPEN |
| | | | | NYKTTPPVLDSDGSFFLYSKLTVDKSRWQQGNVFSCSVMHEALHNHYTQKSLSLSPGK |
| 5 | 1052 | 2A | Bivalent SIRPα | EEELQVIQPDKSVSVAAGESAILHCTVTSLIPVGPIQWFRGAGPARELIYNQKEGHFPRVTTVSESTKRENM |
| | | | WT allele 1 | DFSISISNITPADAGTYYCVKFRKGSPDTEFKSGAGTELSVRAKGGDKTHTCPPCPAPELLGGPSVFLFPPKPK |
| | | | | DTLMISRTPEVTCVVVDVSHEDPEVKFNWYVDGVEVHNAKTKPREEQYNSTYRVVSVLTVLHQDWLNGK |
| | | | | EYKCKVSNKALPAPIEKTISKAKGQPREPQVYTLPPSREEMTKNQVSLTCLVKGFYPSDIAVEWESNGQPEN |
| | | | | NYKTTPPVLDSDGSFFLYSKLTVDKSRWQQGNVFSCSVMHEALHNHYTQKSLSLSPGK |
| 6 | 1053 | 2A | Bivalent SIRPα | EEELQVIQPDKSVLVAAGETATLRCTATSLIPVGPIQWFRGAGPGRELIYNQKEGHFPRVTTVSDLTKRNN |
| | | | WT allele 2 | MDFSIRIGNITPADAGTYYCVKFRKGSPDDVEFKSGAGTELSVRAKGGDKTHTCPPCPAPELLGGPSVFLFP |
| | | | | PKPKDTLMISRTPEVTCVVVDVSHEDPEVKFNWYVDGVEVHNAKTKPREEQYNSTYRVVSVLTVLHQDW |
| | | | | LNGKEYKCKVSNKALPAPIEKTISKAKGQPREPQVYTLPPSREEMTKNQVSLTCLVKGFYPSDIAVEWESNG |
| | | | | QPENNYKTTPPVLDSDGSFFLYSKLTVDKSRWQQGNVFSCSVMHEALHNHYTQKSLSLSPGK |
| 7 | 1054 | 2A | Bivalent SIRPα | EEELQIIQPDKSVLVAAGETATLRCTITSLFPVGPIQWFRGAGPGRVLIYNQRQGPFPRVTTVSDTTKRNNM |
| | | | WT high affinity | DFSIRIGNITPADAGTYYCIKFRKGSPDDVEFKSGAGTELSVRAKGGDKTHTCPPCPAPELLGGPSVFLFPPKP |
| | | | allele 2 | KDTLMISRTPEVTCVVVDVSHEDPEVKFNWYVDGVEVHNAKTKPREEQYNSTYRVVSVLTVLHQDWLNG |
| | | | | KEYKCKVSNKALPAPIEKTISKAKGQPREPQVYTLPPSREEMTKNQVSLTCLVKGFYPSDIAVEWESNGQPE |
| | | | | NNYKTTPPVLDSDGSFFLYSKLTVDKSRWQQGNVFSCSVMHEALHNHYTQKSLSLSPGK |
| 8 | 1057 | 2A | huSIRPα IgV | EEELQIIQPDKSVLVAAGETATLRCTITSLFPVGPIQWFRGAGPGRVLIYNQRQGPFPRVTTVSDTTKRNNM |
| | | | High Affinity | DFSIRIGNITPADAGTYYCIKFRKGSPDDVEFKSGAGTELSVRAKGGDKTHTCPPCPAPELLGGPSVFLFPPKP |
| | | | allele 2 linker | KDTLMISRTPEVTCVVVDVSHEDPEVKFNWYVDGVEVHNAKTKPREEQYNSTYRVVSVLTVLHQDWLNG |
| | | | G2 huIgG1 Fc | KEYKCKVSNKALPAPIEKTISKAKGQPREPQVYTLPPSREEMTKNQVSLSCAVKGFYPSDIAVEWESNGQPE |
| | | | hole | NNYKTTPPVLDSDGSFFLVSKLTVDKSRWQQGNVFSCSVMHEALHNHYTQKSLSLSPGK |
| 9 | 1058 | 3A | M1058 aCCR4 | DVLMTQSPLSLPVTPGEPASISCRSSRNIVHINGDTYLEWYLQKPGQSPQLLIYKVSNRFSGVPDRFSGSGS |
| | | | Mogamab scFv | GTDFTLKISRVEAEDVGVYYCFQGSLLPWTFGQGTKVEIKRGGGGSGGGGSGGGGSEVQLVESGGDLVQP |
| | | | VL-VH linker G4 | GRSLRLSCAASGFIFSNYGMSWVRQAPGKGLEWVATISSASTYSYYPDSVKGRFTISRDNAKNSLYLQMNS |
| | | | huIgG1 Fc knob | LRVEDTALYYCGRHSDGNFAFGYWGQGTLVTVSSGGGGDKTHTCPPCPAPELLGGPSVFLFPPKPKDTLM |
| | | | | ISRTPEVTCVVVDVSHEDPEVKFNWYVDGVEVHNAKTKPREEQYNSTYRVVSVLTVLHQDWLNGKEYKCK |
| | | | | VSNKALPAPIEKTISKAKGQPREPQVYTLPPSREEMTKNQVSLWCLVKGFYPSDIAVEWESNGQPENNYKT |
| | | | | TPPVLDSDGSFFLYSKLTVDKSRWQQGNVFSCSVMHEALHNHYTQKSLSLSPGK |
| 10 | 1060 | 3B, | aCCR4 | EVQLVESGGDLVQPGRSLRLSCAASGFIFSNYGMSWVRQAPGKGLEWVATISSASTYSYYPDSVKGRFTIS |
| | | 3C | Mogamab scFv | RDNAKNSLYLQMNSLRVEDTALYYCGRHSDGNFAFGYWGQGTLVTVSSGGGGSGGGGSGGGGSDVLM |
| | | | VH-VL linker G4 | TQSPLSLPVTPGEPASISCRSSRNIVHINGDTYLEWYLQKPGQSPQLLIYKVSNRFSGVPDRFSGSGSGTDFT |
| | | | huIgG1 Fc knob | LKISRVEAEDVGVYYCFQGSLLPWTFGQGTKVEIKRGGGGDKTHTCPPCPAPELLGGPSVFLFPPKPKDTL |
| | | | linker 2G4S- | MISRTPEVTCVVVDVSHEDPEVKFNWYVDGVEVHNAKTKPREEQYNSTYRVVSVLTVLHQDWLNGKEYK |
| | | | huSIRPα IgV | CKVSNKALPAPIEKTISKAKGQPREPQVYTLPPSREEMTKNQVSLWCLVKGFYPSDIAVEWESNGQPENNY |
| | | | allele 1 | KTTPPVLDSDGSFFLYSKLTVDKSRWQQGNVFSCSVMHEALHNHYTQKSLSLSPGGGGGSGGGGSEEELQ |
| | | | | VIQPDKSVSVAAGESAILHCTVTSLIPVGPIQWFRGAGPARELIYNQKEGHFPRVTTVSESTKRENMDFSISI |
| | | | | SNITPADAGTYYCVKFRKGSPDTEFKSGAGTELSVRAK |
| 11 | 1062 | 3B | huSIRPα IgV | EEELQVIQPDKSVSVAAGESAILHCTVTSLIPVGPIQWFRGAGPARELIYNQKEGHFPRVTTVSESTKRENM |
| | | | allele 1 linker | DFSISISNITPADAGTYYCVKFRKGSPDTEFKSGAGTELSVRAKGGDKTHTCPPCPAPELLGGPSVFLFPPKPK |
| | | | G2-huIgG1 Fc | DTLMISRTPEVTCVVVDVSHEDPEVKFNWYVDGVEVHNAKTKPREEQYNSTYRVVSVLTVLHQDWLNGK |
| | | | hole-linker | EYKCKVSNKALPAPIEKTISKAKGQPREPQVYTLPPSREEMTKNQVSLSCAVKGFYPSDIAVEWESNGQPEN |
| | | | 2G4S huSIRPα | NYKTTPPVLDSDGSFFLVSKLTVDKSRWQQGNVFSCSVMHEALHNHYTQKSLSLSPGGGGGSGGGGSEE |
| | | | IgV allele 1 | ELQVIQPDKSVSVAAGESAILHCTVTSLIPVGPIQWFRGAGPARELIYNQKEGHFPRVTTVSESTKRENMDF |
| | | | | SISISNITPADAGTYYCVKFRKGSPDTEFKSGAGTELSVRAK |
| 12 | 1467 | 2C | SIRPα | EEELQVIQPDKSVSVAAGESAILHCTVTSLIPVGPIQWFRGAGPARELIYNQKEGHFPRVTTVSESTKRENM |
| | | | Hexavalent | DFSISISNITPADAGTYYCVKFRKGSPDTEFKSGAGTELSVRAKGGGGSGGGGSEEELQVIQPDKSVSVAAG |
| | | | | ESAILHCTVTSLIPVGPIQWFRGAGPARELIYNQKEGHFPRVTTVSESTKRENMDFSISISNITPADAGTYYCV |
| | | | | KFRKGSPDTEFKSGAGTELSVRAKGGDKTHTCPPCPAPELLGGPSVFLFPPKPKDTLMISRTPEVTCVVVDV |
| | | | | SHEDPEVKFNWYVDGVEVHNAKTKPREEQYNSTYRVVSVLTVLHQDWLNGKEYKCKVSNKALPAPIEKTIS |
| | | | | KAKGQPREPQVYTLPPSREEMTKNQVSLTCLVKGFYPSDIAVEWESNGQPENNYKTTPPVLDSDGSFFLYS |
| | | | | KLTVDKSRWQQGNVFSCSVMHEALHNHYTQKSLSLSPGGGGGSGGGGSEEELQVIQPDKSVSVAAGESA |
| | | | | ILHCTVTSLIPVGPIQWFRGAGPARELIYNQKEGHFPRVTTVSESTKRENMDFSISISNITPADAGTYYCVKFR |
| | | | | KGSPDTEFKSGAGTELSVRAK |
| 13 | 1468 | 2D | SIRPα | EEELQVIQPDKSVSVAAGESAILHCTVTSLIPVGPIQWFRGAGPARELIYNQKEGHFPRVTTVSESTKRENM |
| | | | Octavalent | DFSISISNITPADAGTYYCVKFRKGSPDTEFKSGAGTELSVRAKGGGGSGGGGSEEELQVIQPDKSVSVAAG |
| | | | | ESAILHCTVTSLIPVGPIQWFRGAGPARELIYNQKEGHFPRVTTVSESTKRENMDFSISISNITPADAGTYYCV |
| | | | | KFRKGSPDTEFKSGAGTELSVRAKGGDKTHTCPPCPAPELLGGPSVFLFPPKPKDTLMISRTPEVTCVVVDV |
| | | | | SHEDPEVKFNWYVDGVEVHNAKTKPREEQYNSTYRVVSVLTVLHQDWLNGKEYKCKVSNKALPAPIEKTIS |
| | | | | KAKGQPREPQVYTLPPSREEMTKNQVSLTCLVKGFYPSDIAVEWESNGQPENNYKTTPPVLDSDGSFFLYS |
| | | | | KLTVDKSRWQQGNVFSCSVMHEALHNHYTQKSLSLSPGGGGGSGGGGSEEELQVIQPDKSVSVAAGESA |
| | | | | ILHCTVTSLIPVGPIQWFRGAGPARELIYNQKEGHFPRVTTVSESTKRENMDFSISISNITPADAGTYYCVKFR |
| | | | | KGSPDTEFKSGAGTELSVRAKGGGGSGGGGSEEELQVIQPDKSVSVAAGESAILHCTVTSLIPVGPIQWFRG |
| | | | | AGPARELIYNQKEGHFPRVTTVSESTKRENMDFSISISNITPADAGTYYCVKFRKGSPDTEFKSGAGTELSVR |
| | | | | AK |
| 14 | 1343 | 4A | Mogamulizumab | EVQLVESGGDLVQPGRSLRLSCAASGFIFSNYGMSWVRQAPGKGLEWVATISSASTYSYYPDSVKGRFTIS |
| | | | (VH)-CH1-CH2- | RDNAKNSLYLQMNSLRVEDTALYYCGRHSDGNFAFGYWGQGTLVTVSSASTKGPSVFPLAPSSKSTSGGT |
| | | | CH3 | AALGCLVKDYFPEPVTVSWNSGALTSGVHTFPAVLQSSGLYSLSSVVTVPSSSLGTQTYICNVNHKPSNTKV |
| | | | | DKKVEPKSCDKTHTCPPCPAPELLGGPSVFLFPPKPKDTLMISRTPEVTCVVVDVSHEDPEVKFNWYVDGV |
| | | | | EVHNAKTKPREEQYNSTYRVVSVLTVLHQDWLNGKEYKCKVSNKALPAPIEKTISKAKGQPREPQVYTLPPS |
| | | | | REEMTKNQVSLTCLVKGFYPSDIAVEWESNGQPENNYKTTPPVLDSDGSFFLYSKLTVDKSRWQQGNVFS |
| | | | | CSVMHEALHNHYTQKSLSLSPG |
| 15 | 1344 | 4B | Mogamulizumab | EVQLVESGGDLVQPGRSLRLSCAASGFIFSNYGMSWVRQAPGKGLEWVATISSASTYSYYPDSVKGRFTIS |
| | | | (VH)-CH1-CH2- | RDNAKNSLYLQMNSLRVEDTALYYCGRHSDGNFAFGYWGQGTLVTVSSASTKGPSVFPLAPSSKSTSGGT |
| | | | CH3-HA SIRPα | AALGCLVKDYFPEPVTVSWNSGALTSGVHTFPAVLQSSGLYSLSSVVTVPSSSLGTQTYICNVNHKPSNTKV |
| | | | | DKKVEPKSCDKTHTCPPCPAPELLGGPSVFLFPPKPKDTLMISRTPEVTCVVVDVSHEDPEVKFNWYVDGV |
| | | | | EVHNAKTKPREEQYNSTYRVVSVLTVLHQDWLNGKEYKCKVSNKALPAPIEKTISKAKGQPREPQVYTLPPS |
| | | | | REEMTKNQVSLTCLVKGFYPSDIAVEWESNGQPENNYKTTPPVLDSDGSFFLYSKLTVDKSRWQQGNVFS |
| | | | | CSVMHEALHNHYTQKSLSLSPGGGGGSGGGGSEEELQIIQPDKSVLVAAGETATLRCTITSLFPVGPIQWFR |
| | | | | GAGPGRVLIYNQRQGPFPRVTTVSDTTKRNNMDFSIRIGNITPADAGTYYCIKFRKGSPDDVEFKSGAGTEL |
| | | | | SVRAK |
| 16 | | 3E | Mogamulizumab | EVQLVESGGDLVQPGRSLRLSCAASGFIFSNYGMSWVRQAPGKGLEWVATISSASTYSYYPDSVKGRFTIS |
| | | | (VH)-CH1-CH2- | RDNAKNSLYLQMNSLRVEDTALYYCGRHSDGNFAFGYWGQGTLVTVSSASTKGPSVFPLAPSSKSTSGGT |
| | | | CH3-WT SIRPα | AALGCLVKDYFPEPVTVSWNSGALTSGVHTFPAVLQSSGLYSLSSVVTVPSSSLGTQTYICNVNHKPSNTKV |
| | | | | DKKVEPKSCDKTHTCPPCPAPELLGGPSVFLFPPKPKDTLMISRTPEVTCVVVDVSHEDPEVKFNWYVDGV |
| | | | | EVHNAKTKPREEQYNSTYRVVSVLTVLHQDWLNGKEYKCKVSNKALPAPIEKTISKAKGQPREPQVYTLPPS |
| | | | | REEMTKNQVSLTCLVKGFYPSDIAVEWESNGQPENNYKTTPPVLDSDGSFFLYSKLTVDKSRWQQGNVFS |
| | | | | CSVMHEALHNHYTQKSLSLSPGGGGGSGGGGSEEELQVIQPDKSVSVAAGESAILHCTVTSLIPVGPIQWF |
| | | | | RGAGPARELIYNQKEGHFPRVTTVSESTKRENMDFSISISNITPADAGTYYCVKFRKGSPDTEFKSGAGTELS |
| | | | | VRAK |
| 17 | 1477 | 4ABC | Mogamulizumab | DVLMTQSPLSLPVTPGEPASISCRSSRNIVHINGDTYLEWYLQKPGQSPQLLIYKVSNRFSGVPDRFSGSGS |
| | | | (VL)-CK | GTDFTLKISRVEAEDVGVYYCFQGSLLPWTFGQGTKVEIKRTVAAPSVFIFPPSDEQLKSGTASVVCLLNNFY |
| | | | | PREAKVQWKVDNALQSGNSQESVTEQDSKDSTYSLSSTLTLSKADYEKHKVYACEVTHQGLSSPVTKSFNR |
| | | | | GEC |
| 18 | 1478 | 3E, | Mogamulizumab | DVLMTQSPLSLPVTPGEPASISCRSSRNIVHINGDTYLEWYLQKPGQSPQLLIYKVSNRFSGVPDRFSGSGS |
| | | 3F | (VL)-CK-WT | GTDFTLKISRVEAEDVGVYYCFQGSLLPWTFGQGTKVEIKRTVAAPSVFIFPPSDEQLKSGTASVVCLLNNFY |
| | | | SIRP | PREAKVQWKVDNALQSGNSQESVTEQDSKDSTYSLSSTLTLSKADYEKHKVYACEVTHQGLSSPVTKSFNR |
| | | | | GECGGGGSGGGGSEEELQVIQPDKSVSVAAGESAILHCTVTSLIPVGPIQWFRGAGPARELIYNQKEGHFP |
| | | | | RVTTVSESTKRENMDFSISISNITPADAGTYYCVKFRKGSPDTEFKSGAGTELSVRAK |
| 19 | 1348 | 3B, | SP-SIRP(wt)- | EEELQVIQPDKSVSVAAGESAILHCTVTSLIPVGPIQWFRGAGPARELIYNQKEGHFPRVTTVSESTKRENM |
| | | 3F | knob (CH2- | DFSISISNITPADAGTYYCVKFRKGSPDTEFKSGAGTELSVRAKGGDKTHTCPPCPAPELLGGPSVFLFPPKPK |
| | | | CH3)-SIRP (WT) | DTLMISRTPEVTCVVVDVSHEDPEVKFNWYVDGVEVHNAKTKPREEQYNSTYRVVSVLTVLHQDWLNGK |
| | | | | EYKCKVSNKALPAPIEKTISKAKGQPREPQVYTLPPSREEMTKNQVSLWCLVKGFYPSDIAVEWESNGQPE |
| | | | | NNYKTTPPVLDSDGSFFLYSKLTVDKSRWQQGNVFSCSVMHEALHNHYTQKSLSLSPGGGGGSGGGGSE |
| | | | | EELQVIQPDKSVSVAAGESAILHCTVTSLIPVGPIQWFRGAGPARELIYNQKEGHFPRVTTVSESTKRENMD |
| | | | | FSISISNITPADAGTYYCVKFRKGSPDTEFKSGAGTELSVRAK |
| 20 | 1349 | 3F | Mogamulizumab | EVQLVESGGDLVQPGRSLRLSCAASGFIFSNYGMSWVRQAPGKGLEWVATISSASTYSYYPDSVKGRFTIS |
| | | | (VH)-Hole-WT | RDNAKNSLYLQMNSLRVEDTALYYCGRHSDGNFAFGYWGQGTLVTVSSASTKGPSVFPLAPSSKSTSGGT |
| | | | SIRPα | AALGCLVKDYFPEPVTVSWNSGALTSGVHTFPAVLQSSGLYSLSSVVTVPSSSLGTQTYICNVNHKPSNTKV |
| | | | | DKKVEPKSCDKTHTCPPCPAPELLGGPSVFLFPPKPKDTLMISRTPEVTCVVVDVSHEDPEVKFNWYVDGV |
| | | | | EVHNAKTKPREEQYNSTYRVVSVLTVLHQDWLNGKEYKCKVSNKALPAPIEKTISKAKGQPREPQVYTLPPS |
| | | | | REEMTKNQVSLSCAVKGFYPSDIAVEWESNGQPENNYKTTPPVLDSDGSFFLVSKLTVDKSRWQQGNVFS |
| | | | | CSVMHEALHNHYTQKSLSLSPGGGGGSGGGGSEEELQVIQPDKSVSVAAGESAILHCTVTSLIPVGPIQWF |
| | | | | RGAGPARELIYNQKEGHFPRVTTVSESTKRENMDFSISISNITPADAGTYYCVKFRKGSPDTEFKSGAGTELS |
| | | | | VRAK |
| 21 | 1351 | | Mogamulizumab | EVQLVESGGDLVQPGRSLRLSCAASGFIFSNYGMSWVRQAPGKGLEWVATISSASTYSYYPDSVKGRFTIS |
| | | | (VH)-knob-WT | RDNAKNSLYLQMNSLRVEDTALYYCGRHSDGNFAFGYWGQGTLVTVSSASTKGPSVFPLAPSSKSTSGGT |
| | | | SIRPα | AALGCLVKDYFPEPVTVSWNSGALTSGVHTFPAVLQSSGLYSLSSVVTVPSSSLGTQTYICNVNHKPSNTKV |
| | | | | DKKVEPKSCDKTHTCPPCPAPELLGGPSVFLFPPKPKDTLMISRTPEVTCVVVDVSHEDPEVKFNWYVDGV |
| | | | | EVHNAKTKPREEQYNSTYRVVSVLTVLHQDWLNGKEYKCKVSNKALPAPIEKTISKAKGQPREPQVYTLPPS |
| | | | | REEMTKNQVSLWCLVKGFYPSDIAVEWESNGQPENNYKTTPPVLDSDGSFFLYSKLTVDKSRWQQGNVF |
| | | | | SCSVMHEALHNHYTQKSLSLSPGGGGGSGGGGSEEELQVIQPDKSVSVAAGESAILHCTVTSLIPVGPIQW |
| | | | | FRGAGPARELIYNQKEGHFPRVTTVSESTKRENMDFSISISNITPADAGTYYCVKFRKGSPDTEFKSGAGTEL |
| | | | | SVRAK |
| 22 | 1054-2 | | SIRPα binding | EEELQIIQPDKSVLVAAGETATLRCTITSLFPVGPIQWFRGAGPGRVLIYNQRQGPFPRVTTVSDTTKRNNM |
| | | | site (High | DFSIRIGNITPADAGTYYCIKFRKGSPDDVEFKSGAGTELSVRAK |
| | | | affinity | |
| | | | example) (e.g., | |
| | | | high-affinity | |
| | | | CD47 binding | |
| | | | domain) | |
| 23 | 1051-2 | | SIRPα binding | EEELQVIQPDKSVSVAAGESAILHCTVTSLIPVGPIQWFRGAGPARELIYNQKEGHFPRVTTVSESTKRENM |
| | | | site (WT) (e.g., | DFSISISNITPADAGTYYCVKFRKGSPDTEFKSGAGTELSVRAK |
| | | | CD47 binding | |
| | | | domain) | |
| 24 | 1055 | | huSIRPα IgV | EEELQVIQPDKSVSVAAGESAILHCTVTSLIPVGPIQWFRGAGPARELIYNQKEGHFPRVTTVSESTKRENM |
| | | | allele 1 linker | DFSISISNITPADAGTYYCVKFRKGSPDTEFKSGAGTELSVRAKGGDKTHTCPPCPAPELLGGPSVFLFPPKPK |
| | | | G2 huIgG1 Fc | DTLMISRTPEVTCVVVDVSHEDPEVKFNWYVDGVEVHNAKTKPREEQYNSTYRVVSVLTVLHQDWLNGK |
| | | | hole | EYKCKVSNKALPAPIEKTISKAKGQPREPQVYTLPPSREEMTKNQVSLSCAVKGFYPSDIAVEWESNGQPEN |
| | | | | NYKTTPPVLDSDGSFFLVSKLTVDKSRWQQGNVFSCSVMHEALHNHYTQKSLSLSPGK |
| 25 | 1056 | | aCCR4 | EVQLVESGGDLVQPGRSLRLSCAASGFIFSNYGMSWVRQAPGKGLEWVATISSASTYSYYPDSVKGRFTIS |
| | | | Mogamab scFv | RDNAKNSLYLQMNSLRVEDTALYYCGRHSDGNFAFGYWGQGTLVTVSSGGGGSGGGGSGGGGSDVLM |
| | | | VH-VL linker G4 | TQSPLSLPVTPGEPASISCRSSRNIVHINGDTYLEWYLQKPGQSPQLLIYKVSNRFSGVPDRFSGSGSGTDFT |
| | | | huIgG1 Fc knob | LKISRVEAEDVGVYYCFQGSLLPWTFGQGTKVEIKRGGGGDKTHTCPPCPAPELLGGPSVFLFPPKPKDTL |
| | | | | MISRTPEVTCVVVDVSHEDPEVKFNWYVDGVEVHNAKTKPREEQYNSTYRVVSVLTVLHQDWLNGKEYK |
| | | | | CKVSNKALPAPIEKTISKAKGQPREPQVYTLPPSREEMTKNQVSLWCLVKGFYPSDIAVEWESNGQPENNY |
| | | | | KTTPPVLDSDGSFFLYSKLTVDKSRWQQGNVFSCSVMHEALHNHYTQKSLSLSPGK |
| 26 | 1059 | | huIgG1 Fc hole- | DKTHTCPPCPAPELLGGPSVFLFPPKPKDTLMISRTPEVTCVVVDVSHEDPEVKFNWYVDGVEVHNAKTKP |
| | | | linker 2G4S | REEQYNSTYRVVSVLTVLHQDWLNGKEYKCKVSNKALPAPIEKTISKAKGQPREPQVYTLPPSREEMTKNQ |
| | | | huSIRPα IgV | VSLSCAVKGFYPSDIAVEWESNGQPENNYKTTPPVLDSDGSFFLVSKLTVDKSRWQQGNVFSCSVMHEAL |
| | | | allele 1 | HNHYTQKSLSLSPGGGGGSGGGGSEEELQVIQPDKSVSVAAGESAILHCTVTSLIPVGPIQWFRGAGPARE |
| | | | | LIYNQKEGHFPRVTTVSESTKRENMDFSISISNITPADAGTYYCVKFRKGSPDTEFKSGAGTELSVRAK |
| 27 | 1061 | | aCCR4 | DVLMTQSPLSLPVTPGEPASISCRSSRNIVHINGDTYLEWYLQKPGQSPQLLIYKVSNRFSGVPDRFSGSGS |
| | | | Mogamab scFv | GTDFTLKISRVEAEDVGVYYCFQGSLLPWTFGQGTKVEIKRGGGGSGGGGSGGGGSEVQLVESGGDLVQP |
| | | | VL-VH linker G4 | GRSLRLSCAASGFIFSNYGMSWVRQAPGKGLEWVATISSASTYSYYPDSVKGRFTISRDNAKNSLYLQMNS |
| | | | huIgG1 Fc knob | LRVEDTALYYCGRHSDGNFAFGYWGQGTLVTVSSGGGGDKTHTCPPCPAPELLGGPSVFLFPPKPKDTLM |
| | | | linker 2G4S- | ISRTPEVTCVVVDVSHEDPEVKFNWYVDGVEVHNAKTKPREEQYNSTYRVVSVLTVLHQDWLNGKEYKCK |
| | | | huSIRPα IgV | VSNKALPAPIEKTISKAKGQPREPQVYTLPPSREEMTKNQVSLWCLVKGFYPSDIAVEWESNGQPENNYKT |
| | | | allele 1 | TPPVLDSDGSFFLYSKLTVDKSRWQQGNVFSCSVMHEALHNHYTQKSLSLSPGGGGGSGGGGSEEELQVI |
| | | | | QPDKSVSVAAGESAILHCTVTSLIPVGPIQWFRGAGPARELIYNQKEGHFPRVTTVSESTKRENMDFSISISN |
| | | | | ITPADAGTYYCVKFRKGSPDTEFKSGAGTELSVRAK |
| 28 | | | aCCR4 | EVQLVESGGDLVQPGRSLRLSCAASGFIFSNYGMSWVRQAPGKGLEWVATISSASTYSYYPDSVKGRFTIS |
| | | | Mogamab scFv | RDNAKNSLYLQMNSLRVEDTALYYCGRHSDGNFAFGYWGQGTLVTVSSGGGGSGGGGSGGGGSDVLM |
| | | | VH-VL linker G4 | TQSPLSLPVTPGEPASISCRSSRNIVHINGDTYLEWYLQKPGQSPQLLIYKVSNRFSGVPDRFSGSGSGTDFT |
| | | | huIgG1 Fc linker | LKISRVEAEDVGVYYCFQGSLLPWTFGQGTKVEIKRGGGGDKTHTCPPCPAPELLGGPSVFLFPPKPKDTL |
| | | | 2G4S-huSIRPα | MISRTPEVTCVVVDVSHEDPEVKFNWYVDGVEVHNAKTKPREEQYNSTYRVVSVLTVLHQDWLNGKEYK |
| | | | IgV allele 1 | CKVSNKALPAPIEKTISKAKGQPREPQVYTLPPSREEMTKNQVSLTCLVKGFYPSDIAVEWESNGQPENNYK |
| | | | | TTPPVLDSDGSFFLYSKLTVDKSRWQQGNVFSCSVMHEALHNHYTQKSLSLSPGGGGGSGGGGSEEELQV |
| | | | | IQPDKSVSVAAGESAILHCTVTSLIPVGPIQWFRGAGPARELIYNQKEGHFPRVTTVSESTKRENMDFSISIS |
| | | | | NITPADAGTYYCVKFRKGSPDTEFKSGAGTELSVRAK |
| 29 | 1559 | | SIRPα- | MDMRVPAQLLGLLLLWLRGARCEEELQVIQPDKSVSVAAGESAILHCTVTSLIPVGPIQWFRGAGPARELIY |
| | | | Mogamulizumab | NQKEGHFPRVTTVSESTKRENMDFSISISNITPADAGTYYCVKFRKGSPDTEFKSGAGTELSVRAKGGGGSG |
| | | | Light Chain | GGGSGGGGSDVLMTQSPLSLPVTPGEPASISCRSSRNIVHINGDTYLEWYLQKPGQSPQLLIYKVSNRFSGV |
| | | | | PDRFSGSGSGTDFTLKISRVEAEDVGVYYCFQGSLLPWTFGQGTKVEIKRTVAAPSVFIFPPSDEQLKSGTAS |
| | | | | VVCLLNNFYPREAKVQWKVDNALQSGNSQESVTEQDSKDSTYSLSSTLTLSKADYEKHKVYACEVTHQGLS |
| | | | | SPVTKSFNRGEC |
| 30 | 1560 | | SIRPα WT | MDMRVPAQLLGLLLLWLRGARCEEELQVIQPDKSVSVAAGESAILHCTVTSLIPVGPIQWFRGAGPARELIY |
| | | | Mogamulizumab | NQKEGHFPRVTTVSESTKRENMDFSISISNITPADAGTYYCVKFRKGSPDTEFKSGAGTELSVRAKGGGGSG |
| | | | Heavy Chain | GGGSGGGGSEVQLVESGGDLVQPGRSLRLSCAASGFIFSNYGMSWVRQAPGKGLEWVATISSASTYSYYP |
| | | | | DSVKGRFTISRDNAKNSLYLQMNSLRVEDTALYYCGRHSDGNFAFGYWGQGTLVTVSSASTKGPSVFPLA |
| | | | | PSSKSTSGGTAALGCLVKDYFPEPVTVSWNSGALTSGVHTFPAVLQSSGLYSLSSVVTVPSSSLGTQTYICNV |
| | | | | NHKPSNTKVDKKVEPKSCDKTHTCPPCPAPELLGGPSVFLFPPKPKDTLMISRTPEVTCVVVDVSHEDPEVK |
| | | | | FNWYVDGVEVHNAKTKPREEQYNSTYRVVSVLTVLHQDWLNGKEYKCKVSNKALPAPIEKTISKAKGQPR |
| | | | | EPQVYTLPPSREEMTKNQVSLTCLVKGFYPSDIAVEWESNGQPENNYKTTPPVLDSDGSFFLYSKLTVDKSR |
| | | | | WQQGNVFSCSVMHEALHNHYTQKSLSLSPG |
| 31 | 1561 | | SSIRPα a WT | MDMRVPAQLLGLLLLWLRGARCEEELQVIQPDKSVSVAAGESAILHCTVTSLIPVGPIQWFRGAGPARELIY |
| | | | Mogamulizumab | NQKEGHFPRVTTVSESTKRENMDFSISISNITPADAGTYYCVKFRKGSPDTEFKSGAGTELSVRAKGGGGSG |
| | | | Heavy Chain | GGGSGGGGSEVQLVESGGDLVQPGRSLRLSCAASGFIFSNYGMSWVRQAPGKGLEWVATISSASTYSYYP |
| | | | SIRPα WT | DSVKGRFTISRDNAKNSLYLQMNSLRVEDTALYYCGRHSDGNFAFGYWGQGTLVTVSSASTKGPSVFPLA |
| | | | | PSSKSTSGGTAALGCLVKDYFPEPVTVSWNSGALTSGVHTFPAVLQSSGLYSLSSVVTVPSSSLGTQTYICNV |
| | | | | NHKPSNTKVDKKVEPKSCDKTHTCPPCPAPELLGGPSVFLFPPKPKDTLMISRTPEVTCVVVDVSHEDPEVK |
| | | | | FNWYVDGVEVHNAKTKPREEQYNSTYRVVSVLTVLHQDWLNGKEYKCKVSNKALPAPIEKTISKAKGQPR |
| | | | | EPQVYTLPPSREEMTKNQVSLTCLVKGFYPSDIAVEWESNGQPENNYKTTPPVLDSDGSFFLYSKLTVDKSR |
| | | | | WQQGNVFSCSVMHEALHNHYTQKSLSLSPGGGGGSGGGGSEEELQVIQPDKSVSVAAGESAILHCTVTSL |
| | | | | IPVGPIQWFRGAGPARELIYNQKEGHFPRVTTVSESTKRENMDFSISISNITPADAGTYYCVKFRKGSPDTEF |
| | | | | KSGAGTELSVRAK |
| 32 | 1562 | | Mogamulizumab | MDMRVPAQLLGLLLLWLRGARCDVLMTQSPLSLPVTPGEPASISCRSSRNIVHINGDTYLEWYLQKPGQS |
| | | | Light chain | PQLLIYKVSNRFSGVPDRFSGSGSGTDFTLKISRVEAEDVGVYYCFQGSLLPWTFGQGTKVEIKRTVAAPSVF |
| | | | 4(G4S) SIRP WT | IFPPSDEQLKSGTASVVCLLNNFYPREAKVQWKVDNALQSGNSQESVTEQDSKDSTYSLSSTLTLSKADYEK |
| | | | (Longer) | HKVYACEVTHQGLSSPVTKSFNRGECGGGGSGGGGSGGGGSGGGGSEEELQVIQPDKSVSVAAGESAILH |
| | | | | CTVTSLIPVGPIQWFRGAGPARELIYNQKEGHFPRVTTVSESTKRENMDFSISISNITPADAGTYYCVKFRKG |
| | | | | SPDTEFKSGAGTELSVRAK |
| 33 | 1563 | | Mogamulizumab | MDMRVPAQLLGLLLLWLRGARCDVLMTQSPLSLPVTPGEPASISCRSSRNIVHINGDTYLEWYLQKPGQS |
| | | | Light chain- | PQLLIYKVSNRFSGVPDRFSGSGSGTDFTLKISRVEAEDVGVYYCFQGSLLPWTFGQGTKVEIKRTVAAPSVF |
| | | | Mogamulizumab | IFPPSDEQLKSGTASVVCLLNNFYPREAKVQWKVDNALQSGNSQESVTEQDSKDSTYSLSSTLTLSKADYEK |
| | | | scFv | HKVYACEVTHQGLSSPVTKSFNRGECGGGGSGGGGSGGGGSEVQLVESGGDLVQPGRSLRLSCAASGFIF |
| | | | | SNYGMSWVRQAPGKGLEWVATISSASTYSYYPDSVKGRFTISRDNAKNSLYLQMNSLRVEDTALYYCGRH |
| | | | | SDGNFAFGYWGQGTLVTVSSGGGGSGGGGSGGGGSDVLMTQSPLSLPVTPGEPASISCRSSRNIVHING |
| | | | | DTYLEWYLQKPGQSPQLLIYKVSNRFSGVPDRFSGSGSGTDFTLKISRVEAEDVGVYYCFQGSLLPWTFGQ |
| | | | | GTKVEIK |
| 34 | 1564 | | Mogamulizumab | MDMRVPAQLLGLLLLWLRGARCEVQLVESGGDLVQPGRSLRLSCAASGFIFSNYGMSWVRQAPGKGLE |
| | | | scFv- | WVATISSASTYSYYPDSVKGRFTISRDNAKNSLYLQMNSLRVEDTALYYCGRHSDGNFAFGYWGQGTLVT |
| | | | Mogamulizumab | VSSGGGGSGGGGSGGGGSDVLMTQSPLSLPVTPGEPASISCRSSRNIVHINGDTYLEWYLQKPGQSPQLLI |
| | | | Light Chain | YKVSNRFSGVPDRFSGSGSGTDFTLKISRVEAEDVGVYYCFQGSLLPWTFGQGTKVEIKGGGGSGGGGSD |
| | | | | VLMTQSPLSLPVTPGEPASISCRSSRNIVHINGDTYLEWYLQKPGQSPQLLIYKVSNRFSGVPDRFSGSGSGT |
| | | | | DFTLKISRVEAEDVGVYYCFQGSLLPWTFGQGTKVEIKRTVAAPSVFIFPPSDEQLKSGTASVVCLLNNFYPR |
| | | | | EAKVQWKVDNALQSGNSQESVTEQDSKDSTYSLSSTLTLSKADYEKHKVYACEVTHQGLSSPVTKSFNRGE |
| | | | | C |
| 35 | 1540 | 2C | SIRPα WT | MDMRVPAQLLGLLLLWLRGARCEEELQVIQPDKSVSVAAGESAILHCTVTSLIPVGPIQWFRGAGPARELIY |
| | | | Hexavalent | NQKEGHFPRVTTVSESTKRENMDFSISISNITPADAGTYYCVKFRKGSPDTEFKSGAGTELSVRAKGGGGSS |
| | | | Longer Linker | SASTDKTHTGGGSEEELQVIQPDKSVSVAAGESAILHCTVTSLIPVGPIQWFRGAGPARELIYNQKEGHFPR |
| | | | | VTTVSESTKRENMDFSISISNITPADAGTYYCVKFRKGSPDTEFKSGAGTELSVRAKGGDKTHTCPPCPAPEL |
| | | | | LGGPSVFLFPPKPKDTLMISRTPEVTCVVVDVSHEDPEVKFNWYVDGVEVHNAKTKPREEQYNSTYRVVS |
| | | | | VLTVLHQDWLNGKEYKCKVSNKALPAPIEKTISKAKGQPREPQVYTLPPSREEMTKNQVSLTCLVKGFYPSD |
| | | | | IAVEWESNGQPENNYKTTPPVLDSDGSFFLYSKLTVDKSRWQQGNVFSCSVMHEALHNHYTQKSLSLSPG |
| | | | | GGGGSGGGGSEEELQVIQPDKSVSVAAGESAILHCTVTSLIPVGPIQWFRGAGPARELIYNQKEGHFPRVT |
| | | | | TVSESTKRENMDFSISISNITPADAGTYYCVKFRKGSPDTEFKSGAGTELSVRAK |
| 36 | 1350 | | SIRPα (WT)-Fc | MDMRVPAQLLGLLLLWLRGARCEEELQVIQPDKSVSVAAGESAILHCTVTSLIPVGPIQWFRGAGPARELIY |
| | | | hole-SIR SIRPα | NQKEGHFPRVTTVSESTKRENMDFSISISNITPADAGTYYCVKFRKGSPDTEFKSGAGTELSVRAKGGDKTH |
| | | | a (WT) | TCPPCPAPELLGGPSVFLFPPKPKDTLMISRTPEVTCVVVDVSHEDPEVKFNWYVDGVEVHNAKTKPREEQ |
| | | | | YNSTYRVVSVLTVLHQDWLNGKEYKCKVSNKALPAPIEKTISKAKGQPREPQVYTLPPSREEMTKNQVSLSC |
| | | | | AVKGFYPSDIAVEWESNGQPENNYKTTPPVLDSDGSFFLVSKLTVDKSRWQQGNVFSCSVMHEALHNHY |
| | | | | TQKSLSLSPGGGGGSGGGGSEEELQVIQPDKSVSVAAGESAILHCTVTSLIPVGPIQWFRGAGPARELIYNQ |
| | | | | KEGHFPRVTTVSESTKRENMDFSISISNITPADAGTYYCVKFRKGSPDTEFKSGAGTELSVRAK |
| 37 | | | 5′UTR | cauagaagucgagacacagccacuaaguaagccacc |
| 38 | | | 3′UTR | gcgccgccuccgggacagugcacccaggcugcggccccucccccguccuggagguuccccagccccacuuaccgcuuaaugcg |
| | | | | ccaauaaaccaaugaacgaagc |
| 39 | 1348 | | Exemplary RNA | auggauaugagaguacccgcccaacuguugggcuugcugcugcuguggcuuagaggggccagaugcgaggaggagcugca |
| | (1836) | | sequence | gguaauucagccagauaagagcgucagcguggcggcaggggagagcgcgaucuugcauuguacagucacaucacugauuc |
| | | | (minus UTRs) | cugugggcccuauucagugguuucggggugcaggccccgccagagagcugaucuauaaccaaaaagaggggcacuucccgc |
| | | | encoding an | ggguaacaaccguuucggagaguacaaagagagaaaacauggauuucagcauuucgaucucaaauauuacccccgccgau |
| | | | amino acid | gccggcaccuacuauugcgugaaguuccggaaagguagcccugacacugaguucaagaguggggcugguacagagcuguc |
| | | | sequence | agucagagccaagggaggagauaaaacucauaccugcccuccuugccccgccccagaguugcuggggggccccagcguauu |
| | | | containing the | ucuguuuccgcccaaaccuaaagauacccugaugaucucgcggacgccagagguuaccugcgucgucguugauguauccca |
| | | | amino acid | cgaggaucccgaagugaaauuuaauugguacguugacggugucgaagugcacaaugccaagacaaaaccccgggaggagca |
| | | | sequence | auauaauagcaccuacagagucgucuccguauugacuguacugcaccaagauuggcugaauggaaaggaauacaaaugua |
| | | | represented by | agguauccaauaaggcuuuaccggcaccuauugagaagaccauauccaaagccaaaggucaacccagggaaccccaaguuu |
| | | | SEQ ID NO: 19 | auacucugccuccaagcagggaggagaugaccaagaaucaggucagccuguggugucucgugaaaggcuuuuaucccagc |
| | | | | gauauugccguugagugggaaucgaaugggcagcccgaaaauaauuacaaaaccaccccucccguccuggauagcgauggu |
| | | | | agcuuuuuucuguacuccaaauugaccguggauaagucuagauggcagcagggcaauguguuuagcugcucuguuaugc |
| | | | | augaggcucugcacaaucacuauacgcagaaaagccugucccugaguccuggcggagggggcggauccggagguggcgguu |
| | | | | ccgaagaagaguugcagguaauccagccugauaaaagugugucggucgcggccggcgagagugcaauucugcacugcaccg |
| | | | | ugaccagucuuauaccgguaggucccauucagugguucagaggcgccggcccugccagggaauugauuuacaaucagaag |
| | | | | gagggacacuuucccagagugacgaccgucagcgaauccacuaaaagggagaacauggauuuuucuaucuccauuagcaa |
| | | | | caucaccccggccgacgcuggcacguauuauugugugaaauuucgaaagggcucccccgauacagaguuuaaaagcggugc |
| | | | | aggaaccgagcugaguguccgggcgaagugauga |
| 40 | 1349 | | Exemplary RNA | auggauaugcgggugccugcccagcugcugggccugcugcugcuguggcugagaggcgcccggugcgagguccagcuggua |
| | | | sequence | gagagcggaggcgaccuggugcagccuggcagaagccuuagacugagcugcgccgcaagcggcuuuaucuucucuaacuac |
| | | | (minus UTRs) | ggcaugagcugggucagacaggccccuggaaagggccuggaauggguggccaccaucagcucugccagcaccuacucauau |
| | | | encoding an | uacccugacagcgugaagggcagauucaccaucagccgggacaacgccaaaaauagccuguaccugcaaaugaacagccucc |
| | | | amino acid | gggucgaggacaccgcccuguacuacugcggccggcacucugauggaaauuucgccuucggcuacuggggccagggaacac |
| | | | sequence | uggugacagugucgucagccagcaccaagggacccagcguguucccccuggccccuagcuccaaguccaccagcggcggaac |
| | | | containing the | agccgcccugggcugucuggugaaagacuacuuccccgagcccgucaccguguccuggaacagcggcgcccucaccagcggc |
| | | | amino acid | gugcauaccuuucccgcuguucugcagagcagcggacuguauucucugagcagcguggugaccgugccuagcuccagccug |
| | | | sequence | ggcacccaaacauacaucugcaacgugaaccauaagccuagcaacaccaagguggacaagaagguggaaccuaagucuugc |
| | | | represented by | gacaagacccacaccuguccuccgugccccgccccugagcuccugggaggcccuagcguguuucuguucccuccuaaaccua |
| | | | SEQ ID NO: 20 | aggacacccugaugaucagccgcaccccugaggugacaugcguggucguggacgugagucacgaggauccugaagugaagu |
| | | | | uuaacugguacguggacggaguggaagugcacaaugccaagacaaaacccagagaagagcaguacaacagcacauacagag |
| | | | | uggugagugugcugaccgugcugcaccaggacuggcugaacggcaaggaauacaagugcaaggugaguaacaaggcccugc |
| | | | | cugcuccuauugagaagacgaucagcaaggccaagggccagccuagggagccucagguguacacccugccuccauccagaga |
| | | | | agagaugacaaagaaccaggugagccucagcugugccgugaagggcuucuaccccucugauaucgccguggaaugggaguc |
| | | | | uaauggacagcccgagaacaacuacaagacaaccccuccugugcuggacagcgacggcagcuucuuccuggugucuaaacu |
| | | | | gacaguggacaaguccagauggcagcagggcaacguguucagcugcagcgugaugcacgaggcccugcacaaccacuacacc |
| | | | | cagaaaucucugagccugucuccaggaggcggcggcggcucugguggcggcggcucugaggaagaacugcaagucauccag |
| | | | | ccugauaaaucggucagcgucgccgcuggcgaguccgcuauccugcacugcaccgugaccagccugauccccgugggcccga |
| | | | | uccagugguuccggggcgcaggacccgcuagagagcucaucuacaaccagaaggaaggccacuucccaagagugacaacagu |
| | | | | uagcgagagcaccaagagagaaaacauggacuucagcauuucuaucagcaauaucaccccugcugaugccgggacauacua |
| | | | | uugugugaaguucagaaagggcagcccagauacagaguucaagagcggcgcuggcaccgagcuguccgugcgggccaaaug |
| | | | | auaa |
| 41 | 1478 | | Exemplary RNA | auggacaugagagugccagcucagcuguugggccugcugcugcuguggcugagaggcgcccggugcgacgugcugaugacc |
| | | | sequence | cagagcccucugucccugccugugacaccuggcgagccugcuagcaucagcugcagaagcagccgcaacaucgugcauauca |
| | | | (minus UTRs) | acggcgacaccuaccuggaaugguaccugcagaagcccggccagucuccccagcugcucaucuauaaggucuccaauagau |
| | | | encoding an | ucagcggcgugcccgauagauuuagcggcucuggcagcggcacagauuuuacccugaaaaucagcagaguggaagccgagg |
| | | | amino acid | acgugggaguguacuacugcuuccagggcagccugcugcccuggaccuucggccagggaacaaagguugagaucaagagaa |
| | | | sequence | ccgucgccgccccuagcguguucaucuucccuccaucugaugagcaacugaaguccggaaccgccagcguggugugccugc |
| | | | containing the | ugaacaacuucuaccccagagaagcuaaagugcaguggaagguggacaacgcccugcagagcggaaauucucaggagagcg |
| | | | amino acid | ugaccgagcaggacagcaaggacaguaccuacagccugagcagcacccugacacugucuaaagccgacuacgagaagcacaa |
| | | | sequence | gguguacgccugcgaggugacccaccagggccuguccagcccugugaccaaaagcuucaaccggggcgaaugcggcggaggc |
| | | | represented by | ggcagcgguggcggcggcagugaagaggaacugcaagugauccaaccugauaagagcgucucuguggccgcuggagaaagc |
| | | | SEQ ID NO: 18 | gccauccugcacuguacagugacgagccuuauccccgugggccccauccagugguuccggggagccggaccugccagagagc |
| | | | | ugaucuacaaccagaaagagggccacuucccuagagugaccaccgugucugagucuacaaagcgggaaaacauggacuuua |
| | | | | gcauuucuaucagcaacauuaccccugcagaugccggcacauacuauuguguuaaguucaggaagggcuccccugacaccg |
| | | | | aauucaagagcggcgccggcacagagcugagcgugcgggccaagugauga |
| 42 | 1343 | | Exemplary RNA | auggacaugcgggugcccgcucagcugcugggacugcugcuccuguggcugcgcggagccagaugcgaggugcagcugguu |
| | | | sequence | gaaagcggcggcgaccugguucaacccggcagaagccugagacugucuugcgccgccagcggauucaucuucagcaacuacg |
| | | | (minus UTRs) | gcaugagcugggugcggcaggccccuggcaagggccuggaaugggucgccacaaucagcagcgccucuaccuacagcuacua |
| | | | encoding an | ccccgacagcgugaaaggcagauucaccaucagcagagauaacgccaagaacagccuguaccugcagaugaacucucuccgg |
| | | | amino acid | guggaagauaccgcccuguauuacugcggcaggcauucugauggcaacuucgccuucggcuacuggggccaaggcacccug |
| | | | sequence | gugaccgugucuucugccagcaccaagggcccuagcgucuuuccucuggccccuagcagcaagagcacaagcggaggcacag |
| | | | containing the | cugcucuggguugucuggucaaggacuacuuccccgagcccgugaccguguccuggaacagcggcgcucugacaagcggag |
| | | | amino acid | ugcacacuuuuccugcugugcugcaaagcagcggccuguacagccugucaucuguggucaccgugccuaguagcucucucg |
| | | | sequence | gaacccagaccuacaucugcaacgugaaccacaagccuuccaauaccaagguggacaagaagguggagccuaaaagcugcg |
| | | | represented by | acaagacacacacauguccuccgugcccugcuccugagcugcugggggucccucuguguuccuguucccaccaaagcccaa |
| | | | SEQ ID NO: 14 | ggauacccugaugaucagccggaccccugaggugacguguguggugguggacgugagccacgaggaccccgaggugaaguu |
| | | | | caacugguacguggacggcguggaagugcacaaugccaaaacaaagccaagagaagagcaguacaacuccaccuauagagu |
| | | | | ggucuccgugcugaccgugcugcaccaggacuggcugaacggcaaggaauacaaaugcaagguguccaacaaggcccugccc |
| | | | | gccccuaucgagaagacaauuucuaaagccaagggccagcccagagagccucagguguauacccugccuccuagcagagag |
| | | | | gaaaugaccaaaaaccaggugucccuuacaugccuggugaaaggcuucuacccuucugauaucgccguggaaugggagag |
| | | | | caauggccagccugagaacaacuacaagaccaccccuccagugcuggacagcgacggcagcuucuuccuguacucuaagcug |
| | | | | accguggauaagucccgguggcagcagggcaauguguuuuccuguagcgugaugcacgaggcccugcacaaccacuacaca |
| | | | | cagaagagccugagccugagcccuggcugauaa |
| 43 | 1345 | | Exemplary RNA | auggacaugagagugccagcccagcugcugggacugcugcuccuguggcugcgaggagccagaugugaagugcagcuggug |
| | | | sequence | gaaagcggcggcgaccugguccagccuggcagaagccugcggcugaguugugccgccagcggcuucaucuucagcaacuacg |
| | | | (minus UTRs) | gcaugagcugggugcggcaggcccccggcaagggccuggaaugggucgccaccaucagcagcgccucuaccuauagcuacua |
| | | | encoding an | uccugauagcguuaagggaagauucaccaucagccgcgacaacgccaagaacagccuguaccugcagaugaacagccugag |
| | | | amino acid | agucgaagauaccgcccuguacuacugcggccggcacucugauggaaauuucgccuucggcuacuggggccagggcacgcu |
| | | | sequence | ggugacagucagcucugcuagcacaaaaggaccuucuguuuucccucuggccccaucuagcaagagcacaucugguggcac |
| | | | containing the | cgccgcccugggcugccuggugaaggacuacuuccccgagcccgugacugugagcuggaacagcggcgccuugacaagcggc |
| | | | amino acid | gugcacaccuucccagcugugcugcaaagcagcggccuguacucucuguccucuguugugacagugccuucuuccucucug |
| | | | sequence | ggcacacagaccuacaucugcaacgugaaccacaagcccucuaauaccaagguggacaaaaagguggaaccuaagagcugc |
| | | | represented by | gacaagacacacacauguccuccaugccccgcaccugagcugcugggggccccagcguguuucuguuucccccgaagccua |
| | | | SEQ ID NO: 16 | aggacacccugaugaucagccggaccccugaagugaccugcgugguaguggacgugucccacgaggaccccgaggugaaau |
| | | | | uuaacugguacguggauggcguggaggugcacaacgccaaaacaaaaccuagagaggaacaguacaacagcaccuauagag |
| | | | | uggucagcgugcugaccgugcugcaccaggacuggcugaacggcaaagaguacaagugcaaggugagcaacaaggcccugc |
| | | | | cugcucccaucgagaagaccaucagcaaggccaagggacaaccuagagagccccagguuuacacacugccuccuaguaggga |
| | | | | ggaaaugaccaagaaccaggugucccugacaugccuggucaaaggcuuuuaccccuccgauaucgccguggaaugggaguc |
| | | | | caacggccagccugaaaacaacuacaagaccaccccuccagugcuggacagcgacggcagcuucuuccuguacuccaagcug |
| | | | | accguggacaagagcagauggcagcagggcaauguguuuagcugcagcgugaugcacgaggcccugcauaaucacuacacc |
| | | | | cagaagucccugagccucucuccuggaggcggcggaggaucuggcggaggcggcagcgaggaagagcugcaggugauccagc |
| | | | | cugacaaaagcgugucuguggccgcuggcgagagcgccauccugcacugcaccgugaccucccugauccccgugggcccuau |
| | | | | ucagugguuccggggcgccggaccugcuagagagcugaucuacaaccaaaaggaaggccacuucccuagagugaccaccgu |
| | | | | guccgagagcaccaaaagagagaacauggauuucagcaucagcaucagcaauaucaccccugcugaugccggcacauacua |
| | | | | cugugugaaguucagaaagggcagcccugacaccgaguucaagucuggagccgguacagagcuguccgugcgggccaagug |
| | | | | auaa |
| 44 | 1063 | | Exemplary RNA | auggauaugcggguccccgcccagcugcugggccugcugcugcuguggcugagaggagcccggugcgaggaggaacugcag |
| | | | sequence | gucauccagccugacaagucuguuucuguggccgcuggcgaaucugccauccuucacugcaccgugacaagccugaucccg |
| | | | (minus UTRs) | gugggaccuauucaaugguuccggggcgccggccccgccagagagcugaucuacaaccagaaagagggccacuuuccacgcg |
| | | | encoding an | ugacaaccgugagcgaguccaccaagcgggagaauauggacuucagcaucagcaucagcaacauuacaccugcugaugccg |
| | | | amino acid | gcacauacuacugcgugaaauucagaaagggcagcccugauaccgaguucaagagcggagccggcaccgagcugucugugc |
| | | | sequence | gggccaagggcggcgacaagacccacacaugcccccccugcccugcuccugaacugcuggggggccaucuguguuccuguu |
| | | | containing the | ccccccuaagccuaaagacacccugaugaucuccaggaccccugaggugaccugugugguggucgacgugagccacgaaga |
| | | | amino acid | uccugagguuaaguucaacugguacguggacggcguggaagugcacaacgccaagaccaaaccuagagaagagcaguacaa |
| | | | sequence | cagcaccuauagagugguguccgugcugacagugcugcaucaggacuggcucaacggcaaggaauacaagugcaagguguc |
| | | | represented by | uaacaaggcacugccugccccuaucgagaagaccaucagcaaggccaaaggccagccuagagaaccccagguguacacacug |
| | | | SEQ ID NO: 2 | ccuccaagcagagaggaaaugaccaagaaccaggugucccugaccugccuggugaagggcuucuaccccagcgacaucgccg |
| | | | | ucgaaugggagucuaauggccagccagaaaacaacuacaaaaccaccccuccuguuuuggacagcgauggaucuuuuuuc |
| | | | | cuguacagcaagcugaccguggauaagagcagauggcagcagggcaacguguuuagcugcagcgugaugcacgaggcccug |
| | | | | cacaaccacuacacccagaagagccugagccugagccccggcggcggcggcggaucuggcggaggagguagugaggaagaac |
| | | | | uccaggugauccaaccugacaagagcgugagcguggccgccggcgagagcgccauccugcacuguacagugaccagccugau |
| | | | | cccugugggccccauccagugguuucggggcgcgggcccugcuagagagcugaucuauaaucaaaaagaaggccacuuccc |
| | | | | uagagugacuaccguguccgagagcacaaagagagagaacauggacuucagcaucagcaucagcaauaucacacccgccga |
| | | | | cgccggcaccuacuacugugugaaguucagaaagggcagcccggacacagaguucaaauccggcgcuggaaccgagcugagc |
| | | | | gucagagccaagugauaa |
| 45 | 1838 | | Exemplary RNA | auggauaugagaguacccgcccaacuguugggucuucuucuacuguggcuuagaggcgcaaggugcgaagaggaacuuca |
| | | | sequence | aguaauccagccugauaagucugugucaguugcggccggcgaguccgcaauccuacauuguacgguuacuagccuuaucc |
| | | | (minus UTRs) | ccguagguccuauucagugguucaggggugcgggaccugccagagagcugauauauaaccaaaaagaaggacauuuuccg |
| | | | encoding an | agagucacgacugugagugagagcaccaagcgugagaacauggauuucucgaucuccauuuccaauaucaccccagccgac |
| | | | amino acid | gccggcacauauuauuguguuaaguucagaaaggguucucccgacacagaguuuaagucgggcgcugguacagagcuaag |
| | | | sequence | cguccgggcuaaagguggaggcgguuccggcggaggcucugaggaagagcugcaaguaauccaaccggacaaaucgguauc |
| | | | containing the | uguugccgccggggagucagccauucugcauuguaccgugacgucguuaauaccaguuggucccauucaaugguuucgcg |
| | | | amino acid | gggccgguccugccagagaacugaucuacaaucagaaggaagggcauuuuccacgugugaccacuguaucugaaucaacca |
| | | | sequence | agagggagaauauggauuuuucaaucucgaucagcaauauuacucccgccgacgcagggaccuacuauugcguaaaguuu |
| | | | represented by | cggaaagguucuccugauaccgaguuuaagucaggagccgggaccgaguugucuguacgugcuaagggaggugauaagac |
| | | | SEQ ID NO: 60 | ccacacguguccaccaugucccgccccugaguuguuagguggucccagcguguuucucuuuccgcccaagcccaaggauac |
| | | | (1468; | ccuuaugaucucuaggacgcccgaaguaaccugcgucgucguggacgucucgcaugaggaccccgaggucaaguucaauug |
| | | | Octavalent) | guaugucgacggaguugaggugcauaacgcaaaaacgaaaccgcgagaagagcaguacaacagcacuuaucgcgugguuu |
| | | | | cuguguuaaccgucuugcaucaggauuggcugaacgggaaagaauacaaguguaaaguaucgaauaaagcucucccagcu |
| | | | | ccaaucgagaaaacgauaucgaaggcuaaggggcagcccagggaaccgcagguguacacacucccgccgucucgggaggaga |
| | | | | ugacuaaaaaccaaguaucucuaacaugcuuggugaaagguuucuauccuucagauaucgccguagaaugggagucaaa |
| | | | | cggccagccagaaaacaacuauaaaacuacccccccgguuuuggacagcgacgggucguuuuuccuuuauuccaaacugac |
| | | | | cguagacaagucccgauggcagcaaggaaacguguucagcuguagugugaugcaugaggccuuacauaaucacuacaccca |
| | | | | gaagagucuuucccugagcccaggaggagggggagguucuggagguggcgggagcgaggaagaacugcaagugauacagcc |
| | | | | ugacaagaguguuaguguugccgcuggagaauccgcgauucuacauugcacggucaccagccucauccccguugguccga |
| | | | | uccaaugguuuagaggcgccggcccggcaagggagcuuauuuauaaccaaaaggagggccacuuuccccgaguuacuaccg |
| | | | | uguccgaauccacaaaacgcgagaauauggacuucagcauaucuauuagcaacaucacgccggcagaugccgggacauauu |
| | | | | auugcgucaaguuucguaaggggagcccagauacugaguuuaagucuggugcugguaccgagcuguccgugcgcgcaaaa |
| | | | | gguggagggggcucuggggguggcucugaagaagagcugcagguuauucaacccgacaagucuguuucggucgccgcggg |
| | | | | cgaauccgccauacugcacuguacuguuaccucacugauccccgugggaccaauacagugguuuaggggugcaggccccgc |
| | | | | gagggaacuuaucuauaaucaaaaggagggucauuucccccgugucacaaccgucagcgaaaguaccaaaagggagaaca |
| | | | | uggacuuuaguaucaguaucaguaauauuaccccagcagaugcggguacguacuacugcgugaaauuccgaaaagguagc |
| | | | | ccggacacggaauuuaaaaguggugcggguacggaguugagugugcgcgccaagugauga |
| 46 | 1467 | | Exemplary RNA | auggacaugcggguccccgcccagcugcugggccugcugcugcuguggcugagaggagcccggugcgaggaagagcugcag |
| | | | sequence | gugauucaaccugacaagagcgugucuguggcugcuggggagagcgcuauccuccauugcacagugacaucucugaucccc |
| | | | (minus UTRs) | gugggcccuauccagugguuuagaggcgccggcccugcuagggaacugauuuacaaccagaaagagggccacuuccccaga |
| | | | encoding an | gugacaaccguguccgagagcaccaagagagaaaacauggacuucuccaucagcaucucuaauaucaccccugcugaugcc |
| | | | amino acid | ggaaccuacuacugcgugaaguucagaaagggcagccccgacaccgaguuuaagagcggcgccggaaccgagcugagcguu |
| | | | sequence | agagccaaaggcggcggcgguagcggcggcggcggcagcgaggaagagcugcaggugauccagccagacaagagcguguccg |
| | | | containing the | uggccgccggagagagcgccauccugcacugcaccgugacaagccugaucccuguuggccccauccagugguucaggggcgc |
| | | | amino acid | aggcccugccagagaacugaucuacaaccagaaggaaggccauuucccacgggugacaacagucagcgagucuaccaagcgg |
| | | | sequence | gaaaauauggacuucagcaucagcaucagcaacaucaccccugccgacgccggcaccuacuauugugugaaauuccggaaa |
| | | | represented by | ggaagcccugauaccgaauucaaguccggagccggcaccgagcugucugugagagccaagggcggagauaagacccacacu |
| | | | SEQ ID NO: 12 | uguccuccgugcccugccccugagcugcucggcggaccuagcgucuuucuguucccuccaaagcccaaggacacccugauga |
| | | | | ucucuagaaccccugaggugaccugcguggucgucgaugugagccacgaagauccugaagugaaguucaacugguacgug |
| | | | | gacgguguugaggugcacaacgccaagaccaagccucgcgaggaacaguacaacagcacauauagaguggugagcgugcug |
| | | | | accgugcugcaccaggacuggcugaacggcaaggaauacaagugcaaggugucuaacaaggcccugcccgcccccaucgaga |
| | | | | agaccaucuccaaggccaagggccagccucgggagccccagguuuacacccugccuccuucuagagaagagaugaccaagaa |
| | | | | ccaggugagccugacaugucuggugaagggcuucuacccuucugauauugccguggaaugggagagcaacggccagccuga |
| | | | | gaacaacuacaagacaaccccuccagugcuggacagcgacggcucauucuuccuguacagcaagcugaccguggacaaaagc |
| | | | | agauggcagcagggcaacguguucagcugcagcgugaugcacgaggcccuccacaaccacuacacccagaaaagccugagcc |
| | | | | ugucucccggcggcggcggaggcagcggcggaggcggaagcgaggaagagcugcaagugauccagccugauaagagcguuu |
| | | | | cugucgccgccggagaaagcgccauccugcacuguacagugaccagccugauccccgugggcccuauccaaugguuccgggg |
| | | | | cgcuggcccugcuagagaacugaucuauaaucagaaggaaggccacuuuccucgggugaccaccgugagcgaaagcaccaa |
| | | | | aagagagaauauggauuuuucuaucuccaucuccaacaucacaccagccgacgccggcacauacuacugcgugaaauucag |
| | | | | aaaaggcagccccgacacagaguucaagagcggcgcuggaacagagcugagcgugcgggccaaguga |
| 47 | 1837 | | Exemplary RNA | auggauaugagaguacccgcccaacuguugggucugcugcuguuguggcugcggggugcuagaugugaggaggagcugca |
| | | | sequence | ggugauucagccugauaaaucugugagcguggcugcuggggagagcgccaucuugcacugcaccgugacuucacugauccc |
| | | | (minus UTRs) | uguggggccuauccaaugguuccggggcgccgguccugccagagagcucaucuacaaccagaaggagggucauuuccccag |
| | | | encoding an | aguaaccaccguaagcgaguccaccaagcgggagaacauggacuuuagcaucagcaucuccaauauuacuccugccgacgc |
| | | | amino acid | cgggacuuacuacugugugaaguucaggaagggcuccccugacaccgaguuuaagucuggugcuggcacugagcugucug |
| | | | sequence | ugagagccaaggggggugacaagacucacaccugcccuccuugccccgcuccagagcugcugggugggcccagcguguuucu |
| | | | containing the | guuuccuccuaagcccaaggauacucugaugaucagccggacuccugaggucaccugcgugguaguggacgucagccacga |
| | | | amino acid | ggacccugaggugaaguucaacugguauguagacgguguggagguacacaacgccaagaccaagcccagagaggagcagua |
| | | | sequence | caacaguaccuauaggguggugucggugcugaccgugcugcaccaggauuggcugaacggcaaggaguacaagugcaagg |
| | | | represented by | ugagcaauaaggcccugcccgcuccuaucgagaagaccaucucuaaggccaaggggcagccccgggaaccccagguauacac |
| | | | SEQ ID NO: 19 | ucugccccccagcagggaggagaugaccaagaaccaaguaucucuguggugccuggucaaggguuucuacccuuccgacau |
| | | | (1348) | cgcuguggagugggagucuaacggucagcccgagaauaauuauaagaccaccccuccuguccuggacagugaugggucuu |
| | | | | ucuuuuuguauagcaagcugacuguggacaagucuagguggcagcaggguaacguguucucuugcucugugaugcacga |
| | | | | ggcccugcacaaucacuauacucagaagagucugagucuguccccuggcgggggugggggaagcggcgguggggguuccga |
| | | | | ggaggaguugcaaguuauacagccugacaagagcgugagcguggccgccggugagucugccauccugcauugcaccgucac |
| | | | | cagcuugauuccugugggccccauccagugguucagaggggccggucccgcucgggagcugauuuacaaccagaaagaggg |
| | | | | ccacuucccuagggugacuaccgugucugagaguaccaagagagagaacauggauuucucuauuucuaucuccaauauca |
| | | | | cccccgcugacgccgguaccuacuauugcgucaaguuucgcaaggguagccccgauaccgaguuuaaaucuggugccggua |
| | | | | cggagcugucuguccgggcuaagugauga |
| 48 | 1839 | | Exemplary RNA | auggauaugagaguacccgcccaacuguugggucugcugcugcuguggcugagaggcgcucggugugaggaagaguugca |
| | | | sequence | agucauucaaccggacaagucuguuagcguugcugcaggugagagcgccauccuccacugcacugucacgucucucauucc |
| | | | (minus UTRs) | uguaggcccuauccagugguuccguggugcuggccccgccagggaacugauuuauaaucagaaagaaggucacuuccccag |
| | | | encoding the | aguuaccaccgucuccgaguccaccaagcgggagaauauggauuucagcaucuccaucucgaacauuacccccgcugaugc |
| | | | amino acid | cggcacguauuauugcguuaaguuuagaaagggcucuccagauaccgaauuuaagucuggcgccggcaccgagcucuccg |
| | | | sequence | uuagagccaaaggcggcggugguaguggugggggcuccgaggaggagcuccaggugauucaaccggauaagagcguguccg |
| | | | represented by | uggcugccggugagucugccauucugcauuguacagugaccagccugaucccuguggggcccauccagugguuucgggga |
| | | | SEQ ID NO: 60 | gccgguccggcucgggaacugaucuacaaucagaaggagggucacuuucccagagugacgaccgugucugagagcaccaag |
| | | | (1468; | agggagaacauggauuucucuaucagcauuagcaacaucacgcccgccgaugcugggaccuauuacugcgugaaguuuag |
| | | | Octavalent) | gaagggcaguccugacacugaguucaaguccggggcugguaccgagcugagcgucagggccaaaggcggugauaagaccca |
| | | | | caccugcccgccuugcccugccccugagcuguugggcgguccgucuguuuuccuguuuccgccaaagcccaaagacacccug |
| | | | | augaucucuagaaccccggaggucacuugcgucguggucgaugugucucacgaggaccccgagguaaaguuuaauuggua |
| | | | | cgucgauggcguggaggugcacaaugccaaaacuaagcccagggaggagcaauacaacuccacuuacaggguggugagcgu |
| | | | | ucugacugugcugcaucaggauugguugaaugguaaggaguacaaaugcaaggucagcaacaaagcucugccggcuccca |
| | | | | ucgagaaaaccauuagcaaggcuaagggucagccuagggaaccccaaguguacacccugccccccuccagagaggagaugac |
| | | | | gaagaaccaaguaucccugacgugccugguaaaggguuucuacccaucggacaucgcuguggagugggagagcaacggcca |
| | | | | gcccgagaacaacuacaagacgaccccuccggucuuggauuccgauggcucuuuuuucuuguacagcaagcugaccgugga |
| | | | | caaaucuagguggcaacaagggaauguguucuccugcuccgugaugcacgaggcucuacacaaccauuacacccagaaaag |
| | | | | ccugagucugagccccgguggggguggggguagcggcggugguggcagcgaggaggaguugcagguuauacagccugacaa |
| | | | | gagcgugucaguugccgccggcgaguccgcaauccuccauugcaccgugacaucucugauucccgugggucccauucagug |
| | | | | guucagaggugcagguccugcgagagagcugaucuacaaccagaaggaggggcauuuuccgaggguaacaacagucagcga |
| | | | | aucuaccaagcgggaaaauauggauuuuucuaucuccauuucuaauaucacgcccgcugaugcgggcacauauuacugcg |
| | | | | ucaaguuuaggaaagguucaccggauacagaguucaagagcggggccggcacugagcugucggugcgggccaagggcggug |
| | | | | gcggcucuggcggugggagugaggaggagcugcaagugauucagcccgacaagucuguaaguguagccgcuggcgagagcg |
| | | | | ccauucugcacugcaccgugaccagucugauccccguuggcccuauucaaugguuccggggugccggaccugccagggagu |
| | | | | ugaucuauaaucaaaaggagggccauuuuccgcgggucacuacaguuucugagucuaccaagagagaaaauauggacuu |
| | | | | uuccaucaguaucuccaacaucaccccugcugaugccggaacguacuacugcgugaaauucagaaaaggcagccccgacac |
| | | | | cgaauuuaaaucuggcgccgguaccgaacugagugugagagcuaagugauga |
| 49 | 1836 | | Exemplary RNA | gaggaggagcugcagguaauucagccagauaagagcgucagcguggcggcaggggagagcgcgaucuugcauuguacaguc |
| | | | sequence | acaucacugauuccugugggcccuauucagugguuucggggugcaggccccgccagagagcugaucuauaaccaaaaagag |
| | | | (minus UTRs) | gggcacuucccgcggguaacaaccguuucggagaguacaaagagagaaaacauggauuucagcauuucgaucucaaauau |
| | | | encoding the | uacccccgccgaugccggcaccuacuauugcgugaaguuccggaaagguagcccugacacugaguucaagaguggggcugg |
| | | | amino acid | uacagagcugucagucagagccaagggaggagauaaaacucauaccugcccuccuugccccgccccagaguugcugggggg |
| | | | sequence | ccccagcguauuucuguuuccgcccaaaccuaaagauacccugaugaucucgcggacgccagagguuaccugcgucgucgu |
| | | | represented by | ugauguaucccacgaggaucccgaagugaaauuuaauugguacguugacggugucgaagugcacaaugccaagacaaaac |
| | | | SEQ ID NO: 19 | cccgggaggagcaauauaauagcaccuacagagucgucuccguauugacuguacugcaccaagauuggcugaauggaaag |
| | | | (1348) | gaauacaaauguaagguauccaauaaggcuuuaccggcaccuauugagaagaccauauccaaagccaaaggucaacccagg |
| | | | | gaaccccaaguuuauacucugccuccaagcagggaggagaugaccaagaaucaggucagccuguggugucucgugaaaggc |
| | | | | uuuuaucccagcgauauugccguugagugggaaucgaaugggcagcccgaaaauaauuacaaaaccaccccucccguccug |
| | | | | gauagcgaugguagcuuuuuucuguacuccaaauugaccguggauaagucuagauggcagcagggcaauguguuuagcu |
| | | | | gcucuguuaugcaugaggcucugcacaaucacuauacgcagaaaagccugucccugaguccuggcggagggggcggauccg |
| | | | | gagguggcgguuccgaagaagaguugcagguaauccagccugauaaaagugugucggucgcggccggcgagagugcaauu |
| | | | | cugcacugcaccgugaccagucuuauaccgguaggucccauucagugguucagaggcgccggcccugccagggaauugauu |
| | | | | uacaaucagaaggagggacacuuucccagagugacgaccgucagcgaauccacuaaaagggagaacauggauuuuucuau |
| | | | | cuccauuagcaacaucaccccggccgacgcuggcacguauuauugugugaaauuucgaaagggcucccccgauacagaguu |
| | | | | uaaaagcggugcaggaaccgagcugaguguccgggcgaagugauga |
| 50 | 1349 | | Exemplary RNA | gagguccagcugguagagagcggaggcgaccuggugcagccuggcagaagccuuagacugagcugcgccgcaagcggcuuu |
| | | | sequence | aucuucucuaacuacggcaugagcugggucagacaggccccuggaaagggccuggaauggguggccaccaucagcucugcc |
| | | | (minus UTRs) | agcaccuacucauauuacccugacagcgugaagggcagauucaccaucagccgggacaacgccaaaaauagccuguaccug |
| | | | encoding the | caaaugaacagccuccgggucgaggacaccgcccuguacuacugcggccggcacucugauggaaauuucgccuucggcuac |
| | | | amino acid | uggggccagggaacacuggugacagugucgucagccagcaccaagggacccagcguguucccccuggccccuagcuccaagu |
| | | | sequence | ccaccagcggcggaacagccgcccugggcugucuggugaaagacuacuuccccgagcccgucaccguguccuggaacagcgg |
| | | | represented by | cgcccucaccagcggcgugcauaccuuucccgcuguucugcagagcagcggacuguauucucugagcagcguggugaccgu |
| | | | SEQ ID NO: 20 | gccuagcuccagccugggcacccaaacauacaucugcaacgugaaccauaagccuagcaacaccaagguggacaagaaggug |
| | | | | gaaccuaagucuugcgacaagacccacaccuguccuccgugccccgccccugagcuccugggaggcccuagcguguuucugu |
| | | | | ucccuccuaaaccuaaggacacccugaugaucagccgcaccccugaggugacaugcguggucguggacgugagucacgagg |
| | | | | auccugaagugaaguuuaacugguacguggacggaguggaagugcacaaugccaagacaaaacccagagaagagcaguac |
| | | | | aacagcacauacagaguggugagugugcugaccgugcugcaccaggacuggcugaacggcaaggaauacaagugcaaggug |
| | | | | aguaacaaggcccugccugcuccuauugagaagacgaucagcaaggccaagggccagccuagggagccucagguguacaccc |
| | | | | ugccuccauccagagaagagaugacaaagaaccaggugagccucagcugugccgugaagggcuucuaccccucugauaucg |
| | | | | ccguggaaugggagucuaauggacagcccgagaacaacuacaagacaaccccuccugugcuggacagcgacggcagcuucu |
| | | | | uccuggugucuaaacugacaguggacaaguccagauggcagcagggcaacguguucagcugcagcgugaugcacgaggccc |
| | | | | ugcacaaccacuacacccagaaaucucugagccugucuccaggaggcggcggcggcucugguggcggcggcucugaggaaga |
| | | | | acugcaagucauccagccugauaaaucggucagcgucgccgcuggcgaguccgcuauccugcacugcaccgugaccagccug |
| | | | | auccccgugggcccgauccagugguuccggggcgcaggacccgcuagagagcucaucuacaaccagaaggaaggccacuucc |
| | | | | caagagugacaacaguuagcgagagcaccaagagagaaaacauggacuucagcauuucuaucagcaauaucaccccugcug |
| | | | | augccgggacauacuauugugugaaguucagaaagggcagcccagauacagaguucaagagcggcgcuggcaccgagcugu |
| | | | | ccgugcgggccaaaugauaa |
| 51 | 1478 | | Exemplary RNA | gacgugcugaugacccagagcccucugucccugccugugacaccuggcgagccugcuagcaucagcugcagaagcagccgca |
| | | | sequence | acaucgugcauaucaacggcgacaccuaccuggaaugguaccugcagaagcccggccagucuccccagcugcucaucuaua |
| | | | (minus UTRs) | aggucuccaauagauucagcggcgugcccgauagauuuagcggcucuggcagcggcacagauuuuacccugaaaaucagca |
| | | | encoding the | gaguggaagccgaggacgugggaguguacuacugcuuccagggcagccugcugcccuggaccuucggccagggaacaaagg |
| | | | amino acid | uugagaucaagagaaccgucgccgccccuagcguguucaucuucccuccaucugaugagcaacugaaguccggaaccgcca |
| | | | sequence | gcguggugugccugcugaacaacuucuaccccagagaagcuaaagugcaguggaagguggacaacgcccugcagagcggaa |
| | | | represented by | auucucaggagagcgugaccgagcaggacagcaaggacaguaccuacagccugagcagcacccugacacugucuaaagccga |
| | | | SEQ ID NO: 18 | cuacgagaagcacaagguguacgccugcgaggugacccaccagggccuguccagcccugugaccaaaagcuucaaccggggc |
| | | | | gaaugcggcggaggcggcagcgguggcggcggcagugaagaggaacugcaagugauccaaccugauaagagcgucucugug |
| | | | | gccgcuggagaaagcgccauccugcacuguacagugacgagccuuauccccgugggccccauccagugguuccggggagccg |
| | | | | gaccugccagagagcugaucuacaaccagaaagagggccacuucccuagagugaccaccgugucugagucuacaaagcggg |
| | | | | aaaacauggacuuuagcauuucuaucagcaacauuaccccugcagaugccggcacauacuauuguguuaaguucaggaag |
| | | | | ggcuccccugacaccgaauucaagagcggcgccggcacagagcugagcgugcgggccaagugauga |
| 52 | 1343 | | Exemplary RNA | gaggugcagcugguugaaagcggcggcgaccugguucaacccggcagaagccugagacugucuugcgccgccagcggauuc |
| | | | sequence | aucuucagcaacuacggcaugagcugggugcggcaggccccuggcaagggccuggaaugggucgccacaaucagcagcgccu |
| | | | (minus UTRs) | cuaccuacagcuacuaccccgacagcgugaaaggcagauucaccaucagcagagauaacgccaagaacagccuguaccugca |
| | | | encoding the | gaugaacucucuccggguggaagauaccgcccuguauuacugcggcaggcauucugauggcaacuucgccuucggcuacug |
| | | | amino acid | gggccaaggcacccuggugaccgugucuucugccagcaccaagggcccuagcgucuuuccucuggccccuagcagcaagagc |
| | | | sequence | acaagcggaggcacagcugcucuggguugucuggucaaggacuacuuccccgagcccgugaccguguccuggaacagcggc |
| | | | represented by | gcucugacaagcggagugcacacuuuuccugcugugcugcaaagcagcggccuguacagccugucaucuguggucaccgug |
| | | | SEQ ID NO: 14 | ccuaguagcucucucggaacccagaccuacaucugcaacgugaaccacaagccuuccaauaccaagguggacaagaaggug |
| | | | | gagccuaaaagcugcgacaagacacacacauguccuccgugcccugcuccugagcugcugggggucccucuguguuccug |
| | | | | uucccaccaaagcccaaggauacccugaugaucagccggaccccugaggugacguguguggugguggacgugagccacgag |
| | | | | gaccccgaggugaaguucaacugguacguggacggcguggaagugcacaaugccaaaacaaagccaagagaagagcaguac |
| | | | | aacuccaccuauagaguggucuccgugcugaccgugcugcaccaggacuggcugaacggcaaggaauacaaaugcaaggug |
| | | | | uccaacaaggcccugcccgccccuaucgagaagacaauuucuaaagccaagggccagcccagagagccucagguguauaccc |
| | | | | ugccuccuagcagagaggaaaugaccaaaaaccaggugucccuuacaugccuggugaaaggcuucuacccuucugauaucg |
| | | | | ccguggaaugggagagcaauggccagccugagaacaacuacaagaccaccccuccagugcuggacagcgacggcagcuucuu |
| | | | | ccuguacucuaagcugaccguggauaagucccgguggcagcagggcaauguguuuuccuguagcgugaugcacgaggccc |
| | | | | ugcacaaccacuacacacagaagagccugagccugagcccuggcugauaa |
| 53 | 1345 | | Exemplary RNA | gaagugcagcugguggaaagcggcggcgaccugguccagccuggcagaagccugcggcugaguugugccgccagcggcuuc |
| | | | sequence | aucuucagcaacuacggcaugagcugggugcggcaggcccccggcaagggccuggaaugggucgccaccaucagcagcgccu |
| | | | (minus UTRs) | cuaccuauagcuacuauccugauagcguuaagggaagauucaccaucagccgcgacaacgccaagaacagccuguaccugc |
| | | | encoding the | agaugaacagccugagagucgaagauaccgcccuguacuacugcggccggcacucugauggaaauuucgccuucggcuacu |
| | | | amino acid | ggggccagggcacgcuggugacagucagcucugcuagcacaaaaggaccuucuguuuucccucuggccccaucuagcaaga |
| | | | sequence | gcacaucugguggcaccgccgcccugggcugccuggugaaggacuacuuccccgagcccgugacugugagcuggaacagcgg |
| | | | represented by | cgccuugacaagcggcgugcacaccuucccagcugugcugcaaagcagcggccuguacucucuguccucuguugugacagu |
| | | | SEQ ID NO: 16 | gccuucuuccucucugggcacacagaccuacaucugcaacgugaaccacaagcccucuaauaccaagguggacaaaaaggu |
| | | | | ggaaccuaagagcugcgacaagacacacacauguccuccaugccccgcaccugagcugcugggggccccagcguguuucug |
| | | | | uuucccccgaagccuaaggacacccugaugaucagccggaccccugaagugaccugcgugguaguggacgugucccacgag |
| | | | | gaccccgaggugaaauuuaacugguacguggauggcguggaggugcacaacgccaaaacaaaaccuagagaggaacaguac |
| | | | | aacagcaccuauagaguggucagcgugcugaccgugcugcaccaggacuggcugaacggcaaagaguacaagugcaaggug |
| | | | | agcaacaaggcccugccugcucccaucgagaagaccaucagcaaggccaagggacaaccuagagagccccagguuuacacac |
| | | | | ugccuccuaguagggaggaaaugaccaagaaccaggugucccugacaugccuggucaaaggcuuuuaccccuccgauaucg |
| | | | | ccguggaaugggaguccaacggccagccugaaaacaacuacaagaccaccccuccagugcuggacagcgacggcagcuucuu |
| | | | | ccuguacuccaagcugaccguggacaagagcagauggcagcagggcaauguguuuagcugcagcgugaugcacgaggcccu |
| | | | | gcauaaucacuacacccagaagucccugagccucucuccuggaggcggcggaggaucuggcggaggcggcagcgaggaagag |
| | | | | cugcaggugauccagccugacaaaagcgugucuguggccgcuggcgagagcgccauccugcacugcaccgugaccucccuga |
| | | | | uccccgugggcccuauucagugguuccggggcgccggaccugcuagagagcugaucuacaaccaaaaggaaggccacuuccc |
| | | | | uagagugaccaccguguccgagagcaccaaaagagagaacauggauuucagcaucagcaucagcaauaucaccccugcuga |
| | | | | ugccggcacauacuacugugugaaguucagaaagggcagcccugacaccgaguucaagucuggagccgguacagagcuguc |
| | | | | cgugcgggccaagugauaa |
| 54 | 1063 | | Exemplary RNA | gaggaggaacugcaggucauccagccugacaagucuguuucuguggccgcuggcgaaucugccauccuucacugcaccgug |
| | | | sequence | acaagccugaucccggugggaccuauucaaugguuccggggcgccggccccgccagagagcugaucuacaaccagaaagagg |
| | | | (minus UTRs) | gccacuuuccacgcgugacaaccgugagcgaguccaccaagcgggagaauauggacuucagcaucagcaucagcaacauua |
| | | | encoding the | caccugcugaugccggcacauacuacugcgugaaauucagaaagggcagcccugauaccgaguucaagagcggagccggca |
| | | | amino acid | ccgagcugucugugcgggccaagggggcgacaagacccacacaugcccccccugcccugcuccugaacugcuggggggcca |
| | | | sequence | ucuguguuccuguuccccccuaagccuaaagacacccugaugaucuccaggaccccugaggugaccugugugguggucgac |
| | | | represented by | gugagccacgaagauccugagguuaaguucaacugguacguggacggcguggaagugcacaacgccaagaccaaaccuaga |
| | | | SEQ ID NO: 2 | gaagagcaguacaacagcaccuauagagugguguccgugcugacagugcugcaucaggacuggcucaacggcaaggaauac |
| | | | | aagugcaaggugucuaacaaggcacugccugccccuaucgagaagaccaucagcaaggccaaaggccagccuagagaacccc |
| | | | | agguguacacacugccuccaagcagagaggaaaugaccaagaaccaggugucccugaccugccuggugaagggcuucuacc |
| | | | | ccagcgacaucgccgucgaaugggagucuaauggccagccagaaaacaacuacaaaaccaccccuccuguuuuggacagcga |
| | | | | uggaucuuuuuuccuguacagcaagcugaccguggauaagagcagauggcagcagggcaacguguuuagcugcagcguga |
| | | | | ugcacgaggcccugcacaaccacuacacccagaagagccugagccugagccccggcggcggcggcggaucuggcggaggagg |
| | | | | uagugaggaagaacuccaggugauccaaccugacaagagcgugagcguggccgccggcgagagcgccauccugcacuguac |
| | | | | agugaccagccugaucccugugggccccauccagugguuucggggcgcgggcccugcuagagagcugaucuauaaucaaaa |
| | | | | agaaggccacuucccuagagugacuaccguguccgagagcacaaagagagagaacauggacuucagcaucagcaucagcaa |
| | | | | uaucacacccgccgacgccggcaccuacuacugugugaaguucagaaagggcagcccggacacagaguucaaauccggcgcu |
| | | | | ggaaccgagcugagcgucagagccaagugauaa |
| 55 | 1838 | | Exemplary RNA | gaagaggaacuucaaguaauccagccugauaagucugugucaguugcggccggcgaguccgcaauccuacauuguacggu |
| | | | sequence | uacuagccuuauccccguagguccuauucagugguucaggggugcgggaccugccagagagcugauauauaaccaaaaag |
| | | | (minus UTRs) | aaggacauuuuccgagagucacgacugugagugagagcaccaagcgugagaacauggauuucucgaucuccauuuccaau |
| | | | encoding the | aucaccccagccgacgccggcacauauuauuguguuaaguucagaaaggguucucccgacacagaguuuaagucgggcgcu |
| | | | amino acid | gguacagagcuaagcguccgggcuaaagguggaggcgguuccggcggaggcucugaggaagagcugcaaguaauccaaccg |
| | | | sequence | gacaaaucgguaucuguugccgccggggagucagccauucugcauuguaccgugacgucguuaauaccaguuggucccau |
| | | | represented by | ucaaugguuucgcggggccgguccugccagagaacugaucuacaaucagaaggaagggcauuuuccacgugugaccacug |
| | | | [SEQ ID NO: 60 | uaucugaaucaaccaagagggagaauauggauuuuucaaucucgaucagcaauauuacucccgccgacgcagggaccuac |
| | | | (1468; | uauugcguaaaguuucggaaagguucuccugauaccgaguuuaagucaggagccgggaccgaguugucuguacgugcua |
| | | | Octavalent) | agggaggugauaagacccacacguguccaccaugucccgccccugaguuguuagguggucccagcguguuucucuuuccgc |
| | | | minus the signal | ccaagcccaaggauacccuuaugaucucuaggacgcccgaaguaaccugcgucgucguggacgucucgcaugaggaccccga |
| | | | sequence] | ggucaaguucaauugguaugucgacggaguugaggugcauaacgcaaaaacgaaaccgcgagaagagcaguacaacagca |
| | | | | cuuaucgcgugguuucuguguuaaccgucuugcaucaggauuggcugaacgggaaagaauacaaguguaaaguaucgaa |
| | | | | uaaagcucucccagcuccaaucgagaaaacgauaucgaaggcuaaggggcagcccagggaaccgcagguguacacacucccg |
| | | | | ccgucucgggaggagaugacuaaaaaccaaguaucucuaacaugcuuggugaaagguuucuauccuucagauaucgccgu |
| | | | | agaaugggagucaaacggccagccagaaaacaacuauaaaacuacccccccgguuuuggacagcgacgggucguuuuuccu |
| | | | | uuauuccaaacugaccguagacaagucccgauggcagcaaggaaacguguucagcuguagugugaugcaugaggccuuac |
| | | | | auaaucacuacacccagaagagucuuucccugagcccaggaggagggggagguucuggagguggcgggagcgaggaagaac |
| | | | | ugcaagugauacagccugacaagaguguuaguguugccgcuggagaauccgcgauucuacauugcacggucaccagccuc |
| | | | | auccccguugguccgauccaaugguuuagaggcgccggcccggcaagggagcuuauuuauaaccaaaaggagggccacuuu |
| | | | | ccccgaguuacuaccguguccgaauccacaaaacgcgagaauauggacuucagcauaucuauuagcaacaucacgccggca |
| | | | | gaugccgggacauauuauugcgucaaguuucguaaggggagcccagauacugaguuuaagucuggugcugguaccgagcu |
| | | | | guccgugcgcgcaaaagguggagggggcucuggggguggcucugaagaagagcugcagguuauucaacccgacaagucug |
| | | | | uuucggucgccgcgggcgaauccgccauacugcacuguacuguuaccucacugauccccgugggaccaauacagugguuua |
| | | | | ggggugcaggccccgcgagggaacuuaucuauaaucaaaaggagggucauuucccccgugucacaaccgucagcgaaagua |
| | | | | ccaaaagggagaacauggacuuuaguaucaguaucaguaauauuaccccagcagaugcggguacguacuacugcgugaaa |
| | | | | uuccgaaaagguagcccggacacggaauuuaaaaguggugcggguacggaguugagugugcgcgccaagugauga |
| 56 | 1467 | | Exemplary RNA | gaggaagagcugcaggugauucaaccugacaagagcgugucuguggcugcuggggagagcgcuauccuccauugcacagu |
| | | | sequence | gacaucucugauccccgugggcccuauccagugguuuagaggcgccggcccugcuagggaacugauuuacaaccagaaaga |
| | | | (minus UTRs) | gggccacuuccccagagugacaaccguguccgagagcaccaagagagaaaacauggacuucuccaucagcaucucuaauau |
| | | | encoding the | caccccugcugaugccggaaccuacuacugcgugaaguucagaaagggcagccccgacaccgaguuuaagagcggcgccgga |
| | | | amino acid | accgagcugagcguuagagccaaaggcggcggcgguagcggcggcggcagcgaggaagagcugcaggugauccagccagaca |
| | | | sequence | agagcguguccguggccgccggagagagcgccauccugcacugcaccgugacaagccugaucccuguuggccccauccagug |
| | | | represented by | guucaggggcgcaggcccugccagagaacugaucuacaaccagaaggaaggccauuucccacgggugacaacagucagcgag |
| | | | SEQ ID NO: 12 | ucuaccaagcgggaaaauauggacuucagcaucagcaucagcaacaucaccccugccgacgccggcaccuacuauugugug |
| | | | | aaauuccggaaaggaagcccugauaccgaauucaaguccggagccggcaccgagcugucugugagagccaagggcggagau |
| | | | | aagacccacacuuguccuccgugcccugccccugagcugcucggcggaccuagcgucuuucuguucccuccaaagcccaagg |
| | | | | acacccugaugaucucuagaaccccugaggugaccugcguggucgucgaugugagccacgaagauccugaagugaaguuca |
| | | | | acugguacguggacgguguugaggugcacaacgccaagaccaagccucgcgaggaacaguacaacagcacauauagagugg |
| | | | | ugagcgugcugaccgugcugcaccaggacuggcugaacggcaaggaauacaagugcaaggugucuaacaaggcccugcccg |
| | | | | cccccaucgagaagaccaucuccaaggccaagggccagccucgggagccccagguuuacacccugccuccuucuagagaaga |
| | | | | gaugaccaagaaccaggugagccugacaugucuggugaagggcuucuacccuucugauauugccguggaaugggagagca |
| | | | | acggccagccugagaacaacuacaagacaaccccuccagugcuggacagcgacggcucauucuuccuguacagcaagcugac |
| | | | | cguggacaaaagcagauggcagcagggcaacguguucagcugcagcgugaugcacgaggcccuccacaaccacuacacccag |
| | | | | aaaagccugagccugucucccggcggcggcggaggcagcggcggaggcggaagcgaggaagagcugcaagugauccagccug |
| | | | | auaagagcguuucugucgccgccggagaaagcgccauccugcacuguacagugaccagccugauccccgugggcccuaucca |
| | | | | augguuccggggcgcuggcccugcuagagaacugaucuauaaucagaaggaaggccacuuuccucgggugaccaccgugag |
| | | | | cgaaagcaccaaaagagagaauauggauuuuucuaucuccaucuccaacaucacaccagccgacgccggcacauacuacug |
| | | | | cgugaaauucagaaaaggcagccccgacacagaguucaagagcggcgcuggaacagagcugagcgugcgggccaagugaua |
| | | | | a |
| 57 | 1837 | | Exemplary RNA | gaggaggagcugcaggugauucagccugauaaaucugugagcguggcugcuggggagagcgccaucuugcacugcaccgu |
| | | | sequence | gacuucacugaucccuguggggccuauccaaugguuccggggcgccgguccugccagagagcucaucuacaaccagaagga |
| | | | (minus UTRs) | gggucauuuccccagaguaaccaccguaagcgaguccaccaagcgggagaacauggacuuuagcaucagcaucuccaauau |
| | | | encoding the | uacuccugccgacgccgggacuuacuacugugugaaguucaggaagggcuccccugacaccgaguuuaagucuggugcugg |
| | | | amino acid | cacugagcugucugugagagccaaggggggugacaagacucacaccugcccuccuugccccgcuccagagcugcuggguggg |
| | | | sequence | cccagcguguuucuguuuccuccuaagcccaaggauacucugaugaucagccggacuccugaggucaccugcgugguagug |
| | | | represented by | gacgucagccacgaggacccugaggugaaguucaacugguauguagacgguguggagguacacaacgccaagaccaagccc |
| | | | SEQ ID NO: 19 | agagaggagcaguacaacaguaccuauaggguggugucggugcugaccgugcugcaccaggauuggcugaacggcaagga |
| | | | (1348) | guacaagugcaaggugagcaauaaggcccugcccgcuccuaucgagaagaccaucucuaaggccaaggggcagccccgggaa |
| | | | | ccccagguauacacucugccccccagcagggaggagaugaccaagaaccaaguaucucuguggugccuggucaaggguuuc |
| | | | | uacccuuccgacaucgcuguggagugggagucuaacggucagcccgagaauaauuauaagaccaccccuccuguccuggac |
| | | | | agugaugggucuuucuuuuuguauagcaagcugacuguggacaagucuagguggcagcaggguaacguguucucuugcu |
| | | | | cugugaugcacgaggcccugcacaaucacuauacucagaagagucugagucuguccccuggcgggggugggggaagcggcg |
| | | | | guggggguuccgaggaggaguugcaaguuauacagccugacaagagcgugagcguggccgccggugagucugccauccugc |
| | | | | auugcaccgucaccagcuugauuccugugggccccauccagugguucagaggggccggucccgcucgggagcugauuuaca |
| | | | | accagaaagagggccacuucccuagggugacuaccgugucugagaguaccaagagagagaacauggauuucucuauuucu |
| | | | | aucuccaauaucacccccgcugacgccgguaccuacuauugcgucaaguuucgcaaggguagccccgauaccgaguuuaaa |
| | | | | ucuggugccgguacggagcugucuguccgggcuaagugauga |
| 58 | 1839 | | Exemplary RNA | gaggaagaguugcaagucauucaaccggacaagucuguuagcguugcugcaggugagagcgccauccuccacugcacuguc |
| | | | sequence | acgucucucauuccuguaggcccuauccagugguuccguggugcuggccccgccagggaacugauuuauaaucagaaaga |
| | | | (minus UTRs) | aggucacuuccccagaguuaccaccgucuccgaguccaccaagcgggagaauauggauuucagcaucuccaucucgaacau |
| | | | encoding the | uacccccgcugaugccggcacguauuauugcguuaaguuuagaaagggcucuccagauaccgaauuuaagucuggcgccg |
| | | | amino acid | gcaccgagcucuccguuagagccaaaggcggcggugguaguggugggggcuccgaggaggagcuccaggugauucaaccgg |
| | | | sequence | auaagagcguguccguggcugccggugagucugccauucugcauuguacagugaccagccugaucccuguggggcccaucc |
| | | | represented by | agugguuucggggagccgguccggcucgggaacugaucuacaaucagaaggagggucacuuucccagagugacgaccgugu |
| | | | [SEQ ID NO: 60 | cugagagcaccaagagggagaacauggauuucucuaucagcauuagcaacaucacgcccgccgaugcugggaccuauuacu |
| | | | (1468; | gcgugaaguuuaggaagggcaguccugacacugaguucaaguccggggcugguaccgagcugagcgucagggccaaaggcg |
| | | | Octavalent) | gugauaagacccacaccugcccgccuugcccugccccugagcuguuggggguccgucuguuuuccuguuuccgccaaagc |
| | | | minus the signal | ccaaagacacccugaugaucucuagaaccccggaggucacuugcgucguggucgaugugucucacgaggaccccgagguaa |
| | | | sequence] | aguuuaauugguacgucgauggcguggaggugcacaaugccaaaacuaagcccagggaggagcaauacaacuccacuuac |
| | | | | aggguggugagcguucugacugugcugcaucaggauugguugaaugguaaggaguacaaaugcaaggucagcaacaaagc |
| | | | | ucugccggcucccaucgagaaaaccauuagcaaggcuaagggucagccuagggaaccccaaguguacacccugccccccucc |
| | | | | agagaggagaugacgaagaaccaaguaucccugacgugccugguaaaggguuucuacccaucggacaucgcuguggagug |
| | | | | ggagagcaacggccagcccgagaacaacuacaagacgaccccuccggucuuggauuccgauggcucuuuuuucuuguacag |
| | | | | caagcugaccguggacaaaucuagguggcaacaagggaauguguucuccugcuccgugaugcacgaggcucuacacaacca |
| | | | | uuacacccagaaaagccugagucugagccccgguggggguggggguagcggcggugguggcagcgaggaggaguugcaggu |
| | | | | uauacagccugacaagagcgugucaguugccgccggcgaguccgcaauccuccauugcaccgugacaucucugauucccgu |
| | | | | gggucccauucagugguucagaggugcagguccugcgagagagcugaucuacaaccagaaggaggggcauuuuccgaggg |
| | | | | uaacaacagucagcgaaucuaccaagcgggaaaauauggauuuuucuaucuccauuucuaauaucacgcccgcugaugcg |
| | | | | ggcacauauuacugcgucaaguuuaggaaagguucaccggauacagaguucaagagcggggccggcacugagcugucggu |
| | | | | gcgggccaagggcgguggcggcucuggcggugggagugaggaggagcugcaagugauucagcccgacaagucuguaagugu |
| | | | | agccgcuggcgagagcgccauucugcacugcaccgugaccagucugauccccguuggcccuauucaaugguuccggggugc |
| | | | | cggaccugccagggaguugaucuauaaucaaaaggagggccauuuuccgcgggucacuacaguuucugagucuaccaaga |
| | | | | gagaaaauauggacuuuuccaucaguaucuccaacaucaccccugcugaugccggaacguacuacugcgugaaauucaga |
| | | | | aaaggcagccccgacaccgaauuuaaaucuggcgccgguaccgaacugagugugagagcuaagugauga |
| 59 | 1467 | | SIRPα | MDMRVPAQLLGLLLLWLRGARCEEELQVIQPDKSVSVAAGESAILHCTVTSLIPVGPIQWFRGAGPARELIY |
| | | | | NQKEGHFPRVTTVSESTKRENMDFSISISNITPADAGTYYCVKFRKGSPDTEFKSGAGTELSVRAKGGGGSG |
| | | | | GGSEEELQVIQPDKSVSVAAGESAILHCTVTSLIPVGPIQWFRGAGPARELIYNQKEGHFPRVTTVSESTKRE |
| | | | | NMDFSISISNITPADAGTYYCVKFRKGSPDTEFKSGAGTELSVRAKGGDKTHTCPPCPAPELLGGPSVFLFPP |
| | | | | KPKDTLMISRTPEVTCVVVDVSHEDPEVKFNWYVDGVEVHNAKTKPREEQYNSTYRVVSVLTVLHQDWL |
| | | | | NGKEYKCKVSNKALPAPIEKTISKAKGQPREPQVYTLPPSREEMTKNQVSLTCLVKGFYPSDIAVEWESNGQ |
| | | | | PENNYKTTPPVLDSDGSFFLYSKLTVDKSRWQQGNVFSCSVMHEALHNHYTQKSLSLSPGGGGGSGGGG |
| | | | | SEEELQVIQPDKSVSVAAGESAILHCTVTSLIPVGPIQWFRGAGPARELIYNQKEGHFPRVTTVSESTKREN |
| | | | | MDFSISISNITPADAGTYYCVKFRKGSPDTEFKSGAGTELSVRAK |
| 60 | 1468 | | Hexavalent | MDMRVPAQLLGLLLLWLRGARCEEELQVIQPDKSVSVAAGESAILHCTVTSLIPVGPIQWFRGAGPARELIY |
| | | | | NQKEGHFPRVTTVSESTKRENMDFSISISNITPADAGTYYCVKFRKGSPDTEFKSGAGTELSVRAKGGGGSG |
| | | | | GGSEEELQVIQPDKSVSVAAGESAILHCTVTSLIPVGPIQWFRGAGPARELIYNQKEGHFPRVTTVSESTKRE |
| | | | | NMDFSISISNITPADAGTYYCVKFRKGSPDTEFKSGAGTELSVRAKGGDKTHTCPPCPAPELLGGPSVFLFPP |
| | | | | KPKDTLMISRTPEVTCVVVDVSHEDPEVKFNWYVDGVEVHNAKTKPREEQYNSTYRVVSVLTVLHQDWL |
| | | | | NGKEYKCKVSNKALPAPIEKTISKAKGQPREPQVYTLPPSREEMTKNQVSLTCLVKGFYPSDIAVEWESNGQ |
| | | | | PENNYKTTPPVLDSDGSFFLYSKLTVDKSRWQQGNVFSCSVMHEALHNHYTQKSLSLSPGGGGGSGGGG |
| | | | | SEEELQVIQPDKSVSVAAGESAILHCTVTSLIPVGPIQWFRGAGPARELIYNQKEGHFPRVTTVSESTKREN |
| | | | | MDFSISISNITPADAGTYYCVKFRKGSPDTEFKSGAGTELSVRAKGGGGSGGGSEEELQVIQPDKSVSVAAG |
| | | | | ESAILHCTVTSLIPVGPIQWFRGAGPARELIYNQKEGHFPRVTTVSESTKRENMDFSISISNITPADAGTYYCV |
| | | | | KFRKGSPDTEFKSGAGTELSVRAK |
| 61 | | | SIRPα | auggauaugagaguacccgcccaacuguugggucugcugcugcuguggcugagaggcgcucggugugaggaagaguugca |
| | | | Octavalent | agucauucaaccggacaagucuguuagcguugcugcaggugagagcgccauccuccacugcacugucacgucucucauucc |
| | | | Exemplary RNA | uguaggcccuauccagugguuccguggugcuggccccgccagggaacugauuuauaaucagaaagaaggucacuuccccag |
| | | | sequence | aguuaccaccgucuccgaguccaccaagcgggagaauauggauuucagcaucuccaucucgaacauuacccccgcugaugc |
| | | | (minus UTRs) | cggcacguauuauugcguuaaguuuagaaagggcucuccagauaccgaauuuaagucuggcgccggcaccgagcucuccg |
| | | | encoding an | uuagagccaaaggcggcggugguaguggugggggcggcuccgaggaggagcuccaggugauucaaccggauaagagcgug |
| | | | amino acid | uccguggcugccggugagucugccauucugcauuguacagugaccagccugaucccuguggggcccauccagugguuucgg |
| | | | sequence | ggagccgguccggcucgggaacugaucuacaaucagaaggagggucacuuucccagagugacgaccgugucugagagcacc |
| | | | containing the | aagagggagaacauggauuucucuaucagcauuagcaacaucacgcccgccgaugcugggaccuauuacugcgugaaguu |
| | | | amino acid | uaggaagggcaguccugacacugaguucaaguccggggcugguaccgagcugagcgucagggccaaaggcggugauaagac |
| | | | sequence | ccacaccugcccgccuugcccugccccugagcuguuggggguccgucuguuuuccuguuuccgccaaagcccaaagacacc |
| | | | represented by | cugaugaucucuagaaccccggaggucacuugcgucguggucgaugugucucacgaggaccccgagguaaaguuuaauug |
| | | | SEQ ID NO: 13 | guacgucgauggcguggaggugcacaaugccaaaacuaagcccagggaggagcaauacaacuccacuuacaggguggugag |
| | | | (Octavalent) | cguucugacugugcugcaucaggauugguugaaugguaaggaguacaaaugcaaggucagcaacaaagcucugccggcuc |
| | | | | ccaucgagaaaaccauuagcaaggcuaagggucagccuagggaaccccaaguguacacccugccccccuccagagaggagau |
| | | | | gacgaagaaccaaguaucccugacgugccugguaaaggguuucuacccaucggacaucgcuguggagugggagagcaacgg |
| | | | | ccagcccgagaacaacuacaagacgaccccuccggucuuggauuccgauggcucuuuuuucuuguacagcaagcugaccgu |
| | | | | ggacaaaucuagguggcaacaagggaauguguucuccugcuccgugaugcacgaggcucuacacaaccauuacacccagaa |
| | | | | aagccugagucugagccccgguggggguggggguagcggcggugguggcagcgaggaggaguugcagguuauacagccuga |
| | | | | caagagcgugucaguugccgccggcgaguccgcaauccuccauugcaccgugacaucucugauucccgugggucccauuca |
| | | | | gugguucagaggugcagguccugcgagagagcugaucuacaaccagaaggaggggcauuuuccgaggguaacaacaguca |
| | | | | gcgaaucuaccaagcgggaaaauauggauuuuucuaucuccauuucuaauaucacgcccgcugaugcgggcacauauuac |
| | | | | ugcgucaaguuuaggaaagguucaccggauacagaguucaagagcggggccggcacugagcugucggugcgggccaagggc |
| | | | | gguggcggcucuggcggugggggcagugaggaggagcugcaagugauucagcccgacaagucuguaaguguagccgcuggc |
| | | | | gagagcgccauucugcacugcaccgugaccagucugauccccguuggcccuauucaaugguuccggggugccggaccugcc |
| | | | | agggaguugaucuauaaucaaaaggagggccauuuuccgcgggucacuacaguuucugagucuaccaagagagaaaauau |
| | | | | ggacuuuuccaucaguaucuccaacaucaccccugcugaugccggaacguacuacugcgugaaauucagaaaaggcagccc |
| | | | | cgacaccgaauuuaaaucuggcgccgguaccgaacugagugugagagcuaaguga |
| 62 | | | Exemplary RNA | gaggaagagcugcaggugauucaaccugacaagagcgugucuguggcugcuggggagagcgcuauccuccauugcacagu |
| | | | sequence | gacaucucugauccccgugggcccuauccagugguuuagaggcgccggcccugcuagggaacugauuuacaaccagaaaga |
| | | | (minus UTRs) | gggccacuuccccagagugacaaccguguccgagagcaccaagagagaaaacauggacuucuccaucagcaucucuaauau |
| | | | encoding the | caccccugcugaugccggaaccuacuacugcgugaaguucagaaagggcagccccgacaccgaguuuaagagcggcgccgga |
| | | | amino acid | accgagcugagcguuagagccaaaggcggcggcgguagcggcggcggcggcagcgaggaagagcugcaggugauccagccag |
| | | | sequence | acaagagcguguccguggccgccggagagagcgccauccugcacugcaccgugacaagccugaucccuguuggccccaucca |
| | | | represented by | gugguucaggggcgcaggcccugccagagaacugaucuacaaccagaaggaaggccauuucccacgggugacaacagucag |
| | | | SEQ ID NO: 12 | cgagucuaccaagcgggaaaauauggacuucagcaucagcaucagcaacaucaccccugccgacgccggcaccuacuauug |
| | | | (Hexavalent) | ugugaaauuccggaaaggaagcccugauaccgaauucaaguccggagccggcaccgagcugucugugagagccaagggcgg |
| | | | | agauaagacccacacuuguccuccgugcccugccccugagcugcucggggaccuagcgucuuucuguucccuccaaagccc |
| | | | | aaggacacccugaugaucucuagaaccccugaggugaccugcguggucgucgaugugagccacgaagauccugaagugaag |
| | | | | uucaacugguacguggacgguguugaggugcacaacgccaagaccaagccucgcgaggaacaguacaacagcacauauaga |
| | | | | guggugagcgugcugaccgugcugcaccaggacuggcugaacggcaaggaauacaagugcaaggugucuaacaaggcccug |
| | | | | cccgcccccaucgagaagaccaucuccaaggccaagggccagccucgggagccccagguuuacacccugccuccuucuagaga |
| | | | | agagaugaccaagaaccaggugagccugacaugucuggugaagggcuucuacccuucugauauugccguggaaugggaga |
| | | | | gcaacggccagccugagaacaacuacaagacaaccccuccagugcuggacagcgacggcucauucuuccuguacagcaagcu |
| | | | | gaccguggacaaaagcagauggcagcagggcaacguguucagcugcagcgugaugcacgaggcccuccacaaccacuacacc |
| | | | | cagaaaagccugagccugucucccggcggcggcggaggcagcggcggaggcggaagcgaggaagagcugcaagugauccagc |
| | | | | cugauaagagcguuucugucgccgccggagaaagcgccauccugcacuguacagugaccagccugauccccgugggcccua |
| | | | | uccaaugguuccggggcgcuggcccugcuagagaacugaucuauaaucagaaggaaggccacuuuccucgggugaccaccg |
| | | | | ugagcgaaagcaccaaaagagagaauauggauuuuucuaucuccaucuccaacaucacaccagccgacgccggcacauacu |
| | | | | acugcgugaaauucagaaaaggcagccccgacacagaguucaagagcggcgcuggaacagagcugagcgugcgggccaagu |
| | | | | ga |
| 63 | | | Exemplary RNA | gaggaagaguugcaagucauucaaccggacaagucuguuagcguugcugcaggugagagcgccauccuccacugcacuguc |
| | | | sequence | acgucucucauuccuguaggcccuauccagugguuccguggugcuggccccgccagggaacugauuuauaaucagaaaga |
| | | | (minus UTRs) | aggucacuuccccagaguuaccaccgucuccgaguccaccaagcgggagaauauggauuucagcaucuccaucucgaacau |
| | | | encoding the | uacccccgcugaugccggcacguauuauugcguuaaguuuagaaagggcucuccagauaccgaauuuaagucuggcgccg |
| | | | amino acid | gcaccgagcucuccguuagagccaaaggcggcggugguaguggugggggcggcuccgaggaggagcuccaggugauucaac |
| | | | sequence | cggauaagagcguguccguggcugccggugagucugccauucugcauuguacagugaccagccugaucccuguggggccca |
| | | | represented by | uccagugguuucggggagccgguccggcucgggaacugaucuacaaucagaaggagggucacuuucccagagugacgaccg |
| | | | SEQ ID NO: 13 | ugucugagagcaccaagagggagaacauggauuucucuaucagcauuagcaacaucacgcccgccgaugcugggaccuauu |
| | | | (Octavalent) | acugcgugaaguuuaggaagggcaguccugacacugaguucaaguccggggcugguaccgagcugagcgucagggccaaag |
| | | | | gcggugauaagacccacaccugcccgccuugcccugccccugagcuguuggggguccgucuguuuuccuguuuccgccaa |
| | | | | agcccaaagacacccugaugaucucuagaaccccggaggucacuugcgucguggucgaugugucucacgaggaccccgagg |
| | | | | uaaaguuuaauugguacgucgauggcguggaggugcacaaugccaaaacuaagcccagggaggagcaauacaacuccacu |
| | | | | uacaggguggugagcguucugacugugcugcaucaggauugguugaaugguaaggaguacaaaugcaaggucagcaaca |
| | | | | aagcucugccggcucccaucgagaaaaccauuagcaaggcuaagggucagccuagggaaccccaaguguacacccugccccc |
| | | | | cuccagagaggagaugacgaagaaccaaguaucccugacgugccugguaaaggguuucuacccaucggacaucgcugugga |
| | | | | gugggagagcaacggccagcccgagaacaacuacaagacgaccccuccggucuuggauuccgauggcucuuuuuucuugua |
| | | | | cagcaagcugaccguggacaaaucuagguggcaacaagggaauguguucuccugcuccgugaugcacgaggcucuacacaa |
| | | | | ccauuacacccagaaaagccugagucugagccccgguggggguggggguagcggcggugguggcagcgaggaggaguugca |
| | | | | gguuauacagccugacaagagcgugucaguugccgccggcgaguccgcaauccuccauugcaccgugacaucucugauucc |
| | | | | cgugggucccauucagugguucagaggugcagguccugcgagagagcugaucuacaaccagaaggaggggcauuuuccga |
| | | | | ggguaacaacagucagcgaaucuaccaagcgggaaaauauggauuuuucuaucuccauuucuaauaucacgcccgcugau |
| | | | | gcgggcacauauuacugcgucaaguuuaggaaagguucaccggauacagaguucaagagcggggccggcacugagcuguc |
| | | | | ggugcgggccaagggcgguggcggcucuggcggugggggcagugaggaggagcugcaagugauucagcccgacaagucugu |
| | | | | aaguguagccgcuggcgagagcgccauucugcacugcaccgugaccagucugauccccguuggcccuauucaaugguuccg |
| | | | | gggugccggaccugccagggaguugaucuauaaucaaaaggagggccauuuuccgcgggucacuacaguuucugagucua |
| | | | | ccaagagagaaaauauggacuuuuccaucaguaucuccaacaucaccccugcugaugccggaacguacuacugcgugaaa |
| | | | | uucagaaaaggcagccccgacaccgaauuuaaaucuggcgccgguaccgaacugagugugagagcuaaguga |
| 64 | | | Exemplary RNA | auggacaugcgggugccugcccagcugcugggccugcugcuccuguggcugagaggagccaggugugaagaggaacugcag |
| | | | sequence | gugauccaacccgacaagagcgugucuguugccgccggcgagucugcuauucugcacuguaccgugacaucucucaucccu |
| | | | (minus UTRs) | gugggaccgauccagugguuccggggcgccggcccugcuagagagcuuaucuacaaccagaaagaaggccacuucccucgg |
| | | | encoding the | gugaccacaguuagcgagucuaccaagagagaaaacauggauuuuucuaucucaaucuccaauaucaccccugcugacgc |
| | | | amino acid | cggaacauacuacugcgugaaguucagaaagggcagcccagauacagaguuuaagagcggagccggcacagagcugagcgu |
| | | | sequence | gcgggccaagggcggcggcgguagcagcagcgcaucuaccgacaagacacacacaggcggcggcagcgaggaggagcugcaa |
| | | | containing the | gugauccagccugacaaaagcguguccguggcugccggcgagagcgccauccugcacugcaccgugaccagccugauccccg |
| | | | amino acid | ugggcccuauccagugguuuagaggcgccggccccgccagagaacugaucuacaaccagaaagagggacacuuccccagggu |
| | | | sequence | gacaaccguguccgaguccaccaaaagagagaacauggauuucucuaucuccaucagcaacaucaccccagccgacgcugga |
| | | | represented by | accuacuacugugugaaguucagaaagggaagcccugauaccgaguucaagagcggcgccggcaccgagcugagcgucaga |
| | | | SEQ ID NO: 35 | gccaagggcggcgacaagacccacaccugccccccaugcccugccccugagcugcugggggaccuucuguguuucuguucc |
| | | | (1540) | cccccaagccuaaggacacccugaugaucagcagaacccccgaggugacuugcgugguggucgacgugagccacgaagaucc |
| | | | | ugaggugaaguucaacugguacguggacggcguggaagugcacaacgccaaaacaaaaccuagagaggaacaguacaacag |
| | | | | cacuuauagaguggugucuguccugaccgugcugcaccaggacuggcugaacggcaaggaguacaagugcaaggugagca |
| | | | | acaaggcccugcccgcuccuaucgagaagaccaucagcaaggccaagggccagccaagagagccucagguguacacccugcc |
| | | | | uccuagccgggaggagaugacaaagaaccaggucagccugacaugucuggugaagggcuucuaccccagcgacauugccgu |
| | | | | ggaaugggagagcaauggccagccugagaacaacuacaagaccaccccccccgugcuggacagcgauggcagcuucuuccug |
| | | | | uacucuaagcugaccguggacaaaagccguuggcagcagggcaacguguucagcugcagcgugaugcacgaggcccugcau |
| | | | | aaucacuacacccagaagucucugagccugucuccuggcggcggaggcgguagcggcggaggcggcuccgaagaagagcugc |
| | | | | aggugauccaaccugauaagagcgucagcguggccgccggagaaagcgccauccugcacugcaccgugaccagcuugauccc |
| | | | | ugugggcccaauccagugguuccggggcgccggcccugcccgggaacugaucuauaaccagaaggaaggccauuucccacgg |
| | | | | gugacaaccgucucugaaagcacaaagagagagaacauggacuucagcaucagcauuucuaauaucaccccugcugaugcc |
| | | | | ggcaccuacuacugcgugaaauuuagaaaaggaagcccugacaccgaguucaagagcggcgcuggcacggaacucuccguu |
| | | | | agagccaaguaguag |
| 65 | | | Exemplary RNA | gaagaggaacugcaggugauccaacccgacaagagcgugucuguugccgccggcgagucugcuauucugcacuguaccgug |
| | | | sequence | acaucucucaucccugugggaccgauccagugguuccggggcgccggcccugcuagagagcuuaucuacaaccagaaagaa |
| | | | (minus UTRs) | ggccacuucccucgggugaccacaguuagcgagucuaccaagagagaaaacauggauuuuucuaucucaaucuccaauau |
| | | | encoding the | caccccugcugacgccggaacauacuacugcgugaaguucagaaagggcagcccagauacagaguuuaagagcggagccgg |
| | | | amino acid | cacagagcugagcgugcgggccaagggcggcggcgguagcagcagcgcaucuaccgacaagacacacacaggcggcggcagcg |
| | | | sequence | aggaggagcugcaagugauccagccugacaaaagcguguccguggcugccggcgagagcgccauccugcacugcaccgugac |
| | | | represented by | cagccugauccccgugggcccuauccagugguuuagaggcgccggccccgccagagaacugaucuacaaccagaaagaggga |
| | | | SEQ ID NO: 35 | cacuuccccagggugacaaccguguccgaguccaccaaaagagagaacauggauuucucuaucuccaucagcaacaucacc |
| | | | (1540) | ccagccgacgcuggaaccuacuacugugugaaguucagaaagggaagcccugauaccgaguucaagagcggcgccggcaccg |
| | | | | agcugagcgucagagccaagggcggcgacaagacccacaccugccccccaugcccugccccugagcugcugggggaccuucu |
| | | | | guguuucuguucccccccaagccuaaggacacccugaugaucagcagaacccccgaggugacuugcgugguggucgacgug |
| | | | | agccacgaagauccugaggugaaguucaacugguacguggacggcguggaagugcacaacgccaaaacaaaaccuagagag |
| | | | | gaacaguacaacagcacuuauagaguggugucuguccugaccgugcugcaccaggacuggcugaacggcaaggaguacaag |
| | | | | ugcaaggugagcaacaaggcccugcccgcuccuaucgagaagaccaucagcaaggccaagggccagccaagagagccucagg |
| | | | | uguacacccugccuccuagccgggaggagaugacaaagaaccaggucagccugacaugucuggugaagggcuucuacccca |
| | | | | gcgacauugccguggaaugggagagcaauggccagccugagaacaacuacaagaccaccccccccgugcuggacagcgaugg |
| | | | | cagcuucuuccuguacucuaagcugaccguggacaaaagccguuggcagcagggcaacguguucagcugcagcgugaugca |
| | | | | cgaggcccugcauaaucacuacacccagaagucucugagccugucuccuggcggcggaggcgguagcggcggaggcggcucc |
| | | | | gaagaagagcugcaggugauccaaccugauaagagcgucagcguggccgccggagaaagcgccauccugcacugcaccguga |
| | | | | ccagcuugaucccugugggcccaauccagugguuccggggcgccggcccugcccgggaacugaucuauaaccagaaggaagg |
| | | | | ccauuucccacgggugacaaccgucucugaaagcacaaagagagagaacauggacuucagcaucagcauuucuaauaucac |
| | | | | cccugcugaugccggcaccuacuacugcgugaaauuuagaaaaggaagcccugacaccgaguucaagagcggcgcuggcac |
| | | | | ggaacucuccguuagagccaaguaguag |
| 66 | | | Signal sequence | MDMRVPAQLLGLLLLWLRGARC |

Underlined sequence-signal sequence.
Exemplary linkers in bold italics

1. An mRNA polynucleotide sequence encoding for a fusion protein, wherein the fusion protein comprises:
a) a first signal regulatory protein alpha (SIRPα) polypeptide that binds to a CD47 protein;
b) an IgG Fc domain, wherein the IqG Fc domain comprises at least one modification that facilitates heterodimerization of the fusion protein with another fusion protein that comprises an IqG Fc domain; and
c) a second SIRPα polypeptide that binds to the CD47 protein,
wherein the IqG Fc domain is linked to the first SIRPα polypeptide or the second SIRPα polypeptide via a linker, wherein the first SIRPα polypeptide and the second SIRPα polypeptide are not directly linked to each other.

2. The mRNA polynucleotide of claim 1, wherein the IgG Fc domain comprises a human IqG1 Fc domain, wherein the human IqG1 Fc domain comprises a CH2 domain and a CH3 domain.

3. The mRNA polynucleotide of claim 1, wherein the IgG Fc domain comprises a knob modification and/or a hole modification.

4. The mRNA polynucleotide of claim 1, wherein the fusion protein comprises an amino acid sequence selected from the group consisting of; SEQ ID NO: 4, SEQ ID NO: 5, and SEQ ID NO: 6.

5. The mRNA polynucleotide of claim 1, wherein the fusion protein comprises;
an amino acid sequence that is at least 95% homologous to the amino acid sequence of any one of SEQ ID NO: 2, SEQ ID NO: 12, SEQ ID NO: 13, SEQ ID NO: 16, SEQ ID NO: 18, SEQ ID NO: 19 or SEQ ID NO: 20.

6. The mRNA polynucleotide of claim 1, wherein the fusion protein comprises:
a sequence comprising amino acids 23 to 627 of SEQ ID NO: 35, a sequence comprising amino acids 23 to 617 of SEQ ID NO: 59, or a sequence comprising amino acids 23 to 742 of SEQ ID NO: 60.

7. The mRNA polynucleotide of claim 1, wherein the mRNA polynucleotide comprises: the nucleotide sequence of any one of SEQ ID NO: 54, SEQ ID NO: 62, SEQ ID NO: 63, SEQ ID NO: 56, SEQ ID NO: 55, SEQ ID NO: 58, SEQ ID NO: 53, SEQ ID NO: 51, SEQ ID NO: 29, SEQ ID NO: 49, SEQ ID NO: 57, or SEQ ID NO: 50.

8. The mRNA polynucleotide of claim 1, wherein the fusion protein is a bivalent fusion protein comprising a human IgG1 Fc domain, wherein the first SIRPα polypeptide is linked to the human IqG1 Fc domain via a first linker and the second SIRPα Polypeptide is linked to the same human IqG1 Fc domain via a second linker.

9. The mRNA polynucleotide of claim 1, wherein the fusion protein is a multivalent fusion protein comprising three, four, five, six, seven, or eight independent SIRPα polypeptides and a human IgG1 Fc domain.

10. The mRNA polynucleotide of claim 1, wherein the fusion protein is a tetravalent fusion protein comprising four independent SIRPα polypeptides and a human IgG1 Fc domain.

11. The mRNA polynucleotide of claim 1, wherein the fusion protein comprises;
a dimer of an amino acid sequence that is at least 95% homologous to the amino acid sequence of SEQ ID NO: 2, SEQ ID NO: 16, SEQ ID NO: 17, SEQ ID NO: 18, SEQ ID NO: 19, SEQ ID NO: 20, SEQ ID NO: 21, a sequence comprising amino acids 23 to 372 of SEQ ID NO: 29, a sequence comprising amino acids 23 to 601 of SEQ ID NO: 30, a sequence comprising amino acids 23 to 727 of SEQ ID NO: 31, a sequence comprising amino acids 23 to 497 of SEQ ID NO: 34, a sequence comprising amino acids 23 to 627 of SEQ ID NO: 35, a sequence comprising amino acids 23 to 492 of SEQ ID NO: 36, or a combination thereof.

12. The mRNA polynucleotide of claim 10, wherein the fusion protein comprises;
a dimer of an amino acid sequence of any one of SEQ ID NO: 2, SEQ ID NO: 16, SEQ ID NO: 17, SEQ ID NO: 18, SEQ ID NO: 19, SEQ ID NO: 20, SEQ ID NO: 21, a sequence comprising amino acids 23 to 372 of SEQ ID NO: 29, a sequence comprising amino acids 23 to 601 of SEQ ID NO: 30, a sequence comprising amino acids 23 to 727 of SEQ ID NO: 31, a sequence comprising amino acids 23 to 497 of SEQ ID NO: 34, a sequence comprising amino acids 23 to 627 of SEQ ID NO: 35, a sequence comprising amino acids 23 to 492 of SEQ ID NO: 36, or a combination thereof.

13. The mRNA polynucleotide of claim 10, wherein the mRNA polynucleotide comprises the nucleotide sequence of any one of SEQ ID NO: 53, SEQ ID NO: 54, SEQ ID NO: 51, SEQ ID NO: 49, SEQ ID NO: 57, SEQ ID NO: 50, or SEQ ID NO: 65.

14. The mRNA polynucleotide of claim 1, wherein the fusion protein is a hexavalent fusion protein comprising six independent SIRPα polypeptides and a human IgG1 Fc domain.

15. The mRNA polynucleotide of claim 14, wherein the fusion protein comprises;
a dimer of an amino acid sequence that is at least 95% homologous to the amino acid sequence of any one of SEQ ID NO: 12, a sequence comprising amino acids 23 to 627 of SEQ ID NO: 35, a sequence comprising amino acids 23 to 617 of SEQ ID NO: 59, or a combination thereof.

16. The mRNA polynucleotide of claim 14, wherein the fusion protein comprises:
a dimer of an amino acid sequence any one of SEQ ID NO: 12, a sequence comprising amino acids 23 to 627 of SEQ ID NO: 35, a sequence comprising amino acids 23 to 617 of SEQ ID NO: 59, or a combination thereof.

17. The mRNA polynucleotide of claim 14, wherein the mRNA polynucleotide comprises the nucleotide sequence of any one of SEQ ID NO: 56 or SEQ ID NO: 62.

18. The mRNA polynucleotide of claim 1, wherein the fusion protein is an octavalent fusion protein comprising eight independent SIRPα domains and a human IgG1 Fc domain.

19.-27. (canceled)

28. A fusion protein encoded by the mRNA polynucleotide of claim 1.

29. A therapeutic composition comprising the mRNA polynucleotide of claim 1; and a delivery vehicle molecule comprising an amino-lipidated peptoid.

30.-70. (canceled)
